SEQUENCE LISTING (428 sequences)

SEQ ID NO: 1 - DNA, 363 bp, Homo sapiens
ttagagacccaattgggacctaattgggacccaaatttctcaagtggagggagaacttttgacgatttccaccggtatctcctcgtgggtattcagggagctgcccagaaacctataaacttgtctaaggcgattgaagtcgtccaggggcatgatgagtcaccaggagtgtttttagagcacctccaggaggcttatcggatttacaccccttttgacctggcagcccccgaaaatagccatgctcttaatttggcatttgtggctcaggcagccccagatagtaaaaggaaactccaaaaactagagggattttgctggaatgaataccagtcagcttttagagatagcctaaaaggtttt

SEQ ID NO: 2 - protein, 121 aa, Homo sapiens [sequence omitted]

SEQ ID NO: 3 - DNA, 1080 bp, Homo sapiens
  misc_feature 681, 685, 706, 720, 741, 752, 758, 780, 789, 824, 840,859, 866, 884, 890, 905, 917, 926, 930, 951, 957, 959, 962, 974,980, 982, 988, 995, 996, 1007, 1010, 1025, 1040, 1051, 1052,1056, 1057, 1078: n = A,T,C or G
tcttagaatcttcataccccgaactcttgggaaaactttaatcagtcacctacagtctaccacccatttaggaggagcaaagctacctcagctcctccggagccgttttaagatcccccatcttcaaagcctaacagatcaagcagctctccggtgcacaacctgcgcccaggtaaatgccaaaaaaggtcctaaacccagcccaggccaccgtctccaagaaaactcaccaggagaaaagtgggaaattgactttacagaagtaaaaccacaccgggctgggtacaaataccttctagtactggtagacaccttctctggatggactgaagcatttgctaccaaaaacgaaactgtcaatatggtagttaagtttttactcaatgaaatcatccctcgacgtgggctgcctgttgccatagggtctgataatggaacggccttcgccttgtctatagtttaatcagtcagtaaggcgttaaacattcaatggaagctccattgtgcctatcgacccagagctctgggcaagtagaacgcatgaactgcaccctaaaaaaacactcttacaaaattaatcttaaaaaccggtgttaattgtgttagtctccttcccttagccctacttagagttaaggtgcaccccttactgggctgggttctttaccttttgaaatcatntttnggaaggggctgcctatctttncttaactaaaaaangcccatttggcaaaaatttcncaactaatttntacgtncctacgtctccccaacaggtanaaaaatctnctgcccttttcaaggaaccatcccatccattcctnaacaaaaggcctgccnttcttcccccagttaactnttttttnttaaaattcccaaaaaangaaccncctgctggaaaaacncccccctccaanccccggccnaagnggaaggttcccttgaatcccncccccncnaanggcccggaaccnttaaantngttccngggggtnnggcctaaaagnccnatttggtaaacctanaaattttttcttttntaaaaaccacnntttnntttttcttaaacaaaaccctntt

SEQ ID NO: 4 - DNA, 1087 bp, Homo sapiens
  misc_feature 559, 574, 576, 581, 582, 587, 589, 593, 594, 609, 627,640, 659, 668, 672, 677, 691, 713, 714, 732, 741, 812, 813, 823,825, 829, 838, 845, 849, 852, 855, 856, 859, 874, 876, 877,892, 902, 907, 916, 917, 938, 950, 951, 952, 953, 960: n = A,T,C or G
  misc_feature 965, 974, 976, 978, 982, 996, 1005, 1012, 1049, 1058,1073, 1074, 1082, 1084, 1086: n = A,T,C or G
tctagagctgcgcctggatcccgccacagtgaggagacctgaagaccagagaaaacacagcaagtaggccctttaaactactcacctgtgttgtcttctaatttattctgttttattttgtttccatcattttaaggggttaaaatcatcttgttcagacctcagcatataaaatgacccatctgtagacctcaggctccaaccataccccaagagttgtctggttttgtttaaattactgccaggtttcagctgcagatatccctggaaggaatattccagattccctgagtagtttccaggttaaaatcctataggcttcttctgttttgaggaagagttcctgtcagagaaaaacatgattttggatttttaactttaatgcttgtgaaacgctataaaaaaaattttctacccctagctttaaagtactgttagtgagaaattaaaattccttcaggaggattaaactgccatttcagttaccctaattccaaatgttttggtggttagaatcttctttaatgttcttgaagaagtgttttatattttcccatcnagataaattctctcncnccttnnttttntntctnnttttttaaaacggantcttgctccgttgtccangctgggaattttnttttggccaatctccgctnccttgcaanaatnctgcntcccaaaattaccncctttttcccacctccaccccnnggaattacctggaattanaggcccccnccccccccccggctaatttgtttttgtttttagtaaaaaacgggtttcctgttttagttaggatggcccanntctgaccccntnatcntccccctcngccctcnaatnttnggnntanggcttaccccccccngnngtttttcctccattnaaattttctntggantcttgaatnncgggttttcccttttaaaccnattttttttttnnnncccccanttttncctcccccntntntaangggggtttcccaanccgggtccncccccangtccccaatttttctccccccccctcttttttctttnccccaaaantcctatcttttcctnnaaatatcnantnt

SEQ ID NO: 5 - DNA, 1010 bp, Homo sapiens
  misc_feature 311, 315, 318, 339, 341, 347, 361, 379, 391, 415, 417,419, 424, 430, 433, 454, 463, 465, 467, 476, 497, 499, 550, 562,564, 587, 591, 595, 597, 598, 612, 625, 631, 640, 641, 645,648, 656, 661, 665, 666, 670, 674, 675, 681, 682, 683: n = A,T,C or G
  misc_feature 687, 688, 692, 710, 721, 778, 788, 811, 820, 830, 860,867, 868, 871, 872, 889, 892, 896, 897, 899, 904, 915, 936, 951,960, 970, 986, 990, 1000: n = A,T,C or G
tctagaccaagaaatgggaggattttagagtgactgatgatttctctatcatctgcagttagtaaacattctccacagtttatgcaaaaagtaacaaaaccactgcagatgacaaacactaggtaacacacatactatctcccaaatacctacccacaagctcaacaattttaaactgttaggatcactggctctaatcaccatgacatgaggtcaccaccaaaccatcaagcgctaaacagacagaatgtttccactcctgatccactgtgtgggaagaagcaccgaacttacccactggggggcctgcntcanaanaaaagcccatgcccccgggtntncctttnaaccggaacgaatnaacccaccatccccacanctcctctgttcntgggccctgcatcttgtggcctcntntnctttngggganacntggggaaggtaccccatttcnttgaccccncnanaaaaccccngtggccctttgccctgattcncntgggccttttctcttttcccttttgggttgtttaaattcccaatgtccccngaaccctctccntnctgcccaaaacctacctaaattnctcnctangnnttttcttggtgttncttttcaaaggtnaccttncctgttcanncccnacnaaaatttnttccntatnntggncccnnaaaaannnatcnncccnaattgcccgaattggttnggtttttcctnctgggggaaaccctttaaatttcccccttggccggccccccttttttcccccctttngaaggcaggnggttcttcccgaacttccaattncaacagccntgcccattgntgaaacccttttcctaaaattaaaaaatanccggttnnggnnggcctctttcccctccnggngggnngngaaantccttaccccnaaaaaggttgcttagcccccngtccccactcccccnggaaaaatnaaccttttcnaaaaaaggaatataantttnccactccttngttctcttcc

SEQ ID NO: 6 - DNA, 950 bp, Homo sapiens
  misc_feature 199, 200, 209, 223, 224, 236, 240, 241, 244, 248, 249,262, 263, 267, 268, 269, 270, 271, 272, 273, 280, 281, 283, 285,286, 287, 288, 289, 290, 291, 293, 295, 296, 300, 302, 303,309, 313, 314, 315, 316, 317, 318, 319, 320, 322, 323: n = A,T,C or G
  misc_feature 326, 327, 331, 332, 339, 342, 343, 344, 346, 349, 352,353, 355, 356, 359, 360, 362, 363, 364, 367, 369, 371, 375, 377,378, 379, 383, 385, 387, 389, 390, 392, 396, 397, 399, 400,401, 402, 405, 406, 408, 409, 410, 412, 413, 414, 415: n = A,T,C or G
  misc_feature 417, 419, 420, 423, 424, 428, 431, 433, 434, 435, 437,438, 439, 443, 447, 449, 450, 455, 456, 458, 459, 462, 465, 467,469, 472, 480, 481, 483, 484, 485, 486, 487, 488, 493, 494,495, 496, 497, 502, 505, 507, 508, 510, 512, 517, 518: n = A,T,C or G
  misc_feature 520, 521, 524, 526, 531, 536, 538, 539, 543, 544, 548,549, 550, 552, 553, 555, 556, 557, 561, 563, 566, 570, 571, 572,576, 577, 579, 580, 582, 583, 585, 588, 590, 591, 592, 594,597, 603, 606, 607, 614, 616, 618, 620, 621, 622, 623: n = A,T,C or G
  misc_feature 625, 628, 629, 630, 632, 634, 637, 638, 641, 645, 651,652, 653, 658, 659, 663, 664, 668, 672, 673, 674, 678, 685, 689,696, 700, 701, 702, 704, 705, 706, 708, 710, 711, 712, 713,715, 719, 722, 725, 727, 731, 734, 735, 737, 739, 742: n = A,T,C or G
  misc_feature 745, 748, 749, 751, 752, 754, 755, 757, 759, 762, 765,767, 769, 773, 774, 775, 778, 780, 783, 785, 787, 790, 793, 797,800, 803, 810, 812, 824, 828, 832, 836, 839, 843, 844, 846,848, 850, 852, 853, 855, 858, 859, 861, 864, 865, 866: n = A,T,C or G
  misc_feature 868, 869, 872, 875, 880, 886, 889, 890, 891, 892, 893,895, 896, 901, 902, 906, 908, 913, 914, 916, 918, 921, 924, 925,930, 932, 935, 940: n = A,T,C or G
tctagagctcgcggccgcgagctctaatacgactcactatagggcgtcgactcgatctcagctcactgcaatctctgcccccggggtcatgcgattctcctgcctcagccttccaagtagctgggattacaggcgtgcaacaccacacccggctaattttgtatttttaatagagatggggttttcccttgttggccannatggtctcnaacccctgacctcnngtgatccccccncccnnganctcnnactgctggggatnnccgnnnnnnncctcccnncncnnnnnnncncnntccntnntccttnctcnnnnnnnncnntcnntccnncttctcnccnnntnttntcnncnnccnncnnnccncntncccncnnnttcncntncnntntccnncnnnntcnncnnncnnnncntnnccnntacntcntnnncnnntccntctntnncctcnncnntcnctncncnttntctcctcnntnnnnnnctccnnnnntctcntcncnncntncctcnntnnccncnccccncctcncnncctnntttnnncnncnnntccntnccnttcnnntccnntnncnncntcncnnncnttnttcccnccnnttccttncncntnnnntntcnnncncntcnntcntttnctcctnnntcccnnctcnnttcncccnnntccnccccccncctntctctcncccnnntnnntntnnnncntccnctntcncnttcntcnntncnttnctntcnncnncnntncnctnccntntntctnnntcncntcncntntcnccntccnttnctntctcctntntccttcccctcncctnctcnttcnccncccnntntntntnncnccnntnctnnncnnccntcntttcntctctnctnnnnntnncctcnncccntnccctnntncnctnctnntaccntnctnctccntcttccttcc

SEQ ID NO: 7 - DNA, 1086 bp, Homo sapiens
  misc_feature 501, 691, 711, 735, 751, 780, 810, 819, 826, 832, 849,889, 890, 904, 913, 920, 926, 937, 940, 953, 957, 960, 985, 993,994, 1000, 1012, 1044, 1060, 1063, 1080, 1081: n = A,T,C or G
tctagagctcgcggccgcgagctcaattaaccctcactaaagggagtcgactcgatcagactgttactgtgtctatgtagaaagaagtagacataagagattccattttgttctgtactaagaaaaattcttctgccttgagatgctgttaatctgtaaccctagccccaaccctgtgctcacagagacatgtgctgtgttgactcaaggttcaatggatttagggctatgctttgttaaaaaagtgcttgaagataatatgcttgttaaaagtcatcaccattctctaatctcaagtacccagggacacaatacactgcggaaggccgcagggacctctgtctaggaaagccaggtattgtccaagatttctccccatgtgatagcctgagatatggcctcatgggaagggtaagacctgactgtcccccagcccgacatcccccagcccgacatcccccagcccgacacccgaaaagggtctgtgctgaggaagattantaaaagaggaaggctctttgcattgaagtaagaagaaggctctgtctcctgctcgtccctgggcaataaaatgtcttggtgttaaacccgaatgtatgttctacttactgagaataggagaaaacatccttagggctggaggtgagacaccctggcggcatactgctctttaatgcacgagatgtttgtntaattgccatccagggccancccctttccttaactttttatganacaaaaactttgttcncttttcctgcgaacctctccccctattancctattggcctgcccatcccctccccaaanggtgaaaanatgttcntaaatncgagggaatccaaaacnttttcccgttggtcccctttccaaccccgtccctgggccnntttcctccccaacntgtcccggntccttcnttcccncccccttcccnganaaaaaaccccgtntganggngccccctcaaattataacctttccnaaacaaannggttcnaaggtggtttgnttccggtgcggctggccttgaggtcccccctncaccccaatttggaanccngttttttttattgcccnntcccc

SEQ ID NO: 8 - DNA, 1177 bp, Homo sapiens
  misc_feature 1, 4, 20, 21, 31, 278, 314, 332, 359, 371, 373, 375,376, 524, 537, 556, 557, 579, 583, 590, 591, 598, 623, 625, 648,700, 703, 719, 738, 742, 746, 749, 751, 752, 800, 808, 820,821, 824, 835, 838, 845, 851, 856, 864, 865, 879, 888: n = A,T,C or G
  misc_feature 911, 920, 926, 935, 945, 950, 952, 956, 969, 972, 977,981, 992, 999, 1023, 1024, 1032, 1038, 1039, 1040, 1062, 1069,1075, 1084, 1089, 1104, 1119, 1123, 1131, 1143, 1146, 1152,1165, 1169, 1172, 1176: n = A,T,C or G
nccntttagatgttgacaanntaaacaagcngctcaggcagctgaaaaaagccactgataaagcatcctggagtatcagagtttactgttagatcagcctcatttgacttcccctcccacatggtgtttaaatccagctacactacttcctgactcaaactccactattcctgttcatgactgtcaggaactgttggaaactactgaaactggccgacctgatcttcaaaatgtgcccctaggaaaggtggatgccaccgtgttcacagacagtaccnccttcctcgagaagggactacgaggggccggtgcanctgttaccaaggagactnatgtgttgtgggctcaggctttaccancaaacacctcancncnnaaggctgaattgatcgccctcactcaggctctcggatggggtaagggatattaacgttaacactgacagcaggtacgcctttgctactgtgcatgtacgtggagccatctaccaggagcgtgggctactcactcggcaggtggctgtnatccactgtaaanggacatcaaaaggaaaacnnggctgttgcccgtggtaaccanaaanctgatcnncagctcnaagatgctgtgttgactttcactcncncctcttaaacttgctgcccacantctcctttcccaaccagatctgcctgacaatccccatactcaaaaaaaaaanaanactggccccgaacccnaaccaataaaaacgggganggtnggtngancnncctgacccaaaaataatggatcccccgggctgcaggaattcaattcanccttatcnatacccccaacnnggnggggggggccngtncccattncccctntattnattctttnnccccccccccggcntcctttttnaactcgtgaaagggaaaacctgncttaccaanttatcncctggaccntccccttccncggtngnttanaaaaaaaagcccncantcccntccnaaatttgcacngaaaggnaaggaatttaacctttattttttnntcctttantttgtnnncccccttttacccaggcgaacngccatcntttaanaaaaaaaanagaangtttatttttccttngaaccatcccaatanaaancacccgcnggggaacggggnggnaggccnctcaccccctttntgtnggngggnc

SEQ ID NO: 9 - DNA, 1146 bp, Homo sapiens
  misc_feature 1, 4, 5, 8, 9, 348, 706, 742, 745, 751, 758, 772, 793,819, 842, 846, 860, 866, 886, 889, 911, 939, 945, 955, 960, 982,999, 1002, 1005, 1009, 1010, 1033, 1047, 1049, 1055, 1058,1069, 1074, 1079, 1081, 1104, 1105, 1111, 1116, 1118: n = A,T,C or G
  misc_feature 1121, 1130, 1135, 1136, 1146: n = A,T,C or G
nccnnttnntgatgttgtctttttggcctctctttggatactttccctctcttcagaggtgaaaagggtcaaaaggagctgttgacagtcatcccaggtgggccaatgtgtccagagtacagactccatcagtgaggtcaaagcctggggcttttcagagaagggaggattatgggttttccaattatacaagtcagaagtagaaagaagggacataaaccaggaagggggtggagcactcatcacccagagggacttgtgcctctctcagtggtagtagaggggctacttcctcccaccacggttgcaaccaagaggcaatgggtgatgagcctacaggggacatanccgaggagacatgggatgaccctaagggagtaggctggttttaaggcggtgggactgggtgagggaaactctcctcttcttcagagagaagcagtacagggcgagctgaaccggctgaaggtcgaggcgaaaacacggtctggctcaggaagaccttggaagtaaaattatgaatggtgcatgaatggagccatggaaggggtgctcctgaccaaactcagccattgatcaatgttagggaaactgatcagggaagccgggaatttcattaacaacccgccacacagcttgaacattgtgaggttcagtgacccttcaaggggccactccactccaactttggccattctactttgcnaaatttccaaaacttccttttttaaggccgaatccntantccctnaaaaacnaaaaaaaatctgcncctattctggaaaaggcccancccttaccaggctggaagaaattttnccttttttttttttttgaaggcntttnttaaattgaacctnaattcncccccccaaaaaaaaacccnccnggggggcggatttccaaaaacnaattcccttaccaaaaaacaaaaacccncccttnttcccttccnccctnttcttttaattagggagagatnaagccccccaatttccnggnctngatnngtttcccccccccccattttccnaaactttttcccancnaggaanccnccctttttttnggtcngattnancaaccttccaaaccatttttccnnaaaaantttgntnggngggaaaaanacctnnttttatagan

SEQ ID NO: 10 - DNA, 545 bp, Homo sapiens
cttcattgggtacgggccccctcgaggtcgacggtatcgataagcttgatatcgaattcctgcagcccgggggatccactagttctagagtcaggaagaaccaccaaccttcctgatttttattggctctgagttctgaggccagttttcttcttctgttgagtatgcgggattgtcaggcagatctggctgtggaaaggagactgtgggcagcaagtttagaggcgtgactgaaagtcacactgcatcttgagctgctgaatcagctttctggttaccacgggcaacagccgtgttttccttttgatgtcctttacagtggattacagccacctgctgaggtgagtagcccacgctcctggtagatggctccacgtacatgcacagtagcaaaggcgtacctgctgtcagtgttaacgttaatatccttaccccatcggagagcctgagtgagggcgatcaattcagcccttttgtgctgaggtgtttgctggttaagccctgaacccacaacacatctgtctccatggtaacagctgcaccgg

SEQ ID NO: 11 - DNA, 196 bp, Homo sapiens
tctcctaggctgggcacagtggctcatacctgtaatcctgaccgtttcagaggctcaggtggggggatcgcttgagcccaagatttcaagactagtctgggtaacatagtgagaccctatctctacgaaaaaataaaaaaatgagcctggtgtagtggcacacaccagctgaggagggagaatcgagcctaggaga

SEQ ID NO: 12 - DNA, 388 bp, Homo sapiens
  misc_feature 82, 162, 287: n = A,T,C or G
tctcctaggcttgggggctctgactagaaattcaaggaacctgggattcaagtccaactgtgacaccaacttacactgtggnctccaataaactgcttctttcctattccctctctattaaataaaataaggaaaacgatgtctgtgtatagccaagtcagntatcctaaaaggagatactaagtgacattaaatatcagaatgtaaaacctgggaaccaggttcccagcctgggattaaactgacagcaagaagactgaacagtactactgtgaaaagcccgaagnggcaatatgttcactctaccgttgaaggatggctgggagaatgaatgctctgtcccccagtcccaagctcacttactatacctcctttatagcctaggaga

SEQ ID NO: 13 - DNA, 337 bp, Homo sapiens
tagtagttgcctataatcatgtttctcattattttcacattttattaaccaatttctgtttaccctgaaaaatatgagggaaatatatgaaacagggaggcaatgttcagataattgatcacaagatatgatttctacatcagatgctctttcctttcctgtttatttcctttttatttcggttgtggggtcgaatgtaatagctttgtttcaagagagagttttggcagtttctgtagcttctgacactgctcatgtctccaggcatctatttgcactttaggaggtgtcgtgggagactgagaggtctattttttccatatttgggcaactacta

SEQ ID NO: 14 - DNA, 571 bp, Homo sapiens
  misc_feature 435, 441, 451, 456, 462, 479, 488, 489, 509, 568: n = A,T,C or G
tagtagttgccatacagtgcctttccatttatttaacccccacctgaacggcataaactgagtgttcagctggtgttttttactgtaaacaataaggagactttgctcttcatttaaaccaaaatcatatttcatattttacgctcgagggtttttaccggttcctttttacactccttaaaacagtttttaagtcgtttggaacaagatattttttctttcctggcagcttttaacattatagcaaatttgtgtctgggggactgctggtcactgtttctcacagttgcaaatcaaggcatttgcaaccaagaaaaaaaaatttttttgttttatttgaaactggaccggataaacggtgtttggagcggctgctgtatatagttttaaatggtttattgcacctccttaagttgcacttatgtgggggggggnttttgnatagaaagtntttantcacanagtcacagggacttttntcttttggnnactgagctaaaaagggctgnttttcgggtgggggcagatgaaggctcacaggaggcctttctcttagaggggggaactncta

SEQ ID NO: 15 - DNA, 548 bp, Homo sapiens
  misc_feature 224, 291, 326, 376, 388, 394, 428, 433, 507, 514: n = A,T,C or G
tatatatttaataacttaaatatattttgatcacccactggggtgataagacaatagatataaaagtatttccaaaaagcataaaaccaaagtatcataccaaaccaaattcatactgcttcccccacccgcactgaaacttcaccttctaactgtctacctaaccaaattctacccttcaagtctttggtgcgtgctcactactctttttttttttttttttnttttggagatggagtctggctgtgcagcccaggggtggagtacaatggcacaacctcagctcactgnaacctccgcctcccaggttcatgagattctcctgnttcagccttcccagtagctgggactacaggtgtgcatcaccatgcctggntaatcttttttngttttngggtagagatgggggttttacatgttggccaggntggtntcgaactcctgacctcaagtgatccacccacctcaggctcccaaagtgctaggattacagacatgagccactgngcccagncctggtgcatgctcacttctctaggcaactacta

SEQ ID NO: 16 - DNA, 638 bp, Homo sapiens
  misc_feature 471, 488: n = A,T,C or G
ttccgttatgcacatgcagaatattctatcggtacttcagctattactcattttgatggcgcaatccgagcctatcctcaagatgagtatttagaaagaattgatttagcgatagaccaagctggtaagcactctgactacacgaaattgttcagatgtgatggatttatgacagttgatctttggaagagattattaagtgattattttaaagggaatccattaattccagaatatcttggtttagctcaagatgatatagaaatagaacagaaagagactacaaatgaagatgtatcaccaactgatattgaagagcctatagtagaaaatgaattagctgcatttattagccttacacatagcgattttcctgatgaatcttatattcagccatcgacatagcattacctgatgggcaaccttacgaataatagaaactgggtgcggggctattgatgaattcatccncagtaaatttggatatnacaaaatataactcgattgcatttggatgatggaatactaaatctggcaaaagtaactttggagctactagtaacctctctttttgagatgcaaaattttcttttagggtttcttattctctactttacggatattggagcataacggga

SEQ ID NO: 17 - DNA, 286 bp, Homo sapiens
actgatggatgtcgccggaggcgaggggccttatctgatgctcggctgcctgttcgtgatgtgcgcggcgattgggctgtttatctcaaacaccgccacggcggtgctgatggcgcctattgccttagcggcggcgaagtcaatgggcgtctcaccctatccttttgccatggtggtggcgatggcggcttcggcggcgtttatgaccccggtctcctcgccggttaacaccctggtgcttggccctggcaagtactcatttagcgattttgtcaaaataggcgtg

SEQ ID NO: 18 - DNA, 262 bp, Homo sapiens
  misc_feature 184, 234, 240: n = A,T,C or G
tcggtcatagcagccccttcttctcaatttcatctgtcactaccctggtgtagtatctcatagccttacatttttatagcctcctccctggtctgtcttttgattttcctgcctgtaatccatatcacacataactgcaagtaaacatttctaaagtgtggttatgctcatgtcactcctgtgncaagaaatagtttccattaccgtcttaataaaattcggatttgttctttnctattntcactcttcacctatgaccgaa

SEQ ID NO: 19 - DNA, 261 bp, Homo sapiens
tcggtcatagcaaagccagtggtttgagctctctactgtgtaaactcctaaaccaaggccatttatgataaatggtggcaggatttttattataaacatgtacccatgcaaatttcctataactctgagatatattcttctacatttaaacaataaaaataatctatttttaaaagcctaatttgcgtagttaggtaagagtgtttaatgagagggtataaggtataaatcaccagtcaacgtttctctgcctatgaccga

SEQ ID NO: 20 - DNA, 294 bp, Homo sapiens
  misc_feature 194, 274, 283, 294: n = A,T,C or G
tacaacgaggcgacgtcggtaaaatcggacatgaagccaccgctggtcttttcgtccgagcgataggcgccggccagccagcggaacggttgcccggatggcgaagcgagccggagttcttcggactgagtatgaatcttgttgtgaaaatactcgccgccttcgttcgacgacgtcgcgtcgaaatcttcganctccttacgatcgaagtcttcgtgggcgacgatcgcggtcagttccgccccaccgaaatcatggttgagccggatgctgnccccgaagncctcgtttgtn

SEQ ID NO: 21 - DNA, 208 bp, Homo sapiens
  misc_feature 116, 132, 140, 160, 164, 191, 197, 199: n = A,T,C or G
ttggtaaagggcatggacgcagacgcctgacgtttggctgaaaatctttcattgattcgtatcaatgaataggaaaattcccaaagagggaatgtcctgttgctcgccagtttttntgttgttctcatgganaaggcaangagctcttcagactattggnattntcgttcggtcttctgccaactagtcgncttgcnangatcttcat

SEQ ID NO: 22 - DNA, 287 bp, Homo sapiens
  misc_feature 1, 4, 25, 121, 168, 207, 212: n = A,T,C or G
nccnttgagctgagtgattgagatntgtaatggttgtaagggtgattcaggcggattagggtggcgggtcacccggcagtgggtctcccgacaggccagcaggatttggggcaggtacggngtgcgcatcgctcgactatatgctatggcaggcgagccgtggaaggnggatcaggtcacggcgctggagctttccacggtccatgnattgngatggctgttctaggcggctgttgccaagcgtgatggtacgctggctggagcattgatttctggtgccaaggtgg

SEQ ID NO: 23 - DNA, 204 bp, Homo sapiens
  misc_feature 40, 121, 131, 162, 184, 197: n = A,T,C or G
ttgggtaaagggagcaaggagaaggcatggagaggctcangctggtcctggcctacgactgggccaagctgtcgccggggatggtggagaactgaagcgggacctcctcgaggtcctccgncgttacttcnccgtccaggaggagggtctttccgtggtctnggaggagcggggggagaagatnctcctcatggtcnacatccc

SEQ ID NO: 24 - DNA, 264 bp, Homo sapiens
  misc_feature 171, 206: n = A,T,C or G
tggattggtcaggagcgggtagagtggcaccattgaggggatattcaaaaatattattttgtcctaaatgatagttgctgagtttttctttgacccatgagttatattggagtttattttttaactttccaatcgcatggacatgttagacttattttctgttaatgattnctatttttattaaattggatttgagaaattggttnttattatatcaatttttggtatttgttgagtttgacattatagcttagtatgtgacca

SEQ ID NO: 25 - DNA, 376 bp, Homo sapiens
  misc_feature 103, 111, 192, 196, 199, 220, 224, 230, 251, 268, 283,317, 352, 370, 374: n = A,T,C or G
ttacaacgaggggaaactccgtctctacaaaaattaaaaaattagccaggtgtggtggtgtgcacccgcaatcccagctacttgggaggttgagacacaagantcacctanatgtgggaggtcaaggttgcatgagtcatgattgtgccactgcactccagcctgggtgacagaccgagaccctgcctcaanaganaangaataggaagttcagaaatcntggntgtggngcccagcaatctgcatctatncaacccctgcaggcaangctgatgcagcctangttcaagagctgctgtttctggaggcagcagttngggcttccatccagtatcacggccacactcgcacnagccatctgtcctccgtntgtnac

SEQ ID NO: 26 - DNA, 372 bp, Homo sapiens
  misc_feature 231, 312, 340: n = A,T,C or G
ttacaacgaggggaaactccgtctctacaaaaattaaaaaattagccaggtgtggtggtgtgcacctgtaatcccagctacttgggcggctgagacacaagaaccacctaaatgtgggagggtcaaggttgcatgagtcatgatcgcgccactgcactccagcctgggtgacagactgagaccctgcctcaaaagaaaaagaataggaagttcagaaaccctgggtgtggngcccagcaatctgcatttaaacaatccctgcaggcaatgctgatgcagcctaagttcaagagctgctgttctggaggcagnagtaagggcttccatccagcatcacggncaacactgcaaaagcacctgtcctcgttggta

SEQ ID NO: 27 - DNA, 477 bp, Homo sapiens
ttctgtccacatctacaagttttatttattttgtgggttttcagggtgactaagtttttccctacattgaaaagagaagttgctaaaaggtgcacaggaaatcatttttttaagtgaatatgataatatgggtccgtgcttaatacaactgagacatatttgttctctgtttttttagagtcacctcttaaagtccaatcccacaatggtgaaaaaaaaatagaaagtatttgttctacctttaaggagactgcagggattctccttgaaaacggagtatggaatcaatcttaaataaatatgaaattggttggtcttctgggataagaaattcccaactcagtgtgctgaaattcacctgactttttttgggaaaaaatagtcgaaaatgtcaatttggtccataaaatacatgttactattaaaagatatttaaagacaaattctttcagagctctaagattggtgtggacagaa

SEQ ID NO: 28 - DNA, 438 bp, Homo sapiens
  misc_feature 4, 16, 30, 255, 413: n = A,T,C or G
tctncaacctcttgantgtcaaaaaccttntaggctatctctaaaagctgactggtattcattccagcaaaatccctctagtttttggagtttccttttactatctggggctgcctgagccacaaatgccaaattaagagcatggctattttcgggggctgacaggtcaaaaggggtgtaaatccgataagcctcctggaggtgctctaaaaacactcctggtgactcatcatgcccctggacgacttcaatcgncttagacaagtttataggtttctgggcagctccctgaatacccacgaggagataccggtggaaatcgtcaaaagttctccctccacttgagaaatttgggtcccaattaggtcccaattgggtctctaatcactattcctctagcttcctcctccggnctattggttgatgtgaggttgaaga

SEQ ID NO: 29 - DNA, 620 bp, Homo sapiens
  misc_feature 391, 481, 483, 490, 497, 510, 527, 532, 540, 545, 593,612: n = A,T,C or G
aagagggtaccagccccaagccttgacaacttccatagggtgtcaagcctgtgggtgcacagaagtcaaaaattgagttttgggatcctcagcctagatttcagaggatataaagaaacacctaacacctagatattcagacaaaagtttactacagggatgaagctttcacggaaaacctctactaggaaagtacagaagagaaatgtgggtttggagcccccaaacagaatcccctctagaacactgcctaatgaaactgtgagaagatggccactgtcatccagacaccagaatgatagacccaccaaaaacttatgccatattgcctataaaacctacagacactcaatgccagccccatgaaaaaaaaactgagaagaagactgtnccctacaatgccaccggagcagaactgccccaggccatggaagcacagctcttatatcaatgtgacctggatgttgagacatggaatccnangaaatcnttttaanacttccacggttnaatgactgccctattanattcngaacttanatccnggcctgtgacctctttgctttggccattccccctttttggaatggctntttttttcccatgcctgtnccctctta

SEQ ID NO: 30 - DNA, 100 bp, Homo sapiens
ttacaacgagggggtcaatgtcataaatgtcacaataaaacaatctcttctttttttttttttttttttttttttttttttttttttttttttttttttt

SEQ ID NO: 31 - DNA, 762 bp, Homo sapiens
  misc_feature 626, 652, 662, 715, 736: n = A,T,C or G
tagtctatgcgccggacagagcagaattaaattggaagttgccctccggactttctacccacactcttcctgaaaagagaaagaaaagaggcaggaaagaggttaggatttcattttcaagagtcagctaattaggagagcagagtttagacagcagtaggcaccccatgatacaaaccatggacaaagtccctgtttagtaactgccagacatgatcctgctcaggttttgaaatctctctgcccataaaagatggagagcaggagtgccatccacatcaacacgtgtccaagaaagagtctcagggagacaagggtatcaaaaaacaagattcttaatgggaaggaaatcaaaccaaaaaattagatttttctctacatatatataatatacagatatttaacacattattccagaggtggctccagtccttggggcttgagagatggtgaaaacttttgttccacattaacttctgctctcaaattctgaagtatatcagaatgggacaggcaatgttttgctccacactggggcacagacccaaatggttctgtgcccgaagaagagaagcccgaaagacatgaaggatgcttaaggggggttgggaaagccaaattggtantatcttttcctcctgcctgtgttccngaagtctccnctgaaggaattcttaaaaccctttgtgaggaaatgcccccttaccatgacaantggtcccattgcttttagggngatggaaacaccaagggttttgatcc

SEQ ID NO: 32 - DNA, 276 bp, Homo sapiens
tagtctatgcgtgtattaacctcccctccctcagtaacaaccaaagaggcaggagctgttattaccaaccccattttacagatgcatcaataatgacagagaagtgaagtgacttgcgcacacaaccagtaaattggcagagtcagatttgaatccatggagtctggtctgcactttcaatcaccgaataccctttctaagaaacgtgtgctgaatgagtgcatggataaatcagtgtctactcaacatctttgcctagatatcccgcatagacta

SEQ ID NO: 33 - DNA, 477 bp, Homo sapiens
tagtagttgccaaatatttgaaaatttacccagaagtgattgaaaactttttggaaacaaaaacaaataaagccaaaaggtaaaataaaaatatctttgcactctcgttattacctatccataactttttcaccgtaagctctcctgcttgttagtgtagtgtggttatattaaactttttagttattattttttattcacttttccactagaaagtcattattgatttagcacacatgttgatctcatttcattttttctttttataggcaaaatttgatgctatgcaacaaaaatactcaagcccattatcttttttccccccgaaatctgaaaattgcaggggacagagggaagttatcccattaaaaaattgtaaatatgttcagtttatgtttaaaaatgcacaaaacataagaaaattgtgtttacttgagctgctgattgtaagcagttttatctcaggggcaactacta

SEQ ID NO: 34 - DNA, 631 bp, Homo sapiens
tagtagttgccaattcagatgatcagaaatgctgctttcctcagcattgtcttgttaaaccgcatgccatttggaactttggcagtgagaagccaaaaggaagaggtgaatgacatatatatatatatatattcaatgaaagtaaaatgtatatgctcatatactttctagttatcagaatgagttaagctttatgccattgggctgctgcatattttaatcagaagataaaagaaaatctgggcatttttagaatgtgatacatgtttttttaaaactgttaaatattatttcgatatttgtctaagaaccggaatgttcttaaaatttactaaaacagtattgtttgaggaagagaaaactgtactgtttgccattattacagtcgtacaagtgcatgtcaagtcacccactctctcaggcatcagtatccacctcatagctttacacattttgacggggaatattgcagcatcctcaggcctgacatctgggaaaggctcagatccacctactgctccttgctcgttgatttgttttaaaatattgtgcctggtgtcacttttaagccacagccctgcctaaaagccagcagagaacagaacccgcaccattctataggcaactacta

SEQ ID NO: 35 - DNA, 578 bp, Homo sapiens
tagtagttgccatcccatattacagaaggctctgtatacatgacttatttggaagtgatctgttttctctccaaacccatttatcgtaatttcaccagtcttggatcaatcttggtttccactgataccatgaaacctacttggagcagacattgcacagttttctgtggtaaaaactaaaggtttatttgctaagctgtcatcttatgcttagtattttttttttacagtggggaattgctgagattacattttgttattcattagatactttgggataacttgacactgtcttctttttttcgcttttaattgctatcatcatgcttttgaaacaagaacacattagtcctcaagtattacataagcttgcttgttacgcctggtggtttaaaggactatctttggcctcaggttcacaagaatgggcaaagtgtttccttatgttctgtagttctcaataaaagattgccaggggccgggtactgtggctcgcactgtaatcccagcactttgggaagctgaggctggcggatcatgttagggcaggtgttcgaaaccagcctgggcaactacta

SEQ ID NO: 36 - DNA, 583 bp, Homo sapiens
tagtagttgcctgtaatcccagcaactcaggaggctggggcaggagaatcagttgaacctgggaggcagaagttgtaattagcaaagatcgcaccattgcacttcagcctgggcaacaagagtgagattccatctcaaaaacaaaaaaaagaaaaagaaaagaaaaggaaaaaacgtataaacccagccaaaacaaaatgatcattcttttaataagcaagactaatttaatgtgtttatttaatcaaagcagttgaatcttctgagttattggtgaaaatacccatgtagttaatttagggttcttacttgggtgaacgtttgatgttcacaggttataaaatggttaacaaggaaaatgatgcataaagaatcttataaactactaaaaataaataaaatataaatggataggtgctatggatggagtttttgtgtaatttaaaatcttgaagtcattttggatgctcattggttgtctggtaatttccattaggaaaaggttatgatatggggaaactgtttctggaaattgcggaatgtttctcatctgtaaaatgctagtatctcagggcaactacta

SEQ ID NO: 37 - DNA, 716 bp, Homo sapiens
  misc_feature 15, 669, 673, 678, 686, 704: n = A,T,C or G
gatctactagtcatntggattctatccatggcagctaagcctttctgaatggattctactgctttcttgttctttaatccagacccttatatatgtttatgttcacaggcagggcaatgtttagtgaaaacaattctaaattttttattttgcattttcatgctaatttccgtcacactccagcaggcttcctgggagaataaggagaaatacagctaaagacattgtccctgcttacttacagcctaatggtatgcaaaaccacttcaataaagtaacaggaaaagtactaaccaggtagaatggaccaaaactgatatagaaaaatcagaggaagagaggaacaaatatttactgagtcctagaatgtacaaggctttttaattacatattttatgtaaggcctgcaaaaaacaggtgagtaatcaacatttgtcccattttacatataaggaaactgaagcttaaattgaataatttaatgcatagattttatagttagaccatgttcaggtccctatgttatacttactagctgtatgaatatgagaaaataattttgttattttcttggcatcagtattttcatctgcaaaataaagctaaagttatttagcaaacagtcagcatagtgcctgatacatagtaggtgctccaaacatgattacnctantattnggtattanaaaaatccaatataggcntggataaaaccg

SEQ ID NO: 38 - DNA, 688 bp, Homo sapiens
  misc_feature 260: n = A,T,C or G
ttctgtccacatatcatcccactttaattgttaatcagcaaaactttcaatgaaaaatcatccattttaaccaggatcacaccaggaaactgaaggtgtatttttttttaccttaaaaaaaaaaaaaaaaaccaaacaaaccaaaacagattaacagcaaagagttctaaaaaatttacatttctcttacaactgtcattcagagaacaatagttcttaagtctgttaaatcttggcattaacagagaaacttgatgaanagttgtacttggaatattgtggatttttttttttgtctaatctccccctattgttttgccaacagtaatttaagtttgtgtggaacatccccgtagttgaagtgtaaacaatgtataggaaggaatatatgataagatgatgcatcacatatgcattacatgtagggaccttcacaacttcatgcactcagaaaacatgcttgaagaggaggagaggacggcccagggtcaccatccaggtgccttgaggacagagaatgcagaagtggcactgttgaaatttagaagaccatgtgtgaatggtttcaggcctgggatgtttgccaccaagaagtgcctccgagaaatttctttcccatttggaatacagggtggcttgatgggtacggtgggtgacccaacgaagaaaatgaaattctgccctttcc

SEQ ID NO: 39 - DNA, 585 bp, Homo sapiens
  misc_feature 14, 15, 24, 53, 108, 135, 465, 477, 495, 499, 504, 517,530, 580, 581: n = A,T,C or G
tagtagttgccgcnnacctaaaanttggaaagcatgatgtctaggaaacatantaaaatagggtatgcctatgtgctacagagagatgttagcatttaaagtgcatanttttatgtattttgacaaatgcatatncctctataatccacaactgattacgaagctattacaattaaaaagtttggccgggcgtggtgggcggtggctgacgcctgtaatcccagcactttgggaggccgaggcacgcggatcacgaggtcgggagttcaagaccatcctggctaacacggtgaaagtccatctctactaaaaatacgaaaaaattaccccggcgtggtggcgggcgcctgtagtcccagctactccggaggctgaggcaggagaatggcgtgaacccaggacacggagcttgcagtgtgccaacatcacgtcactgccctccagcctgggggacaggaacaagantcccgtcctcanaaaagaaaaatactactnatantttcnactttattttaanttacacagaactncctcttggtacccccttaccattcatctcacccacctcctatagggcacnnctaa

SEQ ID NO: 40 - DNA, 475 bp, Homo sapiens
tctgtccacaccaatcttagaagctctgaaaagaatttgtctttaaatatcttttaatagtaacatgtattttatggaccaaattgacattttcgactgttttttccaaaaaagtcaggtgaatttcagcacactgagttgggaatttcttatcccagaagaccaaccaatttcatatttatttaagattgattccatactccgttttcaaggagaatccctgcagtctccttaaaggtagaacaaatacttcctatttttttttcaccattgtgggattggactttaagaggtgactctaaaaaaacagagaacaaatatgtctcagttgtattaagcacggacccatattatcatattcacttaaaaaaatgatttcctgtgcaccttttggcaacttctcttttcaatgtagggaaaaacttagtcaccctgaaaacccacaaaataaataaaacttgtagatgtggacaga

SEQ ID NO: 41 - DNA, 423 bp, Homo sapiens
taagagggtacatcgggtaagaacgtaggcacatctagagcttagagaagtctggggtaggaaaaaaatctaagtatttataagggtataggtaacatttaaaagtagggctagctgacattatttagaaagaacacatacggagagataagggcaaaggactaagaccagaggaacactaatatttagtgatcacttccattcttggtaaaaatagtaacttttaagttagcttcaaggaagatttttggccatgattagttgtcaaaagttagttctcttgggtttatattactaattttgttttaagatccttgttagtgctttaataaagtcatgttatatcaaacgctctaaaacattgtagcatgttaaatgtcacaatatacttaccatttgttgtatatggctgtaccctctcta

SEQ ID NO: 42 - DNA, 527 bp, Homo sapiens
  misc_feature 470, 475, 515, 522: n = A,T,C or G
tctcctaggctaatgtgtgtgtttctgtaaaagtaaaaagttaaaaattttaaaaatagaaaaaagcttatagaataagaatatgaagaaagaaaatatttttgtacatttgcacaatgagtttatgttttaagctaagtgttattacaaaagagccaaaaaggttttaaaaattaaaacgtttgtaaagttacagtacccttatgttaatttataattgaagaaagaaaaacttttttttataaatgtagtgtagcctaagcatacagtatttataaagtctggcagtgttcaataatgtcctaggccttcacattcactcactgactcacccagagcaacttccagtcctgtaagctccattcgtggtaagtgccctatacaggtgcaccatttattttacagtatttttactgtaccttctctatgtttccatatgtttcgatatacaaataccactggttactatngcccnacaggtaattccagtaacacggcctgtatacgtctggtanccctagngaaga

SEQ ID NO: 43 - DNA, 331 bp, Homo sapiens
tcttcaacctcgtaggacaactctcatatgcctgggcactatttttaggttactaccttggctgcccttctttaagaaaaaaaaaagaagaaaaaagaacttttccacaagtttctcttcctctagttggaaaattagagaaatcatgtttttaattttgtgttatttcagatcacaaattcaaacacttgtaaacattaagcttctgttcaatcccctgggaagaggattcattctgatatttacggttcaaaagaagttgtaatattgtgcttggaacacagagaaccagttattaacttcctactactattatataataaataataac

SEQ ID NO: 44 - DNA, 592 bp, Homo sapiens
  misc_feature 473: n = A,T,C or G
ggcttagtagttgccaggcaaaatarcgttgattctcctcaggagccacccccaacacccctgtttgcttctagacctatacctagactaaagtcccagcagacccctagaggtgaggttcagagtgacccttgaggagatgtgctacactagaaaagaactgcttgagttttctaatttatataagcagaaatctggagaagagtcataggaatggatattaagggtgtgagataatggcggaaggaatatagagttggatcaggctggacttattgatttgaacccactaagtagagattctgcttttgatgttgcagctcagggagttaaaaaaggttttaatggttctaatagtttatttgcttggttagctgaaatatggataaaagatggcccactgtgagcaagctggaaatgcctgatctctctcagtttaatgtagaggaagggatccaaaagtttagggaganttggatgctggraktggattggtcactttgrgacctacccwtcccagctgggagggtccagaagatacacccttgaccaacgctttgcgaaatggatttgtgatggcggcaactactaa

SEQ ID NO: 45 - DNA, 567 bp, Homo sapiens
  misc_feature 522, 561, 566: n = A,T,C or G
ggcttagtagttgccattgcgagtgcttgctcaacgagcgttgaacatggcggattgtctagattcaacggatttgagttttaccagcaaagcgaaccaagcgcggcccagagaattatgggttggttggctttgaaaagatggaaatcctgtaggcctagtcagaaaagccttcttgcagaacagttggttctcgggcgaacgctcatcaagatgcccattggaaaggctagcgtgtatttgggagagcctgatagcgtgtcttctgatgatgtttgtgcttggacagtgacaaaagatatgcaaagcaagtccgaactagacgtcaagcttcgtgagcaaattattgtagactcctacttatactgtgaggaatgatagccaagggtggggactttaagactaaggtggtttgtacttgcgccgatgatcccaggcagaaagamctgatcgctagttttatacgggcaactactaagccgaattccagcacactggcggccgttactaattggatccganctcggtaccagcttgatgcatascttgagttwtctatantgtcnc

SEQ ID NO: 46 - DNA, 908 bp, Homo sapiens
  misc_feature 21, 23, 24, 27, 29, 34: n = A,T,C or G
gagcgaaagaccgagggcagngnntangngcgangaagcggagagggccaaaaagcaaccgctttccccggggggtgccgattcattaaggcaggtggaggacaggtttcccgatggaaggcggcaggggcgcaagcaattaatgtgagtaggccattcattagcacccgggcttaacatttaagcttcgggttggtatgtggtgggaattgtgagcggataacaatttcacacaggaaacagctatgaccatgattacgccaagctatttaggtgacattatagaataactcaagttatgcatcaagcttggtaccgagttcggatccactagtaacggccgccagtgtgtggaattcggcttagtagttgccgaccatggagtgctacctaggctagaatacctgagytcctccctagcctcactcacattaaattgtatcttttctacattagatgtcctcagcgccttatttctgctggacwatcgataaattaatcctgataggatgatagcagcagattaattactgagagtatgttaatgtgtcatccctcctatataacgtatttgcattttaatggagcaattctggagataatccctgaaggcaaaggaatgaatcttgagggtgagaaagccagaatcagtgtccagctgcagttgtgggagaaggtgatattatgtatgtctcagaagtgacaccatatgggcaactactaagcccgaattccagcacactggcgggcgttactaatggatccgagctcggtaccaagcttgatgcatagcttgagtatctatagtgtcactaaatagcctggcgttatcatggtcatagctgtttcctgtgtgaaattgttatccgctcccaattccccccaccatacgagccggaacataaagt

SEQ ID NO: 47 - DNA, 480 bp, Homo sapiens
  misc_feature 408, 461: n = A,T,C or G
tgccaacaaggaaagttttaaatttccccttgaggattcttggtgatcatcaaattcagtggtttttaaggttgttttctgtcaaataactctaactttaagccaaacagtatatggaagcacagatakaatattacacagataaaagaggagttgatctaaagtaragatagttgggggctttaatttctggaacctaggtctccccatcttcttctgtgctgaggaacttcttggaagcggggattctaaagttctttggaagacagtttgaaaaccaccatgttgttctcagtacctttatttttaaaaagtaggtgaacattttgagagagaaaagggcttggttgagatgaagtcccccccccccctttttttttttttagctgaaatagataccctatgttnaargaarggattattatttaccatgccaytarscacatgctctttgatgggcnyctccstaccctccttaag

SEQ ID NO: 48 - DNA, 591 bp, Homo sapiens
aagagggtaccgagtggaatttccgcttcactagtctggtgtggctagtcggtttcgtggtggccaacattacgaacttccaactcaaccgttcttggacgttcaagcgggagtaccggcgaggatggtggcgtgaattctggcctttctttgccgtgggatcggtagccgccatcatcggtatgtttatcaagatcttctttactaacccgacctctccgatttacctgcccgagccgtggtttaacgaggggagggggatccagtcacgcgagtactggtcccagatcttcgccatcgtcgtgacaatgcctatcaacttcgtcgtcaataagttgtggaccttccgaacggtgaagcactccgaaaacgtccggtggctgctgtgcggtgactcccaaaatcttgataacaacaaggtaaccgaatcgcgctaaggaaccccggcatctcgggtactctgcatatgcgtaccccttaagccgaattccagcacactggcggccgttactaattggatccgaactccgtaaccaagcctgatgcgtaacttgagttattctatagtgtccctaaaataacctggcgtta

SEQ ID NO: 49 - DNA, 454 bp, Homo sapiens
aagagggtacctgccttgaaatttaaatgtctaaggaaartgggagatgattaagagttggtgtggcytagtcacaccaaaatgtatttattacatcctgctcctttctagttgacaggaaagaaagctgctgtggggaaaggagggataaatactgaagggatttactaaacaaatgtccatcacagagttttcctttttttttttttgagacagagtcttgctctgtcacccaggctggaatgaagwggtatgatctcagttgaatgcaacctctacctcctaggttcaagcgattctcatgcctcagcctcctgagcagctgggactataggcgcatgctaccatgccaggctaatttttatatttttattagagacggggtgttgccatgttggccaggcaggtctcgaactcctgggcctcagatgatctgccccaccgtaccctctta

SEQ ID NO: 50 - DNA, 463 bp, Homo sapiens
aagagggtaccaaaaaaaagaaaaaggaaaaaaagaaaaacaacttgtataaggctttctgctgcatacagctttttttttttaaataaatggtgccaacaaatgtttttgcattcacaccaattgctggttttgaaatcgtactcttcaaaggtatttgtgcagatcaatccaatagtgatgccccgtaggttttgtggactgcccacgttgtctaccttctcatgtaggagccattgagagactgtttggacatgcctgtgttcatgtagccgtgatgtccgggggccgtgtacatcatgttaccgtggggtggggtctgcattggctgctgggcatatggctgggtgcccatcatgcccatctgcatctgcatagggtattggggcgtttgatccatatagccatgattgctgtggtagccactgttcatcattggctgggacatgctgttaccctctta

SEQ ID NO: 51 - DNA, 399 bp, Homo sapiens
cttcaacctcccaaagtgctgggattacaggactgagccaccacgctcagcctaagcctctttttcactaccctctaagcgatctaccacagtgatgaggggctaaagagcagtgcaatttgattacaataatggaacttagatttattaattaacaatttttccttagcatgttggttccataattattaagagtatggacttacttagaaatgagctttcattttaagaatttcatctttgaccttctctattagtctgagcagtatgacactatacgtattttatttaactaacctaccttgagctattactttttaaaaggctatatacatgaatgtgtattgtcaactgtaaagccccacagtatttaattatatcatgatgtctttgaggttg

SEQ ID NO: 52 - DNA, 392 bp, Homo sapiens
cttcaacctcaatcaaccttggtaattgataaaatcatcacttaactttctgatataatggcaataattatctgagaaaaaaaagtggtgaaagattaaacttgcatttctctcagaatcttgaaggatatttgaataattcaaaagcggaatcagtagtatcagccgaagaaactcacttagctagaacgttggacccatggatctaagtccctgcccttccactaaccagctgattggttttgtgtaaacctcctacacgcttgggcttggtcgcctcatttgtcaaagtaaaggctgaaataggaagataatgaaccgtgtctttttggtctcttttccatccattactctgattttacaaagaggcctgtattcccctggtgaggttg

SEQ ID NO: 53 - DNA, 179 bp, Homo sapiens
  misc_feature 135, 143, 179: n = A,T,C or G
ttcgggtgatgcctcctcaggctacagtgaagactggattacagaaaggtgccagcgagatttcagattcctgtaaacctctaaagaaaaggagtcgcgcctcaactgatgtagaaatgactagttcagcatacngagacacntctgactccgattctagaggactgagtgacctgcan

SEQ ID NO: 54 - DNA, 112 bp, Homo sapiens
  misc_feature 31, 49, 54, 55, 75, 91, 107: n = A,T,C or G
ttcgggtgatgcctcctcaggctacatcatnatagaagcaaagtagaanaatcnngtttgtgcattttcccacanacaaaattcaaatgantggaagaaattggganagtat

SEQ ID NO: 55 - DNA, 225 bp, Homo sapiens
tgagcttccgcttctgacaactcaatagataatcaaaggacaactttaacagggattcacaaaggagtatatccaaatgccaataaacatataaaaaggaattcagcttcatcatcatcagaagwatgcaaattaaaaccataatgagaaaccactatgtcccactagaatagataaaatcttaaaagactggtaaaaccaagtgttggtaaggcaagaggagca

SEQ ID NO: 56 - DNA, 175 bp, Homo sapiens
gctcctcttgccttaccaacacattctcaaaaacctgttagagtcctaagcattctcctgttagtattgggattttacccctgtcctataaagatgttatgtaccaaaaatgaagtggagggccataccctgagggaggggagggatctctagtgttgtcagaagcggaagctca

SEQ ID NO: 57 - DNA, 223 bp, Homo sapiens
agccatttaccacccatggatgaatggattttgtaattctagctgttgtattttgtgaatttgttaattttgttgtttttctgtgaaacacatacattggatatgggaggtaaaggagtgtcccagttgctcctggtcactccctttatagccattactgtcttgtttcttgtaactcaggttaggttttggtctctcttgctccactgcaaaaaaaaaaaaa

SEQ ID NO: 58 - DNA, 211 bp, Homo sapiens
gttcgaaggtgaacgtgtaggtagcggatctcacaactggggaactgtcaaagacgaattaactgacttggatcaatcaaatgtgactgaggaaacacctgaaggtgaagaacatcatccagtggcagacactgaaaataaggagaatgaagttgaagaggtaaaagaggagggtccaaaagagatgactttggatgggtggtaaatggct

SEQ ID NO: 59 - DNA, 208 bp, Homo sapiens
gctcctcttgccttaccaactttgcacccatcatcaaccatgtggccaggtttgcagcccaggctgcacatcaggggactgcctcgcaatacttcatgctgttgctgctgactgatggtgctgtgacggatgtggaagccacacgtgaggctgtggtgcgtgcctcgaacctgcccatgtcagtgatcattatgggtggtaaatggct

SEQ ID NO: 60 - DNA, 171 bp, Homo sapiens
agccatttaccacccatactaaattctagttcaaactccaacttcttccataaaacatctaaccactgacaccagttggcaatagcttcttccttctttaacctcttagagtatttatggtcaatgccacacatttctgcaactgaataaagttggtaaggcaagaggagc

SEQ ID NO: 61 - DNA, 134 bp, Homo sapiens
  misc_feature 37, 70, 80, 86, 88, 97, 117, 123, 131: n = A,T,C or G
cgggtgatgcctcctcaggctttggtgtgtccactcnactcactggcctcttctccagcaactggtgaanatgtcctcangaaaancnccacacgcngctcagggtggggtgggaancatcanaatcatcnggc

SEQ ID NO: 62 - DNA, 145 bp, Homo sapiens
agagggtacatatgcaacagtatataaaggaagaagtgcactgagaggaacttcatcaaggccatttaatcaataagtgatagagtcaaggctcaacccaggtgtgacggattccaggtcccaagctccttactggtaccctctt

SEQ ID NO: 63 - DNA, 297 bp, Homo sapiens
tgcactgagaggaattcaaagggtttatgccaaagaacaaaccagtcctctgcagcctaactcatttgtttttgggctgcgaagccatgtagagggcgatcaggcagtagatggtccctcccacagtcagcgccatggtggtccggtaaagcatttggtcaggcaggcctcgtttcaggtagacgggcacacatcagctttctggaaaaacttttgtagctctggagctttgtttttcccagcataatcatacactgtggaatcggaggtcagtttagttggtaaggcaagaggagc

SEQ ID NO: 64 - DNA, 300 bp, Homo sapiens
gcactgagaggaacttccaatactatgttgaataggagtggtgagagagggcatccttgtcttgtgccggttttcaaagggaatgcttccagcttttgcccattcagtataatattaaagaatgttttaccattttctgtcttgcctgtttttctgtgtttttgttggtctcttcattctccatttttaggcctttacatgttaggaatatatttcttttaatgatacttcacctttggtatcttttgtgagactctactcatagtgtgataagcactgggttggtaaggcaagaggagc

SEQ ID NO: 65 - DNA, 203 bp, Homo sapiens
gctcctcttgccttaccaactcacccagtatgtcagcaattttatcrgctttacctacgaaacagcctgtatccaaacacttaacacactcacctgaaaagttcaggcaacaatcgccttctcatgggtctctctgctccagttctgaacctttctcttttcctagaacatgcatttargtcgatagaagttcctctcagtgc

SEQ ID NO: 66 - DNA, 344 bp, Homo sapiens
tacggggacccctgcattgagaaagcgagactcactctgaagctgaaatgctgttgcccttgcagtgctggtagcaggagttctgtgctttgtgggctaaggctcctggatgacccctgacatggagaaggcagagttgtgtgccccttctcatggcctcgtcaaggcatcatggactgccacacacaaaatgccgtttttattaacgacatgaaattgaaggagagaacacaattcactgatgtggctcgtaaccatggatatggtcacatacagaggtgtgattatgtaaaggttaattccacccacctcatgtggaaactagcctcaatgcaggggtccca

SEQ ID NO: 67 - DNA, 157 bp, Homo sapiens
gcactgagaggaacttcgtagggaggttgaactggctgctgaggagggggaacaacagggtaaccagactgatagccattggatggataatatggtggttgaggagggacactacttatagcagagggttgtgtatagcctgaggaggcatcacccg

SEQ ID NO: 68 - DNA, 137 bp, Homo sapiens
gcactgagaggaacttctagaaagtgaaagtctagacataaaataaaataaaaatttaaaactcaggagagacagcccagcacggtggctcacgcctgtaatcccagaactttgggagcctgaggaggcatcacccg

SEQ ID NO: 69 - DNA, 137 bp, Homo sapiens
cgggtgatgcctcctcaggctgtattttgaagactatcgactggacttcttatcaactgaagaatccgttaaaaataccagttgtattatttctacctgtcaaaatccatttcaaatgttgaagttcctctcagtgc

SEQ ID NO: 70 - DNA, 220 bp, Homo sapiens
  misc_feature 89, 112, 129, 171, 172: n = A,T,C or G
agcatgttgagcccagacacgcaatctgaatgagtgtgcacctcaagtaaatgtctacacgctgcctggtctgacatggcacaccatcncgtggagggcacasctctgctcngcctacwacgagggcantctcatwgacaggttccacccaccaaactgcaagaggctcannaagtactrccagggtmyasggacmasggtgggaytycaycacwcatct

SEQ ID NO: 71 - DNA, 353 bp, Homo sapiens
  misc_feature 66, 160, 204, 246, 267, 334, 339, 342: n = A,T,C or G
cgttagggtctctatccactgctaaaccatacacctgggtaaacagggaccatttaacattcccanctaaatatgccaagtgacttcacatgtttatcttaaagatgtccaaaacgcaactgattttctcccctaaacctgtgatggtgggatgattaancctgagtggtctacagcaagttaagtgcaaggtgctaaatgaangtgacctgagatacagcatctacaaggcagtacctctcaacncagggcaactttgcttctcanagggcatttagcagtgtctgaagtaatttctgtattacaactcacggggcggggggtgaatatctantgganagnagaccctaacg

SEQ ID NO: 72 - DNA, 343 bp, Homo sapiens
gcactgagaggaacttccaatacyatkatcagagtgaacargcarccyacagaacaggagaaaatgttygcaatctctccatctgacaaaaggctaatatccagawtctaawaggaacttaaacaaatttatgagaaaagaacaracaacctcawcaaaaagtgggtgaaggawatgctsaaargaagacatytattcagccagtaaacayatgaaaaaaaggctcatsatcactgawcattagagaaatgcaaatcaaaaccacaatgagataccatctyayrccagttagaayggtgatcattaaaarstcaggaaacaacagatgctggacaaggtgtca

SEQ ID NO: 73 - DNA, 321 bp, Homo sapiens
  misc_feature 288: n = A,T,C or G
gcactgagaggaacttcagagagagagagagagttccaccctgtacttggggagagaaacagaaggtgagaaagtctttggttctgaagcagcttctaagatcttttcatttgcttcatttcaaagttcccatgctgccaaagtgccatcctttggggtactgttttctgagctccagtgataactcatttatacaagggagatacccagaaaaaaagtgagcaaatcttaaaaaggtggcttgagttcagccttaaataccatcttgaaatgacacagagaaagaangatgttgggtgggagtggatagagaccctaacg

SEQ ID NO: 74 - DNA, 321 bp, Homo sapiens
gcactgagaggaacttcagagagagagagagagttccaccctgtacttggggagagaaacagaaggtgagaaagtctttggttctgaagcagcttctaagatcttttcatttgcttcatttcaaagttcccatgctgccaaagtgccatcctttggggtactgttttctgagctccagtgataactcatttatacaagggagatacccagaaaaaaagtgagcaaatcttaaaaaggtggcttgagttcagycttaaataccatcttgaaatgamacagagaaagaaggatgttgggtgggagtggatagagaccctaacg

SEQ ID NO: 75 - DNA, 317 bp, Homo sapiens
gcactgagaggaacttccacatgcactgagaaatgcatgttcacaaggactgaagtctggaactcagtttctcagttccaatcctgattcaggtgtttaccagctacacaaccttaagcaagtcagataaccttagcttcctcatatgcaaaatgagaatgaaaagtactcatcgctgaattgttttgaggattagaaaaacatctggcatgcagtagaaattcaattagtattcattttcattcttctaaattaaacaaataggatttttagtggtggaacttcagacaccagaaatgggagtggatagagaccct

SEQ ID NO: 76 - DNA, 244 bp, Homo sapiens
cgttagggtctctatccactcccactactgatcaaactctatttatttaattatttttatcatactttaagttctgggatacacgtgcagcatgcgcaggtttgttgcataggtatacacttgccatggtggtttgctgcacccatcagtccatcatctacattaggtatttctcctaatgctatccctcccctagccccttacacccccaacaggctctagtgtgtgaagttcctctcagtgc

SEQ ID NO: 77 - DNA, 254 bp, Homo sapiens
cgttagggtctctatccactgaaatctgaagcacaggaggaagagaagcagtyctagtgagatggcaagttcwtttaccacactctttaacatttygtttagttttaacctttatttatggataataaaggttaatattaataatgatttattttaaggcattcccraatttgcataattctccttttggagatacccttttatctccagtgcaagtctggatcaaagtgatasamagaagttcctctcagtgc

SEQ ID NO: 78 - DNA, 355 bp, Homo sapiens
  misc_feature 69, 87, 186, 192, 220, 227, 251, 278, 339, 346, 350: n = A,T,C or G
ttcgatacaggcaaacatgaactgcaggagggtggtgacgatcatgatgttgccgatggtccggatggncacgaagacgcactggancacgtgcttacgtccttttgctctgttgatggccctgaggggacgcaggacccttatgaccctcagaatcttcacaacgggagatggcactggattgantcccantgacaccagagacaccccaaccaccagnatatcantatattgatgtagttcctgtaganggcccccttgtggaggaaagctccatnagttggtcatcttcaacaggatctcaacagtttccgatggctgtgatgggcatagtcatanttaaccntgtntcgaa

SEQ ID NO: 79 - DNA, 406 bp, Homo sapiens
taagagggtaccagcagaaaggttagtatcatcagatagcatcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttgcctgtaacaagccagattttttaaaatttatattgtaaataatgtgtgtgtgtgtgtgtgtatatatatatatatgtacagttatctaagttaatttaaaagttgtttggtaccctctta

SEQ ID NO: 80 - DNA, 327 bp, Homo sapiens
tttttttttttttactcggctcagtctaatcctttttgtagtcactcataggccagacttagggctaggatgatgattaataagagggatgacataactattagtggcaggttagttgtttgtagggctcatggtaggggtaaaaggagggcaatttctagatcaaataataagaaggtaatagctactaagaagaattttatggagaaagggacgcgggcgggggatatagggtcgaagccgcactcgtaaggggtggatttttctatgtagccgttgagttgtggtagtcaaaatgtaataattattagtagtaagcctaggaga

SEQ ID NO: 81 - DNA, 318 bp, Homo sapiens
tagtctatgcggttgattcggcaatccattatttgctggattttgtcatgtgttttgccaattgcattcataatttattatgcatttatgcttgtatctcctaagtcatggtatataatccatgctttttatgttttgtctgacataaactcttatcagagccctttgcacacagggattcaataaatattaacacagtctacatttatttggtgaatattgcatatctgctgtactgaaagcacattaagtaacaaaggcaagtgagaagaatgaaaagcactactcacaacagttatcatgattgcgcatagacta

SEQ ID NO: 82 - DNA, 338 bp, Homo sapiens
tcttcaacctctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctctt

SEQ ID NO: 83 - DNA, 111 bp, Homo sapiens
agccatttaccacccatccacaaaaaaaaaaaaaaaaaagaaaaatatcaaggaataaaaatagactttgaacaaaaaggaacatttgctggcctgaggaggcatcacccg

SEQ ID NO: 84 - DNA, 224 bp, Homo sapiens
tcgggtgatgcctcctcaggccaagaagataaagcttcagacccctaacacatttccaaaaaggaagaaaggagaaaaaagggcatcatccccgttccgaagggtcagggaggaggaaattgaggtggattcacgagttgcggacaactcctttgatgccaagcgaggtgcagccggagactggggagagcgagccaatcaggttttgaagttcctctcagtgc

SEQ ID NO: 85 - DNA, 348 bp, Homo sapiens
gcactgagaggaacttcgttggaaacgggtttttttcatgtaaggctagacagaagaattctcagtaacttccttgtgttgtgtgtattcaactcacasagttgaacgatcctttacacagagcagacttgtaacactcttwttgtggaatttgcaagtggagatttcagscgctttgaagtsaaaggtagaaaaggaaatatcttcctataaaaactagacagaatgattctcagaaactcctttgtgatgtgtgcgttcaactcacagagtttaacctttcwtttcatagaagcagttaggaaacactctgtttgtaaagtctgcaagtggatagagaccctaacg

SEQ ID NO: 86 - DNA, 293 bp, Homo sapiens
gcactgagaggaacttcyttgtgwtgtktgyattcaactcacagagttgaasswtsmtttacabagwkcaggcttkcaaacactctttttgtmgaatytgcaagwggakatttsrrccrctttgwggycwwysktmgaawmggrwatatcttcwyatmraamctagacagaaksattctcakaawstyyyytgtgawgwstgcrttcaactcacagagktkaacmwtyctkytsatrgagcagttwkgaaactctmtttctttggattctgcaagtggatagagaccctaacg

SEQ ID NO: 87 - DNA, 10 bp, Artificial Sequence
  PCR primer for amplification from breast cancer tumor cDNA
ctcctaggct

SEQ ID NO: 88 - DNA, 10 bp, Artificial Sequence
  PCR primer for amplification from breast cancer tumor cDNA
agtagttgcc

SEQ ID NO: 89 - DNA, 11 bp, Artificial Sequence
  PCR primer for amplification from breast cancer tumor cDNA
ttccgttatgc

SEQ ID NO: 90 - DNA, 10 bp, Artificial Sequence
  PCR primer for amplification from breast cancer tumor cDNA
tggtaaaggg

SEQ ID NO: 91 - DNA, 10 bp, Artificial Sequence
  PCR primer for amplification from breast cancer tumor cDNA
tcggtcatag

SEQ ID NO: 92 - DNA, 10 bp, Artificial Sequence
  PCR primer for amplification from breast cancer tumor cDNA
tacaacgagg

SEQ ID NO: 93 - DNA, 10 bp, Artificial Sequence
  PCR primer for amplification from breast cancer tumor cDNA
tggattggtc

SEQ ID NO: 94 - DNA, 10 bp, Artificial Sequence
  PCR primer for amplification from breast cancer tumor cDNA
ctttctaccc

SEQ ID NO: 95 - DNA, 10 bp, Artificial Sequence
  PCR primer for amplification from breast cancer tumor cDNA
ttttggctcc

SEQ ID NO: 96 - DNA, 10 bp, Artificial Sequence
  PCR primer for amplification from breast cancer tumor cDNA
ggaaccaatc

SEQ ID NO: 97 - DNA, 10 bp, Artificial Sequence
  PCR primer for amplification from breast cancer tumor cDNA
tcgatacagg

SEQ ID NO: 98 - DNA, 10 bp, Artificial Sequence
  PCR primer for amplification from breast cancer tumor cDNA
ggtactaagg

SEQ ID NO: 99 - DNA, 10 bp, Artificial Sequence
  PCR primer for amplification from breast cancer tumor cDNA
agtctatgcg

SEQ ID NO: 100 - DNA, 10 bp, Artificial Sequence
  PCR primer for amplification from breast cancer tumor cDNA
ctatccatgg

SEQ ID NO: 101 - DNA, 10 bp, Artificial Sequence
  PCR primer for amplification from breast cancer tumor cDNA
tctgtccaca

SEQ ID NO: 102 - DNA, 10 bp, Artificial Sequence
  PCR primer for amplification from breast cancer tumor cDNA
aagagggtac

SEQ ID NO: 103 - DNA, 10 bp, Artificial Sequence
  PCR primer for amplification from breast cancer tumor cDNA
cttcaacctc

SEQ ID NO: 104 - DNA, 20 bp, Artificial Sequence
  PCR primer for amplification from breast cancer tumor cDNA
gctcctcttgccttaccaac

SEQ ID NO: 105 - DNA, 20 bp, Artificial Sequence
  PCR primer for amplification from breast cancer tumor cDNA
gtaagtcgagcagtgtgatg

SEQ ID NO: 106 - DNA, 20 bp, Artificial Sequence
  PCR primer for amplification from breast cancer tumor cDNA
gtaagtcgagcagtctgatg

SEQ ID NO: 107 - DNA, 20 bp, Artificial Sequence
  PCR primer for amplification from breast cancer tumor cDNA
gacttagtggaaagaatgta

SEQ ID NO: 108 - DNA, 20 bp, Artificial Sequence
  PCR primer for amplification from breast cancer tumor cDNA
gtaattccgccaaccgtagt

SEQ ID NO: 109 - DNA, 20 bp, Artificial Sequence
  PCR primer for amplification from breast cancer tumor cDNA
atggttgatcgatagtggaa

SEQ ID NO: 110 - DNA, 20 bp, Artificial Sequence
  PCR primer for amplification from breast cancer tumor cDNA
acggggacccctgcattgag

SEQ ID NO: 111 - DNA, 20 bp, Artificial Sequence
  PCR primer for amplification from breast cancer tumor cDNA
tattctagaccattcgctac

SEQ ID NO: 112 - DNA, 20 bp, Artificial Sequence
  PCR primer for amplification from breast cancer tumor cDNA
acataaccactttagcgttc

SEQ ID NO: 113 - DNA, 20 bp, Artificial Sequence
  PCR primer for amplification from breast cancer tumor cDNA
cgggtgatgcctcctcaggc

SEQ ID NO: 114 - DNA, 20 bp, Artificial Sequence
  PCR primer for amplification from breast cancer tumor cDNA
agcatgttgagcccagacac

SEQ ID NO: 115 - DNA, 20 bp, Artificial Sequence
  PCR primer for amplification from breast cancer tumor cDNA
gacaccttgtccagcatctg

SEQ ID NO: 116 - DNA, 20 bp, Artificial Sequence
  PCR primer for amplification from breast cancer tumor cDNA
tacgctgcaacactgtggag

SEQ ID NO: 117 - DNA, 20 bp, Artificial Sequence
  PCR primer for amplification from breast cancer tumor cDNA
cgttagggtctctatccact

SEQ ID NO: 118 - DNA, 20 bp, Artificial Sequence
  PCR primer for amplification from breast cancer tumor cDNA
agactgactcatgtccccta

SEQ ID NO: 119 - DNA, 20 bp, Artificial Sequence
  PCR primer for amplification from breast cancer tumor cDNA
tcatcgctcggtgactcaag

SEQ ID NO: 120 - DNA, 20 bp, Artificial Sequence
  PCR primer for amplification from breast cancer tumor cDNA
caagattccataggctgacc

SEQ ID NO: 121 - DNA, 20 bp, Artificial Sequence
  PCR primer for amplification from breast cancer tumor cDNA
acgtactggtcttgaaggtc

SEQ ID NO: 122 - DNA, 20 bp, Artificial Sequence
  PCR primer for amplification from breast cancer tumor cDNA
gacgcttggccacttgacac

SEQ ID NO: 123 - DNA, 20 bp, Artificial Sequence
  PCR primer for amplification from breast cancer tumor cDNA
gtatcgacgtagtggtctcc

SEQ ID NO: 124 - DNA, 20 bp, Artificial Sequence
  PCR primer for amplification from breast cancer tumor cDNA
tagtgacattacgacgctgg

SEQ ID NO: 125 - DNA, 20 bp, Artificial Sequence
  PCR primer for amplification from breast cancer tumor cDNA
cgggtgatgcctcctcaggc

SEQ ID NO: 126 - DNA, 23 bp, Artificial Sequence
  PCR primer for amplification from breast cancer tumor cDNA
atggctattttcgggggctgaca

SEQ ID NO: 127 - DNA, 22 bp, Artificial Sequence
  PCR primer for amplification from breast cancer tumor cDNA
ccggtatctcctcgtgggtatt

SEQ ID NO: 128 - DNA, 18 bp, Artificial Sequence
  PCR primer for amplification from breast cancer tumor cDNA
ctgcctgagccacaaatg

SEQ ID NO: 129 - DNA, 24 bp, Artificial Sequence
  PCR primer for amplification from breast cancer tumor cDNA
ccggaggaggaagctagaggaata

SEQ ID NO: 130 - DNA, 14 bp, Artificial Sequence
  PCR primer for amplification from breast cancer tumor cDNA
ttttttttttttag

SEQ ID NO: 131 - protein, 18 aa, Artificial Sequence
  Predicited Th Motifs (B-cell epitopes)
SSGGRTFDDFHRYLLVGI

SEQ ID NO: 132 - protein, 22 aa, Artificial Sequence
  Predicited Th Motifs (B-cell epitopes)
  VARIANT 13: Xaa = Any Amino Acid
QGAAQKPINLSKXIEVVQGHDE

SEQ ID NO: 133 - protein, 23 aa, Artificial Sequence
  Predicited Th Motifs (B-cell epitopes)
SPGVFLEHLQEAYRIYTPFDLSA

SEQ ID NO: 134 - protein, 9 aa, Artificial Sequence
  Predicited HLA A2.1 Motifs (T-cell epitopes)
YLLVGIQGA

SEQ ID NO: 135 - protein, 9 aa, Artificial Sequence
  Predicited HLA A2.1 Motifs (T-cell epitopes)
GAAQKPINL

SEQ ID NO: 136 - protein, 9 aa, Artificial Sequence
  Predicited HLA A2.1 Motifs (T-cell epitopes)
  VARIANT 5: Xaa = Any Amino Acid
NLSKXIEVV

SEQ ID NO: 137 - protein, 9 aa, Artificial Sequence
  Predicited HLA A2.1 Motifs (T-cell epitopes)
EVVQGHDES

SEQ ID NO: 138 - protein, 9 aa, Artificial Sequence
  Predicited HLA A2.1 Motifs (T-cell epitopes)
HLQEAYRIY

SEQ ID NO: 139 - protein, 9 aa, Artificial Sequence
  Predicited HLA A2.1 Motifs (T-cell epitopes)
NLAFVAQAA

SEQ ID NO: 140 - protein, 9 aa, Artificial Sequence
  Predicited HLA A2.1 Motifs (T-cell epitopes)
FVAQAAPDS

SEQ ID NO: 141 - DNA, 9388 bp, Homo sapiens
gctcgcggccgcgagctcaattaaccctcactaaagggagtcgactcgatcagactgttactgtgtctatgtagaaagaagtagacataagagattccattttgttctgtactaagaaaaattcttctgccttgagatgctgttaatctgtaaccctagccccaaccctgtgctcacagagacatgtgctgtgttgactcaaggttcaatggatttagggctatgctttgttaaaaaagtgcttgaagataatatgcttgttaaaagtcatcaccattctctaatctcaagtacccagggacacaatacactgcggaaggccgcagggacctctgtctaggaaagccaggtattgtccaagatttctccccatgtgatagcctgagatatggcctcatgggaagggtaagacctgactgtcccccagcccgacatcccccagcccgacatcccccagcccgacacccgaaaagggtctgtgctgaggaggattagtaaaagaggaaggcctctttgcagttgaggtaagaggaaggcatctgtctcctgctcgtccctgggcaatagaatgtcttggtgtaaaacccgattgtatgttctacttactgagataggagaaaacatccttagggctggaggtgagacacgctggcggcaatactgctctttaatgcaccgagatgtttgtataagtgcacatcaaggcacagcacctttccttaaacttatttatgacacagagacctttgttcacgttttcctgctgaccctctccccactattaccctattggcctgccacatccccctctccgagatggtagagataatgatcaataaatactgagggaactcagagaccagtgtccctgtaggtcctccgtgtgctgagcgccggtcccttgggctcacttttctttctctatactttgtctctgtgtctctttcttttctcagtctctcgttccacctgacgagaaatacccacaggtgtggaggggcaggccaccccttcaataatttactagcctgttcgctgacaacaagactggtggtgcagaaggttgggtcttggtgttcaccgggtggcaggcatgggccaggtgggagggtctccagcgcctggtgcaaatctccaagaaagtgcaggaaacagcaccaagggtgattgtaaattttgatttggcgcggcaggtagccattccagcgcaaaaatgcgcaggaaagcttttgctgtgcttgtaggcaggtaggccccaagcacttcttattggctaatgtggagggaacctgcacatccattggctgaaatctccgtctatttgaggctgactgagcgcgttcctttcttctgtgttgcctggaaacggactgtctgcctagtaacatctgatcacgtttcccattggccgccgtttccggaagcccgccctcccatttccggaagcctggcgcaaggttggtctgcaggtggcctccaggtgcaaagtgggaagtgtgagtcctcagtcttgggctattcggccacgtgcctgccggacatgggacgctggagggtcagcagcgtggagtcctggccttttgcgtccacgggtgggaaattggccattgccacggcgggaactgggactcaggctgccccccggccgtttctcatccgtccaccggactcgtgggcgctcgcactggcgctgatgtagtttcctgacctctgacccgtattgtctccagattaaaggtaaaaacggggctttttcagcccactcgggtaaaacgccttttgatttctaggcaggtgttttgttgcacgcctgggagggagtgacccgcaggttgaggtttattaaaatacattcctggtttatgttatgtttataataaagcaccccaacctttacaaaatctcactttttgccagttgtattatttagtggactgtctctgataaggacagccagttaaaatggaattttgttgttgctaattaaaccaatttttagttttggtgtttgtcctaatagcaacaacttctcaggctttataaaaccatatttcttgggggaaatttctgtgtaaggcacagcgagttagtttggaattgttttaaaggaagtaagttcctggttttgatatcttagtagtgtaatgcccaacctggtttttactaaccctgtttttagactctccctttccttaaatcacctagccttgtttccacctgaattgactctcccttagctaagagcgccagatggactccatcttggctctttcactggcagccccttcctcaaggacttaacttgtgcaagctgactcccagcacatccaagaatgcaattaactgttaagatactgtggcaagctatatccgcagttccgaggaattcatccgattgattatgcccaaaagccccgcgtctatcaccttgtaataatcttaaagcccctgcacctggaactattaactttcctgtaaccatttatccttttaacttttttgcttactttatttctgtaaaattgttttaactagacctcccctcccctttctaaaccaaagtataaaagaagatctagccccttcttcagagcggagagaattttgagcattagccatctcttggcggccagctaaataaatggacttttaatttgtctcaaagtgtggcgttttctctaactcgctcaggtacgacatttggaggccccagcgagaaacgtcaccgggagaaacgtcaccgggcgagagccgggcccgctgtgtgctcccccggaaggacagccagcttgtaggggggagtgccacctgaaaaaaaaatttccaggtccccaaagggtgaccgtcttccggaggacagcggatcgactaccatgcgggtgcccaccaaaattccacctctgagtcctcaactgctgaccccggggtcaggtaggtcagatttgactttggttctggcagagggaagcgaccctgatgagggtgtccctcttttgactctgcccatttctctaggatgctagagggtagagccctggttttctgttagacgcctctgtgtctctgtctgggagggaagtggccctgacaggggccatcccttgagtcagtccacatcccaggatgctgggggactgagtcctggtttctggcagactggtctctctctctctctttttctatctctaatctttccttgttcaggtttcttggagaatctctgggaaagaaaaaagaaaaactgttataaactctgtgtgaatggtgaatgaatgggggaggacaagggcttgcgcttgtcctccagtttgtagctccacggcgaaagctacggagttcaagtgggccctcacctgcggttccgtggcgacctcataaggcttaaggcagcatccggcatagctcgatccgagccgggggtttataccggcctgtcaatgctaagaggagcccaagtcccctaagggggagcggccaggcgggcatctgactgatcccatcacgggaccccctccccttgtttgtctaaaaaaaaaaaaagaagaaactgtcataactgtttacatgccctagggtcaactgtttgttttatgtttattgttctgttcggtgtctattgtcttgtttagtggttgtcaaggttttgcatgtcaggacgtcgatattgcccaagacgtctgggtaagaacttctgcaaggtccttagtgctgattttttgtcacaggaggttaaatttctcatcaatcatttaggctggccaccacagtcctgtcttttctgccagaagcaagtcaggtgttgttacgggaatgagtgtaaaaaaacattcgcctgattgggatttctggcaccatgatggttgtatttagattgtcataccccacatccaggttgattggacctcctctaaactaaactggtggtgggttcaaaacagccaccctgcagatttccttgctcacctctttggtcattctgtaacttttcctgtgcccttaaatagcacactgtgtagggaaacctaccctcgtactgctttacttcgtttagattcttactctgttcctctgtggctactctcccatcttaaaaacgatccaagtggtccttttcctcctccctgccccctaccccacacatctcgttttccagtgcgacagcaagttcagcgtctccaggacttggctctgctctcactccttgaacccttaaaagaaaaagctgggtttgagctatttgcctttgagtcatggagacacaaaaggtatttagggtacagatctagaagaagagagagaacacctagatccaactgacccaggagatctcgggctggcctctagtcctcctccctcaatcttaaagctacagtgatgtggcaagtggtatttagctgttgtggtttttctgctctttctggtcatgttgattctgttctttcgatactccagccccccagggagtgagtttctctgtctgtgctgggtttgatatctatgttcaaatcttattaaattgccttcaaaaaaaaaaaaaaaagggaaacacttcctcccagccttgtaagggttggagccctctccagtatatgctgcagaatttttctctcggtttctcagaggattatggagtccgccttaaaaaaggcaagctctggacactctgcaaagtagaatggccaaagtttggagttgagtggccccttgaagggtcactgaacctcacaattgttcaagctgtgtggcgggttgttactgaaactcccggcctccctgatcagtttccctacattgatcaatggctgagtttggtcaggagcaccccttccatggctccactcatgcaccattcataattttacctccaaggtcctcctgagccagaccgtgttttcgcctcgaccctcagccggttcagctcgccctgtactgcctctctctgaagaagaggagagtctccctcacccagtcccaccgccttaaaaccagcctactcccttagggtcatcccatgtctcctcggctatgtcccctgtaggctcatcacccattgcctcttggttgcaaccgtggtgggaggaagtagcccctctactaccactgagagaggcacaagtccctctgggtgatgagtgctccacccccttcctggtttatgtcccttctttctacttctgacttgtataattggaaaacccataatcctcccttctctgaaaagccccaggctttgacctcactgatggagtctgtactctggacacattggcccacctgggatgactgtcaacagctccttttgacccttttcacctctgaagagagggaaagtatccaaagagaggccaaaaagtacaacctcacatcaaccaataggccggaggaggaagctagaggaatagtgattagagacccaattgggacctaattgggacccaaatttctcaagtggagggagaacttttgacgatttccaccggtatctcctcgtgggtattcagggagctgctcagaaacctataaacttgtctaaggcgactgaagtcgtccaggggcatgatgagtcaccaggagtgtttttagagcacctccaggaggcttatcggatttacaccccttttgacctggcagcccccgaaaatagccatgctcttaatttggcatttgtggctcaggcagccccagatagtaaaaggaaactccaaaaactagagggattttgctggaatgaataccagtcagcttttagagatagcctaaaaggtttttgacagtcaagaggttgaaaaacaaaaacaagcagctcaggcagctgaaaaaagccactgataaagcatcctggagtatcagagtttactgttagatcagcctcatttgacttcccctcccacatggtgtttaaatccagctacactacttcctgactcaaactccactattcctgttcatgactgtcaggaactgttggaaactactgaaactggccgacctgatcttcaaaatgtgcccctaggaaaggtggatgccaccgtgttcacagacagtagcagcttcctcgagaagggactacgaaaggccggtgcagctgttaccatggagacagatgtgttgtgggctcaggctttaccagcaaacacctcagcacaaaaggctgaattgatcgccctcactcaggctctccgatggggtaaggatattaacgttaacactgacagcaggtacgcctttgctactgtgcatgtacgtggagccatctaccaggagcgtgggctactcacctcagcaggtggctgtaatccactgtaaaggacatcaaaaggaaaacacggctgttgcccgtggtaaccagaaagctgattcagcagctcaagatgcagtgtgactttcagtcacgcctctaaacttgctgcccacagtctcctttccacagccagatctgcctgacaatcccgcatactcaacagaagaagaaaactggcctcagaactcagagccaataaaaatcaggaaggttggtggattcttcctgactctagaatcttcataccccgaactcttgggaaaactttaatcagtcacctacagtctaccacccatttaggaggagcaaagctacctcagctcctccggagccgttttaagatcccccatcttcaaagcctaacagatcaagcagctctccggtgcacaacctgcgcccaggtaaatgccaaaaaaggtcctaaacccagcccaggccaccgtctccaagaaaactcaccaggagaaaagtgggaaattgactttacagaagtaaaaccacaccgggctgggtacaaataccttctagtactggtagacaccttctctggatggactgaagcatttgctaccaaaaacgaaactgtcaatatggtagttaagtttttactcaatgaaatcatccctcgacgtgggctgcctgttgccatagggtctgataatggaccggccttcgccttgtctatagtttagtcagtcagtaaggcgttaaacattcaatggaagctccattgtgcctatcgaccccagagctctgggcaagtagaacgcatgaactgcaccctaaaaaacactcttacaaaattaatcttagaaaccggtgtaaattgtgtaagtctccttcctttagccctacttagagtaaggtgcaccccttactgggctgggttcttaccttttgaaatcatgtatgggagggcgctgcctatcttgcctaagctaagagatgcccaattggcaaaaatatcacaaactaatttattacagtacctacagtctccccaacaggtacaagatatcatcctgccacttgttcgaggaacccatcccaatccaattcctgaacagacagggccctgccattcattcccgccaggtgacctgttgtttgttaaaaagttccagagagaaggactccctcctgcttggaagagacctcacaccgtcatcacgatgccaacggctctgaaggtggatggcattcctgcgtggattcatcactcccgcatcaaaaaggccaacggagcccaactagaaacatgggtccccagggctgggtcaggccccttaaaactgcacctaagttgggtgaagccattagattaattctttttcttaattttgtaaaacaatgcatagcttctgtcaaacttatgtatcttaagactcaatataacccccttgttataactgaggaatcaatgatttgattccccaaaaacacaagtggggaatgtagtgtccaacctggtttttactaaccctgtttttagactctccctttcctttaatcactcagccttgtttccacctgaattgactctcccttagctaagagcgccagatggactccatcttggctctttcactggcagccgcttcctcaaggacttaacttgtgcaagctgactcccagcacatccaagaatgcaattaactgataagatactgtggcaagctatatccgcagttcccaggaattcgtccaattgattacacccaaaagccccgcgtctatcaccttgtaataatcttaaagcccctgcacctggaactattaacgttcctgtaaccatttatccttttaacttttttgcctactttatttctgtaaaattgttttaactagaccccccctctcctttctaaaccaaagtataaaagcaaatctagccccttcttcaggccgagagaatttcgagcgttagccgtctcttggccaccagctaaataaacggattcttcatgtgtctcaaagtgtggcgttttctctaactcgctcaggtacgaccgtggtagtattttccccaacgtcttatttttagggcacgtatgtagagtaacttttatgaaagaaaccagttaaggaggttttgggatttcctttatcaactgtaatactggttttgattatttatttatttatttattttttttgagaaggagtttcactcttgttgcccaggctggagtgcaatggtgcgatcttggctcactgcaacttccgcctcccaggttcaagcgattctcctgcctcagcctcgagagtagctgggattataggcatgcgccaccacacccagctaattttgtatttttagtaaagatggggtttcttcatgttggtcaagctggtctggaactccccgcctcgggtgatctgcccgcctcggcctccgaaagtgctgggattacaggtgtgatccaccacacccagccgatttatatgtatataaatcacattcctctaaccaaaatgtagtgtttccttccatcttgaatataggctgtagaccccgtgggtatgggacattgttaacagtgagaccacagcagtttttatgtcatctgacagcatctccaaatagccttcatggttgtcactgcttcccaagacaattccaaataacacttcccagtgatgacttgctacttgctattgttacttaatgtgttaaggtggctgttacagacactattagtatgtcaggaattacaccaaaatttagtggctcaaacaatcattttattatgtatgtggattctcatggtcaggtcaggatttcagacagggcacaagggtagcccacttgtctctgtctatgatgtctggcctcagcacaggagactcaacagctggggtctgggaccatttggaggcttgttccctcacatctgatacctggcttgggatgttggaagagggggtgagctgagactgagtgcctatatgtagtgtttccatatggccttgacttccttacagcctggcagcctcagggtagtcagaattcttaggaggcacagggctccagggcagatgctgaggggtcttttatgaggtagcacagcaaatccacccaggatc

SEQ ID NO: 142 - DNA, 419 bp, Homo sapiens
tgtaagtcgagcagtgtgatggaaggaatggtctttggagagagcatatccatctcctcctcactgcctcctaatgtcatgaggtacactgagcagaattaaacagggtagtcttaaccacactatttttagctaccttgtcaagctaatggttaaagaacacttttggtttacacttgttgggtcatagaagttgctttccgccatcacgcaataagtttgtgtgtaatcagaaggagttaccttatggtttcagtgtcattctttagttaacttgggagctgtgtaatttaggctttgcgtattatttcacttctgttctccacttatgaagtgattgtgtgttcgcgtgtgtgtgcgtgcgcatgtgcttccggcagttaacataagcaaatacccaacatcacactgctcgactt

SEQ ID NO: 143 - DNA, 402 bp, Homo sapiens
tgtaagtcgagcagtgtgatgtccactgcagtgtgttgctgggaacagttaatgagcaaattgtatacaatggctagtacattgaccgggatttgttgaagctggtgagtgttatgacttagcctgttagactagtctatgcacatggctctggtcaactaccgctctctcatttctccagataaatcccccatgctttatattctcttccaaacatactatcctcatcaccacatagttcctttgttaatgctttgttctagactttcccttttctgttttcttattcaaacctatatctctttgcatagattgtaaattcaaatgccctcagggtgcaggcagttcatgtaagggagggaggctagccagtgagatctgcatcacactgctcgacttaca

SEQ ID NO: 144 - DNA, 224 bp, Homo sapiens; identical to SEQ ID NO: 84

SEQ ID NO: 145 - DNA, 111 bp, Homo sapiens; identical to SEQ ID NO: 83

SEQ ID NO: 146 - DNA, 585 bp, Homo sapiens
tagcatgttgagcccagacacttgtagagagaggaggacagttagaagaagaagaaaagtttttaaatgctgaaagttactataagaaagctttggctttggatgagacttttaaagatgcagaggatgctttgcagaaacttcataaatatatgcaggtgattccttatttcctcctagaaatttagtgatatttgaaataatgcccaaacttaattttctcctgaggaaaactattctacattacttaagtaaggcattatgaaaagtttctttttaggtatagtttttcctaattgggtttgacattgcttcatagtgcctctgtttttgtccataatcgaaagtaaagatagctgtgagaaaactattacctaaatttggtatgttgttttgagaaatgtccttatagggagctcacctggtggtttttaaattattgttgctactataattgagctaattataaaaacctttttgagacatattttaaattgtcttttcctgtaatactgatgatgatgttttctcatgcattttcttctgaattgggaccattgctgctgtgtctgggctcacatgcta

SEQ ID NO: 147 - DNA, 579 bp, Homo sapiens
  misc_feature 383, 453, 465, 501: n = A,T,C or G
tagcatgttgagcccagacactgggcagcgggggtggccacggcagctcctgccgagcccaagcgtgtttgtctgtgaaggaccctgacgtcacctgccaggctagggaggggtcaatgtggagtgaatgttcaccgactttcgcaggagtgtgcagaagccaggtgcaacttggtttgcttgtgttcatcacccctcaagatatgcacactgctttccaaataaagcatcaactgtcatctccagatggggaagactttttctccaaccagcaggcaggtccccatccactcagacaccagcacgtccaccttctcgggcagcaccacgtcctccaccttctgctggtacacggtgatgatgtcagcaaagccgttctgcangaccagctgccccgtgtgctgtgccatctcactggcctccaccgcgtacaccgctctaggccgcgcatantgtgcacagaanaaatgatgatccagtcccacagcccacgtccaagangactttatccgtcagggattctttattctgcaggatgacctgtggtattaattgttcgtgtctgggctcaacatgcta

SEQ ID NO: 148 - DNA, 249 bp, Homo sapiens
tgacaccttgtccagcatctgcaagccaggaagagagtcctcaccaagatccccaccccgttggcaccaggatcttggacttccaatctccagaactgtgagaaataagtatttgtcgctaaataaatctttgtggtttcagatatttagctatagcagatcaggctgactaagagaaaccccataagagttacatactcattaatctccgtctctatccccaggtctcagatgctggacaaggtgtca

SEQ ID NO: 149 - DNA, 255 bp, Homo sapiens
tgacaccttgtccagcatctgctattttgtgactttttaataatagccattctgactggtgtgagatggtaactcattgtgggtttggtctgcatttctctaatgatcagtgatattaagctttttttaaatatgcttgttgaccacatgtatatcatcttttgagaagtgtctgttcatatcctttgcccactttttaatttttttatcttgtaaatttgtttaatttccttacagatgctggacaaggtgtca

SEQ ID NO: 150 - DNA, 318 bp, Homo sapiens
ttacgctgcaacactgtggaggccaagctgggatcacttcttcattctaactggagaggagggaagttcaagtccagcagagggtgggtgggtagacagtggcactcagaaatgtcagctggacccctgtccccgcataggcaggacagcaaggctgtggctctccagggccagctgaagaacaggacactgtctccgctgccacaaagcgtcagagactcccatctttgaagcacggccttcttggtcttcctgcacttccctgttctgttagagacctggttatagacaaggcttctccacagtgttgcagcgtaa

SEQ ID NO: 151 - DNA, 323 bp, Homo sapiens
  misc_feature 2, 7, 10, 13, 14, 23, 26, 32, 44, 54, 56, 67, 74, 75,81, 87, 104, 105, 109, 111, 120, 123, 124, 136, 137, 138, 151,155, 162, 168, 171, 176, 184, 186, 196, 215, 231, 239, 252,265, 288, 318: n = A,T,C or G
tnacgcngcnacnntgtagaganggnaaggcnttccccacattnccccttcatnanagaattattcnaccaagnntgaccnatgccntttatgacttacatgcnnactncntaatctgtntcnngccttaaaagcnnntccactacatgcntcancactgtntgtgtnacntcatnaactgtcngnaataggggcncataactacagaaatgcanttcatactgcttccantgccatcngcgtgtggccttncctactcttcttntattccaagtagcatctctggantgcttccccactctccacattgttgcagcnataat

SEQ ID NO: 152 - DNA, 311 bp, Homo sapiens
tcaagattccataggctgaccagtccaaggagagttgaaatcatgaaggagagtctatctggagagagctgtagttttgagggttgcaaagacttaggatggagttggtgggtgtggttagtctctaaggttgattttgttcataaatttcatgccctgaatgccttgcttgcctcaccctggtccaagccttagtgaacacctaaaagtctctgtcttcttgctctccaaacttctcctgaggatttcctcagattgtctacattcagatcgaagccagttggcaaacaagatgcagtccagagggtcag

SEQ ID NO: 153 - DNA, 332 bp, Homo sapiens
caagattccataggctgaccaggaggctattcaagatctctggcagttgaggaagtctctttaagaaaatagtttaaacaatttgttaaaatttttctgtcttacttcatttctgtagcagttgatatctggctgtcctttttataatgcagagtgggaactttccctaccatgtttgataaatgttgtccaggctccattgccaataatgtgttgtccaaaatgcctgtttagtttttaaagacggaactccaccctttgcttggtcttaagtatgtatggaatgttatgataggacatagtagtagcggtggtcagcctatggaatcttg

SEQ ID NO: 154 - DNA, 345 bp, Homo sapiens
  misc_feature 154, 224, 297, 330: n = A,T,C or G
tcaagattccataggctgacctggacagagatctcctgggtctggcccaggacagcaggctcaagctcagtggagaaggtttccatgaccctcagattcccccaaaccttggattgggtgacattgcatctcctcagagagggaggagatgtangtctgggcttccacagggacctggtattttaggatcagggtaccgctggcctgaggcttggatcattcanagcctgggggtggaatggctggcagcctgtggccccattgaaataggctctggggcactccctctgttcctanttgaacttgggtaaggaacaggaatgtggtcancctatggaatcttga

SEQ ID NO: 155 - DNA, 295 bp, Homo sapiens
  misc_feature 46, 199, 252, 266: n = A,T,C or G
gacgcttggccacttgacacattaaacagttttgcataatcactancatgtatttctagtttgctgtctgctgtgatgccctgccctgattctctggcgttaatgatggcaagcataatcaaacgctgttctgttaattccaagttataactggcattgattaaagcattatctttcacaactaaactgttcttcatanaacagcccatattattatcaaattaagagacaatgtattccaatatcctttanggccaatatatttnatgtcccttaattaagagctactgtccgt

SEQ ID NO: 156 - DNA, 406 bp, Homo sapiens
  misc_feature 172, 178, 332, 338, 342, 381, 400, 402: n = A,T,C or G
gacgcttggccacttgacactgcagtgggaaaaccagcatgagccgctgcccccaaggaacctcgaagcccaggcagaggaccagccatcccagcctgcaggtaaagtgtgtcacctgtcaggtgggcttggggtgagtgggtgggggaagtgtgtgtgcaaagggggtgtnaatgtntatgcgtgtgagcatgagtgatggctagtgtgactgcatgtcagggagtgtgaacaagcgtgcgggggtgtgtgtgcaagtgcgtatgcatatgagaatatgtgtctgtggatgagtgcatttgaaagtctgtgtgtgtgcgtgtggtcatganggtaanttantgactgcgcaggatgtgtgagtgtgcatggaacactcantgtgtgtgtcaagtggccnancgtc

SEQ ID NO: 157 - DNA, 208 bp, Homo sapiens
  misc_feature 115, 119, 182, 187: n = A,T,C or G
tgacgcttggccacttgacacactaaagggtgttactcatcactttcttctctcctcggtggcatgtgagtgcatctattcacttggcactcatttgtttggcagtgactgtaanccanatctgatgcatacaccagcttgtaaattgaataaatgtctctaatactatgtgctcacaatanggtangggtgaggagaaggggagaga

SEQ ID NO: 158 - DNA, 547 bp, Homo sapiens
  misc_feature 235: n = A,T,C or G
cttcaacctccttcaacctccttcaacctcctggattcaaacaatcatcccacctcagactccttagtagctgagactacagactcacgccactacatctggctaaatttttgtagagatagggtttcatcatgttgccctggctggtctcaaactcctgacctcaagcaatgtgcccacctcagcctcccaaagtgctgggattacaggcataagccaccatgcccagtccatntttaatctttcctaccacattcttaccacactttcttttatgtttagatacataaatgcttaccattatgatacaattgcccacagtattaagacagtaacatgctgcacaggtttgtagcctaggaacagtaggcaataccacatagcttaggtgtgtggtagactataccatctaggtttgtgtaagttacactttatgctgtttacacaatgacaaaaccatctaatgatgcatttctcagaatgtatccttgtcagtaagctatgatgtacagggaacactgcccaaggacacagatattgtacctgt

SEQ ID NO: 159 - DNA, 203 bp, Homo sapiens; identical to SEQ ID NO: 65

SEQ ID NO: 160 - DNA, 402 bp, Homo sapiens
tgtaagtcgagcagtgtgatgggtggaacagggttgtaagcagtaattgcaaactgtatttaaacaataataataatatttagcatttatagagcactttatatcttcaaagtacttgcaaacattayctaattaaataccctctctgattataatctggatacaaatgcacttaaactcaggacagggtcatgagaraagtatgcatttgaaagttggtgctagctatgctttaaaaacctatacaatgatgggraagttagagttcagattctgttggactgtttttgtgcatttcagttcagcctgatggcagaattagatcatatctgcactcgatgactytgcttgataacttatcactgaaatctgagtgttgatcatcacactgctcgacttaca

SEQ ID NO: 161 - DNA, 193 bp, Homo sapiens
agcatgttgagcccagacactgaccaggagaaaaaccaaccaatagaaacacgcccagacactgaccaggagaaaaaccaaccaataaaaacaggcccggacataagacaaataataaaattagcggacaaggacatgaaaacagctattgtaagagcggatatagtggtgtgtgtctgggctcaacatgcta

SEQ ID NO: 162 - DNA, 147 bp, Homo sapiens
tgttgagcccagacactgaccaggagaaaaaccaaccaataaaaacaggcccggacataagacaaataataaaattagcggacaaggacatgaaaacagctattgtaagagcggatatagtggtgtgtgtctgggctcaacatgcta

SEQ ID NO: 163 - DNA, 294 bp, Homo sapiens
tagcatgttgagcccagacacaaatctttccttaagcaataaatcatttctgcatatgtttttaaaaccacagctaagccatgattattcaaaaggactattgtattgggtattttgatttgggttcttatctccctcacattatcttcatttctatcattgacctcttatcccagagactctcaaacttttatgttatacaaatcacattctgtctcaaaaaatatctcacccacttctcttctgtttctgcgtgtgtatgtgtgtgtgtgtgtgtctgggctcaacatgcta

SEQ ID NO: 164 - DNA, 412 bp, Homo sapiens
  misc_feature 292: n = A,T,C or G
cgggattggctttgagctgcagatgctgcctgtgaccgcacccggcgtggaacagaaagccacctggctgcaagtgcgccagagccgccctgactacgtgctgctgtggggctggggcgtgatgaactccaccgccctgaaggaagcccaggccaccggatacccccgcgacaagatgtacggcgtgtggtgggccggtgcggagcccgatgtgcgtgacgtgggcgaaggcgccaagggctacaacgcgctggctctgaacggctacggcacgcagtccaaggtgatccangacatcctgaaacacgtgcacgacaagggccagggcacggggcccaaagacgaagtgggctcggtgctgtacacccgcggcgtgatcatccagatgctggacaaggtgtcaatcactaat

SEQ ID NO: 165 - DNA, 361 bp, Homo sapiens
ttgacaccttgtccagcatctgcatctgatgagagcctcagatggctaccactaatggcagaaggcaaaggagaacaggcattgtatggcaagaaaggaagaaagagagaggggagaaaggtgctaggttcttttcaacaaccagttcttgatggaactgagagtaagagctcaaggccaggtgtggtgactccaaccagtaatcccaacattttaggaggctgaggcaggcagatgtcttgaccccatgagtttgtgaccagcctgaacaacatcatgagactccatctctacaataattacaaaaattaatcaggcattgtggtatgccctgtagtcccagatgctggacaaggtgtca

SEQ ID NO: 166 - DNA, 427 bp, Homo sapiens
twgactgactcatgtcccctacacccaactatcttctccaggtggccaggcatgatagaatctgatcctgacttaggggaatattttctttttacttcccatcttgattccctgccggtgagtttcctggttcagggtaagaaaggagctcaggccaaagtaatgaacaaatccatcctcacagacgtacagaataagagaacwtggacwtagccagcagaacmcaaktgaaamcagaacmcttamctaggatracaamcmcrraratarktgcycmcmcwtataatagaaaccaaacttgtatctaattaaatatttatccacygtcagggcattagtggttttgataaatacgctttggctaggattcctgaggttagaatggaaraacaattgcamcgagggtaggggacatgagtcaktctaa

SEQ ID NO: 167 - DNA, 500 bp, Homo sapiens
  misc_feature 288, 303, 318, 326: n = A,T,C or G
aacgtcgcatgctcccggccgccatggccgcgggatagactgactcatgtcccctaagatagaggagacacctgctaggtgtaaggagaagatggttaggtctacggaggctccagggtgggagtagttccctgctaagggagggtagactgttcaacctgttcctgctccggcctccactatagcagatgcgagcaggagtaggagagagggaggtaagagtcagaagcttatgttgtttatgcggggaaacgccrtatcgggggcagccragttattaggggacantrtagwyartcwagntagcatccaaagcgngggagttntcccatatggttggacctgcaggcggccgcattagtgattagcatgtgagccccagacacgcatagcaacaaggacctaaactcagatcctgtgctgattacttaacatgaattattgtatttatttaacaactttgagttatgaggcatattattaggtccatattacctgga

SEQ ID NO: 168 - DNA, 358 bp, Homo sapiens
ttcatcgctcggtgactcaagcctgtaatcccagaactttgggaggccgaggggagcagatcacctgaggttgggagtttgagaccagcctggccaacatggtgacaacccgtctctgctaaaaatacaaaaattagccaagcatggtggcatgcacttgtaatcccagctactcgggaggctgaggcaggagaatcacttgaggccaggaggcagaggttgcagtgaggcagaggttgagatcatgccactgcactccagcctgggcaacagagtaagactccatctcaaaaaaaaaaaaaaaaaagaatgatcagagccacaaatacagaaaaccttgagtcaccgagcgatgaaa

SEQ ID NO: 169 - DNA, 1265 bp, Homo sapiens
ttctgtccacaccaatcttagagctctgaaagaatttgtctttaaatatcttttaatagtaacatgtattttatggaccaaattgacattttcgactattttttcccaaaaaaagtcaggtgaatttcagcacactgagttgggaatttcttatcccagaagwcggcacgagcaatttcatatttatttaagattgattccatactccgttttcaaggagaatccctgcagtctccttaaaggtagaacaaatactttctatttttttttcaccattgtgggattggactttaagaggtgactctaaaaaaacagagaacaaatatgtctcagttgtattaagcacggacccatattatcatattcacttaaaaaaatgatttcctgtgcaccttttggcaacttctcttttcaatgtagggaaaaacttagtcaccctgaaaacccacaaaataaataaaacttgtagatgtgggcagaargtttgggggtggacattgtatgtgtttaaattaaaccctgtatcactgagaagctgttgtatgggtcagagaaaatgaatgcttagaagctgttcacatcttcaagagcagaagcaaaccacatgtctcagctatattattatttattttttatgcataaagtgaatcatttcttctgtattaatttccaaagggttttaccctctatttaaatgctttgaaaaacagtgcattgacaatgggttgatatttttctttaaaagaaaaatataattatgaaagccaagataatctgaagcctgttttattttaaaactttttatgttctgtggttgatgttgtttgtttgtttgtttctattttgttggttttttactttgttttttgttttgttttgttttggtttdgcatactacatgcagtttctttaaccaatgtctgtttggctaatgtaattaaagttgttaatttatatgagtgcatttcaactatgtcaatggtttcttaatatttattgtgtagaagtactggtaatttttttatttacaatatgtttaaagagataacagtttgatatgttttcatgtgtttatagcagaagttatttatttctatggcattccagcggatattttggtgtttgcgaggcatgcagtcaatattttgtacagttagtggacagtattcagcaacgcctgatagcttctttggccttatgttaaataaaaagacctgtttgggatgtaaaaaaaaaaaaaaaaaaaaaaaaaaaaaaaaaaaaaa

SEQ ID NO: 170 - DNA, 383 bp, Homo sapiens
tgtaagtcgagcagtgtgatgacgatattcttcttattaatgtggtaattgaacaaatgatctgtgatactgatcctgagctaggaggcgctgttcagttaatgggacttcttcgtactctaattgatccagagaacatgctggctacaactaataaaaccgaaaaaagtgaatttctaaattttttctacaaccattgtatgcatgttctcacagcaccacttttgaccaatacttcagaagacaaatgtgaaaaggataatatagttggatcaaacaaaaacaacacaatttgtcccgataattatcaaacagcacagctacttgccttaattttagagttactcacattttgtgtggaacatcacactgctcgacttaca

SEQ ID NO: 171 - DNA, 383 bp, Homo sapiens
tgggcaccttcaatatcgcaagttaaaaataatgttgagtttattatacttttgacctgtttagctcaacagggtgaaggcatgtaaagaatgtggacttctgaggaattttcttttaaaaagaacataatgaagtaacattttaattactcaaggactacttttggttgaagtttataatctagatacctctactttttgtttttgctgttcgacagttcacaaagaccttcagcaatttacagggtaaaatcgttgaagtagtggaggtgaaactgaaatttaaaattattctgtaaatactatagggaaagaggctgagcttagaatcttttggttgttcatgtgttctgtgctcttatcatcacactgctcgacttaca

SEQ ID NO: 172 - DNA, 699 bp, Homo sapiens
  misc_feature 641: n = A,T,C or G
tcgggtgatgcctcctcaggcttgtcgttagtgtacacagagctgctcatgaagcgacagcggctgcccctggcacttcagaacctcttcctctacacttttggtgcgcttctgaatctaggtctgcatgctggcggcggctctggcccaggcctcctggaaagtttctcaggatgggcagcactcgtggtgctgagccaggcactaaatggactgctcatgtctgctgtcatggagcatggcagcagcatcacacgcctctttgtggtgtcctgctcgctggtggtcaacgccgtgctctcagcagtcctgctacggctgcagctcacagccgccttcttcctggccacattgctcattggcctggccatgcgcctgtactatggcagccgctagtccctgacaacttccaccctgattccggaccctgtagattgggcgccaccaccagatccccctcccaggccttcctccctctcccatcagcggccctgtaacaagtgccttgtgagaaaagctggagaagtgagggcagccaggttattctctggaggttggtggatgaaggggtacccctaggagatgtgaagtgtgggtttggttaaggaaatgcttaccatcccccacccccaaccaagttnttccagactaaagaattaaggtaacatcaatacctaggcctgaggaggcatcacccga

SEQ ID NO: 173 - DNA, 701 bp, Homo sapiens
tcgggtgatgcctcctcaggccagatcaaacttggggttgaaaactgtgcaaagaaatcaatgtcggagaaagaattttgcaaaagaaaaatgcctaatcagtactaatttaataggtcacattagcagtggaagaagaaatgttgatattttatgtcagctattttataatcaccagagtgcttagcttcatgtaagccatctcgtattcattagaaataagaacaattttattcgtcggaaagaacttttcaatttatagcatcttaattgctcaggattttaaattttgataaagaaagctccacttttggcaggagtagggggcagggagagaggaggctccatccacaaggacagagacaccagggccagtagggtagctggtggctggatcagtcacaacggactgacttatgccatgagaagaaacaacctccaaatctcagttgcttaatacaacacaagctcatttcttgctcacgttacatgtcctatgtagatcaacagcaggtgactcagggacccaggctccatctccatatgagcttccatagtcaccaggacacgggctctgaaagtgtcctccatgcagggacacatgcctcttcctttcattgggcagagcaagtcacttatggccagaagtcacactgcagggcagtgccatcctgctgtatgcctgaggaggcatcacccga

SEQ ID NO: 174 - DNA, 700 bp, Homo sapiens
  misc_feature 19: n = A,T,C or G
tcgggtgatgcctcctcangcccctaaatcagagtccagggtcagagccacaggagacagggaaagacatagattttaaccggcccccttcaggagattctgaggctcagttcactttgttgcagtttgaacagaggcagcaaggctagtggttaggggcacggtctctaaagctgcactgcctggatctgcctcccagctctgccaggaaccagctgcgtggccttgagctgctgacacgcagaaagccccctgtggacccagtctcctcgtctgtaagatgaggacaggactctaggaaccctttcccttggtttggcctcactttcacaggctcccatcttgaactctatctactcttttcctgaaaccttgtaaaagaaaaaagtgctagcctgggcaacatggcaaaaccctgtctctacaaaaaatacaaaaattagttgggtgtggtggcatgtgcctgtagtcccagccacttgggaggtgctgaggtgggaggatcacttgagcccgggaggtggaggttgcagtgagccaagatcatgccactgcactccagcctgagtaatagagtaagactctgtctcaaaaacaacaacaacaacagtgagtgtgcctctgtttccgggttggatggggcaccacatttatgcatctctcagatttggacgctgcagcctgaggaggcatcacccga

SEQ ID NO: 175 - DNA, 484 bp, Homo sapiens
  misc_feature 30: n = A,T,C or G
tatagggcgaattgggcccgagttgcatgntcccggccgccatggccgcgggattcgggtgatgcctcctcaggcttgtctgccacaagctacttctctgagctcagaaagtgccccttgatgagggaaaatgtcctactgcactgcgaatttctcagttccattttacctcccagtcctccttctaaaccagttaataaattcattccacaagtatttactgattacctgcttgtgccagggactattctcaggctgaagaaggtgggaggggagggcggaacctgaggagccacctgagccagctttatatttcaaccatggctggcccatctgagagcatctccccactctcgccaacctatcggggcatagcccagggatgcccccaggcggcccaggttagatgcgtccctttggcttgtcagtgatgacatacaccttagctgcttagctggtgctggcctgaggaggcatcacccga

SEQ ID NO: 176 - DNA, 432 bp, Homo sapiens
tcgggtgatgcctcctcagggctcaagggatgagaagtgacttctttctggagggaccgttcatgccacccaggatgaaaatggatagggacccacttggaggacttgctgatatgtttggacaaatgccaggtagcggaattggtactggtccaggagttatccaggatagattttcacccaccatgggacgtcatcgttcaaatcaactcttcaatggccatgggggacacatcatgcctcccacacaatcgcagtttggagagatgggaggcaagtttatgaaaagccaggggctaagccagctctaccataaccagagtcagggactcttatcccagctgcaaggacagtcgaaggatatgccacctcggttttctaagaaaggacagcttaatgcagatgagattagcctgaggaggcatcacccga

SEQ ID NO: 177 - DNA, 788 bp, Homo sapiens
tagcatgttgagcccagacacagtagcatttgtgccaatttctggttggaatggtgacaacatgctggagccaagtgctaacatgccttggttcaagggatggaaagtcacccgtaaggatggcaatgccagtggaaccacgctgcttgaggctctggactgcatcctaccaccaactcgcccaactgacaagcccttgcgcctgcctctccaggatgtctacaaaattggtggtattggtactgttcctgttggccgagtggagactggtgttctcaaacccggtatggtggtcacctttgctccagtcaacgttacaacggaagtaaaatctgtcgaaatgcaccatgaagctttgagtgaagctcttcctggggacaatgtgggcttcaatgtcaagaatgtgtctgtcaaggatgttcgtcgtggcaacgttgctggtgacagcaaaaatgacccaccaatggaagcagctggcttcactgctcaggtgattatcctgaaccatccaggccaaataagtgccggctatgcccctgtattggattgccacacggctcacattgcatgcaagtttgctgagctgaaggaaaagattgatcgccgttctggtaaaaagctggaagatggccctaaattcttgaagtctggtgatgctgccattgttgatatggttcctggcaagcccatgtgtgttgagagcttctcagactatccacctttgggtcgctttgctgttcgtgatatgagacagacagttgcggtgggtgtctgggctcaacatgcta

SEQ ID NO: 178 - DNA, 786 bp, Homo sapiens
tagcatgttgagcccagacacctgtgtttctgggagctctggcagtggcggattcataggcacttgggctgcactttgaatgacacacttggctttattagattcactagtttttaaaaaattgttgttcgtttcttttcattaaaggtttaatcagacagatcagacagcataattttgtatttaatgacagaaacgttggtacatttcttcatgaatgagcttgcattctgaagcaagagcctacaaaaggcacttgttataaatgaaagttctggctctagaggccagtactctggagtttcagagcagccagtgattgttccagtcagtgatgcctagttatatagaggaggagtacactgtgcactcttctaggtgtaagggtatgcaactttggatcttaaaattctgtacacatacacactttatatatatgtatgtatgtatgaaaacatgaaattagtttgtcaaatatgtgtgtgtttagtattttagcttagtgcaactatttccacattatttattaaattgatctaagacactttcttgttgacaccttgaatattaatgttcaagggtgcaatgtgtattcctttagattgttaaagcttaattactatgatttgtagtaaattaacttttaaaatgtatttgagcccttctgtagtgtcgtagggctcttacagggtgggaaagattttaattttccagttgctaattgaacagtatggcctcattatatattttgatttataggagtttgtgtctgggctcaacatgcta

SEQ ID NO: 179 - DNA, 796 bp, Homo sapiens
tagcatgttgagcccagacactggttacaagaccagacctgcttcctccatatgtaaacagcttttaaaaagccagtgaacctttttaatactttggcaaccttctttcacaggcaaagaacacccccatccgccccttgtttggagtgcagagtttggctttggttctttgccttgcctggagtatacttctaattcctgttgtcctgcacaagctgaataccgagctacccaccgccacccaggccaggtttccactcatttattactttatgtttctgttccattgctggtccacagaaataagttttcctttggaggaatgtgattatacccctttaatttcctccttttgcttttttttaatatcattggtatgtgtttggcccagaggaaactgaaattcaccatcatcttgactggcaatcccattaccatgctttttttaaaaaacgtaatttttcttgccttacattggcagagtagcccttcctggctactggcttaatgtagtcactcagtttctaggtggcattaggcatgagacctgaagcacagactgtcttaccacaaaaggtgacaagatctcaaaccttagccaaagggctatgtcaggtttcaatgctatctgcttctgttcctgctcactgttctggattttgtccttcttcatccctagcaccagaatttcccagtctccctccctaccttcccttgttttaattctaatctatcagcaaaataacttttcaaatgttttaaccggtatctccatgtgtctgggctcaacatgcta

SEQ ID NO: 180 - DNA, 488 bp, Homo sapiens
ggatgtgctgcaaggcgattaagttgggtaacgccagggttttcccagtcacgacgttgtaaaacgacggccagtgaattgtaatacgactcactatagggcgaattgggcccgacgtcgcatgctcccggccgccatggccgcgggatagcatgttgagcccagacacctgcaggtcatttggagagatttttcacgttaccagcttgatggtctttttcaggaggagagacactgagcactcccaaggtgaggttgaagatttcctctagatagccggataagaagactaggagggatgcctagaaaatgattagcatgcaaatttctacctgccatttcagaactgtgtgtcagcccacattcagctgcttcttgtgaactgaaaagagagaggtattgagacttttctgatggccgctctaacattgtaacacagtaatctgtgtgtgtgtgggtgtgtgtgtgtgtctgggctcaacatgcta

SEQ ID NO: 181 - DNA, 317 bp, Homo sapiens
tagcatgttgagcccagacacggcgacggtacctgatgagtggggtgatggcacctgtgaaaaggaggaacgtcatcccccatgatattggggacccagatgatgaaccatggctccgcgtcaatgcatatttaatccatgatactgctgattggaaggacctgaacctgaagtttgtgctgcaggtttatcgggactattacctcacgggtgatcaaaacttcctgaaggacatgtggcctgtgtgtctagtaagggatgcacatgcagtggccagtgtgccaggggtatggttggtgtctgggctcaacatgcta

SEQ ID NO: 182 - DNA, 507 bp, Homo sapiens
  misc_feature 493: n = A,T,C or G
tagcatgttgagcccagacactggctgttagccaaatcctctctcagctgctccctgtggtttggtgactcaggattacagaggcatcctgtttcagggaacaaaaagattttagctgccagcagagagcaccacatacattagaatggtaaggactgccacctccttcaagaacaggagtgagggtggtggtgaatgggaatggaagcctgcattccctgatgcatttgtgctctctcaaatcctgtcttagtcttaggaaaggaagtaaagtttcaaggacggttccgaactgctttttgtgtctgggctcaacatgctatcccgcggccatggcggccgggagcatgcgacgtcgggcccaattcgccctatagtgagtcgtattacaattcactggccgtcgttttacaacgtcgtgactgggaaaaccctggcgttacccaacttaatcgccttgcagcacatccccctttcccagctggcgtaatancgaaaaggcccgca

SEQ ID NO: 183 - DNA, 227 bp, Homo sapiens
gatttacgctgcaacactgtggaggtagccctggagcaaggcaggcatggatgcttctgcaatccccaaatggagcctggtatttcagccaggaatctgagcagagccccctctaattgtagcaatgataagttattctctttgttcttcaaccttccaatagccttgagcttccaggggagtgtcgttaatcattacagcctggtctccacagtgttgcagcgtaa

SEQ ID NO: 184 - DNA, 225 bp, Homo sapiens
ttacgctgcaacactgtggagcagattaacatcagacttttctatcaacatgactggggttactaaaaagacaacaaatcaatggcttcaaaagtctaaggaataatttcgatacttcaactttataaaacctgacaaaactatcaatcaagcataaagacagatgaagaacatttccagattttggccaatcagatattttacctccacagtgttgcagcgtaa

SEQ ID NO: 185 - DNA, 597 bp, Homo sapiens
ggcccgacgtcgcatgctcccggccgccatggccgcgggattcgttagggtctctatccactgggacccataggctagtcagagtatttagagttgagttcctttctgcttcccagaatttgaaagaaaaggagtgaggtgatagagctgagagatcagatttgcctctgaagcctgttcaagatgtatgtgctcagaccccaccactggggcctgtgggtgaggtcctgggcatctatttgaatgaattgctgaaggggagcactatgccaaggaaggggaacccatcctggcactggcacaggggtcaccttatccagtgctcagtgcttctttgctgctacctggttttctctcatatgtgaggggcaggtaagaagaagtgcccrgtgttgtgcgagttttagaacatctaccagtaagtggggaagtttcacaaagcagcagctttgttttgtgtattttcaccttcagttagaagaggaaggctgtgagatgaatgttagttgagtggaaaagacgggtaagcttagtggatagagaccctaacgaatcactagtgcggccgccttgcaggtcgaccatatgggagagctc

SEQ ID NO: 186 - DNA, 597 bp, Homo sapiens
ggcccgaagttgcatgttcccggccgccatggccgcgggattcgttagggtctctatccactacctaaaaaatcccaaacatataactgaactcctcacacccaattggaccaatccatcaccccagaggcctacagatcctcctttgatacataagaaaatttccccaaactacctaactatatcattttgcaagatttgttttaccaaattttgatggcctttctgagcttgtcagtgtgaaccactattacgaacgatcggatattaactgcccctcaccgtccaggtgtagctggcaacatcaagtgcagtaaatattcattaagttttcacctactaaggtgcttaaacaccctagggtgccatgtcggtagcagatcttttgatttgtttttatttcccataagggtcctgttcaaggtcaatcatacatgtagtgtgagcagctagtcactatcgcatgacttggagggtgataatagaggcctcctttgctgttaaagaactcttgtcccagcctgtcaaagtggatagagaccctaacgaatcactagtgcggccgcctgcaggtcgaccatatgggagagctcccaa

SEQ ID NO: 187 - DNA, 324 bp, Homo sapiens
tcgttagggtctctatccacttgcaggtaaaatccaatcctgtgtatatcttatagtcttccatatgtagtggttcaagagactgcagttccagaaagactagccgagcccatccatgtcttccacttaaccctgctttgggttacacatcttaacttttctgttcaagtttctctgtgtagtttatagcatgagtattgggawaatgccctgaaacctgacatgagatctgggaaacacaaacttactcaataagaatttctcccatatttttatgatggaaaaatttcacatgcacagaggagtggatagagaccctaacga

SEQ ID NO: 188 - DNA, 178 bp, Homo sapiens
  misc_feature 46: n = A,T,C or G
gcgcggggattcggggtgatacctcctcatgccaaaatacaacgtntaatttcacaacttgccttccaatttacgcattttcaatttgctctccccatttgttgagtcacaacaaacaccattgcccagaaacatgtattacctaacatgcacatactcttaaaactactcatccctt

SEQ ID NO: 189 - DNA, 367 bp, Homo sapiens
tgacaccttgtccagcatctgacacagtcttggctcttggaaaatattggataaatgaaaatgaatttctttagcaagtggtataagctgagaatatacgtatcacatatcctcattctaagacacattcagtgtccctgaaattagaataggacttacaataagtgtgttcactttctcaatagctgttattcaattgatggtaggccttaaaagtcaaagaaatgagagggcatgtgaaaaaaagctcaacatcactgatcattagaaaacttccattcaaacccccaatgagataccatctcataccagtcagaatggctattattaaaaagtcaaaaaataacagatgctggacaaggtgtca

SEQ ID NO: 190 - DNA, 369 bp, Homo sapiens
  misc_feature 323: n = A,T,C or G
gacaccttgtccagcatctgacaacgctaacagcctgaggagatctttatttatttatttagtttttactctggctaggcagatggtggctaaaacattcatttacccatttattcatttaattgttcctgcaaggcctatggatagagtattgtccagcactgctctggaagctaggagcatggggatgaacaagataggctacatcctgttcccacagaacttccactttagtctgggaaacagatgatatatacaaatatataaatgaattcaggtagttttaagtacgaaaagaataagaaagcagagtcatgatttanaatgctggaaacaggggctattgcttgagatattgaaggtgcccaa

SEQ ID NO: 191 - DNA, 369 bp, Homo sapiens
tgacaccttgtccagcatctgcacagggaaaagaaactattatcagagtgaacaggcaacctacagaatgggagaaaatttttgcaatctatccatctgacaaagggctaatatccagaatctacaaagaacttatacaaatttacaagaaacaaacaaacaaacaactcctcaaaaagtgggtgaaggatgtgaacagacacttctcaaaagaagacatttatggggccaacaaacatatgaaaaaaagctcatcatcactggtcactagataaatgcaaatcaaaaccacaatgagataccatctcattccagttagaatggcaatcattaaaaagtcaggaaacaacagatgctggacaaggtgtc

SEQ ID NO: 192 - DNA, 449 bp, Homo sapiens
tgacgcttggccacttgacacttcatctttgcacagaaaaacttctttacagatttaattcaagactggtctagtgacagtcctccagacattttttcatttgttccatatacgtggaattttaaaatcatgtttcatcagtttgaaatgatttgggctgctaatcaacacaattggatcgactgttctactaaacaacaggaaaatgtgtatctggcagcctgtggagaaacactaaacattgatttttctttgccttttacggactttgttccagctacatgtaataccaagttctctttaagaggagaagatgttgatcttcatttgtttctaccagactgccaccctagtaaatattctttatttatgctggtaaaaaattgccatccaaataagatgattcatgatactggtattcctgctgagtgtcaagtggccaagcgtca

SEQ ID NO: 193 - DNA, 372 bp, Homo sapiens
tgacgcttggccacttgacaccagggatgtakcagttgaatataatcctgcaattgtacatattggcaatttcccatcaaacattctagaaagagacaaccaggattgctaggccataaaagctgcaataaataactggtaattgcagtaatcatttcaggccaattcaatccagtttggctcagaggtgcctttggctgagagaagaggtgagatataatgtgttttcttgcaacttcttggaagaataactccacaatagtctgaggactagatacaaacctatttgccattaaagcaccagagtctgttaattccagtactgataagtgttggagattagactccagtgtgtcaagtggccaagcgtca

SEQ ID NO: 194 - DNA, 309 bp, Homo sapiens
  misc_feature 140, 205: n = A,T,C or G
tgacgcttggccacttgacacttatgtagaatccatcgtgggctgatgcaagccctttatttaggcttagtgttgtgggcaccttcaatatcacactagagacaaacgccacaagatctgcagaaacattcagttctgancactcgaatggcaggataactttttgtgttgtaatccttcacatatacaaaaacaaactctgcantctcacgttacaaaaaaacgtactgctgtaaaatattaagaaggggtaaaggataccatctataacaaagtaacttacaactagtgtcaagtggccaagcgtca

SEQ ID NO: 195 - DNA, 312 bp, Homo sapiens
  misc_feature 100, 270: n = A,T,C or G
tgacgcttggccacttgacacccaatctcgcacttcatcctcccagcacctgatgaagtaggactgcaactatccccacttcccagatgaggggaccaangtacacattaggacccggatgggagcacagatttgtccgatcccagactccaagcactcagcgtcactccaggacagcggctttcagataaggtcacaaacatgaatggctccgacaaccggagtcagtccgtgctgagttaaggcaatggtgacacggatgcacgtgtnacctgtaatggttcatcgtaagtgtcaagtggccaagcgtca

SEQ ID NO: 196 - DNA, 288 bp, Homo sapiens
tgtatcgacgtagtggtctcctcagccatgcagaactgtgactcaattaaacctctttcctttatgaattacccaatctcgggtagtgtctttatagtagtgtgagaatggactaatacaagtacattttacttagtaataataataaacaaatatattacatttttgtgtatttactacaccatattttttattgttattgtagtgtacaccttctacttattaaaagaaataggcccgaggcgggcagatcacgaggtcaggagatggagaccactacgtcgatac

SEQ ID NO: 197 - DNA, 289 bp, Homo sapiens
ttgggcaccttcaatatcatgacaggtgatgtgataaccaagaaggctactaagtgattaatgggtgggtaatgtatacagagtaggtacactggacagaggggtaattcatagccaaggcaggagaagcagaatggcaaaacatttcatcacactactcaggatagcatgcagtttaaaacctataagtagtttatttttggaattttccacttaatattttcagactgcaggtaactaaactgtggaacacaagaacatagataaggggagaccactacgtcgatac

SEQ ID NO: 198 - DNA, 288 bp, Homo sapiens
gtatcgacgtagtggtctcccaagcagtgggaagaaaacgtgaaccaattaaaatgtatcagataccccaaagaaaggcgcttgagtaaagattccaagtgggtcacaatctcagatcttaaaattcaggctgtcaaagagatttgctatgaggttgctctcaatgacttcaggcacagtcggcaggagattgaagccctggccattgtcaagatgaaggagctttgtgccatgtatggcaagaaagaccccaatgagcgggactcctggagaccactacgtcgatac

SEQ ID NO: 199 - DNA, 1027 bp, Homo sapiens
  misc_feature 17, 21, 36, 39, 40, 42, 63, 98, 116, 145, 162, 173, 865,885, 891, 916, 924, 927, 929, 934, 942, 949, 976, 983, 988,989, 1009, 1014: n = A,T,C or G
gctttttgggaaaaacncaantgggggaaagggggnttnntngcaaggggataaagggggaancccagggtttccccattcagggaggtgtaaaaagncggccaggggattgtaanaggattcaataatagggggaatgggcccngaagttgcaaggttccngcccgccatgnccgcgggatttagtgacattacgacgstggtaataaagtgggsccaawaaatatttgtgatgtgatttttsgaccagtgaacccattgwacaggacctcatttcctytgagatgrtagccataatcagataaaagrttagaagtytttctgcacgttaacagcatcattaaatggagtggcatcaccaatttcaccctttgttagccgataccttccccttgaaggcattcaattaagtgaccaatcgtcatacgagaggggatggcatggggattgatgatgatatcaggggtgataccttcacaggtgaaaggcatatcctcttgtctatactgaataccacaagtacccttttgaccatgtcgactagcaaatttgtctccaatctgtgtwatccctaacagagcgtacccttattttacaaaatttatatccttcctgattgagagttaccataacctgatccacaatgcccgtctcgctwgttctgagaaaagtgctacagtctctcttggtatagcgtctattggtgctctccaattcatcttcatttttcaggcaaggtgaactgttttgcctataataacmtcatctcctgatacmcgaaacccckggarctatcaaaccatcatcatccagcgttcktwatgtymctaaatccctattgcggccgcctgcaggtcaacatatnggaaaaccccccaccccttnggagcntaccttgaattttccatatgtcccntaaattanctngncttancctggccntaacctnttccggtttaaattgtttccgcccccnttccccnccttnnaaccggaaaccttaattttnaaccnggggttcctatcc

SEQ ID NO: 200 - DNA, 207 bp, Homo sapiens
agtgacattacgacgctggccatcttgaatcctagggcatgaagttgccccaaagttcagcacttggttaagcctgatccctctggtttatcacaaagaataggatgggataaagaaagtggacacttaaataagctataaattatatggtccttgtctagcaggagacaactgcacaggtatactaccagcgtcgtaatgtcacta

SEQ ID NO: 201 - DNA, 209 bp, Homo sapiens
tgggcaccttcaatatctattaaaagcacaaatactgaagaacacaccaagactatcaatgaggttacatctggagtcctcgatatatcaggaaaaaatgaagtgaacattcacagagttttacttctttgggaactcaaatgctagaaaagaaaagggtgccctctttctctggcttcctggtcctatccagcgtcgtaatgtcacta

SEQ ID NO: 202 - DNA, 349 bp, Homo sapiens
  misc_feature 1: n = A,T,C or G
ntacgctgcaacactgtggagccactggtttttattcccggcaggttatccagcaaacagtcactgaacacaccgaagaccgtggtatggtaaccgttcacagtaatcgttccagtcgtctgcgggaccccgacgagcgtcactgggtacagaccagattcagccggaagagaaagcgccgcagggagagactcgaactccactccgctggtgagcagccccatgttttcaactcgaagttcaaacggcattgggttatataccatcagctgaacttcacacacatctccttgaacccactggaaatctattttcttgttccgctcttctccacagtgttgcagcgtaa

SEQ ID NO: 203 - DNA, 241 bp, Homo sapiens
tgctcctcttgccttaccaacccaaagcccactgtgaaatatgaagtgaatgacaaaattcagttttcaacgcaatatagtatagtttatctgattcttttgatctccaggacactttaaacaactgctaccaccaccaccaacctagggatttaggattctccacagaccagaaattatttctcctttgagtttcaggctcctctgggactcctgttcatcaatgggtggtaaatggcta

SEQ ID NO: 204 - DNA, 248 bp, Homo sapiens
tagccatttaccacccatctgcaaaccswgacmwwcargrcywgwackyaggcgatttgaagtactggtaatgctctgatcatgttagttacataagtgtggtcagtttacaaaaattcacagaactaaatactcaatgctatgtgttcatgtctgtgtttatgtgtgtgtaatgtttcaattaagtttttttaaaaaaaagagatgatttccaaataagaaagccgtgttggtaaggcaagaggagc

SEQ ID NO: 205 - DNA, 505 bp, Homo sapiens
  misc_feature 447: n = A,T,C or G
tacgctgcaacactgtggagccattcatacaggtccctaattaaggaacaagtgattatgctacctttgcacggttagggtaccgcggccgttaaacatgtgtcactgggcaggcggtgcctctaatactggtgatgctagaggtgatgtttttggtaaacaggcggggtaagatttgccgagttccttttactttttttaacctttccttatgagcatgcctgtgttgggttgacagtgggggtaataatgacttgttggttgattgtagatattgggctgttaattgtcagttcagtgttttaatctgacgcaggcttatgcggaggagaatgttttcatgttacttatactaacattagttcttctatagggtgatagattggtccaattgggtgtgaggagttcagttatatgtttgggattttttaggtagtgggtgttgancttgaacgctttcttaattggtggctgcttttargcctactatgggtggtaaatggct

SEQ ID NO: 206 - DNA, 179 bp, Homo sapiens
tagactgactcatgtcccctaccaaagcccatgtaaggagctgagttcttaaagactgaagacagactattctctggagaaaaataaaatggaaattgtactttaaaaaaaaaaaaaatcggccgggcatggtagcacacacctgtaatcccagctactaggggacatgagtcagtcta

SEQ ID NO: 207 - DNA, 176 bp, Homo sapiens
agactgactcatgtcccctaccccaccttctgctgtgctgccgtgttcctaacaggtcacagactggtactggtcagtggcctgggggttggggacctctattatatgggatacaaatttaggagttggaattgacacgatttagtgactgatgggatatgggtggtaaatggcta

SEQ ID NO: 208 - DNA, 196 bp, Homo sapiens
agactgactcatgtcccctatttaacagggtctctagtgctgtgaaaaaaaaaaatgctgaacattgcatataacttatattgtaagaaatactgtacaatgactttattgcatctgggtagctgtaaggcatgaaggatgccaagaagtttaaggaatatgggtggtaaatggctaggggacatgagtcagtcta

SEQ ID NO: 209 - DNA, 345 bp, Homo sapiens
  misc_feature 53, 56: n = A,T,C or G
gacgcttggccacttgacaccttttattttttaaggattcttaagtcatttangtnactttgtaagtttttcctgtgcccccataagaatgatagctttaaaaattatgctggggtagcaaagaagatacttctagctttagaatgtgtaggtatagccaggattcttgtgaggaggggtgatttagagcaaatttcttattctccttgcctcatctgtaacatggggataataatagaactggcttgacaaggttggaattagtattacatggtaaatacatgtaaaatgtttagaatggtgccaagtatctaggaagtacttgggcatgggtggtaaatggct

SEQ ID NO: 210 - DNA, 178 bp, Homo sapiens
gacgcttggccacttgacactagagtagggtttggccaactttttctataaaggaccagagagtaaatatttcaggctttgtgggttgtgcagtctctcttgcaactactcagctctgccattgtagcatagaaatcagccatagacaggacagaaatgaatgggtggtaaatggcta

SEQ ID NO: 211 - DNA, 454 bp, Homo sapiens
tgggcaccttcaatatctatccagcgcatctaaattcgcttttttcttgattaaaaatttcaccacttgctgtttttgctcatgtataccaagtagcagtggtgtgaggccatgcttgttttttgattcgatatcagcaccgtataagagcagtgctttggccattaatttatcttcattgtagacagcatagtgtagagtggtatctccatactcatctggaatatttggatcagtgccatgttccagcaacattaacgcacattcatcttcctggcattgtacggcctttgtcagagctgtcctctttttgttgtcaaggacattaagttgacatcgtctgtccagcacgagttttactacttctgaattcccattggcagaggccagatgtagagcagtcctcttttgcttgtccctcttgttcacatcagtgtccctgagcataacggaa

SEQ ID NO: 212 - DNA, 337 bp, Homo sapiens
tccgttatgccacccagaaaacctactggagttacttattaacatcaaggctggaacctatttgcctcagtcctatctgattcatgagcacatggttattactgatcgcattgaaaacattgatcacctgggtttctttatttatcgactgtgtcatgacaaggaaacttacaaactgcaacgcagagaaactattaaaggtattcagaaacgtgaagccagcaattgtttcgcaattcggcattttgaaaacaaatttgccgtggaaactttaatttgttcttgaacagtcaagaaaaacattattgaggaaaattaatatcacagcataacggaa

SEQ ID NO: 213 - DNA, 715 bp, Homo sapiens
  misc_feature 552, 630, 649, 657, 691, 693, 697: n = A,T,C or G
tcgggtgatgcctcctcaggcatcttccatccatctcttcaagattagctgtcccaaatgtttttccttctcttctttactgataaatttggactccttcttgacactgatgacagctttagtatccttcttgtcaccttgcagactttaaacataaaaatactcattggttttaaaaggaaaaaagtatacattagcactattaagcttggccttgaaacattttctatcttttattaaatgtcggttagctgaacagaattcattttacaatgcagagtgagaaaagaagggagctatatgcatttgagaatgcaagcattgtcaaataaacattttaaatgctttcttaaagtgagcacatacagaaatacattaagatattagaaagtgtttttgcttgtgtactactaattagggaagcaccttgtatagttcctcttctaaaattgaagtagattttaaaaacccatgtaatttaattgagctctcagttcagattttaggagaattttaacagggatttggttttgtctaaattttgtcaatttntttagttaatctgtataattttataaatgtcaaactgtatttagtccgttttcatgctgctatgaaagaaatacccangacagggttatttataaanggaaagangttaatttgactcccagttcacaggcctgaggangnatcncccgaaatccttattgcg

SEQ ID NO: 214 - DNA, 345 bp, Homo sapiens
  misc_feature 6, 8, 15: n = A,T,C or G
ggtaangngcatacntcggtgctccggccgccggagtcgggggattcgggtgatgcctcctcaggcccacttgggcctgcttttcccaaatggcagctcctctggacatgccattccttctcccacctgcctgattcttcatatgttgggtgtccctgtttttctggtgctatttcctgactgctgttcagctgccactgtcctgcaaagcctgcctttttaaatgcctcaccattccttcatttgtttcttaaatatgggaagtgaaagtgccacctgaggccgggcacagtggctcacgcctgtaatcccagcactttgggagcctgaggaggcatcacccga

SEQ ID NO: 215 - DNA, 429 bp, Homo sapiens
ggtgatgcctcctcaggcgaagctcagggaggacagaaacctcccgtggagcagaagggcaaaagctcgcttgatcttgattttcagtacgaatacagaccgtgaaagcggggcctcacgatccttctgaccttttgggttttaagcaggaggtgtcagaaaagttaccacagggataactggcttgtggcggccaagcgttcatagcgacgtcgctttttgatccttcgatgtcggctcttcctatcattgtgaagcagaattcaccaagcgttggattgttcacccactaatagggaacgtgagctgggtttagaccgtcgtgagacaggttagttttaccctactgatgatgtgtkgttgccatggtaatcctgctcagtacgagaggaaccgcaggttcasacatttggtgtatgtgcttgcctt

SEQ ID NO: 216 - DNA, 593 bp, Homo sapiens
  misc_feature 15, 429, 446, 498, 512, 538, 543, 557: n = A,T,C or G
tgacacctatgtccngcatctgttcacagtttccacaaatagccagcctttggccacctctctgtcctgaggtatacaagtatatcaggaggtgtataccttctcttctcttccccaccaaagagaacatgcaggctctggaagctgtcttaggagcctttgggctcagaatttcagagtcttgggtaccttggatgtggtctggaaggagaaacattggctctggataaggagtacagccggaggagggtcacagagccctcagctcaagcccctgtgccttagtctaaaagcagctttggatgaggaagcaggttaagtaacatacgtaagcgtacacaggtagaaagtgctgggagtcagaattgcacagtgtgtaggagtagtacctcaatcaatgagggcaaatcaactgaaagaagaagaccnattaatgaattgcttanggggaaggatcaaggctatcatggagatctttctaggaagattattgtttanaattatgaaaggantagggcagggacagggccagaagtanaaganaacattgcctatancccttgtcttgcacccagatgctggacaaggtgtca

SEQ ID NO: 217 - DNA, 335 bp, Homo sapiens
tgacaccttgtccagcatctgacgtgaagatgagcagctcagaggaggtgtcctggatttcctggttctgtgggctccgtggcaatgaattcttctgtgaagtggatgaagactacatccaggacaaatttaatcttactggactcaatgagcaggtccctcactatcgacaagctctagacatgatcttggacctggagcctgatgaagaactggaagacaaccccaaccagagtgacctgattgagcaggcagccgagatgctttatggattgatccacgcccgctacatccttaccaaccgtggcatcgcccagatgctggacaaggtgtca

SEQ ID NO: 218 - DNA, 248 bp, Homo sapiens
tacgtactggtcttgaaggtcttaggtagagaaaaaatgtgaatatttaatcaaagactatgtatgaaatgggactgtaagtacagagggaagggtggcccttatcgccagaagttggtagatgcgtccccgtcatgaaatgttgtgtcactgcccgacatttgccgaattactgaaattccgtagaattagtgcaaattctaacgttgttcatctaagattatggttccatgtttctagtactttta

SEQ ID NO: 219 - DNA, 530 bp, Homo sapiens
  misc_feature 49, 216, 265, 275, 281, 296, 371, 407, 424, 429, 454,456, 458, 464, 474, 476, 506, 509, 527, 530: n = A,T,C or G
tgacgcttggccacttgacacaagtaggggataaggacaaagacccatnaggtggcctgtcagccttttgttactgttgcttccctgtcaccacggccccctctgtaggggtgtgctgtgctctgtggacattggtgcattttcacacataccattctctttctgcttcacagcagtcctgaggcgggagcacacaggactaccttgtcagatgangataatgatgtctggccaactcaccccccaaccttctcactagttatangaagagccangcctanaaccttctatcctgnccccttgccctatgacctcatccctgttccatgccctattctgatttctggtgaactttggagcagcctggtttntcctcctcactccagcctctctccataccatggtangggggtgctgttccacncaaanggtcaggtgtgtctggggaatcctnananctgccnggagtttccnangcattcttaaaaaccttcttgcctaatcanatngtgtccagtggccaaccntcn

SEQ ID NO: 220 - DNA, 531 bp, Homo sapiens
tgacgcttggccacttgacactaaatagcatcttctaaaggcctgattcagagttgtggaaaattctcccagtgtcagggattgtcaggaacagggctgctcctgtgctcactttacctgctgtgtttctgctggaaaaggagggaagaggaatggctgatttttacctaatgtctcccagtttttcatattcttcttggatcctcttctctgacaactgttcccttttggtcttcttcttcttgctcagagagcaggtctctttaaaactgagaagggagaatgagcaaatgattaaagaaaacacacttctgaggcccagagatcaaatattaggtaaatactaaaccgcttgcctgctgtggtcacttttctcctctttcacatgctctatccctctatcccccacctattcatatggcttttatctgccaagttatccggcctctcatcaaccttctcccctagcctactgggggatatccatctgggtctgtctctggtgtattggtgtcaagtggccaagcgtca

SEQ ID NO: 221 - DNA, 530 bp, Homo sapiens
attgacgcttggccacttgacacccgcctgcctgcaatactggggcaagggccttcactgctttcctgccaccagctgccactgcacacagagatcagaaatgctaccaaccaagactgttggtcctcagcctctctgaggagaaagagcagaagcctggaagtcagaagagaagctagatcggctacggccttggcagccagcttccccacctgtggcaataaagtcgtgcatggcttaacaatgggggcacctcctgagaaacacattgttaggcaattcggcgtgtgttcatcagagcatatttacacaaacctcgatagtgcagcctactatccactattgctcctacgctgcaaacctgaacagcatgggactgtactgaatactggaagcagctggtgatggtacttatttgtgtatctaaacacagagaaggtacagtaagaatatggtatcataaacttacagggaccgccatcctatatgcagtctgttgtgaccaaaatgtgtcaagtggccaagcgtca

SEQ ID NO: 222 - DNA, 578 bp, Homo sapiens
  misc_feature 308, 381, 561, 570, 573: n = A,T,C or G
tgtatcgacgtagtggtctccgggctactaggccgttgtgtgctggtagtacctggttcactgaaaggcgcatctccctccccgcgtcgccctgaagcagggggaggacttcgcccagccaaggcagttgtatgagttttagctgcggcacttcgagacctctgagcccacctccttcaggagccttccccgattaaggaagccagggtaaggattccttcctcccccagacaccacgaacaaaccaccaccccccctattctggcagcccatatacatcagaacgaaacaaaaataacaaataaacnaaaaccaaaaaaaaaagagaaggggaaatgtatatgtctgtccatcctgttgctttagcctgtcagctcctanagggcagggaccgtgtcttccgaatggtctgtgcagcgccgactgcgggaagtatcggaggaggaagcagagtcagcagaagttgaacggtgggcccggcggctcttgggggctggtgttgtacttcgagaccgctttcgctttttgtcttagatttacgtttgctctttggagtggganaccactacntcnataca

SEQ ID NO: 223 - DNA, 578 bp, Homo sapiens
tgtatcgacgtagtggtctcctcttgcaaaggactggctggtgaatggtttccctgaattatggacttaccctaaacatatcttatcatcattaccagttgcaaaatattagaatgtgttgtcactgtttcatttgattcctagaaggttagtcttagatatgttactttaacctgtatgctgtagtgctttgaatgcattttttgtttgcatttttgtttgcccaacctgtcaattatagctgcttaggtctggactgtcctggataaagctgttaaaatattcaccagtccagccatcttacaagctaattaagtcaactaaatgcttccttgttttgccagacttgttatgtcaatcctcaatttctgggttcattttgggtgccctaaatcttagggtgtgactttcttagcatcctgtaacatccattcccaagcaagcacaacttcacataatactttccagaagttcattgctgaagcctttccttcacccagcggagcaacttgattttctacaacttccctcatcagagccacaagagtatgggatatggagaccactacgtcgataca

SEQ ID NO: 224 - DNA, 345 bp, Homo sapiens
  misc_feature 13: n = A,T,C or G
tgtatcgacgtantggtctcccaaggtgctgggattgcaggcatgagccaccactcccaggtggatctttttctttatacttacttcattaggtttctgttattcaagaagtgtagtggtaaaagtcttttcaatctacatggttaaataatgatagcctgggaaataaatagaaattttttctttcatctttaggttgaataaagaaacagaaaaaatagaacatactgaaaataatctaagttccaaccatagaagaactgcagaagaaatgaagaaagtgatgatgatttagattttgatattgatttagaagacacaggaggagaccactacgtcgataca

SEQ ID NO: 225 - DNA, 347 bp, Homo sapiens
tgtatcgacgtagtggtctccaaactgaggtatgtgtgccactagcacacaaagccttccaacagggacgcaggcacaggcagtttaaagggaatctgtttctaaattaatttccaccttctctaagtattctttcctaaaactgatcaaggtgtgaagcctgtgctctttcccaactcccctttgacaacagccttcaactaacacaagaaaaggcatgtctgacactcttcctgagtctgactctgatacgttgttctgatgtctaaagagctccagaacaccaaagggacaattcagaatgctggtgtataacagactccaatggagaccactacgtcgataca

SEQ ID NO: 226 - DNA, 281 bp, Homo sapiens
  misc_feature 4, 6, 11: n = A,T,C or G
aggngngggantgtatcgacgtagtggtctcccaacagtctgtcattcagtctgcaggtgtcagtgttttggacaatgaggcaccattgtcacttattgactcctcagctctaaatgctgaaattaaatcttgtcatgacaagtctggaattcctgatgaggttttacaaagtattttggatcaatactccaacaaatcagaaagccagaaagaggatcctttcaatattgcagaaccacgagtggatttacacacctcaggagaccactacgtcgataca

SEQ ID NO: 227 - DNA, 3646 bp, Homo sapiens
gggaaacacttcctcccagccttgtaagggttggagccctctccagtatatgctgcagaatttttctctcggtttctcagaggattatggagtccgccttaaaaaaggcaagctctggacactctgcaaagtagaatggccaaagtttggagttgagtggccccttgaagggtcactgaacctcacaattgttcaagctgtgtggcgggttgttactgaaactcccggcctccctgatcagtttccctacattgatcaatggctgagtttggtcaggagcaccccttccgtggctccactcatgcaccattcataattttacctccaaggtcctcctgagccagaccgtgttttcgcctcgaccctcagccggttcggctcgccctgtactgcctctctctgaagaagaggagagtctccctcacccagtcccaccgccttaaaaccagcctactcccttagggtcatcccatgtctcctcggctatgtcccctgtaggctcatcacccattgcctcttggttgcaaccgtggtgggaggaagtagcccctctactaccactgagagaggcacaagtccctctgggtgatgagtgctccacccccttcctggtttatgtcccttctttctacttctgacttgtataattggaaaacccataatcctcccttctctgaaaagccccaggctttgacctcactgatggagtctgtactctggacacattggcccacctgggatgactgtcaacagctccttttgacccttttcacctctgaagagagggaaagtatccaaagagaggccaaaaagtacaacctcacatcaaccaataggccggaggaggaagctagaggaatagtgattagagacccaattgggacctaattgggacccaaatttctcaagtggagggagaacttttgacgatttccaccggtatctcctcgtgggtattcagggagctgctcagaaacctataaacttgtctaaggcgactgaagtcgtccaggggcatgatgagtcaccaggagtgtttttagagcacctccaggaggcttatcagatttacaccccttttgacctggcagcccccgaaaatagccatgctcttaatttggcatttgtggctcaggcagccccagatagtaaaaggaaactccaaaaactagagggattttgctggaatgaataccagtcagcttttagagatagcctaaaaggtttttgacagtcaagaggttgaaaaacaaaaacaagcagctcaggcagctgaaaaaagccactgataaagcatcctggagtatcagagtttactgttagatcagcctcatttgacttcccctcccacatggtgtttaaatccagctacactacttcctgactcaaactccactattcctgttcatgactgtcaggaactgttggaaactactgaaactggccgacctgatcttcaaaatgtgcccctaggaaaggtggatgccaccatgttcacagacagtagcagcttcctcgagaagggactacgaaaggccggtgcagctgttaccatggagacagatgtgttgtgggctcaggctttaccagcaaacacctcagcacaaaaggctgaattgatcgccctcactcaggctctccgatggggtaaggatattaacgttaacactgacagcaggtacgcctttgctactgtgcatgtacgtggagccatctaccaggagcgtgggctactcacctcagcaggtggctgtaatccactgtaaaggacatcaaaaggaaaacacggctgttgcccgtggtaaccagaaagctgattcagcagctcaagatgcagtgtgactttcagtcacgcctctaaacttgctgcccacagtctcctttccacagccagatctgcctgacaatcccgcatactcaacagaagaagaaaactggcctcagaactcagagccaataaaaatcaggaaggttggtggattcttcctgactctagaatcttcataccccgaactcttgggaaaactttaatcagtcacctacagtctaccacccatttaggaggagcaaagctacctcagctcctccggagccgttttaagatcccccatcttcaaagcctaacagatcaagcagctctccggtgcacaacctgcgcccaggtaaatgccaaaaaaggtcctaaacccagcccaggccaccgtctccaagaaaactcaccaggagaaaagtgggaaattgactttacagaagtaaaaccacaccgggctgggtacaaataccttctagtactggtagacaccttctctggatggactgaagcatttgctaccaaaaacgaaactgtcaatatggtagttaagtttttactcaatgaaatcatccctcgacatgggctgcctgtttgccatagggtctgataatggaccggccttcgccttgtctatagtttagtcagtcagtaaggcgttaaacattcaatggaagctccattgtgcctatcgaccccagagctctgggcaagtagaacgcatgaactgcaccctaaaaaacactcttacaaaattaatcttagaaaccggtgtaaattgtgtaagtctccttcctttagccctacttagagtaaggtgcaccccttactgggctgggttcttaccttttgaaatcatgtatgggagggtgctgcctatcttgcctaagctaagagatgcccaattggcaaaaatatcacaaactaatttattacagtacctacagtctccccaacaggtacaagatatcatcctgccacttgttcgaggaacccatcccaatccaattcctgaacagacagggccctgccattcattcccgccaggtgacctgttgtttgttaaaaagttccagagagaaggactccctcctgcttggaagagacctcacaccgtcatcacgatgccaacggctctgaaggtggatggcattcctgcgtggattcatcactcccgcatcaaaaaggccaacagagcccaactagaaacatgggtccccagggctgggtcaggccccttaaaactgcacctaagttgggtgaagccattagattaattctttttcttaattttgtaaaacaatgcatagcttctgtcaaacttatgtatcttaagactcaatataacccccttgttataactgaggaatcaatgatttgattcccccaaaaacacaagtggggaatgtagtgtccaacctggtttttactaaccctgtttttagactctccctttcctttaatcactcagcttgtttccacctgaattgactctcccttagctaagagcgccagatggactccatcttggctctttcactggcagccgcttcctcaaggacttaacttgtgcaagctgactcccagcacatccaagaatgcaattaactgataagatactgtggcaagctatatccgcagttcccaggaattcgtccaattgatcacagcccctctacccttcagcaaccaccaccctgatcagtcagcagccatcagcaccgaggcaaggccctccaccagcaaaaagattctgactcactgaagacttggatgatcattagtatttttagcagtaaagtttttttttctttttctttctttttttctcgtgcc

SEQ ID NO: 228 - DNA, 419 bp, Homo sapiens
  misc_feature 402: n = A,T,C or G
taagagggtacaagatctaagcacagccgtcaatgcagaacacagaacgtagcctggtaagtgtgttaagagtgggaatttttggagtacagagtaaggcacctaaccctagctggggtttggtgacggtcccagatggcttacagaagaaagtgtcctgagatgagtttttaagaatgaataaggatagacacaagtgaggactgacttggcagtggtgaatggtgggtggcaaaaaacttcgcatgtatggaaactgcacgtacaggaatgaagaatgagactgtgtggtgtttaatgagctgcaaatactaattttatcctgaaagttttgaagagttaactaaaaagtattttttagtaaggaaataaccctacatttcagggttattgtttgtttanatattgaaggtgcccaa

SEQ ID NO: 229 - DNA, 148 bp, Homo sapiens
aagagggtacctgtatgtagccatggtggcaatgagagactgattactacctgctggagattgtttaagtgagttaatatattaaggataaagggagccaggttttttgactgttggagaaggaaattacagatattgaaggtcccaa

SEQ ID NO: 230 - DNA, 257 bp, Homo sapiens
taagagggtacmaaaaaaaaaaaatagaacgaatgagtaagacctactatttgatagtacaacagggtgactatagtcaatgataacttaattatacatttaacatagagtgtaattggattgtttgtaactcgaaggataaatgcttgagaggatggataccccattctccatgatgtacttatttcacattacatgcctgtatcaaagcatctcatataccctataaatatgtacacctactatgtaccctctta

SEQ ID NO: 231 - DNA, 260 bp, Homo sapiens
taagagggtacgggtatttgctgatgggatttttttttctttctttttctttggaaaacaaaatgaaagccagaacaaaattattgaacaaaagacagggactaaatctggagaaatgaagtcccctcacctgactgccatttcattctatctgaccttccagtctaggttaggagaatagggggtggaggggattaatctgatacaggtatatttaaagcaactctgcatgtgtgccagaagtccatggtaccctctta

SEQ ID NO: 232 - DNA, 596 bp, Homo sapiens
  misc_feature 437, 440, 461, 536, 541, 565, 580, 587, 590, 595: n = A,T,C or G
tgctcctcttgccttaccaaccacaaattagaaccataatgagatgtcacctcatacctggtgggattaacattatttaaaaaatcagaagtattgacaaggatgtgaagaaattagaacatctgtgcactgttggtgggaatgtaaaaaaggtgtggccactatgggtaacagcatgaaggttcctcaaaaaaaattttttttaatctactctatgatcgatcttgaggttgtttatgcaaaagaactgaaatcaggattttgaggaaatattcacattcccacatccatttctgctttattcataatactcaagagatggaaacaacctaaatgtccatcccgggatgaatggataaacacagtgtggtatatgcatacaatggaatattatttagtctttaaaaagaaaaattctatcatatactacaacttanatnaaccttgaggacacaatgctnagtgaaataagccacggaaggacgaatactgcattattcccttatatgaagtatctaaagtggtcaaactcttanagcanaaagtaaaaatgggtggttgccanacagttggttaggcnagaaganaancctant

SEQ ID NO: 233 - DNA, 96 bp, Homo sapiens
tcttctgaagacctttcgcgactcttaagctcgtggttggtaaggcaagaggagcgttggtaaggcaagaggagcgttggtaaggcaagaggagca

SEQ ID NO: 234 - DNA, 313 bp, Homo sapiens
tgtaagtcgagcagtgtgatgataaaacttgaatggatcaatagttgcttcttatggatgagcaaagaaagtagtttcttgtgatggaatctgctcctggcaaaaatgctgtgaacgttgttgaaaagacaacaaagagtttagagtagtacataaatttagaatagtacataaacttagaatagtacataaacttagtacataaataatgcacgaagcaggggcagggcttgagagaattgacttcaatttggaaagagtatctactgtaggttagatgctctcaaacagcatcacactgctcgacttacaa

SEQ ID NO: 235 - DNA, 550 bp, Homo sapiens
aacgaggacagatccttaaaaagaatgttgagtgaaaaaagtagaaaataagataatctccaaagtccagtagcattatttaaacatttttaaaaaatacactgataaaaattttgtacatttcccaaaaatacatatggaagcacagcagcatgaatgcctatgggrttgaggataggggttgggagtagggatggggataaagggggaaaataaaaccagagaggagtcttacacatttcatgaaccaaggagtataattatttcaactatttgtaccwgaagtccagaaagagtggaggcagaagggggagaagagggcgaagaaacgtttttgggagaggggtcccasaagagagattttcgcgatgtggcgctacatacgtttttccaggatgccttaagctctgcaccctatttttctcatcactaatattagattaaaccctttgaagacagcgtctgtggtttctctacttcagctttccctccgtgtcttgcacacagtagctgttttacaagggttgaactgactgaagtgagattattc

SEQ ID NO: 236 - DNA, 325 bp, Homo sapiens
tagactgactcatgtcccctaccagagtagctagaattaatagcacaagcctctacacccaggaactcactattgaatacataaatggaatttattcagccttaaaaagtttggaaggaaattctgacatatgctaaaacatggatgaaccttgaagactttatgataagtaaaagaagccagtcataaaaggaaaaatattgcatgattccacttatatgaggtacctagagtagtcaatttcatagaaacacaaaatagaatggtgtttgccagggcttttgaggaaaagggaatgacaagttaggggacatgagtcagtcta

SEQ ID NO: 237 - DNA, 373 bp, Homo sapiens
  misc_feature 355: n = A,T,C or G
tagactgactcatgtcccctatctactcaacatttccacttgaagtctgataggcatctcagacttatcttgtcccaaagcaaactctttatttcttttcatcctagtctttatttcttgtgctgtcttacccatctcaaaagagtgccaaaatccaccaagttgctgaaacagaaatctaagaaatatccttgattcttctttttcccatctacttcacttctaattcattagtaaataatctgtttcagaaaaccaaacacctcatgttctcactcataagggggagttgaacaatgagaacacacagacacagggaggggaacatcacacaccacggcccgtcagggagtangggacatgagtcagtcta

SEQ ID NO: 238 - DNA, 492 bp, Homo sapiens
  misc_feature 272, 310, 380, 435, 474, 484, 488: n = A,T,C or G
tagactgactcatgtcccctataatgctcccaggcatcagaaagcatctcaaactggagctgacaccatggcagaggtttcaggtaagtcacaaaaggggtcctaaagaatttgccctcaatatcagagtgattagaagaagtggacagagctacccaagttaaacatatgcgagataaaaaaaatatggcacttgtgaacacacactacaggaggaaaataaggaacataatagcatattgtgctattatgatgatgaagaacctctctanaagaaaacataaccaaagaaacaaagaaaattcctgcnaatgtttaatgctatagaagaaattaacaaaaacatatattcaatgaattcagaaaagttagcaggtcanaagaaaacaaatcaaagaccagaataatcccattttagattgtcgagtaaactanaacagaaagaataccactggaaattgaattcctacgtangggacatgantcantcta

SEQ ID NO: 239 - DNA, 482 bp, Homo sapiens
  misc_feature 245: n = A,T,C or G
tggaaagtatttaatgatgggcaacttgctgtttacttcctacatatcccatcatcttctgtatttttttaaataacttttttttggatttttaaagtaaccttattctgagaggtaacatggattacatacttctaagccattaggagactctatgttaaaccaaaaggaaatgttactagatcttcatttgatcaataggatgtgataatcatcatctttctgctctaatggaaaagtactanaaacatggaaccataatcttagatgaacaacgttagaatttgcactaattctacggaatttcagtaattcggcaaatgtcgggcagtgacacaacatttcatgacggggacgcatctaccaacttctggcgataagggccacccttccctctgtacttacagtcccatttcatacacagtctttgattaaatattcacattttttctctacctaaagaccttcaagaccagtacgta

SEQ ID NO: 240 - DNA, 519 bp, Homo sapiens
  misc_feature 491: n = A,T,C or G
tgtatcgacgtagtggtctccccatgtgatagtctgaaatatagcctcatgggatgagaggctgtgccccagcccgacacccgtaaagggtctgtgctgaggtggattagtaaaagaggaaagccttgcagttgagatagaggaagggcactgtctcctgcctgcccctgggaactgaatgtctcggtataaaacccgattgtacatttgttcaattctgagataggagaaaaaccaccctatggcgggaggcgagacatgttggcagcaatgctgccttgttatgctttactccacagatgtttgggcggagggaaacataaatctggcctacgtgcacatccaggcatagtacctccctttgaacttaattatgacacagattcctttgctcacatgtttttttgctgaccttctccttattatcaccctgctctcctaccgcattccttgtgctgagataatgaaaataatatcaataaaaacttganggaactcggagaccactacgtcgataca

SEQ ID NO: 241 - DNA, 771 bp, Homo sapiens
  misc_feature 304, 402, 442, 463, 510, 541, 550, 567, 571, 596, 617,624, 644, 648, 652, 667, 682, 686, 719, 722, 729, 732, 751, 752,757, 758, 760, 763, 766, 769: n = A,T,C or G
tgtatcgacgtagtggtctccactcccgccttgacggggctgctatctgccttccaggccactgtcacggctcccgggtagaagtcacttatgagacacaccagtgtggccttgttggcttgaagctcctcagaggagggtgggaacagagtgaccgagggggcagccttgggctgacctaggacggtcagcttggtccctccgccaaacacgagagtgctgctgcttgtatatgagctgcagtaataatcagcctcgtcctcagcctggagcccagagatggtcagggaggccgtgttgccanacttggagccagagaagcgattagaaacccctgagggccgattaccgacctcataaatcatgaatttgggggctttgcctgggtgctgttggtaccangagacattattataaccaccaacgtcactgctggttccantgcagggaaaatggttgatcnaactgtccaagaaaaccactacgtccataccaatccactaattgccngccgcctgcaggttcaaccatattggggaanaactccccnccgccgtttgggattgncatnaacctttgaaattttttcctattanttgtccccctaaaataaaccnttgggcnttaatccattgggtccatancttntttncccggtttttaaaanttgtttatcccgccncccnatttcccccccaactttccaaaacccgaaaccnttnaaatttnttnaaaccctggggggttcccnnaattnnanttnaanctncc

SEQ ID NO: 242 - DNA, 167 bp, Homo sapiens
tgggcaccttcaatatcgggctcatcgataacatcacgctgctgatgctgctgttgctggtcctctctaggaacctctggattttcaaattctttgaggaattcatccaaattatctgcctctcctcctttcctcctttttctaaggtcttctggtacaagcggtca

SEQ ID NO: 243 - DNA, 338 bp, Homo sapiens
ttgggcaccttcaatatctactgatctaaatagtgtggtttgaggcctcttgttcctggctaaaaatccttggcaagagtcaatctccactttacaatagaggtaaaaatcttacaatggatattcttgacaaagctagcatagagacagcaattttacacaaggtatttttcacctgtttaataacagtggttttcctacacccatagggtgccaccaagggaggagtgcacagttgcagaaacaaattaagatactgaagacaacactacttaccatttcccgtatagctaaccaccagttcaactgtacatgtatgttcttatgggcaatcaaga

SEQ ID NO: 244 - DNA, 346 bp, Homo sapiens
tttttggctcccatacagcacactctcatgggaaatgtctgttctaaggtcaacccataatgcaaaaatcatcaatatacttgaagatccccgtgtaaggtacaatgtatttaatattatcactgatacaattgatccaataccagttttagtctggcattgaatcaaatcactgtttttgttgtataaaaagagaaatatttagcttatatttaagtaccatattgtaagaaaaaagatgcttatctttacatgctaaaatcatgatctgtacattggtgcagtgaatattactgtaaaagggaagaaggaatgaagacgagctaaggatattgaaggtgcccaa

SEQ ID NO: 245 - DNA, 521 bp, Homo sapiens
  misc_feature 252, 337, 434, 455, 466, 478, 494, 510, 516: n = A,T,C or G
accaatcccacacggatactgagggacaagtatatcatcccatttcatccctacagcagcaacttcatgaggcaggagttattagtcccattttacagaagaggaaactgagacttagggagatcaagtaatttgcccaggtcgcacaattagtgatagagccagggcttgaagcgacgtctgtcttaagccaatgacccctgcagattattagagcaactgttctccacaacagtgtaagcctcttgctanaagctcaggtccacaagggcagagatttttgtctgttttgctcattgctccttccccattgcttagagcagggtctgccacgaancaggttctcaatgcatagttattaaatgtatataagagcaaacatatgttacagagaactttctgtatgcttgtcacttacatgaatcacctgtganatgggtatgcttgttccccantgttgcagatnaagatattgaangtgcccaaatcactanttgcgggcgcctgcangtccancatat

SEQ ID NO: 246 - DNA, 482 bp, Homo sapiens
  misc_feature 464: n = A,T,C or G
tggaaccaatccaaatacccatcaatgatagactggataaagaaaatttggcacatgttcaccatgaaatactatgcagccataaaaaaggatgagttcatatcctttgcagggacatggatgaagctggagaccatcattctcagcaaactaacaagggaacagaaaaccaaacactgcatgttctcactcttaagtgggagctgaacaatgagaacacatggacacagggaggggaacatcacacagtggggcctgctggtgggtaggggtctaggggagggatagcattaggagaaatacctaatgtagatgacgggttgatgggtgcagcaaaccaccatgacacgtgtatacctatgtaacaaacctgcatgttctgcacatgtaccccagaacttaaagtgttaataaaaaaattaagaaaaaagttaagtatgtcatagatacataaaatattgtanatattgaaggtgcccaaa

SEQ ID NO: 247 - DNA, 474 bp, Homo sapiens
  misc_feature 220, 255, 287, 312, 339, 374, 382, 403, 414, 426, 427,428, 432, 433, 434, 435, 436, 465: n = A,T,C or G
ttcgatacaggcacagagtaagcagaaaaatggctgtggtttaaccaagtgagtacagttaagtgagagaggggcagagaagacaagggcatatgcagggggtgattataacaggtggttgtgctgggaagtgagggtactcggggatgaggaacagtgaaaaagtggcaaaaagtggtaagatcagtgaattgtacttctccagaatttgatttctggnggagtcaaataactatccagtttggggtatcatanggcaacagttgaggtataggaggtagaagtcncagtgggataattgaggttatgaanggtttggtactgactggtactgacaangtctgggttatgaccatgggaatgaatgactgtanaagcgtanaggatgaaactattccacganaaaggggtccnaaaactaaaaannnaagnnnnnggggaatattatttatgtggatattgaangtgcccaaa

SEQ ID NO: 248 - DNA, 355 bp, Homo sapiens; identical to SEQ ID NO: 78
  misc_feature 69, 87, 186, 192, 220, 227, 251, 278, 339, 346, 350: n = A,T,C or G

SEQ ID NO: 249 - DNA, 434 bp, Homo sapiens
ttggattggtcctccaggagaacaaggggaaaaaggtgaccgagggctccctggaactcaaggatctccaggagcaaaaggggatgggggaattcctggtcctgctggtcccttaggtccacctggtcctccaggcttaccaggtcctcaaggcccaaagggtaacaaaggctctactggacccgctggccagaaaggtgacagtggtcttccagggcctcctgggcctccaggtccacctggtgaagtcattcagcctttaccaatcttgtcctccaaaaaaacgagaagacatactgaaggcatgcaagcagatgcagatgataatattcttgattactcggatggaatggaagaaatatttggttccctcaattccctgaaacaagacatcgagcatatgaaatttccaatgggtactcagaccaatccaa

SEQ ID NO: 250 - DNA, 430 bp, Homo sapiens
  misc_feature 301, 430: n = A,T,C or G
tggattggtcacatggcagagacaggattccaaggcagtgagaggaggatacaatgcttctcactagttattattatttattttatttttgagatgaagtctcgctttgtctcccaggctggagagcggtggtgcgatcttggctctctgcaacccccgcctcaagcaattctcctgtcttagcctcgcgggtagatggaattacaggcgcccaccgccatgcccaactaatttttttgtgtcttcagtagagacagggtttcgccatgttgggcaggctggtcttgaactcctgacctcnagtgatctgccctcctcggcctcacaaagtgctggaattacaggcatgggctgctgcacccagtcaacttctcactagttatggccttatcattttcaccacattctattggcccaaaaaaaaaaaaan

SEQ ID NO: 251 - DNA, 329 bp, Homo sapiens
tggtactccaccatyatggggtcaaccgccatcctcgccctcctcctggctgttctccaaggagtctgtgccgaggtgcagctgrtgcagtctggagcagaggtgaaaaagtccggggagtctctgaagatctcctgtaagggttctggatacacctttaagatctactggatcgcctgggtgcgccagttgcccgggaaaggcctggagtggatggggctcatctttcctgatgactctgataccagatacagcccgtccttccaaggccaggtcaccatctcagtcgataagtccatcagcaccgcctatctgcagtggagtaccaa

SEQ ID NO: 252 - DNA, 536 bp, Homo sapiens
tggtactccactcagcccaaccttaattaagaattaagagggaacctattactattctcccaggctcctctgctctaaccaggcttctgggacagtattagaaaaggatgtctcaacaagtatgtagatcctgtactggcctaagaagttaaactgagaatagcataaatcagaccaaacttaatggtcgttgagacttgtgtcctggagcagctgggataggaaaacttttgggcagcaagaggaagaactgcctggaagggggcatcatgttaaaaattacaaggggaacccacaccaggcccccttcccagctctcagcctagagtattagcatttctcagctagagactcacaacttccttgcttagaatgtgccaccggggggagtccctgtgggtgatgaggctctcaagagtgagagtggcatcctatcttctgtgtgcccacaggagcctggcccgagacttagcaggtgaagtttctggtccaggctttgcccttgactcactatgtgacctctggtggagtaccaa

SEQ ID NO: 253 - DNA, 507 bp, Homo sapiens
  misc_feature 1: n = A,T,C or G
ntgttgcgatcccagtaactcgggaagctgaggcgggaggatcacctgagctcaggaggttgaggccgcagtgagccgggaccacgccactacactccagcctggggcatagagtgagaccctccaagacagaaaagaaaagaaaggaagggaaagggaaagggaaaaggaaaaggaaaaggaaaaggaaaaggaaaagacaagacaaaacaagacttgaatttggatctcctgacttcaattttatgttctttctacaccacaattcctctgcttactaagatgataatttagaaacccctcgttccattctttacagcaagctggaagtttggtcaagtaattacaataatagtaacaaatttgaatattatatgccaggtgtttttcattcctgctctcacttaattctcaccactctgatataaatacaattgctgccgggtgtggtggctcatgcctgtaatcccggcactttgggagaccgaggtgggcggatsgcaacaa

SEQ ID NO: 254 - DNA, 222 bp, Homo sapiens
  misc_feature 167: n = A,T,C or G
ttggattggtcactgtgaggaagccaaatcggatccgagagtctttttctaaaggccagtactggccacactttctcctgccgccttcctcaaagctgaagacacacagagcaaggcgcttctgttttactccccaatggtaactccaaaccatagatggttagctnccctgctcatctttccacatccctgctattcagtatagtccgtggaccaatccaa

SEQ ID NO: 255 - DNA, 463 bp, Homo sapiens
tgttgcgatccataaatgctgaaatggaaataaacaacatgatgagggaggattaagttggggagggagcacattaaggtggccatgaagtttgttggaagaagtgacttttgaacaaggccttggtgttaagagctgatgagagtgtcccagacagaggggccactggtacaatagacgagatgggagagggcttggaaggtgtgcgaaataggaaggagtttgttctggtatgagtctagtgaacacagaggcgagaggccctggtgggtgcagctggagagttatgcagaataacattaggccctgtgggggactgtagactgtcagcaataatccacagtttggattttattctaagagtgatgggaagccgtggaaagggggttaagcaaggagtgaaattatcagatttacagtgataaaaataaattggtctggctactggggaaaaaaaaaaaaa

SEQ ID NO: 256 - DNA, 262 bp, Homo sapiens
ttggattggtcaacctgctcaactctacytttcctccttcttcctaaaaaattaatgaatccaatacattaatgccaaaacccttgggttttatcaatatttctgttaaaaagtattatccagaactggacataatactacataataatacataacaaccccttcatctggatgcaaacatctattaatatagcttaagatcactttcactttacagaagcaacatcctgttgatgttattttgatgtttggaccaatccaa

SEQ ID NO: 257 - DNA, 461 bp, Homo sapiens
  misc_feature 2, 5, 6, 7, 8, 9, 10, 11, 12, 13, 25, 32, 38, 71, 72: n = A,T,C or G
gnggnnnnnnnnncaattcgactcngttcccntggtanccggtcgacatggccgcgggattaccgcttgtnnctgggggtgtatgggggactatgaccgcttgtagctgggggtgtatgggggactatgaccgcttgtagmtggkggtgtatgggggactatgaccgcttgtcgggtggtcggataaaccgacgcaagggacgtgatcgaagctgcgttcccgctctttcgcatcggtagggatcatggacagcaatatccgcattcgyctgaaggcgttcgaccatcgcgtgctcgatcaggcgaccggcgacatcgccgacaccgcacgccgtaccggcgcgctcatccgcggtccgatcccgcttcccacgcgcatcgagaagttcacggtcaaccgtggcccgcacgtcgacaagaagtcgcgcgagcagttcgaggtgcgtacctacaagcggtca

SEQ ID NO: 258 - DNA, 332 bp, Homo sapiens
  misc_feature 251: n = A,T,C or G
tgaccgcttgtagctgggggtgtatgggggactacgaccgcttgtagctgggggtgtatgggggactatgaccgcttgtagctgggggtgtatgggggactatgaccgcttgtagctgggggtgtatgggggactaggaccgcttgtagctgggggtgtatgggggactatgaccgcttgtagctgggggtgtatgggggactacgaccgcttgtagctgggggtgtatgggggactatgaccgcttgtanctgggggtgtatgggggactatgaccgcttgtgctgcctgggggatgggaggagagttgtggttggggaaaaaaaaaaaaa

SEQ ID NO: 259 - DNA, 291 bp, Homo sapiens
  misc_feature 141, 144, 167, 168, 171, 175, 194, 201, 202, 205, 209,212, 235, 236, 245, 246, 258, 266, 268, 270, 273, 277, 285, 290: n = A,T,C or G
taccgcttgtgaccgcttgtgaccgcttgtgaccgcttgtgaccgcttgtgaccgcttgtgaccgcttgtgaccgcttgtgaccgcttgtgaccgcttgtgaccgcttgtgaccgcttgtgaccgcttgtgaccgcttgtnacngggggtgtctgggggactatganngantgtnactgggggtgtctgggggnctatganngantgtnacngggggtgtctgggggactatganngactgtgcnncctgggggatcngaggagantngnggntagngatggttngggana

SEQ ID NO: 260 - DNA, 238 bp, Homo sapiens
taagagggtactggttaaaatacaggaaatctggggtaatgaggcagagaaccaggatactttgaggtcagggatgaaaactagaatttttttctttttttttgcctgagaaacttgctgctctgaagaggcccatgtattaattgctttgatcttccttttcttacagccctttcaagggcagagccctccttatcctgaaggaatcttatccttagctatagtatgtaccctctta

SEQ ID NO: 261 - DNA, 746 bp, Homo sapiens
  misc_feature 662, 680, 685, 698, 707, 709, 734, 740, 741: n = A,T,C or G
ttgggcaccttcaatatcaatagctaacatttattgagtgtttatcgtatcataaaacactgttctaagcctttaaacgtactaattcatttaatgctcataatcactttagaaggtgggtactagtattagtctcatttacagatgcaacatgcaggcacagagaggttaattaacttgcccaaggtaacacagctaagaaatagaaaaaatattgaatctggaaagttgggcttctgggtaacccacagagtcttcaatgagcctggggcctcactcagtttgcttttacaaagcgaatgagtaacatcacttaattcagtgagtaggccaaatggaggtcagctacgagtttctgctgttcttgcagtggactgacagatgtttacaacgtctggccatcagtwaatggactgattatcattgggawgtgggtgggctgaatgttggccagtgaagtttattcawgccatatttttatgtttaggatgacttttggctggtcctagggcaagctctgtctgscacggaacacagaatwacacagggaccccctcaatttctggtgtggctagaaccatgaaccactggttgggggaacaagcggtcaaaacctaagtgcggccggctggcagggtccacccatatggggaaaactcccnacgcgtttggaatgcctnagctngaattattctaanagttgtccncntaaaattagcctgggcgttaatcangggtcnnaagcc

SEQ ID NO: 262 - DNA, 588 bp, Homo sapiens
  misc_feature 485, 488, 489, 492, 493, 494, 496, 497, 498, 499, 502,503, 504, 506, 521, 537, 550, 564: n = A,T,C or G
tgaccgcttgtcatctcacatggggtcctgcacgcttttgcctttgtaggaaacctgacatttgtctgtttcttctttctcttttccttcccatatcctcctaatttacgtttgacttgtttgctgaggaggcaggagctagagactgctgtgagctcataggggtgggaagtttatccttcaagtcccgcccactcatcactgcttctcaccttcccctgaccaggcttacaagtgggttcttgcctgctttccctttggacccaacaagcccctgtaatgagtgtgcatgactctgacagctgtggactcagggtccttggctacagctgccatgtaaaatatctcatccagttctcgcaaattgttaaaataaccacatttcttagattccagtacccaaatcatgtctttacgaactgctcctcacacccagaagtggcacaataattcttggggaattattacttttttttttctctctnttnncgnnngnnnnggnnngnccaggaattaccacnttggaagacctggccngaatttattatanaggggagccgattntttttcctaacacaaagcgggtca

SEQ ID NO: 263 - DNA, 730 bp, Homo sapiens
  misc_feature 124, 510, 534, 559, 604, 605, 635, 711, 729: n = A,T,C or G
tttttttttttttggcctgagcaactgaaattatgaaatttccatatactcaaaagagtaagactgcaaaaagattaaatgtaaaagttgtcttgtatacagtaatgtttaagatacctattanatttataaatggaaaattagggcatttggatatacaagttgaaaattcaggagtgaggttgggctggctgggtatatactgaaaactgtcagtacacagatgacatctaaaaccacaaatctggttttattttagcagtgatatgtgtcactcccacaaaagccttcccaattggcctcagcatacacaacaagtcacctccccacagccctctacacataaacaaattccttagtttagttcaggaggaaatgcgcccttttccttccgctctaggtgaccgcaaggcccagttctcgtcaccaagatgttaagggaagtctgccaaagaggcatctgaaaggaaataaggggaatgggagtgaccacaaaggaaagccaagganaaactttggagaccgtttctaganccctggcatttcacaacaaaactcnggaacaaaccttgtctcatcaatcatttaagcccttcgtttggannagactttctgaactgggcgctgaacataancctcattgaatgtcttcacagtctcccagctgaaggcacaccttgggccagaaggggaatcttccaggtcctcaanacagggctcgccctttgnc

SEQ ID NO: 264 - DNA, 715 bp, Homo sapiens
  misc_feature 364, 451, 476, 494, 495, 515, 519, 524, 633, 635, 636,645, 647, 649, 657, 692, 695, 701, 707, 710, 713: n = A,T,C or G
tttttttttttttggccagtatgatagtctctaccactatattgaagctcttaggtcatttacacttaatgtggttatagatgctgttgagcttacttctaccaccttgctatttctcccgtctcttttttgttccttttctcttcttttcctcccttattttataattgaattttttaggattctattttatatagatttatcagctataacactttgtattcttttgttttgtggttcttctgtcatttcaatgtgcatcttaaactcatcacaatctattttcaaataatatcatataaccttacatataatgtaagaatctaccaccatatatttccatttctcccttccatcctatgtntgtcatattttttcctttatatatgttttaaagacataatagtatatgggaggtttttgcttaaaatgtgatcaatattccttcaangaaacgtaaaaattcaaaataaatntctgtttattctcaaatnnacctaatatttcctaccatntctnatacntttcaagaatctgaaggcattggttttttccggcttaagaacctcctctaaagcactctaagcagaattaagtcttctgggagaggaattctcccaagcttgggccttnanntgtactccntnanggttaaantttggccgggaaatagaaattccaagttaacaggntantttttntttttnttntcncc

SEQ ID NO: 265 - DNA, 152 bp, Homo sapiens
tttttttttttttcccaacacaaagcaccattatctttcctcacaattttcaacatagtttgattcccatgaagaggttatgatttctaaagaaaacatggctactatactatcaatcagggttaaatcttttttttttgagacggagttta

SEQ ID NO: 266 - DNA, 193 bp, Homo sapiens
  misc_feature 180: n = A,T,C or G
taaactccgtccccttcttaatcaatatggaggctacccactccacattaccttcttttcaagggactgtttccgtaactgttgtgggtattcacgaccaggcttctaaacctcttaaaactccccaattctggtgccaacttggacaacatgctttttttttttttttttttttttttngagacggagttta

SEQ ID NO: 267 - DNA, 460 bp, Homo sapiens
tgttgcgatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgacccataaaaggaggatgtggatcgcaaca

SEQ ID NO: 268 - DNA, 533 bp, Homo sapiens
  misc_feature 450, 470: n = A,T,C or G
tgttgcgatccgttgatagaatagcgacgtggtaatgagtgcatggcacgcctccgacttaccttcgcccgtggggaccccgagtacgtctacggcgtcgtcacttagagtaccctctggacgcccgggcgcgttcgatttaccggaagcgcgagctgcagtgggcttgcgcccccggccaaattctttggggggtttaaggccgcggggaatttgaggtatctctatcagtatgtagccaagttggaacagtcgccattcccgaaatcgctttctttgaatccgcaccgcctccagcattgcctcattcatcaacctgaaggcacgcataagtgacggttgtgtcttcagcagctccactccataactagcgcgctcgacctcgtcttcgtacgcgccaggtccgtgcgtgcgaattcccaactccggtgagttgcgcatttcaagttncgaaactgttcgcctccacnatttggcatgttcacgcatgacacggaataaactcgtccagtaccgggaatgggatcgcaaca

SEQ ID NO: 269 - DNA, 50 bp, Homo sapiens
ttttttttttttcgcctgaattagctacagatcctcctcacaagcggtca

SEQ ID NO: 270 - DNA, 519 bp, Homo sapiens
tgttgcgatccaaataacccaccagcttcttgcacacttcgcagaagccaccgtcctttggctgagtcacgtgaacggtcagtgcaagcagccgcgtgccagagcagaggtgcagcatgctgcacaccagctcagggctgacctcctccagcaggatggacaggatggagctgccgtacgtgtccaccacctcctggcactcttccgacagggacttcggcagcttcgagcacattttgtcaaaagcgtcgagtatttctttctcagtcttgttgttgtcaatcagcttggtcacctccttcaccaggaattcacacacctcacagtaaacatcagactttgctgggacctcgtgcttcttaatgggctccaccagttccagggcagggatgacattcttggaggccactttggcggggaccagagtctgcatgggcatctctttcacctcatcacagaacccaaccagcgcacagatctccttgggttgcatgtgcatcatcatctgggatcgcaaca

SEQ ID NO: 271 - DNA, 457 bp, Homo sapiens
ttttttttttttcgggcggcgaccggacgtgcactcctccagtagcggctgcacgtcgtgccaatggcccgctatgaggaggtgagcgtgtccggcttcgaggagttccaccgggccgtggaacagcacaatggcaagaccattttcgcctactttacgggttctaaggacgccggggggaaaagctggtgccccgactgcgtgcaggctgaaccagtcgtacgagaggggctgaagcacattagtgaaggatgtgtgttcatctactgccaagtaggagaagagccttattggaaagatccaaataatgacttcagaaaaaacttgaaagtaacagcagtgcctacactacttaagtatggaacacctcaaaaactggtagaatctgagtgtcttcaggccaacctggtggaaatgttgttctctgaagattaagattttaggatggcaatcaaga

SEQ ID NO: 272 - DNA, 102 bp, Homo sapiens
ttttttttttttgggcaacaacctgaataccttttcaaggctctggcttgggctcaagcccgcaggggaaatgcaactggccaggtcacagggcaatcaaga

SEQ ID NO: 273 - DNA, 455 bp, Homo sapiens
  misc_feature 380, 415, 454: n = A,T,C or G
ttttttttttttggcaatcaacaggtttaagtcttcggccgaagttaatctcgtgtttttggcaatcaacaggtttaagtcttcggccgaagttaatctcgtgtttttggcaatcaacaggtttaagtcttcggccgaagttaatctcgtgtttttggcaatcaacaggtttaagtcttcggccgaagttaatctcgtgtttttggcaatcaacaggtttaagtcttcggccgaagttaatctcgtgtttttggcaatcaacaggtttaagtcttcggccgaagttaatctcgtgtttttggcaatcaagaggtttaagtcttcggccgaagttaatctcgtgtttttggcaatcaacaggtttaagtcttcggccgaanttaatctcgtgtttttggcaatcaacaggtttaantcttcggccgaagttaatctcgtgtttttggcaatcaana

SEQ ID NO: 274 - DNA, 461 bp, Homo sapiens
ttttttttttttggccaatacccttgatgaacatcaatgtgaaaatcctcggtaaaatactggcaaaccaaatccagcagcacatcaaaaagcttatccaccatgatcaagtgggcttcatccctgggatgcaaggctggttcaacataagaaaatcaataaatgtaatccatcacataaacagaaccaaagacaaaaaccacatgattatctcaatagatgcagaaaaggccttggacaaattcaacagcccttcatgctaaacactcttaataaactagatattgatggaatgtatctcaaaataataagagctatttatgacaaacccacagccaatatcatactgaatgggcaaagactggaagcattccctttgaaaactggcacaagacaaggatgccctctctcaccgctcctattcaacatagtattggaagttctggccagggcaatcaaga

SEQ ID NO: 275 - DNA, 729 bp, Homo sapiens
  misc_feature 164, 193, 207, 215, 216, 220, 223, 241, 244, 254, 269,271, 275, 290, 295, 298, 309, 318, 325, 326, 331, 352, 380, 401,411, 420, 424, 426, 431, 433, 435, 438, 440, 442, 443, 448,453, 464, 465, 468, 474, 475, 481, 487, 491, 503, 516: n = A,T,C or G
  misc_feature 519, 530, 531, 542, 547, 549, 559, 561, 564, 582, 586,587, 588, 589, 592, 595, 612, 614, 620, 631, 632, 635, 636, 644,646, 649, 650, 651, 655, 657, 660, 661, 662, 663, 666, 672,673, 674, 682, 687, 691, 693, 697, 700, 701, 704, 705: n = A,T,C or G
  misc_feature 713, 715, 717, 718, 722, 726, 727: n = A,T,C or G
ttttttttttttggccaacaccaagtcttccacgtgggaggttttattatgttttacaaccatgaaaacataggaaggtggctgttacagcaaacatttcagatagacgaatcggccaagctccccaaaccccaccttcacagcctcttccacacgtctcccanagattgttgtccttcacttgcaaattcanggatgttggaagtngacatttnnagtngcnggaaccccatcagtgaancantaagcagaantacgatgactttgananacanctgatgaagaacacnctacnganaaccctttctntcgtgttangatctcnngtccntcactaatgcggccccctgcnggtccaccatttgggagaactccccccncgttggatccccccttgagtntcccattctngtcccccanaccngncttgngngncantncnncctcncaccntgtttccctgnngtnaaaatnngttttnccgccncccnaattcccacccnaatcacagcgaanccngaaggccttcnnaagtgtttaangcccngnggtttcctcntntanttgcagcctaccctcccncttnnnnttncgngttggtcgcgccctggncncgcctngttcctctttnnggnnacaacctngntcnnnggcncntcnnnnctnttcctnnnactagctngcctntccncnccgnggnncanngcacattncncnnactntgtnncc

SEQ ID NO: 276 - DNA, 339 bp, Homo sapiens
tgacctgacatgtagtagatacttaataaatatttgtggaatgaatggatgaagtggagttacagagaaaaatagaaaagtacaaattgttgtcagtgttttgaaggaaaattatgatctttcccaaagttctgacttcattctaagacagggttagtatctccatacataattttacttgcttttgaaaatcaaatgagataatctatttagattgataatttatttagactggctataaactattaagtgctagcaaatatacattttaatctcattttccacctcttgtgatatagctatgtaggtgttgactttaatggatgtcaggtcaatccc

SEQ ID NO: 277 - DNA, 664 bp, Homo sapiens
  misc_feature 267, 534, 590, 601, 646, 657: n = A,T,C or G
tgacctgacatccataacaaaatctttctccattatattcttctaggggaatttcttgaaaagcatccaaaggaaacaaatgatggtaagaccgtgccaagtggggagcagacaccaaagtaagaccacagattttacattcaacaggtagctcacagtactttgcccgacactgtgggcagaaatagcctcctaatgtaagccctggctcagtattgccatccaaatgcgccatgctgaaagagggttttgcatcctggtcagatnaagaagcaatggtgtgctgaggaaatcccatacgaataagtgagcattcagaacttgagctagcaggaggaggactaagatgatgtgtgagcaactctttgtaatggctttcatctaaaataacatggtacgtgccaccagtttcacgagcaagtacagtgcaaacgcgaacttctgcagacaatccaataacagatactctaattttagctgcctttagggtcttgattaaatcataaatattagatggatcgcaagttgtaaggntgctaaaagatgattagtacttctcgacttgtatgtccaggcatgttgttttaaantctgccttagnccctgcttaggggaatttttaaagaagatggctctccatgttcanggtcaatcacnaattgcc

SEQ ID NO: 278 - DNA, 452 bp, Homo sapiens
  misc_feature 430: n = A,T,C or G
tgacctgacattgaggaagagcacacacctctgaaattccttaggttcagaagggcatttgacacagagtgggcctctgataattcatgaaatgcattctgaagtcatccagaatggaggctgcaatctgctgtgctttgggggttgcctcactgtgctcctggatatcacacaaaagctgcaatccttcttcttcaactaacattttgcagtatttgctgggatttttactgcagacatgatacatagcccatagtgcccagagctgaacctctggttgagagaagttgccaaggagcgggaaaaatgtcttgaaagatctataggtcaccaatgctgtcatcttacaacttgaacttggccaattctgtatggttgcatgcagatcttggagaagagtacgcctctggaagtcacgggatatccaaanctgtctgtcagatgtcaggtca

SEQ ID NO: 279 - DNA, 274 bp, Homo sapiens
ttttttttttttcggcaaggcaaatttacttctgcaaaagggtgctgcttgcacttttggccactgcgagagcacaccaaacaaagtagggaaggggtttttatccctaacgcggttattccctggttctgtgtcgtgtccccattggctggagtcagactgcacaatctacactgacccaactggctactgtttaaaattgaatatgaataattaggtaggaagggggaggctgtttgttacggtacaagacgtgtttgggcatgtcaggtca

SEQ ID NO: 280 - DNA, 272 bp, Homo sapiens
tacctgacatggagaaataacttgtagtattttgcgtgcaatggaatactatatgagggtgaaaatgaatgaactagcaatgcgtgtatcaacatgaataaatccccaaaacataataatgttgaatggaaaaggtgagtttcagaaggatatatatgccctctaaatccatttatgtaaacctttaaaaaactacattatttatggtcataagtccatccagaaaatatttaaaaacctacatgggattgataactactgatgtcaggtca

SEQ ID NO: 281 - DNA, 431 bp, Homo sapiens
  misc_feature 339, 420, 430, 431: n = A,T,C or G
ttttttttttttggccaatagcatgatttaaacattggaaaaagtcaaatgagcaatgcgaatttttatgttctcttgaataatcaaaagagtaggcaacattggttcctcattcttgaatagcattaatcagaaaatattgcatagcctctagcctccttagagtaggtgtgctctctcaaatatatcatagtcccacagtttatttcatgtatattttctgcctgaatcacatagacatttgaatttgcaacgcctgatgtaaatatataaattcttaccaatcagaaacatagcaagaaattcagggacttggtcatyatcagggtatgacagcanatccctgtaraaacactgatacacactcacacacgtatgcaacgtggagatgtcgcyttwwkkktwywcwmrmrycrwcgnaatcacttann

SEQ ID NO: 282 - DNA, 98 bp, Homo sapiens
attcgattcgatgcttgagcccaggagttcaagactgcagtgagccactgcacttcaggctggacaacagagcgagtccctgtgccaaaaaaaaaaaa

SEQ ID NO: 283 - DNA, 764 bp, Homo sapiens
  misc_feature 372, 374, 379, 380, 381, 382, 384, 387, 389, 392, 402,409, 411, 419, 421, 432, 440, 447, 452, 457, 466, 470, 471, 480,483, 492, 503, 506, 510, 512, 518, 520, 521, 524, 531, 534,536, 542, 545, 547, 550, 552, 553, 562, 566, 567, 575: n = A,T,C or G
  misc_feature 580, 581, 584, 586, 587, 595, 598, 601, 603, 604, 606,624, 629, 630, 646, 651, 652, 653, 656, 659, 664, 665, 681, 691,700, 706, 709, 721, 724, 731, 732, 737, 741, 744, 745, 750,753, 754, 758: n = A,T,C or G
ttttttttttttcgcaagcacgtgcactttattgaatgacactgtagacaggtgtgtgggtataaactgctgtatctaggggcaggaccaagggggcaggggcaacagccccagcgtgcagggccascattgcacagtggastgcaaaggttgcaggctatgggcggctactavtaaccccgtttttcctgtattatctgtaacataatatggtagactgtcacagagccgaatwccarthacasgatgaatccaawggtcaygaggatgcccasaatcagggcccasatsttcaggcacttggcggtgggggcatasgcctgkgccccggtcacgtcsccaaccwtctycctgtccctacmcttgawtccncnccttnnnntnccntnatntgcccgcccncctcctngngtcaaccngnatctgcactanctccctcnccccttntggantctcntccttcaantaannttatccttnacncccccctcncctttcccctnccncccntnatcccngnnccnctatcantcntnccctcnctntnctncnnatcgttccncctnntaactacnctttnnacnanncctcactnatnccngnnanttctttccttccctcccnacgcnntgcgtgcgcccgtctngcctnnnctncgnacccnnactttatttacctttncaccctagcnctctacttnacccanccnctcctacctccnggnccacccnnccctnatcnctnnctctntcnnctcnttcccc

SEQ ID NO: 284 - DNA, 157 bp, Homo sapiens
caagtgtaggcacagtgatgaaagcctggagcaaacacaatctgtgggtaattaacgtttatttctccccttccaggaacgtcttgcatggatgatcaaagatcagctcctggtcaacataaataagctagtttaagatacgttcccctacacttga

SEQ ID NO: 285 - DNA, 150 bp, Homo sapiens
attcgattgtactcagacaacaatatgctaagtggaagaagtcagtcacaaaagaccacatactgtatgacttcatttacattaagtgtccagaataggcaaatccgtagagacagaaagtagatgagcagctgcctaggtctgagtaca

SEQ ID NO: 286 - DNA, 219 bp, Homo sapiens
attcgatttttttttttttggccatgatgaaattcttactccctcagattttttgtctggataaatgcaagtctcaccaccagatgtgaaattacagtaaactttgaaggaatctcctgagcaaccttggttaggatcaatccaatattcaccatctgggaagtcaggatggctgagttgcaggtctttacaagttcgggctggattggtctgagtaca

SEQ ID NO: 287 - DNA, 196 bp, Homo sapiens
attcgattcttgaggctaccaggagctaggagaagaggcatggaacaaattttccctcatatccatactcagaaggaaccaaccctgctgacaccttaatttcagcttctggcctctagaactgtgagagagtacatttctcttggtttaagccaagagaatctgtcttttggtactttatatcatagcctcaaga

SEQ ID NO: 288 - DNA, 199 bp, Homo sapiens
attcgatttcagtccagtcccagaacccacattgtcaattactactctgtaraagattcatttgttgaaattcattgagtaaaacatttatgatcccttaatatatgccaattaccatgctaggtactgaagattcaagtgaccgagatgctagcccttgggttcaagtgatccctctcccagagtgcactggactgaa

SEQ ID NO: 289 - DNA, 182 bp, Homo sapiens
attcgattcttgaggctacaaacctgtacagtatgttactctactgaatactgtaggcaatagtaatacagaagcaagtatctgtatatgtaaacattaaaaaggtacagtgaaacttcagtattataatcttagggaccaccattatatatgtggtccatcattggccaaaaaaaaaaaaa

SEQ ID NO: 290 - DNA, 1646 bp, Homo sapiens
ggcacgaggagaaatgtaattccatattttatttgaaacttattccatattttaattggatattgagtgattgggttatcaaacacccacaaactttaattttgttaaatttatatggctttgaaatagaagtataagttgctaccattttttgataacattgaaagatagtattttaccatctttaatcatcttggaaaatacaagtcctgtgaacaaccactctttcacctagcagcatgaggccaaaagtaaaggctttaaattataacatatgggattcttagtagtatgtttttttcttgaaactcagtggctctatctaaccttactatctcctcactctttctctaagactaaactctaggctcttaaaaatctgcccacaccaatcttagaagctctgaaaagaatttgtctttaaatatcttttaatagtaacatgtattttatggaccaaattgacattttcgactattttttccaaaaaagtcaggtgaatttcagcacactgagttgggaatttcttatcccagaagaccaaccaatttcatatttatttaagattgattccatactccgttttcaaggagaatccctgcagtctccttaaaggtagaacaaatactttctatttttttttcaccattgtgggattggactttaagaggtgactctaaaaaaacagagaacaaatatgtctcagttgtattaagcacggacccatattatcatattcacttaaaaaaatgatttcctgtgcaccttttggcaacttctcttttcaatgtagggaaaaacttagtcaccctgaaaacccacaaaataaataaaacttgtagatgtgggcagaaggtttgggggtggacattgtatgtgtttaaattaaaccctgtatcactgagaagctgttgtatgggtcagagaaaatgaatgcttagaagctgttcacatcttcaagagcagaagcaaaccacatgtctcagctatattattatttattttttatgcataaagtgaatcatttcttctgtattaatttccaaagggttttaccctctatttaaatgctttgaaaaacagtgcattgacaatgggttgatatttttctttaaaagaaaaatataattatgaaagccaagataatctgaagcctgttttattttaaaactttttatgttctgtggttgatgttgtttgtttgtttgtttctattttgttggttttttactttgttttttgttttgttttgttttgtttkgcatactacatgcagttctttaaccaatgtctgtttggctaatgtaattaaagttgttaatttatatgagtgcatttcaactatgtcaatggtttcttaatatttattgtgtagaagtactggtaatttttttatttacaatatgtttaaagagataacagtttgatatgttttcatgtgtttatagcagaagttatttatttctatggcattccagcggatattttggtgtttgcgaggcatgcagtcaatattttgtacagttagtggacagtattcagcaacgcctgatagcttctttggccttatgttaaataaaaagacctgtttgggatgtattttttatttttaaaaaaaaaaaaaaaaaaaaaaaaaaaaaaaaaaa

SEQ ID NO: 291 - DNA, 1851 bp, Homo sapiens
tcatcaccattgccagcagcggcaccgttagtcaggttttctgggaatcccacatgagtacttccgtgttcttcattcttcttcaatagccataaatcttctagctctggctggctgttttcacttcctttaagcctttgtgactcttcctctgatgtcagctttaagtcttgttctggattgctgttttcagaagagatttttaacatctgtttttctttgtagtcagaaagtaactggcaaattacatgatgatgactagaaacagcatactctctggccgtctttccagatcttgagaagatacatcaacattttgctcaagtagagggctgactatacttgctgatccacaacatacagcaagtatgagagcagttcttccatatctatccagcgcatttaaattcgcttttttcttgattaaaaatttcaccacttgctgtttttgctcatgtataccaagtagcagtggtgtgaggccatgcttgttttttgattcgatatcagcaccgtataagagcagtgctttggccattaatttatcttcattgtagacagcatagtgtagagtggtatttccatactcatctggaatatttggatcagtgccatgttccagcaacattaacgcacattcatcttcctggcattgtacggcctttgtcagagctgtcctctttttgttgtcaaggacattaagttgacatcgtctgtccagcacgagttttactacttctgaattcccattggcagaggccagatgtagagcagtcctcttttgcttgtccctcttgttcacatccgtgtccctgagcatgacgatgagatcctttctggggactttaccccaccaggcagctctgtggagcttgtccagatcttctccatggacgtggtacctgggatccatgaaggcgctgtcatcgtagtctccccaagcgaccacgttgctcttgccgctcccctgcagcaggggaagcagtggcagcaccacttgcacctcttgctcccaagcgtcttcacagaggagtcgttgtggtctccagaagtgcccacgttgctcttgccgctccccctgtccatccagggaggaagaaatgcaggaaatgaaagatgcatgcacgatggtatactcctcagccatcaaacttctggacagcaggtcacttccagcaaggtggagaaagctgtccacccacagaggatgagatccagaaaccacaatatccattcacaaacaaacacttttcagccagacacaggtactgaaatcatgtcatctgcggcaacatggtggaacctacccaatcacacatcaagagatgaagacactgcagtatatctgcacaacgtaatactcttcatccataacaaaataatataattttcctctggagccatatggatgaactatgaaggaagaactccccgaagaagccagtcgcagagaagccacactgaagctctgtcctcagccatcagcgccacggacaggartgtgtttcttccccagtgatgcagcctcaagttatcccgaagctgccgcagcacacggtggctcctgagaaacaccccagctcttccggtctaacacaggcaagtcaataaatgtgataatcacataaacagaattaaaagcaaagtcacataagcatctcaacagacacagaaaaggcatttgacaaaatccagcatccttgtatttattgttgcagttctcagaggaaatgcttctaacttttccccatttagtattatgttggctgtgggcttgtcataggtggtttttattactttaaggtatgtcccttctatgcctgttttgctgagggttttaattctcgtgcc

SEQ ID NO: 292 - DNA, 1851 bp, Homo sapiens; identical to SEQ ID NO: 291

SEQ ID NO: 293 - DNA, 668 bp, Homo sapiens
cttgagcttccaaatayggaagactggcccttacacasgtcaatgttaaaatgaatgcatttcagtattttgaagataaaattrgtagatctataccttgttttttgattcgatatcagcaccrtataagagcagtgctttggccattaatttatctttcattrtagacagcrtagtgyagagtggtatttccatactcatctggaatatttggatcagtgccatgttccagcaacattaacgcacattcatcttcctggcattgtacggcctgtcagtattagacccaaaaacaaattacatatcttaggaattcaaaataacattccacagctttcaccaactagttatatttaaaggagaaaactcatttttatgccatgtattgaaatcaaacccacctcatgctgatatagttggctactgcatacctttatcagagctgtcctctttttgttgtcaaggacattaagttgacatcgtctgtccagcaggagttttactacttctgaattcccattggcagaggccagatgtagagcagtcctatgagagtgagaagactttttaggaaattgtagtgcactagctacagccatagcaatgattcatgtaactgcaaacactgaatagcctgctattactctgccttcaaaaaaaaaaaaaaa

SEQ ID NO: 294 - DNA, 1512 bp, Homo sapiens
gggtcgcccagggggsgcgtgggctttcctcgggtgggtgtgggttttccctgggtggggtgggctgggctrgaatcccctgctggggttggcaggttttggctgggattgacttttytcttcaaacagattggaaacccggagttacctgctagttggtgaaactggttggtagacgcgatctgttggctactactggcttctcctggctgttaaaagcagatggtggttgaggttgattccatgccggctgcttcttctgtgaagaagccatttggtctcaggagcaagatgggcaagtggtgctgccgttgcttcccctgctgcagggagagcggcaagagcaacgtgggcacttctggagaccacgacgactctgctatgaagacactcaggagcaagatgggcaagtggtgccgccactgcttcccctgctgcagggggagtggcaagagcaacgtgggcgcttctggagaccacgacgaytctgctatgaagacactcaggaacaagatgggcaagtggtgctgccactgcttcccctgctgcagggggagcrgcaagagcaaggtgggcgcttggggagactacgatgacagtgccttcatggagcccaggtaccacgtccgtggagaagatctggacaagctccacagagctgcctggtggggtaaagtccccagaaaggatctcatcgtcatgctcagggacactgacgtgaacaagaaggacaagcaaaagaggactgctctacatctggcctctgccaatgggaattcagaagtagtaaaactcstgctggacagacgatgtcaacttaatgtccttgacaacaaaaagaggacagctctgayaaaggccgtacaatgccaggaagatgaatgtgcgttaatgttgctggaacatggcactgatccaaatattccagatgagtatggaaataccactctrcactaygctrtctayaatgaagataaattaatggccaaagcactgctcttatayggtgctgatatcgaatcaaaaaacaaggtatagatctactaattttatcttcaaaatactgaaatgcattcattttaacattgacgtgtgtaagggccagtcttccgtatttggaagctcaagcataacttgaatgaaaatattttgaaatgacctaattatctmagactttattttaaatattgttattttcaaagaagcattagagggtacagttttttttttttaaatgcacttctggtaaatacttttgttgaaaacactgaatttgtaaaaggtaatacttactatttttcaatttttccctcctaggatttttttcccctaatgaatgtaagatggcaaaatttgccctgaaataggttttacatgaaaactccaagaaaagttaaacatgtttcagtgaatagagatcctgctcctttggcaagttcctaaaaaacagtaatagatacgaggtgatgcgcctgtcagtggcaaggtttaagatatttctgatctcgtgcc

SEQ ID NO: 295 - DNA, 1853 bp, Homo sapiens
gggtcgcccagggggsgcgtgggctttcctcgggtgggtgtgggttttccctgggtggggtgggctgggctrgaatcccctgctggggttggcaggttttggctgggattgacttttytcttcaaacagattggaaacccggagttacctgctagttggtgaaactggttggtagacgcgatctgttggctactactggcttctcctggctgttaaaagcagatggtggttgaggttgattccatgccggctgcttcttctgtgaagaagccatttggtctcaggagcaagatgggcaagtggtgctgccgttgcttcccctgctgcagggagagcggcaagagcaacgtgggcacttctggagaccacgacgactctgctatgaagacactcaggagcaagatgggcaagtggtgccgccactgcttcccctgctgcagggggagtggcaagagcaacgtgggcgcttctggagaccacgacgaytctgctatgaagacactcaggaacaagatgggcaagtggtgctgccactgcttcccctgctgcagggggagcrgcaagagcaaggtgggcgcttggggagactacgatgacagygccttcatggakcccaggtaccacgtccrtggagaagatctggacaagctccacagagctgcctggtggggtaaagtccccagaaaggatctcatcgtcatgctcagggacackgaygtgaacaagarggacaagcaaaagaggactgctctacatctggcctctgccaatgggaattcagaagtagtaaaactcstgctggacagacgatgtcaacttaatgtccttgacaacaaaaagaggacagctctgayaaaggccgtacaatgccaggaagatgaatgtgcgttaatgttgctggaacatggcactgatccaaatattccagatgagtatggaaataccactctrcactaygctrtctayaatgaagataaattaatggccaaagcactgctcttatayggtgctgatatcgaatcaaaaaacaagcatggcctcacaccactgytacttggtrtacatgagcaaaaacagcaagtsgtgaaatttttaatyaagaaaaaagcgaatttaaaatgcrctggatagatatggaagractgctctcatacttgctgtatgttgtggatcagcaagtatagtcagccytctacttgagcaaaatrttgatgtatcttctcaagatctggaaagacggccagagagtatgctgtttctagtcatcatcatgtaatttgccagttactttctgactacaaagaaaaacagatgttaaaaatctcttctgaaaacagcaatccagaacaagacttaaagctgacatcagaggaagagtcacaaaggcttaaaggaagtgaaaacagccagccagaggcatggaaacttttaaatttaaacttttggtttaatgttttttttttttgccttaataatattagatagtcccaaatgaaatwacctatgagactaggctttgagaatcaatagattctttttttaagaatcttttggctaggagcggtgtctcacgcctgtaattccagcaccttgagaggctgaggtgggcagatcacgagatcaggagatcgagaccatcctggctaacacggtgaaaccccatctctactaaaaatacaaaaacttagctgggtgtggtggcgggtgcctgtagtcccagctactcaggargctgaggcaggagaatggcatgaacccgggaggtggaggttgcagtgagccgagatccgccactacactccagcctgggtgacagagcaagactctgtctcaaaaaaaaaaaaaaaaaaaaa

SEQ ID NO: 296 - DNA, 2184 bp, Homo sapiens
ggcacgagaattaaaaccctcagcaaaacaggcatagaagggacataccttaaagtaataaaaaccacctatgacaagcccacagccaacataatactaaatggggaaaagttagaagcatttcctctgagaactgcaacaataaatacaaggatgctggattttgtcaaatgccttttctgtgtctgttgagatgcttatgtgactttgcttttaattctgtttatgtgattatcacatttattgacttgcctgtgttagaccggaagagctggggtgtttctcaggagccaccgtgtgctgcggcagcttcgggataacttgaggctgcatcactggggaagaaacacaytcctgtccgtggcgctgatggctgaggacagagcttcagtgtggcttctctgcgactggcttcttcggggagttcttccttcatagttcatccatatggctccagaggaaaattatattattttgttatggatgaagagtattacgttgtgcagatatactgcagtgtcttcatctcttgatgtgtgattgggtaggttccaccatgttgccgcagatgacatgatttcagtacctgtgtctggctgaaaagtgtttgtttgtgaatggatattgtggtttctggatctcatcctctgtgggtggacagctttctccaccttgctggaagtgacctgctgtccagaagtttgatggctgaggagtataccatcgtgcatgcatctttcatttcctgcatttcttcctccctggatggacagggggagcggcaagagcaacgtgggcacttctggagaccacaacgactcctctgtgaagacgcttgggagcaagaggtgcaagtggtgctgccactgcttcccctgctgcaggggagcggcaagagcaacgtggtcgcttggggagactacgatgacagcgccttcatggatcccaggtaccacgtccatggagaagatctggacaagctccacagagctgcctggtggggtaaagtccccagaaaggatctcatcgtcatgctcagggacacggatgtgaacaagagggacaagcaaaagaggactgctctacatctggcctctgccaatgggaattcagaagtagtaaaactcgtgctggacagacgatgtcaacttaatgtccttgacaacaaaaagaggacagctctgacaaaggccgtacaatgccaggaagatgaatgtgcgttaatgttgctggaacatggcactgatccaaatattccagatgagtatggaaataccactctacactatgctgtctacaatgaagataaattaatggccaaagcactgctcttatacggtgctgatatcgaatcaaaaaacaagcatggcctcacaccactgctacttggtatacatgagcaaaaacagcaagtggtgaaatttttaatcaagaaaaaagcgaatttaaatgcgctggatagatatggaagaactgctctcatacttgctgtatgttgtggatcagcaagtatagtcagccctctacttgagcaaaatgttgatgtatcttctcaagatctggaaagacggccagagagtatgctgtttctagtcatcatcatgtaatttgccagttactttctgactacaaagaaaaacagatgttaaaaatctcttctgaaaacagcaatccagaacaagacttaaagctgacatcagaggaagagtcacaaaggcttaaaggaagtgaaaacagccagccagaggcatggaaacttttaaatttaaacttttggtttaatgttttttttttttgccttaataatattagatagtcccaaatgaaatwacctatgagactaggctttgagaatcaatagattctttttttaagaatcttttggctaggagcggtgtctcacgcctgtaattccagcaccttgagaggctgaggtgggcagatcacgagatcaggagatcgagaccatcctggctaacacggtgaaaccccatctctactaaaaatacaaaaacttagctgggtgtggtggcgggtgcctgtagtcccagctactcaggargctgaggcaggagaatggcatgaacccgggaggtggaggttgcagtgagccgagatccgccactacactccagcctgggtgacagagcaagactctgtctcaaaaaaaaaaaaaaaaaaaaa

SEQ ID NO: 297 - DNA, 1855 bp, Homo sapiens
  misc_feature 606: n = A,T,C or G
tgcacgcatcggccagtgtctgtgccacgtacactgacgccccctgagatgtgcacgccgcacgcgcacgttgcacgcgcggcagcggcttggctggcttgtaacggcttgcacgcgcacgccgcccccgcataaccgtcagactggcctgtaacggcttgcaggcgcacgccgcacgcgcgtaacggcttggctgccctgtaacggcttgcacgtgcatgctgcacgcgcgttaacggcttggctggcatgtagccgcttggcttggctttgcattytttgctkggctkggcgttgktytcttggattgacgcttcctccttggatkgacgtttcctccttggatkgacgtttcytytytcgcgttcctttgctggacttgaccttttytctgctgggtttggcattcctttggggtgggctgggtgttttctccgggggggktkgcccttcctggggtgggcgtgggkcgcccccagggggcgtgggctttccccgggtgggtgtgggttttcctggggtggggtgggctgtgctgggatccccctgctggggttggcagggattgacttttttcttcaaacagattggaaacccggagtaacntgctagttggtgaaactggttggtagacgcgatctgctggtactactgtttctcctggctgttaaaagcagatggtggctgaggttgattcaatgccggctgcttcttctgtgaagaagccatttggtctcaggagcaagatgggcaagtggtgcgccactgcttcccctgctgcagggggagcggcaagagcaacgtgggcacttctggagaccacaacgactcctctgtgaagacgcttgggagcaagaggtgcaagtggtgctgcccactgcttcccctgctgcaggggagcggcaagagcaacgtggkcgcttggggagactacgatgacagcgccttcatggakcccaggtaccacgtccrtggagaagatctggacaagctccacagagctgcctggtggggtaaagtccccagaaaggatctcatcgtcatgctcagggacactgaygtgaacaagarggacaagcaaaagaggactgctctacatctggcctctgccaatgggaattcagaagtagtaaaactcgtgctggacagacgatgtcaacttaatgtccttgacaacaaaaagaggacagctctgacaaaggccgtacaatgccaggaagatgaatgtgcgttaatgttgctggaacatggcactgatccaaatattccagatgagtatggaaataccactctacactatgctgtctacaatgaagataaattaatggccaaagcactgctcttatacggtgctgatatcgaatcaaaaaacaaggtatagatctactaattttatcttcaaaatactgaaatgcattcattttaacattgacgtgtgtaagggccagtcttccgtatttggaagctcaagcataacttgaatgaaaatattttgaaatgacctaattatctaagactttattttaaatattgttattttcaaagaagcattagagggtacagttttttttttttaaatgcacttctggtaaatacttttgttgaaaacactgaatttgtaaaaggtaatacttactatttttcaatttttccctcctaggatttttttcccctaatgaatgtaagatggcaaaatttgccctgaaataggttttacatgaaaactccaagaaaagttaaacatgtttcagtgaatagagatcctgctcctttggcaagttcctaaaaaacagtaatagatacgaggtgatgcgcctgtcagtggcaaggtttaagatatttctgatctcgtgcc

SEQ ID NO: 298 - DNA, 1059 bp, Homo sapiens
gcaacgtgggcacttctggagaccacaacgactcctctgtgaagacgcttgggagcaagaggtgcaagtggtgctgcccactgcttcccctgctgcaggggagcggcaagagcaacgtgggcgcttgrggagactmcgatgacagygccttcatggagcccaggtaccacgtccgtggagaagatctggacaagctccacagagctgccctggtggggtaaagtccccagaaaggatctcatcgtcatgctcagggacactgaygtgaacaagarggacaagcaaaagaggactgctctacatctggcctctgccaatgggaattcagaagtagtaaaactcstgctggacagacgatgtcaacttaatgtccttgacaacaaaaagaggacagctctgayaaaggccgtacaatgccaggaagatgaatgtgcgttaatgttgctggaacatggcactgatccaaatattccagatgagtatggaaataccactctrcactaygctrtctayaatgaagataaattaatggccaaagcactgctcttatayggtgctgatatcgaatcaaaaaacaaggtatagatctactaattttatcttcaaaatactgaaatgcattcattttaacattgacgtgtgtaagggccagtcttccgtatttggaagctcaagcataacttgaatgaaaatattttgaaatgacctaattatctaagactttattttaaatattgttattttcaaagaagcattagagggtacagttttttttttttaaatgcacttctggtaaatacttttgttgaaaacactgaatttgtaaaaggtaatacttactatttttcaatttttccctcctaggatttttttcccctaatgaatgtaagatggcaaaatttgccctgaaataggttttacatgaaaactccaagaaaagttaaacatgtttcagtgaatagagatcctgctcctttggcaagttcctaaaaaacagtaatagatacgaggtgatgcgcctgtcagtggcaaggtttaagatatttctgatctcgtgcc

SEQ ID NO: 299 - protein, 329 aa, Homo sapiens
MDIVVSGSHPLWVDSFLHLAGSDLLSRSLMAEEYTIVHASFISCISSSLDGQGERQEQRGHFWRPQRLLCEDAWEQEVQVVLPLLPLLQGSGKSNVVAWGDYDDSAFMDPRYHVHGEDLDKLHRAAWWGKVPRKDLIVMLRDTDVNKRDKQKRTALHLASANGNSEVVKLVLDRRCQLNVLDNKKRTALTKAVQCQEDECALMLLEHGTDPNIPDEYGNTTLHYAVYNEDKLMAKALLLYGADIESKNKHGLTPLLLGIHEQKQQVVKFLIKKKANLNALDRYGRTALILAVCCGSASIVSPLLEQNVDVSSQDLERRPESMLFLVIIM

SEQ ID NO: 300 - protein, 148 aa, Homo sapiens
  VARIANT 3, 46, 69, 88, 124: Xaa = Any Amino Acid
MTXPSWSPGTTSVEKIWTSSTELPWWGKVPRKDLIVMLRDTDVNKXDKQKRTALHLASANGNSEVVKLXLDRRCQLNVLDNKKRTALXKAVQCQEDECALMLLEHGTDPNIPDEYGNTTLHYAXYNEDKLMAKALLLYGADIESKNKV

SEQ ID NO: 301 - DNA, 1155 bp, Homo sapiens
atggtggttgaggttgattccatgccggctgcctcttctgtgaagaagccatttggtctcaggagcaagatgggcaagtggtgctgccgttgcttcccctgctgcagggagagcggcaagagcaacgtgggcacttctggagaccacgacgactctgctatgaagacactcaggagcaagatgggcaagtggtgccgccactgcttcccctgctgcagggggagtggcaagagcaacgtgggcgcttctggagaccacgacgactctgctatgaagacactcaggaacaagatgggcaagtggtgctgccactgcttcccctgctgcagggggagcggcaagagcaaggtgggcgcttggggagactacgatgacagtgccttcatggagcccaggtaccacgtccgtggagaagatctggacaagctccacagagctgcctggtggggtaaagtccccagaaaggatctcatcgtcatgctcagggacactgacgtgaacaagaaggacaagcaaaagaggactgctctacatctggcctctgccaatgggaattcagaagtagtaaaactcctgctggacagacgatgtcaacttaatgtccttgacaacaaaaagaggacagctctgataaaggccgtacaatgccaggaagatgaatgtgcgttaatgttgctggaacatggcactgatccaaatattccagatgagtatggaaataccactctgcactacgctatctataatgaagataaattaatggccaaagcactgctcttatatggtgctgatatcgaatcaaaaaacaagcatggcctcacaccactgttacttggtgtacatgagcaaaaacagcaagtcgtgaaatttttaatcaagaaaaaagcgaatttaaatgcactggatagatatggaaggactgctctcatacttgctgtatgttgtggatcagcaagtatagtcagccttctacttgagcaaaatattgatgtatcttctcaagatctatctggacagacggccagagagtatgctgtttctagtcatcatcatgtaatttgccagttactttctgactacaaagaaaaacagatgctaaaaatctcttctgaaaacagcaatccagaaaatgtctcaagaaccagaaataaataa

SEQ ID NO: 302 - DNA, 2000 bp, Homo sapiens
atggtggttgaggttgattccatgccggctgcctcttctgtgaagaagccatttggtctcaggagcaagatgggcaagtggtgctgccgttgcttcccctgctgcagggagagcggcaagagcaacgtgggcacttctggagaccacgacgactctgctatgaagacactcaggagcaagatgggcaagtggtgccgccactgcttcccctgctgcagggggagtggcaagagcaacgtgggcgcttctggagaccacgacgactctgctatgaagacactcaggaacaagatgggcaagtggtgctgccactgcttcccctgctgcagggggagcggcaagagcaaggtgggcgcttggggagactacgatgacagtgccttcatggagcccaggtaccacgtccgtggagaagatctggacaagctccacagagctgcctggtggggtaaagtccccagaaaggatctcatcgtcatgctcagggacactgacgtgaacaagaaggacaagcaaaagaggactgctctacatctggcctctgccaatgggaattcagaagtagtaaaactcctgctggacagacgatgtcaacttaatgtccttgacaacaaaaagaggacagctctgataaaggccgtacaatgccaggaagatgaatgtgcgttaatgttgctggaacatggcactgatccaaatattccagatgagtatggaaataccactctgcactacgctatctataatgaagataaattaatggccaaagcactgctcttatatggtgctgatatcgaatcaaaaaacaagcatggcctcacaccactgttacttggtgtacatgagcaaaaacagcaagtcgtgaaatttttaatcaagaaaaaagcgaatttaaatgcactggatagatatggaaggactgctctcatacttgctgtatgttgtggatcagcaagtatagtcagccttctacttgagcaaaatattgatgtatcttctcaagatctatctggacagacggccagagagtatgctgtttctagtcatcatcatgtaatttgccagttactttctgactacaaagaaaaacagatgctaaaaatctcttctgaaaacagcaatccagaacaagacttaaagctgacatcagaggaagagtcacaaaggttcaaaggcagtgaaaatagccagccagagaaaatgtctcaagaaccagaaataaataaggatggtgatagagaggttgaagaagaaatgaagaagcatgaaagtaataatgtgggattactagaaaacctgactaatggtgtcactgctggcaatggtgataatggattaattcctcaaaggaagagcagaacacctgaaaatcagcaatttcctgacaacgaaagtgaagagtatcacagaatttgcgaattagtttctgactacaaagaaaaacagatgccaaaatactcttctgaaaacagcaacccagaacaagacttaaagctgacatcagaggaagagtcacaaaggcttgagggcagtgaaaatggccagccagagctagaaaattttatggctatcgaagaaatgaagaagcacggaagtactcatgtcggattcccagaaaacctgactaatggtgccactgctggcaatggtgatgatggattaattcctccaaggaagagcagaacacctgaaagccagcaatttcctgacactgagaatgaagagtatcacagtgacgaacaaaatgatactcagaagcaattttgtgaagaacagaacactggaatattacacgatgagattctgattcatgaagaaaagcagatagaagtggttgaaaaaatgaattctgagctttctcttagttgtaagaaagaaaaagacatcttgcatgaaaatagtacgttgcgggaagaaattgccatgctaagactggagctagacacaatgaaacatcagagccagctaaaaaaaaaaaaaaaaaaaaaaaaaaaaaaaaa

SEQ ID NO: 303 - DNA, 2040 bp, Homo sapiens
atggtggttgaggttgattccatgccggctgcctcttctgtgaagaagccatttggtctcaggagcaagatgggcaagtggtgctgccgttgcttcccctgctgcagggagagcggcaagagcaacgtgggcacttctggagaccacgacgactctgctatgaagacactcaggagcaagatgggcaagtggtgccgccactgcttcccctgctgcagggggagtggcaagagcaacgtgggcgcttctggagaccacgacgactctgctatgaagacactcaggaacaagatgggcaagtggtgctgccactgcttcccctgctgcagggggagcggcaagagcaaggtgggcgcttggggagactacgatgacagtgccttcatggagcccaggtaccacgtccgtggagaagatctggacaagctccacagagctgcctggtggggtaaagtccccagaaaggatctcatcgtcatgctcagggacactgacgtgaacaagaaggacaagcaaaagaggactgctctacatctggcctctgccaatgggaattcagaagtagtaaaactcctgctggacagacgatgtcaacttaatgtccttgacaacaaaaagaggacagctctgataaaggccgtacaatgccaggaagatgaatgtgcgttaatgttgctggaacatggcactgatccaaatattccagatgagtatggaaataccactctgcactacgctatctataatgaagataaattaatggccaaagcactgctcttatatggtgctgatatcgaatcaaaaaacaagcatggcctcacaccactgttacttggtgtacatgagcaaaaacagcaagtcgtgaaatttttaatcaagaaaaaagcgaatttaaatgcactggatagatatggaaggactgctctcatacttgctgtatgttgtggatcagcaagtatagtcagccttctacttgagcaaaatattgatgtatcttctcaagatctatctggacagacggccagagagtatgctgtttctagtcatcatcatgtaatttgccagttactttctgactacaaagaaaaacagatgctaaaaatctcttctgaaaacagcaatccagaacaagacttaaagctgacatcagaggaagagtcacaaaggttcaaaggcagtgaaaatagccagccagagaaaatgtctcaagaaccagaaataaataaggatggtgatagagaggttgaagaagaaatgaagaagcatgaaagtaataatgtgggattactagaaaacctgactaatggtgtcactgctggcaatggtgataatggattaattcctcaaaggaagagcagaacacctgaaaatcagcaatttcctgacaacgaaagtgaagagtatcacagaatttgcgaattagtttctgactacaaagaaaaacagatgccaaaatactcttctgaaaacagcaacccagaacaagacttaaagctgacatcagaggaagagtcacaaaggcttgagggcagtgaaaatggccagccagagaaaagatctcaagaaccagaaataaataaggatggtgatagagagctagaaaattttatggctatcgaagaaatgaagaagcacggaagtactcatgtcggattcccagaaaacctgactaatggtgccactgctggcaatggtgatgatggattaattcctccaaggaagagcagaacacctgaaagccagcaatttcctgacactgagaatgaagagtatcacagtgacgaacaaaatgatactcagaagcaattttgtgaagaacagaacactggaatattacacgatgagattctgattcatgaagaaaagcagatagaagtggttgaaaaaatgaattctgagctttctcttagttgtaagaaagaaaaagacatcttgcatgaaaatagtacgttgcgggaagaaattgccatgctaagactggagctagacacaatgaaacatcagagccagctaaaaaaaaaaaaaaaaaaaaaaaaaaaa

SEQ ID NO: 304 - protein, 384 aa, Homo sapiens
MVVEVDSMPAASSVKKPFGLRSKMGKWCCRCFPCCRESGKSNVGTSGDHDDSAMKTLRSKMGKWCRHCFPCCRGSGKSNVGASGDHDDSAMKTLRNKMGKWCCHCFPCCRGSGKSKVGAWGDYDDSAFMEPRYHVRGEDLDKLHRAAWWGKVPRKDLIVMLRDTDVNKKDKQKRTALHLASANGNSEVVKLLLDRRCQLNVLDNKKRTALIKAVQCQEDECALMLLEHGTDPNIPDEYGNTTLHYAIYNEDKLMAKALLLYGADIESKNKHGLTPLLLGVHEQKQQVVKFLIKKKANLNALDRYGRTALILAVCCGSASIVSLLLEQNIDVSSQDLSGQTAREYAVSSHHHVICQLLSDYKEKQMLKISSENSNPENVSRTRNK

SEQ ID NO: 305 - protein, 656 aa, Homo sapiens
MVVEVDSMPAASSVKKPFGLRSKMGKWCCRCFPCCRESGKSNVGTSGDHDDSAMKTLRSKMGKWCRHCFPCCRGSGKSNVGASGDHDDSAMKTLRNKMGKWCCHCFPCCRGSGKSKVGAWGDYDDSAFMEPRYHVRGEDLDKLHRAAWWGKVPRKDLIVMLRDTDVNKKDKQKRTALHLASANGNSEVVKLLLDRRCQLNVLDNKKRTALIKAVQCQEDECALMLLEHGTDPNIPDEYGNTTLHYAIYNEDKLMAKALLLYGADIESKNKHGLTPLLLGVHEQKQQVVKFLIKKKANLNALDRYGRTALILAVCCGSASIVSLLLEQNIDVSSQDLSGQTAREYAVSSHHHVICQLLSDYKEKQMLKISSENSNPEQDLKLTSEEESQRFKGSENSQPEKMSQEPEINKDGDREVEEEMKKHESNNVGLLENLTNGVTAGNGDNGLIPQRKSRTPENQQFPDNESEEYHRICELVSDYKEKQMPKYSSENSNPEQDLKLTSEEESQRLEGSENGQPELENFMAIEEMKKHGSTHVGFPENLTNGATAGNGDDGLIPPRKSRTPESQQFPDTENEEYHSDEQNDTQKQFCEEQNTGILHDEILIHEEKQIEVVEKMNSELSLSCKKEKDILHENSTLREEIAMLRLELDTMKHQSQL

SEQ ID NO: 306 - protein, 671 aa, Homo sapiens
MVVEVDSMPAASSVKKPFGLRSKMGKWCCRCFPCCRESGKSNVGTSGDHDDSAMKTLRSKMGKWCRHCFPCCRGSGKSNVGASGDHDDSAMKTLRNKMGKWCCHCFPCCRGSGKSKVGAWGDYDDSAFMEPRYHVRGEDLDKLHRAAWWGKVPRKDLIVMLRDTDVNKKDKQKRTALHLASANGNSEVVKLLLDRRCQLNVLDNKKRTALIKAVQCQEDECALMLLEHGTDPNIPDEYGNTTLHYAIYNEDKLMAKALLLYGADIESKNKHGLTPLLLGVHEQKQQVVKFLIKKKANLNALDRYGRTALILAVCCGSASIVSLLLEQNIDVSSQDLSGQTAREYAVSSHHHVICQLLSDYKEKQMLKISSENSNPEQDLKLTSEEESQRFKGSENSQPEKMSQEPEINKDGDREVEEEMKKHESNNVGLLENLTNGVTAGNGDNGLIPQRKSRTPENQQFPDNESEEYHRICELVSDYKEKQMPKYSSENSNPEQDLKLTSEEESQRLEGSENGQPEKRSQEPEINKDGDRELENFMAIEEMKKHGSTHVGFPENLTNGATAGNGDDGLIPPRKSRTPESQQFPDTENEEYHSDEQNDTQKQFCEEQNTGILHDEILIHEEKQIEVVEKMNSELSLSCKKEKDILHENSTLREEIAMLRLELDTMKHQSQL

SEQ ID NO: 307 - DNA, 800 bp, Homo sapiens
atkagcttccgcttctgacaacactagagatccctcccctccctcagggtatggccctccacttcatttttggtacataacatctttataggacaggggtaaaatcccaatactaacaggagaatgcttaggactctaacaggtttttgagaatgtgttggtaagggccactcaatccaatttttcttggtcctccttgtggtctaggaggacaggcaagggtgcagattttcaagaatgcatcagtaagggccactaaatccgaccttcctcgttcctccttgtggtctgggaggaaaactagtgtttctgttgctgtgtcagtgagcacaactattccgatcagcagggtccagggaccactgcaggttcttgggcagggggagaaacaaaacaaaccaaaaccatgggcrgttttgtctttcagatgggaaacactcaggcatcaacaggctcacctttgaaatgcatcctaagccaatgggacaaatttgacccacaaaccctggaaaaagaggtggctcattttttttgcactatggcttggccccaacattctctctctgatggggaaaaatggccacctgagggaagtacagattacaatactatcctgcagcttgaccttttctgtaagagggaaggcaaatggagtgaaataccttatgtccaagctttcttttcattgaaggagaatacactatgcaaagcttgaaatttacatcccacaggaggacctctcagcttacccccatatcctagcctccctatagctccccttcctattagtgataagcctc

SEQ ID NO: 308 - protein, 102 aa, Homo sapiens
  VARIANT 3: Xaa = Any Amino Acid
MGXFVFQMGNTQASTGSPLKCILSQWDKFDPQTLEKEVAHFFCTMAWPQHSLSDGEKWPPEGSTDYNTILQLDLFCKREGKWSEIPYVQAFFSLKENTLCKA

SEQ ID NO: 309 - protein, 9 aa, Artificial Sequence
  Made in the lab
LMAEEYTIV

SEQ ID NO: 310 - protein, 9 aa, Artificial Sequence
  Made in the lab
KLMAKALLL

SEQ ID NO: 311 - protein, 9 aa, Artificial Sequence
  Made in the lab
GLTPLLLGI

SEQ ID NO: 312 - protein, 10 aa, Artificial Sequence
  Made in the lab
KLVLDRRCQL

SEQ ID NO: 313 - DNA, 1852 bp, Homo sapiens
ggcacgagaattaaaaccctcagcaaaacaggcatagaagggacataccttaaagtaataaaaaccacctatgacaagcccacagccaacataatactaaatggggaaaagttagaagcatttcctctgagaactgcaacaataaatacaaggatgctggattttgtcaaatgccttttctgtgtctgttgagatgcttatgtgactttgcttttaattctgtttatgtgattatcacatttattgacttgcctgtgttagaccggaagagctggggtgtttctcaggagccaccgtgtgctgcggcagcttcgggataacttgaggctgcatcactggggaagaaacacaytcctgtccgtggcgctgatggctgaggacagagcttcagtgtggcttctctgcgactggcttcttcggggagttcttccttcatagttcatccatatggctccagaggaaaattatattattttgttatggatgaagagtattacgttgtgcagatatactgcagtgtcttcatctcttgatgtgtgattgggtaggttccaccatgttgccgcagatgacatgatttcagtacctgtgtctggctgaaaagtgtttgtttgtgaatggatattgtggtttctggatctcatcctctgtgggtggacagctttctccaccttgctggaagtgacctgctgtccagaagtttgatggctgaggagtataccatcgtgcatgcatctttcatttcctgcatttcttcctccctggatggacagggggagcggcaagagcaacgtgggcacttctggagaccacaacgactcctctgtgaagacgcttgggagcaagaggtgcaagtggtgctgccactgcttcccctgctgcagggggagcggcaagagcaacgtggtcgcttggggagactacgatgacagcgccttcatggatcccaggtaccacgtccatggagaagatctggacaagctccacagagctgcctggtggggtaaagtccccagaaaggatctcatcgtcatgctcagggacacggatgtgaacaagagggacaagcaaaagaggactgctctacatctggcctctgccaatgggaattcagaagtagtaaaactcgtgctggacagacgatgtcaacttaatgtccttgacaacaaaaagaggacagctctgacaaaggccgtacaatgccaggaagatgaatgtgcgttaatgttgctggaacatggcactgatccaaatattccagatgagtatggaaataccactctacactatgctgtctacaatgaagataaattaatggccaaagcactgctcttatacggtgctgatatcgaatcaaaaaacaagcatggcctcacaccactgctacttggtatacatgagcaaaaacagcaagtggtgaaatttttaatcaagaaaaaagcgaatttaaatgcgctggatagatatggaagaactgctctcatacttgctgtatgttgtggatcagcaagtatagtcagccctctacttgagcaaaatgttgatgtatcttctcaagatctggaaagacggccagagagtatgctgtttctagtcatcatcatgtaatttgccagttactttctgactacaaagaaaaacagatgttaaaaatctcttctgaaaacagcaatccagaacaagacttaaagctgacatcagaggaagagtcacaaaggcttaaaggaagtgaaaacagccagccagagctagaagatttatggctattgaagaagaatgaagaacacggaagtactcatgtgggattcccagaaaacctgactaacggtgccgctgctggcaatggtgatga

SEQ ID NO: 314 - DNA, 879 bp, Homo sapiens
atgcatctttcatttcctgcatttcttcctccctggatggacagggggagcggcaagagcaacgtgggcacttctggagaccacaacgactcctctgtgaagacgcttgggagcaagaggtgcaagtggtgctgccactgcttcccctgctgcagggggagcggcaagagcaacgtggtcgcttggggagactacgatgacagcgccttcatggatcccaggtaccacgtccatggagaagatctggacaagctccacagagctgcctggtggggtaaagtccccagaaaggatctcatcgtcatgctcagggacacggatgtgaacaagagggacaagcaaaagaggactgctctacatctggcctctgccaatgggaattcagaagtagtaaaactcgtgctggacagacgatgtcaacttaatgtccttgacaacaaaaagaggacagctctgacaaaggccgtacaatgccaggaagatgaatgtgcgttaatgttgctggaacatggcactgatccaaatattccagatgagtatggaaataccactctacactatgctgtctacaatgaagataaattaatggccaaagcactgctcttatacggtgctgatatcgaatcaaaaaacaagcatggcctcacaccactgctacttggtatacatgagcaaaaacagcaagtggtgaaatttttaatcaagaaaaaagcgaatttaaatgcgctggatagatatggaagaactgctctcatacttgctgtatgttgtggatcagcaagtatagtcagccctctacttgagcaaaatgttgatgtatcttctcaagatctggaaagacggccagagagtatgctgtttctagtcatcatcatgtaa

SEQ ID NO: 315 - protein, 292 aa, Homo sapiens
MHLSFPAFLPPWMDRGSGKSNVGTSGDHNDSSVKTLGSKRCKWCCHCFPCCRGSGKSNVVAWGDYDDSAFMDPRYHVHGEDLDKLHRAAWWGKVPRKDLIVMLRDTDVNKRDKQKRTALHLASANGNSEVVKLVLDRRCQLNVLDNKKRTALTKAVQCQEDECALMLLEHGTDPNIPDEYGNTTLHYAVYNEDKLMAKALLLYGADIESKNKHGLTPLLLGIHEQKQQVVKFLIKKKANLNALDRYGRTALILAVCCGSASIVSPLLEQNVDVSSQDLERRPESMLFLVIIM

SEQ ID NO: 316 - DNA, 584 bp, Homo sapiens
agttgggccaaattcccctccccctacagcttgaaggggacataaccaatagcctggggtttttttgtggtcctttggagatttctttgcttattttcttctgggtgggggtgattagaggaggcttatcactaataggaaggggagctatagggaggctaggatatgggggtaagctgagaggtcctcctgtgggatgtaaatttcaagctttgcatagtgtattctccttcaatgaaaagaaagcttggacataaggtatttcactccatttgccttccctcttacagaaaaggtcaagctgcaggatagtattgtaatctgtacttccctcaggtggccatttttccccatcagagagagaatgttggggccaagccatagtgcagaaaaaaaaatgagccacctctttttccagggtttgtgggtcaaatttgtcccattggcttaggatgcatttcaaaggtgagcctgttgatgcctgagtgtttcccatctgaaagacaaaactgcccatggttttggtttgttttgtttctccccctgcccaagaactatcaaactcctgagccaacaactaaaaa

SEQ ID NO: 317 - DNA, 829 bp, Homo sapiens
attagcttccgcttctgacaacactagagatccctcccctccctcagggtatggccctccacttcatttttggtacataacatctttataggacaggggtaaaatcccaatactaacaggagaatgcttaggactctaacaggtttttgagaatgtgttggtaagggccactcaatccaatttttcttggtcctccttgtggtctaggaggacaggcaagggtgcagattttcaagaatgcatcagtaagggccactaaatccgaccttcctcgttcctccttgtggtctgggaggaaaactagtgtttctgttgctgtgtcagtgagcacaactattccgatcagcagggtccagggaccactgcaggttcttgggcagggggagaaacaaaacaaaccaaaaccatgggcagttttgtctttcagatgggaaacactcaggcatcaacaggctcacctttgaaatgcatcctaagccaatgggacaaatttgacccacaaaccctggaaaaagaggtggctcattttttttgcactatggcttggccccaacattctctctctgatggggaaaaatggccacctgagggaagtacagattacaatactatcctgcagcttgaccttttctgtaagagggaaggcaaatggagtgaaataccttatgtccaagctttcttttcattgaaggagaatacactatgcaaagcttgaaatttacatcccacaggaggacctctcagcttacccccatatcctagcctccctatagctccccttcctattagtgataagcctcctctaatcacccccacccagaagaaaata

SEQ ID NO: 318 - protein, 30 aa, Homo sapien
TAASDNFQLSQGGQGFAIPIGQAMAIAGQI

SEQ ID NO: 319 - DNA, 41 bp, Artificial Sequence
  PCR primer
ggcctctgccaatgggaactcagaagtagtaaaactcctgc

SEQ ID NO: 320 - DNA, 41 bp, Artificial Sequence
  PCR primer
gcaggagttttactacttctgagttcccattggcagaggcc

SEQ ID NO: 321 - DNA, 60 bp, Artificial Sequence
  PCR primer
ggggaattcccgctggtgccgcgcggcagccctatggtggttgaggttgattccatgccg

SEQ ID NO: 322 - DNA, 42 bp, Artificial Sequence
  PCR primer
cccgaattcttatttatttctggttcttgagacattttctgg

SEQ ID NO: 323 - DNA, 1590 bp, Homo sapiens
atgcatcaccatcaccatcacacggccgcgtccgataacttccagctgtcccagggtgggcagggattcgccattccgatcgggcaggcgatggcgatcgcgggccagatcaagcttcccaccgttcatatcgggcctaccgccttcctcggcttgggtgttgtcgacaacaacggcaacggcgcacgagtccaacgcgtggtcgggagcgctccggcggcaagtctcggcatctccaccggcgacgtgatcaccgcggtcgacggcgctccgatcaactcggccaccgcgatggcggacgcgcttaacgggcatcatcccggtgacgtcatctcggtgacctggcaaaccaagtcgggcggcacgcgtacagggaacgtgacattggccgagggacccccggccgaattcccgctggtgccgcgcggcagccctatggtggttgaggttgattccatgccggctgcttcttctgtgaagaagccatttggtctcaggagcaagatgggcaagtggtgctgccgttgcttcccctgctgcagggagagcggcaagagcaacgtgggcacttctggagaccacgacgactctgctatgaagacactcaggagcaagatgggcaagtggtgccgccactgcttcccctgctgcagggggagtggcaagagcaacgtgggcgcttctggagaccacgacgactctgctatgaagacactcaggaacaagatgggcaagtggtgctgccactgcttcccctgctgcagggggagcggcaagagcaaggtgggcgcttggggagactacgatgacagygccttcatggagcccaggtaccacgtccgtggagaagatctggacaagctccacagagctgcctggtggggtaaagtccccagaaaggatctcatcgtcatgctcagggacactgacgtgaacaagaaggacaagcaaaagaggactgctctacatctggcctctgccaatgggaattcagaagtagtaaaactcctgctggacagacgatgtcaacttaatgtccttgacaacaaaaagaggacagctctgataaaggccgtacaatgccaggaagatgaatgtgcgttaatgttgctggaacatggcactgatccaaatattccagatgagtatggaaataccactctgcactacgctatctataatgaagataaattaatggccaaagcactgctcttatatggtgctgatatcgaatcaaaaaacaagcatggcctcacaccactgttacttggtgtacatgagcaaaaacagcaagtcgtgaaatttttaatcaagaaaaaagcgaatttaaatgcactggatagatatggaaggactgctctcatacttgctgtatgttgtggatcagcaagtatagtcagccttctacttgagcaaaatattgatgtatcttctcaagatctatctggacagacggccagagagtatgctgtttctagtcatcatcatgtaatttgccagttactttctgactacaaagaaaaacagatgctaaaaatctcttctgaaaacagcaatccagaaaatgtctcaagaaccagaaataaataa

SEQ ID NO: 324 - protein, 529 aa, Homo sapiens
MHHHHHHTAASDNFQLSQGGQGFAIPIGQAMAIAGQIKLPTVHIGPTAFLGLGVVDNNGNGARVQRVVGSAPAASLGISTGDVITAVDGAPINSATAMADALNGHHPGDVISVTWQTKSGGTRTGNVTLAEGPPAEFPLVPRGSPMVVEVDSMPAASSVKKPFGLRSKMGKWCCRCFPCCRESGKSNVGTSGDHDDSAMKTLRSKMGKWCRHCFPCCRGSGKSNVGASGDHDDSAMKTLRNKMGKWCCHCFPCCRGSGKSKVGAWGDYDDSAFMEPRYHVRGEDLDKLHRAAWWGKVPRKDLIVMLRDTDVNKKDKQKRTALHLASANGNSEVVKLLLDRRCQLNVLDNKKRTALIKAVQCQEDECALMLLEHGTDPNIPDEYGNTTLHYAIYNEDKLMAKALLLYGADIESKNKHGLTPLLLGVHEQKQQVVKFLIKKKANLNALDRYGRTALILAVCCGSASIVSLLLEQNIDVSSQDLSGQTAREYAVSSHHHVICQLLSDYKEKQMLKISSENSNPENVSRTRNK

SEQ ID NO: 325 - DNA, 1155 bp, Homo sapiens
atggtggctgaggtttgttcaatgcccactgcctctactgtgaagaagccatttgatctcaggagcaagatgggcaagtggtgccaccaccgcttcccctgctgcagggggagcggcaagagcaacatgggcacttctggagaccacgacgactcctttatgaagatgctcaggagcaagatgggcaagtgttgccgccactgcttcccctgctgcagggggagcggcacgagcaacgtgggcacttctggagaccatgaaaactcctttatgaagatgctcaggagcaagatgggcaagtggtgctgtcactgcttcccctgctgcagggggagcggcaagagcaacgtgggcgcttggggagactacgaccacagcgccttcatggagccgaggtaccacatccgtcgagaagatctggacaagctccacagagctgcctggtggggtaaagtccccagaaaggatctcatcgtcatgctcagggacactgacatgaacaagagggacaaggaaaagaggactgctctacatttggcctctgccaatggaaattcagaagtagtacaactcctgctggacagacgatgtcaacttaatgtccttgacaacaaaaaaaggacagctctgataaaggccatacaatgccaggaagatgaatgtgtgttaatgttgctggaacatggcgctgatcgaaatattccagatgagtatggaaataccgctctacactatgctatctacaatgaagataaattaatggccaaagcactgctcttatatggtgctgatattgaatcaaaaaacaaggttggcctcacaccacttttgcttggcgtacatgaacaaaaacagcaagtggtgaaatttttaatcaagaaaaaagctaatttaaatgtacttgatagatatggaaggactgccctcatacttgctgtatgttgtggatcagcaagtatagtcaatcttctacttgagcaaaatgttgatgtatcttctcaagatctatctggacagacggccagagagtatgctgtttctagtcatcatcatgtaatttgtgaattactttctgactataaagaaaaacagatgctaaaaatctcttctgaaaacagcaatccagaaaatgtctcaagaaccagaaataaataa

SEQ ID NO: 326 - protein, 384 aa, Homo sapiens
MVAEVCSMPTASTVKKPFDLRSKMGKWCHHRFPCCRGSGKSNMGTSGDHDDSFMKMLRSKMGKCCRHCFPCCRGSGTSNVGTSGDHENSFMKMLRSKMGKWCCHCFPCCRGSGKSNVGAWGDYDHSAFMEPRYHIRREDLDKLHRAAWWGKVPRKDLIVMLRDTDMNKRDKEKRTALHLASANGNSEVVQLLLDRRCQLNVLDNKKRTALIKAIQCQEDECVLMLLEHGADRNIPDEYGNTALHYAIYNEDKLMAKALLLYGADIESKNKVGLTPLLLGVHEQKQQVVKFLIKKKANLNVLDRYGRTALILAVCCGSASIVNLLLEQNVDVSSQDLSGQTAREYAVSSHHHVICELLSDYKEKQMLKISSENSNPENVSRTRNK

SEQ ID NO: 327 - DNA, 634 bp, Homo sapiens
gactgctctacatctggcctctgccaatggaaattcagaagtagtaaaactcctgctggacagacgatgtcaacttaatatccttgacaacaaaaagaggacagctctgacaaaggccgtacaatgccaggaagatgaatgtgcgttaatgttgctggaacatggcactgatccgaatattccagatgagtatggaaataccgctctacactatgctatctacaatgaagataaattaatggccaaagcactgctcttatacggtgctgatatcgaatcaaaaaacaagcatggcctcacaccactgttacttggtgtacatgagcaaaaacagcaagtggtgaaatttttaatcaagaaaaaagcaaatttaaatgcactggatagatatggaagaactgctctcatacttgctgtatgttgtggatcggcaagtatagtcagccttctacttgagcaaaacattgatgtatcttctcaagatctatctggacagacggccagagagtatgctgtttctagtcgtcataatgtaatttgccagttactttctgactacaaagaaaaacagatactaaaagtctcttctgaaaacagcaatccaggaaatgtctcaagaaccagaaataaataa

SEQ ID NO: 328 - DNA, 1155 bp, Homo sapiens
atggtggttgaggttgattccatgccggctgcctcttctgtgaagaagccatttggtctcaggagcaagatgggcaagtggtgctgccgttgcttcccctgctgcagggagagcggcaagagcaacgtgggcacttctggagaccacgacgactctgctatgaagacactcaggagcaagatgggcaagtggtgccgccactgcttcccctgctgcagggggagtggcaagagcaacgtgggcgcttctggagaccacgacgactctgctatgaagacactcaggaacaagatgggcaagtggtgctgccactgcttcccctgctgcagggggagcagcaagagcaaggtgggcgcttggggagactacgatgacagtgccttcatggagcccaggtaccacgtccgtggagaagatctggacaagctccacagagctgcctggtggggtaaagtccccagaaaggatctcatcgtcatgctcagggacactgacgtgaacaagcaggacaagcaaaagaggactgctctacatctggcctctgccaatgggaattcagaagtagtaaaactcctgctggacagacgatgtcaacttaatgtccttgacaacaaaaagaggacagctctgataaaggccgtacaatgccaggaagatgaatgtgcgttaatgttgctggaacatggcactgatccaaatattccagatgagtatggaaataccactctgcactacgctatctataatgaagataaattaatggccaaagcactgctcttatatggtgctgatatcgaatcaaaaaacaagcatggcctcacaccactgttacttggtgtacatgagcaaaaacagcaagtcgtgaaatttttaattaagaaaaaagcgaatttaaatgcactggatagatatggaaggactgctctcatacttgctgtatgttgtggatcagcaagtatagtcagccttctacttgagcaaaatattgatgtatcttctcaagatctatctggacagacggccagagagtatgctgtttctagtcatcatcatgtaatttgccagttactttctgactacaaagaaaaacagatgctaaaaatctcttctgaaaacagcaatccagaaaatgtctcaagaaccagaaataaataa

SEQ ID NO: 329 - DNA, 1155 bp, Homo sapiens
atggtggctgaggtttgttcaatgcccgctgcctctgctgtgaagaagccatttgatctcaggagcaagatgggcaagtggtgccaccaccgcttcccctgctgcagggggagcggcaagagcaacatgggcacttctggagaccacgacgactcctttatgaagacgctcaggagcaagatgggcaagtgttgccaccactgcttcccctgctgcagggggagcggcacgagcaatgtgggcacttctggagaccatgacaactcctttatgaagacactcaggagcaagatgggcaagtggtgctgtcactgcttcccctgctgcagggggagcggcaagagcaacgtgggcacttggggagactacgacgacagcgccttcatggagccgaggtaccacgtccgtcgagaagatctggacaagctccacagagctgcctggtggggtaaagtccccagaaaggatctcatcgtcatgctcagggacactgacatgaacaagagggacaagcaaaagaggactgctctacatttggcctctgccaatggaaattcagaagtagtacaactcctgctggacagacgatgtcaacttaacgtccttgacaacaaaaaaaggacagctctgataaaggccgtacaatgccaggaagatgaatgtgtgttaatgttgctggaacatggcgctgatggaaatattcaagatgagtatggaaataccgctctacactatgctatctacaatgaagataaattaatggccaaagcactgctcttatatggtgctgatattgaatcaaaaaacaagtgtggcctcacaccacttttgcttggcgtacatgaacaaaaacagcaagtggtgaaatttttaatcaagaaaaaagctaatttaaatgcacttgatagatatggaagaactgccctcatacttgctgtatgttgtggatcagcaagtatagtcaatcttctacttgagcaaaatgttgatgtatcttctcaagatctatctggacagacggccagagagtatgctgtttctagtcatcatcatgtaatttgtgaattactttctgactataaagaaaaacagatgctaaaaatctcttctgaaaacagcaatccagaaaatgtctcaagaaccagaaataaataa

SEQ ID NO: 330 - DNA, 1155 bp, Homo sapiens
atggtggctgaggtttgttcaatgcccactgcctctactgtgaagaagccatttgatctcaggagcaagatgggcaagtggtgccaccaccgcttcccctgctgcagggggagcggcaagagcaacatgggcacttctggagaccacgacgactcctttatgaagatgctcaggagcaagatgggcaagtgttgccgccactgcttcccctgctgcagggggagcggcacgagcaacgtgggcacttctggagaccatgaaaactcctttatgaagatgctcaggagcaagatgggcaagtggtgctgtcactgcttcccctgctgcagggggagcggcaagagcaacgtgggcgcttggggagactacgaccacagcgccttcatggagccgaggtaccacatccgtcgagaagatctggacaagctccacagagctgcctggtggggtaaagtccccagaaaggatctcatcgtcatgctcagggacactgacatgaacaagagggacaaggaaaagaggactgctctacatttggcctctgccaatggaaattcagaagtagtacaactcctgctggacagacgatgtcaacttaatgtccttgacaacaaaaaaaggacagctctgataaaggccatacaatgccaggaagatgaatgtgtgttaatgttgctggaacatggcgctgatcgaaatattccagatgagtatggaaataccgctctacactatgctatctacaatgaagataaattaatggccaaagcactgctcttatatggtgctgatattgaatcaaaaaacaagtgtggcctcacaccacttttgcttggcgtacatgaacaaaaacagcaagtggtgaaatttttaatcaagaaaaaagctaatttaaatgtacttgatagatatggaagaactgccctcatacttgctgtatgttgtggatcagcaagtatagtcaatcttctacttgagcaaaatgttgatgtatcttctcaagatctatctggacagacggccagagagtatgctgtttctagtcatcatcatgtaatttgtgaattactttctgactataaagaaaaacagatgctaaaaatctcttctgaaaacagcaatccagaaaatgtctcaagaaccagaaataaataa

SEQ ID NO: 331 - protein, 210 aa, Homo sapiens
TALHLASANGNSEVVKLLLDRRCQLNILDNKKRTALTKAVQCQEDECALMLLEHGTDPNIPDEYGNTALHYAIYNEDKLMAKALLLYGADIESKNKHGLTPLLLGVHEQKQQVVKFLIKKKANLNALDRYGRTALILAVCCGSASIVSLLLEQNIDVSSQDLSGQTAREYAVSSRHNVICQLLSDYKEKQILKVSSENSNPGNVSRTRNK

SEQ ID NO: 332 - protein, 384 aa, Homo sapiens
MVAEVCSMPTASTVKKPFDLRSKMGKWCHHRFPCCRGSGKSNMGTSGDHDDSFMKMLRSKMGKCCRHCFPCCRGSGTSNVGTSGDHENSFMKMLRSKMGKWCCHCFPCCRGSGKSNVGAWGDYDHSAFMEPRYHIRREDLDKLHRAAWWGKVPRKDLIVMLRDTDMNKRDKEKRTALHLASANGNSEVVQLLLDRRCQLNVLDNKKRTALIKAIQCQEDECVLMLLEHGADRNIPDEYGNTALHYAIYNEDKLMAKALLLYGADIESKNKCGLTPLLLGVHEQKQQVVKFLIKKKANLNVLDRYGRTALILAVCCGSASIVNLLLEQNVDVSSQDLSGQTAREYAVSSHHHVICELLSDYKEKQMLKISSENSNPENVSRTRNK

SEQ ID NO: 333 - protein, 384 aa, Homo sapiens
MVAEVCSMPAASAVKKPFDLRSKMGKWCHHRFPCCRGSGKSNMGTSGDHDDSFMKTLRSKMGKCCHHCFPCCRGSGTSNVGTSGDHDNSFMKTLRSKMGKWCCHCFPCCRGSGKSNVGTWGDYDDSAFMEPRYHVRREDLDKLHRAAWWGKVPRKDLIVMLRDTDMNKRDKQKRTALHLASANGNSEVVQLLLDRRCQLNVLDNKKRTALIKAVQCQEDECVLMLLEHGADGNIQDEYGNTALHYAIYNEDKLMAKALLLYGADIESKNKCGLTPLLLGVHEQKQQVVKFLIKKKANLNALDRYGRTALILAVCCGSASIVNLLLEQNVDVSSQDLSGQTAREYAVSSHHHVICELLSDYKEKQMLKISSENSNPENVSRTRNK

SEQ ID NO: 334 - protein, 384 aa, Homo sapiens
MVVEVDSMPAASSVKKPFGLRSKMGKWCCRCFPCCRESGKSNVGTSGDHDDSAMKTLRSKMGKWCRHCFPCCRGSGKSNVGASGDHDDSAMKTLRNKMGKWCCHCFPCCRGSSKSKVGAWGDYDDSAFMEPRYHVRGEDLDKLHRAAWWGKVPRKDLIVMLRDTDVNKQDKQKRTALHLASANGNSEVVKLLLDRRCQLNVLDNKKRTALIKAVQCQEDECALMLLEHGTDPNIPDEYGNTTLHYAIYNEDKLMAKALLLYGADIESKNKHGLTPLLLGVHEQKQQVVKFLIKKKANLNALDRYGRTALILAVCCGSASIVSLLLEQNIDVSSQDLSGQTAREYAVSSHHHVICQLLSDYKEKQMLKISSENSNPENVSRTRNK

SEQ ID NO: 335 - DNA, 1185 bp, Homo sapiens
atggtggttgaggttgattccatgccggctgcctcttctgtgaagaagccatttggtctcaggagcaagatgggcaagtggtgctgccgttgcttcccctgctgcagggagagcggcaagagcaacgtgggcacttctggagaccacgacgactctgctatgaagacactcaggagcaagatgggcaagtggtgccgccactgcttcccctgctgcagggggagtggcaagagcaacgtgggcgcttctggagaccacgacgactctgctatgaagacactcaggaacaagatgggcaagtggtgctgccactgcttcccctgctgcagggggagcggcaagagcaaggtgggcgcttggggagactacgatgacagtgccttcatggagcccaggtaccacgtccgtggagaagatctggacaagctccacagagctgcctggtggggtaaagtccccagaaaggatctcatcgtcatgctcagggacactgacgtgaacaagaaggacaagcaaaagaggactgctctacatctggcctctgccaatgggaattcagaagtagtaaaactcctgctggacagacgatgtcaacttaatgtccttgacaacaaaaagaggacagctctgataaaggccgtacaatgccaggaagatgaatgtgcgttaatgttgctggaacatggcactgatccaaatattccagatgagtatggaaataccactctgcactacgctatctataatgaagataaattaatggccaaagcactgctcttatatggtgctgatatcgaatcaaaaaacaagcatggcctcacaccactgttacttggtgtacatgagcaaaaacagcaagtcgtgaaatttttaatcaagaaaaaagcgaatttaaatgcactggatagatatggaaggactgctctcatacttgctgtatgttgtggatcagcaagtatagtcagccttctacttgagcaaaatattgatgtatcttctcaagatctatctggacagacggccagagagtatgctgtttctagtcatcatcatgtaatttgccagttactttctgactacaaagaaaaacagatgctaaaaatctcttctgaaaacagcaatccagaaaatgtctcaagaaccagaaataaacatcatcaccatcatcatcaccatcaccattaa

SEQ ID NO: 336 - protein, 394 aa, Homo sapiens
MVVEVDSMPAASSVKKPFGLRSKMGKWCCRCFPCCRESGKSNVGTSGDHDDSAMKTLRSKMGKWCRHCFPCCRGSGKSNVGASGDHDDSAMKTLRNKMGKWCCHCFPCCRGSGKSKVGAWGDYDDSAFMEPRYHVRGEDLDKLHRAAWWGKVPRKDLIVMLRDTDVNKKDKQKRTALHLASANGNSEVVKLLLDRRCQLNVLDNKKRTALIKAVQCQEDECALMLLEHGTDPNIPDEYGNTTLHYAIYNEDKLMAKALLLYGADIESKNKHGLTPLLLGVHEQKQQVVKFLIKKKANLNALDRYGRTALILAVCCGSASIVSLLLEQNIDVSSQDLSGQTAREYAVSSHHHVICQLLSDYKEKQMLKISSENSNPENVSRTRNKHHHHHHHHHH

SEQ ID NO: 337 - DNA, 34 bp, Artificial Sequence
  PCR primer
cggcggatccaccatggtggttgaggttgattcc

SEQ ID NO: 338 - DNA, 74 bp, Artificial Sequence
  PCR primer
cggctctagattaatggtgatggtgatgatgatggtgatgatgtttatttctggttcttgagacattttctgga

SEQ ID NO: 339 - DNA, 1166 bp, Homo sapiens
atggtggctgaggctggttcaatgccggctgcctcctctgtgaagaagccatttggtctcagaagcaagatgggcaagtggtgccgccactgcttcccctggtgcagggggagcggcaagagcaacgtgggcacttctggagaccacgacgattctgctatgaagacactcaggagcaagatgggcaagtggtgccgccactgcttcccctggtgcagggggagcagcaagagcaacgtgggcacttctggagaccacgacgactctgctatgaagacactcaggagcaagatgggcaagtggtgctgccactgcttcccctgctgcagggggagcggcaagagcaaagtgggcccttggggagactacgacgacagcgctttcatggagccgaggtaccacgtccgtcgagaagatctggacaagctccacagagctgcctggtggggtaaagtccccagaaaggatctcatcgtcatgctcaaggacactgacatgaacaagaaggacaagcaaaagaggactgctctacatctggcctctgccaatggaaattcagaagtagtaaaactcctgctggacagacgatgtcaacttaatatccttgacaacaaaaagaggacagctctgacaaaggccgtacaatgccgggaagatgaatgtgcgttaatgttgctggaacatggcactgatccgaatattccagatgagtatggaaataccgctctacactatgctatctacaatgaagataaattaatggccaaagcactgctcttatacggtgctgatatcgaatcaaaaaacaagcatggcctcacaccactgttacttggtgtacatgagcaaaaacagcaagtggtgaaattcttaatcaagaaaaaagcaaatttaaatgcactggatagatatggaagaactgctctcatacttgctgtatgttgtggatcggcaagtatagtcagccttctacttgagcaaaacattgatgtatcttctcaagatctatctggacagacggccagagagtatgctgtttctagtcatcataatgtaatttgccagttactttctgactacaaagaaaaacagatgctaaaagtctcttctgaaaacagcaatccaggaaatgtctcaagaaccagaaataaataagggtggtgata

SEQ ID NO: 340 - protein, 384 aa, Homo sapiens
MVAEAGSMPAASSVKKPFGLRSKMGKWCRHCFPWCRGSGKSNVGTSGDHDDSAMKTLRSKMGKWCRHCFPWCRGSSKSNVGTSGDHDDSAMKTLRSKMGKWCCHCFPCCRGSGKSKVGPWGDYDDSAFMEPRYHVRREDLDKLHRAAWWGKVPRKDLIVMLKDTDMNKKDKQKRTALHLASANGNSEVVKLLLDRRCQLNILDNKKRTALTKAVQCREDECALMLLEHGTDPNIPDEYGNTALHYAIYNEDKLMAKALLLYGADIESKNKHGLTPLLLGVHEQKQQVVKFLIKKKANLNALDRYGRTALILAVCCGSASIVSLLLEQNIDVSSQDLSGQTAREYAVSSHHNVICQLLSDYKEKQMLKVSSENSNPGNVSRTRNK

SEQ ID NO: 341 - DNA, 876 bp, Homo sapiens
atgcatctttcatttcctgcatttcttcctccctggatggacagggggagcggcaagagcaacgtgggcacttctggagaccacaacgactcctctgtgaagacgcttgggagcaagaggtgcaagtggtgctgccactgcttcccctgctgcagggggagcggcaagagcaacgtggtcgcttggggagactacgatgacagcgccttcatggatcccaggtaccacgtccatggagaagatctggacaagctccacagagctgcctggtggggtaaagtccccagaaaggatctcatcgtcatgctcagggacacggatgtgaacaagagggacaagcaaaagaggactgctctacatctggcctctgccaatgggaattcagaagtagtaaaactcgtgctggacagacgatgtcaacttaatgtccttgacaacaaaaagaggacagctctgacaaaggccgtacaatgccaggaagatgaatgtgcgttaatgttgctggaacatggcactgatccaaatattccagatgagtatggaaataccactctacactatgctgtctacaatgaagataaattaatggccaaagcactgctcttatacggtgctgatatcgaatcaaaaaacaagcatggcctcacaccactgctacttggtatacatgagcaaaaacagcaagtggtgaaatttttaatcaagaaaaaagcgaatttaaatgcgctggatagatatggaagaactgctctcatacttgctgtatgttgtggatcagcaagtatagtcagccctctacttgagcaaaatgttgatgtatcttctcaagatctggaaagacggccagagagtatgctgtttctagtcatcatcatg

SEQ ID NO: 342 - DNA, 876 bp, Homo sapiens
atgcatctttcatttcctgcatttcttcctccctggatggacagggggagcggcaagagcaacgtgggcacttctggagaccacaacgactcctctgtgaagacgcttgggagcaagaggtgcaagtggtgctgccactgcttcccctgctgcagggggagcggcaagagcaacgtgggcgcttggggagactacgatgacagcgccttcatggatcccaggtaccacgtccatggagaagatctggacaagctccacagagctgcctggtggggtaaagtccccagaaaggatctcatcgtcatgctcagggacactgatgtgaacaagagggacaagcaaaagaggactgctctacatctggcctctgccaatgggaattcagaagtagtaaaactcgtgctggacagacgatgtcaacttaatgtccttgacaacaaaaagaggacagctctgacaaaggccgtacaatgccaggaagatgaatgtgcgttaatgttgctggaacatggcactgatccaaatattccagatgagtatggaaataccactctacactatgctgtctacaatgaagataaattaatggccaaagcactgctcttatacggtgctgatatcgaatcaaaaaacaagcatggcctcacaccactgctacttggtatacatgagcaaaaacagcaagtggtgaaatttttaatcaagaaaaaagcgaatttaaatgcgctggatagatatggaagaactgctctcatacttgctgtatgttgtggatcagcaagtatagtcagccctctacttgagcaaaatgttgatgtatcttctcaagatctggaaagacggccagagagtatgctgtttctagtcatcatcatg

SEQ ID NO: 343 - DNA, 933 bp, Homo sapiens
atggtggttgaggttgattcaatgccggctgcctcttctgtgaagaagccatttggtctcaggagcaagatgggcaagtggtgctgctttccctgctgcagggggagcggcaagagcaacgtgggcacttctggagaccacaacgactcctctgtgaagacgcttgggagcaagaggtgcaagtggtgctgccactgcttcccctgctgcagggggagcggcaagagcaacgtgggcgcttggggagactacgatgacagcgccttcatggatcccaggtaccacgtccatggagaagatctggacaagctccacagagctgcctggtggggtaaagtccccagaaaggatctcatcgtcatgctcagggacactgatgtgaacaagagggacaagcaaaagaggactgctctacatctggcctctgccaatgggaattcagaagtagtaaaactcgtgctggacagacgatgtcaacttaatgtccttgacaacaaaaagaggacagctctgacaaaggccgtacaatgccaggaagatgaatgtgcgttaatgttgctggaacatggcactgatccaaatattccagatgagtatggaaataccactctacactatgctgtctacaatgaagataaattaatggccaaagcactgctcttatacggtgctgatatcgaatcaaaaaacaagcatggcctcacaccactgctacttggtatacatgagcaaaaacagcaagtggtgaaatttttaatcaagaaaaaagcgaatttaaatgcgctggatagatatggaagaactgctctcatacttgctgtatgttgtggatcagcaagtatagtcagccctctacttgagcaaaatgttgatgtatcttctcaagatctggaaagacggccagagagtatgctgtttctagtcatcatcatg

SEQ ID NO: 344 - DNA, 939 bp, Homo sapiens
atggtggttgaggttgattcaatgccggctgcctcttctgtgaagaagccatttggtctcaggagcaagatgggcaagtggtgctgccactgctttccctgctgcagggggagcggcaagagcaacgtgggcacttctggagaccacaacgactcctctgtgaagacgcttgggagcaagaggtgcaagtggtgctgccactgcttcccctgctgcagggggagcggcaagagcaacgtggtcgcttggggagactacgatgacagcgccttcatggatcccaggtaccacgtccatggagaagatctggacaagctccacagagctgcctggtggggtaaagtccccagaaaggatctcatcgtcatgctcagggacacggatgtgaacaagagggacaagcaaaagaggactgctctacatctggcctctgccaatgggaattcagaagtagtaaaactcgtgctggacagacgatgtcaacttaatgtccttgacaacaaaaagaggacagctctgacaaaggccgtacaatgccaggaagatgaatgtgcgttaatgttgctggaacatggcactgatccaaatattccagatgagtatggaaataccactctacactatgctgtctacaatgaagataaattaatggccaaagcactgctcttatacggtgctgatatcgaatcaaaaaacaagcatggcctcacaccactgctacttggtatacatgagcaaaaacagcaagtggtgaaatttttaatcaagaaaaaagcgaatttaaatgcgctggatagatatggaagaactgctctcatacttgctgtatgttgtggatcagcaagtatagtcagccctctacttgagcaaaatgttgatgtatcttctcaagatctggaaagacggccagagagtatgctgtttctagtcatcatcatg

SEQ ID NO: 345 - protein, 292 aa, Homo sapiens; identical to SEQ ID NO: 315

SEQ ID NO: 346 - protein, 292 aa, Homo sapiens
MHLSFPAFLPPWMDRGSGKSNVGTSGDHNDSSVKTLGSKRCKWCCHCFPCCRGSGKSNVGAWGDYDDSAFMDPRYHVHGEDLDKLHRAAWWGKVPRKDLIVMLRDTDVNKRDKQKRTALHLASANGNSEVVKLVLDRRCQLNVLDNKKRTALTKAVQCQEDECALMLLEHGTDPNIPDEYGNTTLHYAVYNEDKLMAKALLLYGADIESKNKHGLTPLLLGIHEQKQQVVKFLIKKKANLNALDRYGRTALILAVCCGSASIVSPLLEQNVDVSSQDLERRPESMLFLVIIM

SEQ ID NO: 347 - protein, 311 aa, Homo sapiens
MVVEVDSMPAASSVKKPFGLRSKMGKWCCFPCCRGSGKSNVGTSGDHNDSSVKTLGSKRCKWCCHCFPCCRGSGKSNVGAWGDYDDSAFMDPRYHVHGEDLDKLHRAAWWGKVPRKDLIVMLRDTDVNKRDKQKRTALHLASANGNSEVVKLVLDRRCQLNVLDNKKRTALTKAVQCQEDECALMLLEHGTDPNIPDEYGNTTLHYAVYNEDKLMAKALLLYGADIESKNKHGLTPLLLGIHEQKQQVVKFLIKKKANLNALDRYGRTALILAVCCGSASIVSPLLEQNVDVSSQDLERRPESMLFLVIIM

SEQ ID NO: 348 - protein, 313 aa, Homo sapiens
MVVEVDSMPAASSVKKPFGLRSKMGKWCCHCFPCCRGSGKSNVGTSGDHNDSSVKTLGSKRCKWCCHCFPCCRGSGKSNVVAWGDYDDSAFMDPRYHVHGEDLDKLHRAAWWGKVPRKDLIVMLRDTDVNKRDKQKRTALHLASANGNSEVVKLVLDRRCQLNVLDNKKRTALTKAVQCQEDECALMLLEHGTDPNIPDEYGNTTLHYAVYNEDKLMAKALLLYGADIESKNKHGLTPLLLGIHEQKQQVVKFLIKKKANLNALDRYGRTALILAVCCGSASIVSPLLEQNVDVSSQDLERRPESMLFLVIIM

SEQ ID NO: 349 - protein, 30 aa, Homo sapiens
VNKKDKQKRTALHLASANGNSEVVKLLLDR

SEQ ID NO: 350 - protein, 30 aa, Homo sapiens
ALHLASANGNSEVVKLLLDRRCQLNVLDNK

SEQ ID NO: 351 - protein, 25 aa, Homo sapiens
GSASIVSLLLEQNIDVSSQDLSGQT

SEQ ID NO: 352 - protein, 20 aa, Homo sapiens
VVEVDSMPAASSVKKPFGLR

SEQ ID NO: 353 - protein, 20 aa, Homo sapiens
SMPAASSVKKPFGLRSKMGK

SEQ ID NO: 354 - protein, 20 aa, Homo sapiens
SSVKKPFGLRSKMGKWCCRC

SEQ ID NO: 355 - protein, 20 aa, Homo sapiens
PFGLRSKMGKWCCRCFPCCR

SEQ ID NO: 356 - protein, 20 aa, Homo sapiens
SKMGKWCCRCFPCCRESGKS

SEQ ID NO: 357 - protein, 20 aa, Homo sapiens
WCCRCFPCCRESGKSNVGTS

SEQ ID NO: 358 - protein, 20 aa, Homo sapiens
FPCCRESGKSNVGTSGDHDD

SEQ ID NO: 359 - protein, 20 aa, Homo sapiens
ESGKSNVGTSGDHDDSAMKT

SEQ ID NO: 360 - protein, 20 aa, Homo sapiens
NVGTSGDHDDSAMKTLRSKM

SEQ ID NO: 361 - protein, 20 aa, Homo sapiens
GDHDDSAMKTLRSKMGKWCR

SEQ ID NO: 362 - protein, 20 aa, Homo sapiens
SAMKTLRSKMGKWCRHCFPC

SEQ ID NO: 363 - protein, 20 aa, Homo sapiens
LRSKMGKWCRHCFPCCRGSG

SEQ ID NO: 364 - protein, 20 aa, Homo sapiens
GKWCRHCFPCCRGSGKSNVG

SEQ ID NO: 365 - protein, 20 aa, Homo sapiens
HCFPCCRGSGKSNVGASGDH

SEQ ID NO: 366 - protein, 20 aa, Homo sapiens
CRGSGKSNVGASGDHDDSAM

SEQ ID NO: 367 - protein, 20 aa, Homo sapiens
KSNVGASGDHDDSAMKTLRN

SEQ ID NO: 368 - protein, 20 aa, Homo sapiens
ASGDHDDSAMKTLRNKMGKW

SEQ ID NO: 369 - protein, 20 aa, Homo sapiens
DDSAMKTLRNKMGKWCCHCF

SEQ ID NO: 370 - protein, 20 aa, Homo sapiens
KTLRNKMGKWCCHCFPCCRG

SEQ ID NO: 371 - protein, 20 aa, Homo sapiens
KMGKWCCHCFPCCRGSGKSK

SEQ ID NO: 372 - protein, 20 aa, Homo sapiens
CCHCFPCCRGSGKSKVGAWG

SEQ ID NO: 373 - protein, 20 aa, Homo sapiens
PCCRGSGKSKVGAWGDYDDS

SEQ ID NO: 374 - protein, 20 aa, Homo sapiens
SGKSKVGAWGDYDDSAFMEP

SEQ ID NO: 375 - protein, 20 aa, Homo sapiens
VGAWGDYDDSAFMEPRYHVR

SEQ ID NO: 376 - protein, 20 aa, Homo sapiens
DYDDSAFMEPRYHVRGEDLD

SEQ ID NO: 377 - protein, 20 aa, Homo sapiens
AFMEPRYHVRGEDLDKLHRA

SEQ ID NO: 378 - protein, 20 aa, Homo sapiens
RYHVRGEDLDKLHRAAWWGK

SEQ ID NO: 379 - protein, 20 aa, Homo sapiens
GEDLDKLHRAAWWGKVPRKD

SEQ ID NO: 380 - protein, 20 aa, Homo sapiens
KLHRAAWWGKVPRKDLIVML

SEQ ID NO: 381 - protein, 20 aa, Homo sapiens
AWWGKVPRKDLIVMLRDTDV

SEQ ID NO: 382 - protein, 20 aa, Homo sapiens
VPRKDLIVMLRDTDVNKKDK

SEQ ID NO: 383 - protein, 20 aa, Homo sapiens
LIVMLRDTDVNKKDKQKRTA

SEQ ID NO: 384 - protein, 20 aa, Homo sapiens
RDTDVNKKDKQKRTALHLAS

SEQ ID NO: 385 - protein, 20 aa, Homo sapiens
NKKDKQKRTALHLASANGNS

SEQ ID NO: 386 - protein, 20 aa, Homo sapiens
QKRTALHLASANGNSEVVKL

SEQ ID NO: 387 - protein, 20 aa, Homo sapiens
LHLASANGNSEVVKLLLDRR

SEQ ID NO: 388 - protein, 20 aa, Homo sapiens
ANGNSEVVKLLLDRRCQLNV

SEQ ID NO: 389 - protein, 20 aa, Homo sapiens
EVVKLLLDRRCQLNVLDNKK

SEQ ID NO: 390 - protein, 20 aa, Homo sapiens
LLDRRCQLNVLDNKKRTALI

SEQ ID NO: 391 - protein, 20 aa, Homo sapiens
CQLNVLDNKKRTALIKAVQC

SEQ ID NO: 392 - protein, 20 aa, Homo sapiens
LDNKKRTALIKAVQCQEDEC

SEQ ID NO: 393 - protein, 20 aa, Homo sapiens
RTALIKAVQCQEDECALMLL

SEQ ID NO: 394 - protein, 20 aa, Homo sapiens
KAVQCQEDECALMLLEHGTD

SEQ ID NO: 395 - protein, 20 aa, Homo sapiens
QEDECALMLLEHGTDPNIPD

SEQ ID NO: 396 - protein, 20 aa, Homo sapiens
ALMLLEHGTDPNIPDEYGNT

SEQ ID NO: 397 - protein, 20 aa, Homo sapiens
EHGTDPNIPDEYGNTTLHYA

SEQ ID NO: 398 - protein, 20 aa, Homo sapiens
PNIPDEYGNTTLHYAIYNED

SEQ ID NO: 399 - protein, 20 aa, Homo sapiens
EYGNTTLHYAIYNEDKLMAK

SEQ ID NO: 400 - protein, 20 aa, Homo sapiens
TLHYAIYNEDKLMAKALLLY

SEQ ID NO: 401 - protein, 20 aa, Homo sapiens
IYNEDKLMAKALLLYGADIE

SEQ ID NO: 402 - protein, 20 aa, Homo sapiens
KLMAKALLLYGADIESKNKH

SEQ ID NO: 403 - protein, 20 aa, Homo sapiens
ALLLYGADIESKNKHGLTPL

SEQ ID NO: 404 - protein, 20 aa, Homo sapiens
GADIESKNKHGLTPLLLGVH

SEQ ID NO: 405 - protein, 20 aa, Homo sapiens
SKNKHGLTPLLLGVHEQKQQ

SEQ ID NO: 406 - protein, 20 aa, Homo sapiens
GLTPLLLGVHEQKQQVVKFL

SEQ ID NO: 407 - protein, 20 aa, Homo sapiens
LLGVHEQKQQVVKFLIKKKA

SEQ ID NO: 408 - protein, 20 aa, Homo sapiens
EQKQQVVKFLIKKKANLNAL

SEQ ID NO: 409 - protein, 20 aa, Homo sapiens
VVKFLIKKKANLNALDRYGR

SEQ ID NO: 410 - protein, 20 aa, Homo sapiens
IKKKANLNALDRYGTRALIL

SEQ ID NO: 411 - protein, 20 aa, Homo sapiens
NLNALDRYGRTALILAVCCG

SEQ ID NO: 412 - protein, 20 aa, Homo sapiens
DRYGRTALILAVCCGSASIV

SEQ ID NO: 413 - protein, 20 aa, Homo sapiens
TALILAVCCGSASIVSLLLE

SEQ ID NO: 414 - protein, 20 aa, Homo sapiens
AVCCGSASIVSLLLEQNIDV

SEQ ID NO: 415 - protein, 20 aa, Homo sapiens
SASIVSLLLEQNIDVSSQDL

SEQ ID NO: 416 - protein, 20 aa, Homo sapiens
SLLLEQNIDVSSQDLSGQTA

SEQ ID NO: 417 - protein, 20 aa, Homo sapiens
QNIDVSSQDLSGQTAREYAV

SEQ ID NO: 418 - protein, 20 aa, Homo sapiens
SSQDLSGQTAREYAVSSHHH

SEQ ID NO: 419 - protein, 20 aa, Homo sapiens
SGQTAREYAVSSHHHVICQL

SEQ ID NO: 420 - protein, 20 aa, Homo sapiens
REYAVSSHHHVICQLLSDYK

SEQ ID NO: 421 - protein, 20 aa, Homo sapiens
SSHHHVICQLLSDYKEKQML

SEQ ID NO: 422 - protein, 20 aa, Homo sapiens
VICQLLSDYKEKQMLKISSE

SEQ ID NO: 423 - protein, 20 aa, Homo sapiens
LSDYKEKQMLKISSENSNPE

SEQ ID NO: 424 - protein, 20 aa, Homo sapiens
EKQMLKISSENSNPENVSRT

SEQ ID NO: 425 - protein, 20 aa, Homo sapiens
MLKISSENSNPENVSRTRNK

SEQ ID NO: 426 - protein, 33 aa, Homo sapiens
SKMGKWCCRCFPCCRESGKSNVGTSGDHDDSAM

SEQ ID NO: 427 - protein, 33 aa, Homo sapiens
SKMGKWCRHCFPCCRGSGKSNVGASGDHDDSAM

SEQ ID NO: 428 - protein, 33 aa, Homo sapiens
NKMGKWCCHCFPCCRGSGKSKVGAWGDYDDSAF

(19) United States
(12) Patent Application Publication
(10) Pub. No.: US 2006/0083749 A1
(43) Pub. Date: Apr. 20, 2006

(54) Compositions and methods for the therapy and diagnosis of breast cancer

(75) Inventors: Gary R. Fanger, Mill Creek, WA (US); Shannon Kathleen Hirst, Kirkland, WA (US); Davin C. Dillon, Issaquah, WA (US); Teresa M. Foy, Federal Way, WA (US); Raymond L. Houghton, Bothell, WA (US); David H. Persing, Redmond, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Corixa Corporation, Seattle, WA (US)

(21) Appl. No.: 11/139,041

(22) Filed: May 25, 2005

Related U.S. Application Data
(63) Continuation of application No. 10/079,137, filed on Feb. 20, 2002, now abandoned.
Application No. 10/079,137 is a continuation-in-part of application No. 09/924,400, filed on Aug. 7, 2001.
Application No. 09/924,400 is a continuation-in-part of application No. 09/810,936, filed on Mar. 16, 2001, now abandoned.
Application No. 09/810,936 is a continuation-in-part of application No. 09/699,295, filed on Oct. 26, 2000, now Pat. No. 6,828,431.
Application No. 09/699,295 is a continuation-in-part of application No. 09/590,583, filed on Jun. 8, 2000, now abandoned.
Application No. 09/590,583 is a continuation-in-part of application No. 09/577,505, filed on May 24, 2000, now abandoned.
Application No. 09/577,505 is a continuation-in-part of application No. 09/534,825, filed on Mar. 23, 2000, now Pat. No. 6,861,506.
Application No. 09/534,825 is a continuation-in-part of application No. 09/429,755, filed on Oct. 28, 1999, now Pat. No. 6,656,480.
Application No. 09/429,755 is a continuation-in-part of application No. 09/289,198, filed on Apr. 9, 1999, now Pat. No. 6,586,570.
Application No. 09/289,198 is a continuation-in-part of application No. 09/062,451, filed on Apr. 17, 1998, now Pat. No. 6,344,550.
Application No. 09/062,451 is a continuation-in-part of application No. 08/991,789, filed on Dec. 11, 1997, now Pat. No. 6,225,054.
Application No. 08/991,789 is a continuation-in-part of application No. 08/838,762, filed on Apr. 9, 1997, now abandoned.
Application No. 08/838,762 is a continuation-in-part of application No. 08/700,014, filed on Aug. 20, 1996, now abandoned.
Application No. 08/700,014 is a continuation-in-part of application No. 08/585,392, filed on Jan. 11, 1996, now abandoned.

(51) Int. Cl. A61K 39/00; C07H 21/04; C12P 21/06; C07K 14/82

(52) U.S. Cl. 424/184.1; 435/69.1; 435/320.1; 435/325; 530/350; 536/23.5

(57) ABSTRACT

Compositions and methods for the therapy and diagnosis of cancer, particularly breast cancer, are disclosed. Illustrative compositions comprise one or more breast tumor polypeptides, immunogenic portions thereof, polynucleotides that encode such polypeptides, antigen presenting cell that expresses such polypeptides, and T cells that are specific for cells expressing such polypeptides. The disclosed compositions are useful, for example, in the diagnosis, prevention and/or treatment of diseases, particularly breast cancer.

### BACKGROUND OF THE INVENTION

1. Field of the Invention

The present invention relates generally to therapy and diagnosis of cancer, such as breast cancer. The invention is more specifically related to polypeptides, comprising at least a portion of a breast tumor protein, and to polynucleotides encoding such polypeptides. Such polypeptides and polynucleotides are useful in pharmaceutical compositions, e.g., vaccines, and other compositions for the diagnosis and treatment of breast-cancer.

2. Description of the Related Art

Cancer is a significant health problem throughout the world. Although advances have been made in detection and therapy of cancer, no vaccine or other universally successful method for prevention and/or treatment is currently available. Current therapies, which are generally based on a combination of chemotherapy or surgery and radiation, continue to prove inadequate in many patients.

Breast cancer is a significant health problem for women in the United States and throughout the world. Although advances have been made in detection and treatment of the disease, breast cancer remains the second leading cause of cancer-related deaths in women, affecting more than 180,000 women in the United States each year. For women in North America, the life-time odds of getting breast cancer are now one in eight.

No vaccine or other universally successful method for the prevention or treatment of breast cancer is currently available. Management of the disease currently relies on a combination of early diagnosis (through routine breast screening procedures) and aggressive treatment, which may include one or more of a variety of treatments such as surgery, radiotherapy, chemotherapy and hormone therapy. The course of treatment for a particular breast cancer is often selected based on a variety of prognostic parameters, including an analysis of specific tumor markers. See, e.g., Porter-Jordan and Lippman, Breast Cancer 8:73-100 (1994). However, the use of established markers often leads to a result that is difficult to interpret, and the high mortality observed in breast cancer patients indicates that improvements are needed in the treatment, diagnosis and prevention of the disease.

In spite of considerable research into therapies for these and other cancers, breast cancer remains difficult to diagnose and treat effectively. Accordingly, there is a need in the art for improved methods for detecting and treating such cancers. The present invention fulfills these needs and further provides other related advantages.

### SUMMARY OF THE INVENTION

In one aspect, the present invention provides polynucleotide compositions comprising a sequence selected from the group consisting of:

(a) sequences provided in SEQ ID NO:1, 3-86, 142-298, 301-303,-307, 313, 314, 316, 317, 323, 325, 327-330, 335, 339, and 341-344;

(b) complements of the sequences provided in SEQ ID NO:1, 3-86, 142-298, 301-303, 307, 313, 314, 316, 317, 323, 325, 327-330, 335, 339, and 341-344;

(c) sequences consisting of at least 20, 25, 30, 35, 40, 45, 50, 75 and 100 contiguous residues of a sequence provided in SEQ ID NO:1, 3-86, 142-298, 301-303, 307, 313, 314, 316, 317, 323, 325, 327-330, 335, 339, and 341-344;

(d) sequences that hybridize to a sequence provided in SEQ ID NO:1, 3-86, 142-298, 301-303, 307, 313, 314, 316, 317, 323, 325, 327-330, 335, 339, and 341-344, under moderate or highly stringent conditions;

(e) sequences having at least 75%, 80%, 85%, 90%, 95%, 96%, 97%, 98% or 99% identity to a sequence of SEQ ID NO:1, 3-86, 142-298, 301-303, 307, 313, 314, 316, 317, 323, 325, 327-330, 335, 339, and 341-344;

(f) degenerate variants of a sequence provided in SEQ ID NO:1, 3-86, 142-298, 301-303, 307, 313, 314, 316, 317,1323, 325, 327-330, 335, 339, and 341-344.

In one preferred embodiment, the polynucleotide compositions of the invention are expressed in at least about 20%, more preferably in at least about 30%, and most preferably in at least about 50% of breast tumors samples tested, at a level that is at least about 2-fold, preferably at least about 5-fold, and most preferably at least about 10-fold higher than that for normal tissues.

The present invention, in another aspect, provides polypeptide compositions comprising an amino acid sequence that is encoded by a polynucleotide sequence described above.

The present invention further provides polypeptide compositions comprising an amino acid sequence selected from the group consisting of sequences recited in SEQ ID NO:131-140, 299, 300, 304-306, 308-312, 315, 318, 324, 326, 331-334, 336, 340, and 345-428.

In certain preferred embodiments, the polypeptides and/or polynucleotides of the present invention are immunogenic, i.e., they are capable of eliciting an immune response, particularly a humoral and/or cellular immune response, as further described herein.

The present invention further provides fragments, variants and/or derivatives of the disclosed polypeptide and/or polynucleotide sequences, wherein the fragments, variants and/or derivatives preferably have a level of immunogenic activity of at least about 50%, preferably at least about 70% and more preferably at least about 90% of the level of immunogenic activity of a polypeptide sequence set forth in SEQ ID NO:131-140, 299, 300, 304-306, 308-312, 315, 318, 324, 326, 331-334, 336, 340, and 345-428 or a polypeptide sequence encoded by a polynucleotide sequence set forth in SEQ ID NO:1, 3-86, 142-298, 301-303, 307, 313, 314, 316, 317, 323, 325, 327-330, 335, 339, and 341-344.

The present invention further provides polynucleotides that encode a polypeptide described above, expression vectors comprising such polynucleotides and host cells transformed or transfected with such expression vectors.

Within other aspects, the present invention provides pharmaceutical compositions comprising a polypeptide or polynucleotide as described above and a physiologically acceptable carrier.

Within a related aspect of the present invention, the pharmaceutical compositions, e.g., vaccine compositions, are provided for prophylactic or therapeutic applications. Such compositions generally comprise an immunogenic polypeptide or polynucleotide of the invention and an immunostimulant, such as an adjuvant.

The present invention further provides pharmaceutical compositions that comprise: (a) an antibody or antigen-binding fragment thereof that specifically binds to a polypeptide of the present invention, or a fragment thereof; and (b) a physiologically acceptable carrier.

Within further aspects, the present invention provides pharmaceutical compositions comprising: (a) an antigen presenting cell that expresses a polypeptide as described above and (b) a pharmaceutically acceptable carrier or excipient. Illustrative antigen presenting cells include dendritic cells, macrophages, monocytes, fibroblasts and B cells.

Within related aspects, pharmaceutical compositions are provided that comprise: (a) an antigen presenting cell that expresses a polypeptide as described above and (b) an immunostimulant.

The present invention further provides, in other aspects, fusion proteins that comprise at least one polypeptide as described above, as well as polynucleotides encoding such fusion proteins, typically in the form of pharmaceutical compositions, e.g., vaccine compositions, comprising a physiologically acceptable carrier and/or an immunostimulant. The fusions proteins may comprise multiple immunogenic polypeptides or portions/variants thereof, as described herein, and may further comprise one or more polypeptide segments for facilitating the expression, purification and/or immunogenicity of the polypeptide(s).

Within further aspects, the present invention provides methods for stimulating an immune response in a patient, preferably a T cell response in a human patient, comprising administering a pharmaceutical composition described herein. The patient may be afflicted with breast cancer, in which case the methods provide treatment for the disease, or patient considered at risk for such a disease may be treated prophylactically.

Within further aspects, the present invention provides methods for inhibiting the development of a cancer in a patient, comprising administering to a patient a pharmaceutical composition as recited above. The patient may be afflicted with breast cancer, in which case the methods provide treatment for the disease, or patient considered at risk for such a disease may be treated prophylactically.

The present invention further provides, within other aspects, methods for removing tumor cells from a biological sample, comprising contacting a biological sample with T cells that specifically react with a polypeptide of the present invention, wherein the step of contacting is performed under conditions and for a time sufficient to permit the removal of cells expressing the protein from the sample.

Within related aspects, methods are provided for inhibiting the development of a cancer in a patient, comprising administering to a patient a biological sample treated as described above.

Methods are further provided, within other aspects, for stimulating and/or expanding T cells specific for a polypeptide of the present invention, comprising contacting T cells with one or more of: (i) a polypeptide as described above; (ii) a polynucleotide encoding such a polypeptide; and/or (iii) an antigen presenting cell that expresses such a polypeptide; under conditions and for a time sufficient to permit the stimulation and/or expansion of T cells. Isolated T cell populations comprising T cells prepared as described above are also provided.

Within further aspects, the present invention provides methods for inhibiting the development of a cancer in a patient, comprising administering to a patient an effective amount of a T cell population as described above.

The present invention further provides methods for inhibiting the development of a cancer in a patient, comprising the steps of: (a) incubating CD4⁺ and/or CD8⁺ T cells isolated from a patient with one or more of: (i) a polypeptide comprising at least an immunogenic portion of polypeptide disclosed herein; (ii) a polynucleotide encoding such a polypeptide; and (iii) an antigen-presenting cell that expressed such a polypeptide; and (b) administering to the patient an effective amount of the proliferated T cells, and thereby inhibiting the development of a cancer in the patient. Proliferated cells may, but need not, be cloned prior to administration to the patient.

Within further aspects, the present invention provides methods for determining the presence or absence of a cancer, preferably a breast cancer, in a patient comprising: (a) contacting a biological sample obtained from a patient with a binding agent that binds to a polypeptide as recited above; (b) detecting in the sample an amount of polypeptide that binds to the binding agent; and (c) comparing the amount of polypeptide with a predetermined cut-off value, and therefrom determining the presence or absence of a cancer in the patient. Within preferred embodiments, the binding agent is an antibody, more preferably a monoclonal antibody.

The present invention also provides, within other aspects, methods for monitoring the progression of a cancer in a patient. Such methods comprise the steps of: (a) contacting a biological sample obtained from a patient at a first point in time with a binding agent that binds to a polypeptide as recited above; (b) detecting in the sample an amount of polypeptide that binds to the binding agent; (c) repeating steps (a) and (b) using a biological sample obtained from the patient at a subsequent point in time; and (d) comparing the amount of polypeptide detected in step (c) with the amount detected in step (b) and therefrom monitoring the progression of the cancer in the patient.

The present invention further provides, within other aspects, methods for determining the presence or absence of a cancer in a patient, comprising the steps of: (a) contacting a biological sample, e.g., tumor sample, serum sample, etc., obtained from a patient with an oligonucleotide that hybridizes to a polynucleotide that encodes a polypeptide of the present invention; (b) detecting in the sample a level of a polynucleotide, preferably mRNA, that hybridizes to the oligonucleotide; and (c) comparing the level of polynucleotide that hybridizes to the oligonucleotide with a predetermined cut-off value, and therefrom determining the presence or absence of a cancer in the patient. Within certain embodiments, the amount of mRNA is detected via polymerase chain reaction using, for example, at least one oligonucleotide primer that hybridizes to a polynucleotide encoding a polypeptide as recited above, or a complement of such a polynucleotide. Within other embodiments, the amount of mRNA is detected using a hybridization technique, employing an oligonucleotide probe that hybridizes to a polynucleotide that encodes a polypeptide as recited above, or a complement of such a polynucleotide.

In related aspects, methods are provided for monitoring the progression of a cancer in a patient, comprising the steps of: (a) contacting a biological sample obtained from a patient with an oligonucleotide that hybridizes to a polynucleotide that encodes a polypeptide of the present invention; (b) detecting in the sample an amount of a polynucleotide that hybridizes to the oligonucleotide; (c) repeating steps (a) and (b) using a biological sample obtained from the patient at a subsequent point in time; and (d) comparing the amount of polynucleotide detected in step (c) with the amount detected in step (b) and therefrom monitoring the progression of the cancer in the patient.

Within further aspects, the present invention provides antibodies, such as monoclonal antibodies, that bind to a polypeptide as described above, as well as diagnostic kits comprising such antibodies. Diagnostic kits comprising one or more oligonucleotide probes or primers as described above are also provided.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 shows the differential display PCR products, separated by gel electrophoresis, obtained from cDNA prepared from normal breast tissue (lanes 1 and 2) and from cDNA prepared from breast tumor tissue from the same patient (lanes 3 and 4). The arrow indicates the band corresponding to B18Ag1.

FIG. 2 is a northern blot comparing the level of B18Ag1 mRNA in breast tumor tissue (lane 1) with the level in normal breast tissue.

FIG. 3 shows the level of B18Ag1 mRNA in breast tumor tissue compared to that in various normal and non-breast tumor tissues as determined by RNase protection assays.

FIG. 4 is a genomic clone map showing the location of additional retroviral sequences obtained from ends of XbaI restriction digests (provided in SEQ ID NO:3-SEQ ID NO:10) relative to B18Ag1.

FIGS. 5A and 5B show the sequencing strategy, genomic organization and predicted open reading frame for the retroviral element containing B18Ag1.

FIG. 6 shows the nucleotide sequence of the representative breast tumor-specific cDNA B18Ag1.

FIG. 7 shows the nucleotide sequence of the representative breast tumor-specific cDNA B17Ag1.

FIG. 8 shows the nucleotide sequence of the representative breast tumor-specific cDNA B17Ag2.

FIG. 9 shows the nucleotide sequence of the representative breast tumor-specific cDNA B13Ag2a.

FIG. 10 shows the nucleotide sequence of the representative breast tumor-specific cDNA B13Ag1b.

FIG. 11 shows the nucleotide sequence of the representative breast tumor-specific cDNA B13Ag1a.

FIG. 12 shows the nucleotide sequence of the representative breast tumor-specific cDNA B11Ag1.

FIG. 13 shows the nucleotide sequence of the representative breast tumor-specific cDNA B3CA3c.

FIG. 14 shows the nucleotide sequence of the representative breast tumor-specific cDNA B9CG1.

FIG. 15 shows the nucleotide sequence of the representative breast tumor-specific cDNA B9CG3.

FIG. 16 shows the nucleotide sequence of the representative breast tumor-specific cDNA B2CA2.

FIG. 17 shows the nucleotide sequence of the representative breast tumor-specific cDNA B3CA1.

FIG. 18 shows the nucleotide sequence of the representative breast tumor-specific cDNA B3CA2.

FIG. 19 shows the nucleotide sequence of the representative breast tumor-specific cDNA B3CA3.

FIG. 20 shows the nucleotide sequence of the representative breast tumor-specific cDNA B4CA1.

FIG. 21 A depicts RT-PCR analysis of breast tumor genes in breast tumor tissues (lanes 1-8) and normal breast tissues (lanes 9-13) and H₂O (lane 14).

FIG. 21B depicts RT-PCR analysis of breast tumor genes in prostate tumors (lane 1, 2), colon tumors (lane 3), lung tumor (lane 4), normal prostate (lane 5), normal colon (lane 6), normal kidney (lane 7), normal liver (lane 8), normal lung (lane 9), normal ovary (lanes 10, 18), normal pancreases (lanes 11, 12), normal skeletal muscle (lane 13), normal skin (lane 14), normal stomach (lane 15), normal testes (lane 16), normal small intestine (lane 17), HBL-100 (lane 19), MCF-12A (lane 20), breast tumors (lanes 21-23), H₂O (lane 24), and colon tumor (lane 25).

FIG. 22 shows the recognition of a B11Ag1 peptide (referred to as B11-8) by an anti-B11-8 CTL line.

FIG. 23 shows the recognition of a cell line transduced with the antigen, B11Ag1 by the B11-8 specific clone A1.

FIG. 24 shows recognition of a lung adenocarcinoma line (LT-140-22) and a breast adenocarcinoma line (CAMA-1) by the B11-8 specific clone A1.

### DETAILED DESCRIPTION OF THE INVENTION

The present invention is directed generally to compositions and their use in the therapy and diagnosis of cancer, particularly breast cancer. As described further below, illustrative compositions of the present invention include, but are not restricted to, polypeptides, particularly immunogenic polypeptides, polynucleotides encoding such polypeptides, antibodies and other binding agents, antigen presenting cells (APCs) and immune system cells (e.g., T cells).

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

All publications, patents and patent applications cited herein, whether supra or infra, are hereby incorporated by reference in their entirety.

As used in this specification and the appended claims, the singular forms “a,” “an” and “the” include plural references unless the content clearly dictates otherwise.

Polypeptide Compositions

As used herein, the term “polypeptide” is used in its conventional meaning, i.e., as a sequence of amino acids. The polypeptides are not limited to a specific length of the product; thus, peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof. Particular polypeptides of interest in the context of this invention are amino acid subsequences comprising epitopes, i.e., antigenic determinants substantially responsible for the immunogenic properties of a polypeptide and being capable of evoking an immune response.

Particularly illustrative polypeptides of the present invention comprise those encoded by a polynucleotide sequence set forth in any one of SEQ,ID NO:1, 3-86, 142-298, 301-303, 307, 313, 314, 316, 317, 323, 325, 327-330, 335, 339, and 341-344, or a sequence that hybridizes under moderately stringent conditions, or, alternatively, under highly stringent conditions, to a polynucleotide sequence set forth in any one of SEQ ID NO:1, 3-86, 142-298, 301-303, 307, 313, 314, 316, 317, 323, 325, 327-330, 335, 339, and 341-344. Certain other illustrative polypeptides of the invention comprise amino acid sequences as set forth in any one of SEQ ID NO:131-140, 299, 300, 304-306, 308-312, 315, 318, 324, 326, 331-334, 336, 340, and 345-428.

The polypeptides of the present invention are sometimes herein referred to as breast tumor proteins or breast tumor polypeptides, as an indication that their identification has been based at least in part upon their increased levels of expression in breast tumor samples. Thus, a “breast tumor polypeptide” or “breast tumor protein,” refers generally to a polypeptide sequence of the present invention, or a polynucleotide sequence encoding such a polypeptide, that is expressed in a substantial proportion of breast tumor samples, for example preferably greater than about 20%, more preferably greater than about 30%, and most preferably greater than about 50% or more of breast tumor samples tested, at a level that is at least two fold, and preferably at least five fold, greater than the level of expression in normal tissues, as determined using a representative assay provided herein. A breast tumor polypeptide sequence of the invention, based upon its increased level of expression in tumor cells, has particular utility both as a diagnostic marker as well as a therapeutic target, as further described below.

In certain preferred embodiments, the polypeptides of the invention are immunogenic, i.e., they react detectably within an immunoassay (such as an ELISA or T-cell stimulation assay) with antisera and/or T-cells from a patient with breast cancer. Screening for immunogenic activity can be performed using techniques well known to the skilled artisan. For example, such screens can be performed using methods such as those described in Harlow and Lane, Antibodies: A Laboratory Manual, Cold Spring Harbor Laboratory, 1988. In one illustrative example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

As would be recognized by the skilled artisan, immunogenic portions of the polypeptides disclosed herein are also encompassed by the present invention. An “immunogenic portion,” as used herein, is a fragment of an immunogenic polypeptide of the invention that itself is immunologically reactive (i.e., specifically binds) with the B-cells and/or T-cell surface antigen receptors that recognize the polypeptide. Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well-known techniques.

In one preferred embodiment, an immunogenic portion of a polypeptide of the present invention is a portion that reacts with antisera and/or T-cells at a level that is not substantially less than the reactivity of the full-length polypeptide (e.g., in an ELISA and/or T-cell reactivity assay). Preferably, the level of immunogenic activity of the immunogenic portion is at least about 50%, preferably at least about 70% and most preferably greater than about 90% of the immunogenicity for the full-length polypeptide. In some instances, preferred immunogenic portions will be identified that have a level of immunogenic activity greater than that of the corresponding full-length polypeptide, e.g., having greater than about 100% or 150% or more immunogenic activity.

In certain other embodiments, illustrative immunogenic portions may include peptides in which an N-terminal leader sequence and/or transmembrane domain have been deleted. Other illustrative immunogenic portions will contain a small N— and/or C-terminal deletion (e.g., 1-30 amino acids, preferably 5-15 amino acids), relative to the mature protein.

In another embodiment, a polypeptide composition of the invention may also comprise one or more polypeptides that are immunologically reactive with T cells and/or antibodies generated against a polypeptide of the invention, particularly a polypeptide having an amino acid sequence disclosed herein, or to an immunogenic fragment or variant thereof.

In another embodiment of the invention, polypeptides are provided that comprise one or more polypeptides that are capable of eliciting T cells and/or antibodies that are immunologically reactive with one or more polypeptides described herein, or one or more polypeptides encoded by contiguous nucleic acid sequences contained in the polynucleotide sequences disclosed herein, or immunogenic fragments or variants thereof, or to one or more nucleic acid sequences which hybridize to one or more of these sequences under conditions of moderate to high stringency.

The present invention, in another aspect, provides polypeptide fragments comprising at least about 5, 10, 15, 20, 25, 50, or 100 contiguous amino acids, or more, including all intermediate lengths, of a polypeptide compositions set forth herein, such as those set forth in SEQ ID NO:131-140, 299, 300, 304-306, 308-312, 315, 318, 324, 326, 331-334, 336, 340, and 345-428, or those encoded by a polynucleotide sequence set forth in a sequence of SEQ ID NO:1, 3-86, 142-298, 301-303, 307, 313, 314, 316, 317, 323, 325, 327-330, 335, 339, and 341-344.

In another aspect, the present invention provides variants of the polypeptide compositions described herein. Polypeptide variants generally encompassed by the present invention will typically exhibit at least about 70%, 75%, 80%, 85%, 90%, 91%, 92%, 93%, 94%, 95%, 96%, 97%, 98%, or 99% or more identity (determined as described below), along its length, to a polypeptide sequences set forth herein.

In one preferred embodiment, the polypeptide fragments and variants provided by the present invention are immunologically reactive with an antibody and/or T-cell that reacts with a full-length polypeptide specifically set forth herein.

In another preferred embodiment, the polypeptide fragments and variants provided by the present invention exhibit a level of immunogenic activity of at least about 50%, preferably at least about 70%, and most preferably at least about 90% or more of that exhibited by a full-length polypeptide sequence specifically set forth herein.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating their immunogenic activity as described herein and/or using any of a number of techniques well known in the art.

For example, certain illustrative variants of the polypeptides of the invention include those in which one or more portions, such as an N-terminal leader sequence or transmembrane domain, have been removed. Other illustrative variants include variants in which a small portion (e.g., 1-30 amino acids, preferably 5-15 amino acids) has been removed from the N— and/or C-terminal of the mature protein.

In many instances, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics, e.g., with immunogenic characteristics. When it is desired to alter the amino acid sequence of a polypeptide to create an equivalent, or even an improved, immunogenic variant or portion of a polypeptide of the invention, one skilled in the art will typically change one or more of the codons of the encoding DNA sequence according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their biological utility or activity.

TABLE 1

| | |
|---|---|
| Amino Acids | Codons |

| | | | | | | | | |
|---|---|---|---|---|---|---|---|---|
| Alanine | Ala | A | GCA | GCC | GCG | GCU | | |
| Cysteine | Gys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Iie | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Praline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

In addition, any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

When comparing polypeptide sequences, two sequences are said to be “identical” if the sequence of amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Saitou, N. Nei, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one preferred approach, the “percentage of sequence identity” is determined by comparing two optimally aligned sequences over a window of comparison of at least 20 positions, wherein the portion of the polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e., the window size) and multiplying the results by 100 to yield the percentage of sequence identity.

Within other illustrative embodiments, a polypeptide may be a xenogeneic polypeptide that comprises an polypeptide having substantial sequence identity, as described above, to the human polypeptide (also termed autologous antigen) which served as a reference polypeptide, but which xenogeneic polypeptide is derived from a different, non-human species. One skilled in the art will recognize that “self” antigens are often poor stimulators of CD8+ and CD4+ T-lymphocyte responses, and therefore efficient immunotherapeutic strategies directed against tumor polypeptides require the development of methods to overcome immune tolerance to particular self tumor polypeptides. For example, humans immunized with prostase protein from a xenogeneic (non human) origin are capable of mounting an immune response against the counterpart human protein, e.g. the human prostase tumor protein present on human tumor cells. Accordingly, the present invention provides methods for purifying the xenogeneic form of the tumor proteins set forth herein, such as the polypeptides set forth in SEQ ID NO:131-140, 299, 300, 304-306, 308-312, 315, 318, 324, 326, 331-334, 336, 340, and 345-428, or those encoded by polynucleotide sequences set forth in SEQ ID NO:1, 3-86, 142-298, 301-303, 307, 313, 314, 316, 317, 323, 325, 327-330, 335, 339, and 341-344.

Therefore, one aspect of the present invention provides xenogeneic variants of the polypeptide compositions described herein. Such xenogeneic variants generally encompassed by the present invention will typically exhibit at least about 70%, 75%, 80%, 85%, 90%, 91%, 92%, 93%, 94%, 95%, 96%, 97%, 98%, or 99% or more identity along their lengths, to a polypeptide sequences set forth herein.

More particularly, the invention is directed to mouse, rat, monkey, porcine and other non-human polypeptides which can be used as xenogeneic forms of human polypeptides set forth herein, to induce immune responses directed against tumor polypeptides of the invention.

Within other illustrative embodiments, a polypeptide may be a fusion polypeptide that comprises multiple polypeptides as described herein, or that comprises at least one polypeptide as described herein and an unrelated sequence, such as a known tumor protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the polypeptide or to enable the polypeptide to be targeted to desired intracellular compartments. Still further fusion partners include affinity tags, which facilitate purification of the polypeptide.

Fusion polypeptides may generally be prepared using standard techniques, including chemical conjugation. Preferably, a fusion polypeptide is expressed as a recombinant polypeptide, allowing the production of increased levels, relative to a non-fused polypeptide, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion polypeptide that retains the biological activity of both component polypeptides.

A peptide linker sequence may be employed to separate the first and second polypeptide components by a distance sufficient to ensure that each-polypeptide folds into its secondary and tertiary structures. Such a peptide linker sequence is incorporated into the fusion polypeptide using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

The fusion polypeptide can comprise a polypeptide as described herein together with an unrelated immunogenic protein, such as an immunogenic protein capable of eliciting a recall response. Examples of such proteins include tetanus, tuberculosis and hepatitis proteins (see, for example, Stoute et al. New Engl. J. Med., 336:86-91, 1997).

In one preferred embodiment, the immunological fusion partner is derived from a Mycobacterium sp., such as a Mycobacterium tuberculosis-derived Ra12 fragment. Ra12 compositions and methods for their use in enhancing the expression and/or immunogenicity of heterologous polynucleotide/polypeptide sequences is described in U.S. Patent Application 60/158,585, the disclosure of which is incorporated herein by reference in its entirety. Briefly, Ra12 refers to a polynucleotide region that is a subsequence of a Mycobacterium tuberculosis MTB32A nucleic acid. MTB32A is a serine protease of 32 KD molecular weight encoded by a gene in virulent and avirulent strains of M. tuberculosis. The nucleotide sequence and amino acid sequence of MTB32A have been described (for example, U.S. Patent Application 60/158,585; see also, Skeiky et al., Infection and Immun. (1999) 67:3998-4007, incorporated herein by reference). C-terminal fragments of the MTB32A coding sequence express at high levels and remain as a soluble polypeptides throughout the purification process. Moreover, Ra12 may enhance the immunogenicity of heterologous immunogenic polypeptides with which it is fused. One preferred Ra12 fusion polypeptide comprises a 14 KD C-terminal fragment corresponding to amino acid residues 192 to 323 of MTB32A. Other preferred Ra12 polynucleotides generally comprise at least about 15 consecutive nucleotides, at least about 30 nucleotides, at least about 60 nucleotides, at least about 100 nucleotides, at least about 200 nucleotides, or at least about 300 nucleotides that encode a portion of a Ra12 polypeptide. Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide. Variants preferably exhibit at least about 70% identity, more preferably at least about 80% identity and most preferably at least about 90% identity to a polynucleotide sequence that encodes a native Ra12 polypeptide or a portion thereof.

Within other preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion polypeptide. A repeat portion is found in the C-terminal region starting at residue 178. A particularly preferred repeat portion incorporates residues 188-305.

Yet another illustrative embodiment involves fusion polypeptides, and the polynucleotides encoding them, wherein the fusion partner comprises a targeting signal capable of directing a polypeptide to the endosomal/lysosomal compartment, as described in U.S. Pat. No. 5,633,234. An immunogenic polypeptide of the invention, when fused with this targeting signal, will associate more efficiently with MHC class II molecules and thereby provide enhanced in vivo stimulation of CD4⁺ T-cells specific for the polypeptide.

Polypeptides of the invention are prepared using any of a variety of well known synthetic and/or recombinant techniques, the latter of which are further described below. Polypeptides, portions and other variants generally less than about 150 amino acids can be generated by synthetic means, using techniques well known to those of ordinary skill in the art. In one illustrative example, such polypeptides are synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

In general, polypeptide compositions (including fusion polypeptides) of the invention are isolated. An “isolated” polypeptide is one that is removed from its original environment. For example, a naturally-occurring protein or polypeptide is isolated if it is separated from some or all of the coexisting materials in the natural system. Preferably, such polypeptides are also purified, e.g., are at least about 90% pure, more preferably at least about 95% pure and most preferably at least about 99% pure.

Polynucleotide Compositions

The present invention, in other aspects, provides polynucleotide compositions. The terms “DNA” and “polynucleotide” are used essentially interchangeably herein to refer to a DNA molecule that has been isolated free of total genomic DNA of a particular species. “Isolated,” as used herein, means that a polynucleotide is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man.

As will be understood by those skilled in the art, the polynucleotide compositions of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

As will be also recognized by the skilled artisan, polynucleotides of the invention may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a polypeptide/protein of the invention or a portion thereof) or may comprise a sequence that encodes a variant or derivative, preferably and immunogenic variant or derivative, of such a sequence.

Therefore, according to another aspect of the present invention, polynucleotide compositions are provided that comprise some or all of a polynucleotide sequence set forth in any one of SEQ ID NO:1, 3-86, 142-298, 301-303, 307, 313, 314, 316, 317, 323, 325, 327-330, 335, 339, and 341-344, complements of a polynucleotide sequence set forth in any one of SEQ ID NO:1, 3-86, 142-298, 301-303, 307, 313, 314, 316, 317, 323, 325, 327-330, 335, 339, and 341-344, and degenerate variants of a polynucleotide sequence set forth in any one of SEQ ID NO:1, 3-86, 142-298, 301-303, 307, 313, 314, 316, 317, 323, 325, 327-330, 335, 339, and 341-344. In certain preferred embodiments, the polynucleotide sequences set forth herein encode immunogenic polypeptides, as described above.

In other related embodiments, the present invention provides polynucleotide variants having substantial identity to the sequences disclosed herein in SEQ ID NO:1, 3-86, 142-298, 301-303, 307, 313, 314, 316, 317, 323, 325, 327-330, 335, 339, and 341-344, for example those comprising at least 70% sequence identity, preferably at least 75%, 80%, 85%, 90%, 95%, 96%, 97%, 98%, or 99% or higher, sequence identity compared to a polynucleotide sequence of this invention using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the immunogenicity of the polypeptide encoded by the variant polynucleotide is not substantially diminished relative to a polypeptide encoded by a polynucleotide sequence specifically set forth herein). The term “variants” should also be understood to encompasses homologous genes of xenogenic origin.

In additional embodiments, the present invention provides polynucleotide fragments comprising or consisting of various lengths of contiguous stretches of sequence identical to or complementary to one or more of the sequences disclosed herein. For example, polynucleotides are provided by this invention that comprise or consist of at least about 10, 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like. A polynucleotide sequence as described here may be extended at one or both ends by additional nucleotides not found in the native sequence. This additional sequence may consist of 1, 2, 3, 4, 5, 6, 7, 8, 9, 10, 11, 12, 13, 14, 15, 16, 17, 18, 19, or 20 nucleotides at either end of the disclosed sequence or at both ends of the disclosed sequence.

In another embodiment of the invention, polynucleotide compositions are provided that are capable of hybridizing under moderate to high stringency conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5×SSC, 0.5% SDS, 1.0 mM EDTA (pH 8.0); hybridizing at 50° C.-60° C., 5×SSC, overnight; followed by washing twice at 65° C. for 20 minutes with each of 2×, 0.5× and 0.2×SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60-65° C. or 65-70° C.

In certain preferred embodiments, the polynucleotides described above, e.g., polynucleotide variants, fragments and hybridizing sequences, encode polypeptides that are immunologically cross-reactive with a polypeptide sequence specifically set forth herein. In other preferred embodiments, such polynucleotides encode polypeptides that have a level of immunogenic activity of at least about 50%, preferably at least about 70%, and more preferably at least about 90% of that for a polypeptide sequence specifically set forth herein.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides of the invention. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

Preferably, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Therefore, in another embodiment of the invention, a mutagenesis approach, such as site-specific mutagenesis, is employed for the preparation of immunogenic variants and/or derivatives of the polypeptides described herein. By this approach, specific modifications in a polypeptide sequence can be made through mutagenesis of the underlying polynucleotides that encode them. These techniques provides a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments of the present invention, the inventors contemplate the mutagenesis of the disclosed polynucleotide sequences to alter one or more properties of the encoded polypeptide, such as the immunogenicity of a polypeptide vaccine. The techniques of site-specific mutagenesis are well-known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al, 1982, each incorporated herein by reference, for that purpose.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In another approach for the production of polypeptide variants of the present invention, recursive sequence recombination, as described in U.S. Patent No. 5,837,458, may be employed. In this approach, iterative cycles of recombination and screening or selection are performed to “evolve” individual polynucleotide variants of the invention having, for example, enhanced immunogenic activity.

In other embodiments of the present, invention, the polynucleotide sequences provided herein can be advantageously used as probes or primers for nucleic acid hybridization. As such, it is contemplated that nucleic acid segments that comprise or consist of a sequence region of at least about a 15 nucleotide long contiguous sequence that has the same sequence as, or is complementary to, a 15 nucleotide long contiguous sequence disclosed herein will find particular utility. Longer contiguous identical or complementary sequences, e.g., those of about 20, 30, 40, 50, 100, 200, 500, 1000 (including all intermediate lengths) and even up to full length sequences will also be of use in certain embodiments.

The ability of such nucleic acid probes to specifically hybridize to a sequence of interest will enable them to be of use in detecting the presence of complementary sequences in a given sample. However, other uses are also envisioned, such as the use of the sequence information for the preparation of mutant species primers, or primers for use in preparing other genetic constructions.

Polynucleotide molecules having sequence regions consisting of contiguous nucleotide stretches of 10-14, 15-20, 30, 50, or even of 100-200 nucleotides or so (including intermediate lengths as well), identical or complementary to a polynucleotide sequence disclosed herein, are particularly contemplated as hybridization probes for use in, e.g., Southern and Northern blotting. This would allow a gene product, or fragment thereof, to be analyzed, both in diverse cell types and also in various bacterial cells. The total size of fragment, as well as the size of the complementary stretch(es), will ultimately depend on the intended use or application of the particular nucleic acid segment. Smaller fragments will generally find use in hybridization embodiments, wherein the length of the contiguous complementary region may be varied, such as between about 15 and about 100 nucleotides, but larger contiguous complementarity stretches, may be used, according to the length complementary sequences one wishes to detect.

The use of a hybridization probe of about 15-25 nucleotides in length allows the formation of a duplex molecule that is both stable and selective. Molecules having contiguous complementary sequences over stretches greater than 15 bases in length are generally preferred, though, in order to increase stability and selectivity of the hybrid, and thereby improve the quality and degree of specific hybrid molecules obtained. One will generally prefer to design nucleic acid molecules having gene-complementary stretches of 15 to 25 contiguous nucleotides, or even longer where desired.

Hybridization probes may be selected from any portion of any of the sequences disclosed herein. All that is required is to review the sequences set forth herein, or to any continuous portion of the sequences, from about 15-25 nucleotides in length up to and including the full length sequence, that one wishes to utilize as a probe or primer. The choice of probe and primer sequences may be governed by various factors. For example, one may wish to employ primers from towards the termini of the total sequence.

Small polynucleotide segments or fragments may be readily prepared by, for example, directly synthesizing the fragment by chemical means, as is commonly practiced using an automated oligonucleotide synthesizer. Also, fragments may be obtained by application of nucleic acid reproduction technology, such as the PCR™ technology of U.S. Pat. No. 4,683,202 (incorporated herein by reference), by introducing selected sequences into recombinant vectors for recombinant production, and by other recombinant DNA techniques generally known to those of skill in the art of molecular biology.

The nucleotide sequences of the invention may be used for their ability to selectively form duplex molecules with complementary stretches of the entire gene or gene fragments of interest. Depending on the application envisioned, one will typically desire to employ varying conditions of hybridization to achieve varying degrees of selectivity of probe towards target sequence. For applications requiring high selectivity, one will typically desire to employ relatively stringent conditions to form the hybrids, e.g., one will select relatively low salt and/or high temperature conditions, such as provided by a salt concentration of from about 0.02 M to about 0.15 M salt at temperatures of from about 50° C. to about 700° C. Such selective conditions tolerate little, if any, mismatch between the probe and the template or target strand, and would be particularly suitable for isolating related sequences.

Of course, for some applications, for example, where one desires to prepare mutants employing a mutant primer strand hybridized to an underlying template, less stringent (reduced stringency) hybridization conditions will typically be needed in order to allow formation of the heteroduplex. In these circumstances, one may desire to employ salt conditions such as those of from about 0.15 M to about 0.9 M salt, at temperatures ranging from about 20° C. to about 55° C. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results.

According to another embodiment of the present invention, polynucleotide compositions comprising antisense oligonucleotides are provided. Antisense oligonucleotides have been demonstrated to be effective and targeted inhibitors of protein synthesis, and, consequently, provide a therapeutic approach by which a disease can be treated by inhibiting the synthesis of proteins that contribute to the disease. The efficacy of antisense oligonucleotides for inhibiting protein synthesis is well established. For example, the synthesis of polygalactauronase and the muscarine type 2 acetylcholine receptor are inhibited by antisense oligonucleotides directed to their respective mRNA sequences (U.S. Pat. No. 5,739,119 and U.S. Pat. No. 5,759,829). Further, examples of antisense inhibition have been demonstrated with the nuclear protein cyclin, the multiple drug resistance gene (MDG1), ICAM-1, E-selectin, STK-1, striatal GABA_{A} receptor and human EGF (Jaskulski et al., Science. Jun. 10, 1988;240(4858):1544-6; Vasanthakumar and Ahmed, Cancer Commun. 1989;1(4):225-32; Peris et al., Brain Res Mol Brain Res. Jun. 15, 1998;57(2):310-20; U.S. Pat. No. 5,801,154; U.S. Pat. No. 5,789,573; U.S. Pat. No. 5,718,709 and U.S. Pat. No. 5,610,288). Antisense constructs have also been described that inhibit and can be used to treat a variety of abnormal cellular proliferations, e.g. cancer (U.S. Pat. No. 5,747,470; U.S. Pat. No. 5,591,317 and U.S. Pat. No. 5,783,683).

Therefore, in certain embodiments, the present invention provides oligonucleotide sequences that comprise all, or a portion of, any sequence that is capable of specifically binding to polynucleotide sequence described herein, or a complement thereof. In one embodiment, the antisense oligonucleotides comprise DNA or derivatives thereof. In another embodiment, the oligonucleotides comprise RNA or derivatives thereof. In a third embodiment, the oligonucleotides are modified DNAs comprising a phosphorothioated modified backbone. In a fourth embodiment, the oligonucleotide sequences comprise peptide nucleic acids or derivatives thereof. In each case, preferred compositions comprise a sequence region that is complementary, and more preferably substantially-complementary, and even more preferably, completely complementary to one or more portions of polynucleotides disclosed herein. Selection of antisense compositions specific for a given gene sequence is based upon analysis of the chosen target sequence and determination of secondary structure, Tₘ, binding energy, and relative stability. Antisense compositions may be selected based upon their relative inability to form dimers, hairpins, or other secondary structures that would reduce or prohibit specific binding to the target mRNA in a host cell. Highly preferred target regions of the mRNA, are those which are at or near the AUG translation initiation codon, and those sequences which are substantially complementary to 5′ regions of the mRNA. These secondary structure analyses and target site selection considerations can be performed, for example, using v.4 of the OLIGO primer analysis software and/or the BLASTN 2.0.5 algorithm software (Altschul et al., Nucleic Acids Res. 1997, 25(17):3389-402).

The use of an antisense delivery method employing a short peptide vector, termed MPG (27 residues), is also contemplated. The MPG peptide contains a hydrophobic domain derived from the fusion sequence of HIV gp41 and a hydrophilic domain from the nuclear localization sequence of SV40 T-antigen (Morris et al., Nucleic Acids Res. Jul. 15, 1997;25(14):2730-6). It has been demonstrated that several molecules of the MPG peptide coat the antisense oligonucleotides and can be delivered into cultured mammalian cells in less than 1 hour with relatively high efficiency (90%). Further, the interaction with MPG strongly increases both the stability of the oligonucleotide to nuclease and the ability to cross the plasma membrane.

According to another embodiment of the invention, the polynucleotide compositions described herein are used in the design and preparation of ribozyme molecules for inhibiting expression of the tumor polypeptides and proteins of the present invention in tumor cells. Ribozymes are RNA-protein complexes that cleave nucleic acids in a site-specific fashion. Ribozymes have specific catalytic domains that possess endonuclease activity (Kim and Cech, Proc Natl Acad Sci USA. December 1987;84(24):8788-92; Forster and Symons, Cell. Apr. 24, 1987;49(2):211-20). For example, a large number of ribozymes accelerate phosphoester transfer reactions with a high degree of specificity, often cleaving only one of several phosphoesters in an oligonucleotide substrate (Cech et al, Cell. December 1981;27(3 Pt 2):487-96; Michel and Westhof, J Mol Biol. December 5, 1990;216(3):585-610; Reinhold-Hurek and Shub, Nature. May 14, 1992;357(6374):173-6). This specificity has been attributed to the requirement that the substrate bind via specific base-pairing interactions to the internal guide sequence (“IGS”) of the ribozyme prior to chemical reaction.

Six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme is advantageous over many technologies, such as antisense technology (where a nucleic acid molecule simply binds to a nucleic acid target to block its translation) since the concentration of ribozyme necessary to affect a therapeutic treatment is lower than that of an antisense oligonucleotide. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme. Similar mismatches in antisense molecules do not prevent their action (Woolf et al., Proc Natl Acad Sci USA. Aug. 15, 1992;89(16):7305-9). Thus, the specificity of action of a ribozyme is greater than that of an antisense oligonucleotide binding the same RNA site.

The enzymatic nucleic acid molecule may be formed in a hammerhead, hairpin, a hepatitis δ virus, group I intron or RNaseP RNA (in association with an RNA guide sequence) or Neurospora VS RNA motif. Examples of hammerhead motifs are described by Rossi et al. Nucleic Acids Res. Sep. 11, 1992;20(17):4559-65. Examples of hairpin motifs are described by Hampel et al. (Eur. Pat. Appl. Publ. No. EP 0360257), Hampel and Tritz, Biochemistry Jun. 13, 1989;28(12):4929-33; Hampel et al., Nucleic Acids Res. Jan. 25, 1990;18(2):299-304 and U.S. Pat. No. 5,631,359. An example of the hepatitis δ virus motif is described by Perrotta and Been, Biochemistry. Dec. 1, 1992;31(47): 11843-52; an example of the RNaseP motif is described by Guerrier-Takada et al., Cell. Decembr 1983;35(3 Pt 2):849-57; Neurospora VS RNA ribozyme motif is described by Collins (Saville and Collins, Cell. May 18, 1990;61(4):685-96; Saville and Collins, Proc Natl Acad Sci USA. Oct. 1, 1991;88(19):8826-30; Collins and Olive, Biochemistry. Mar. 23, 1993;32(11):2795-9); and an example of the Group I intron is described in (U.S. Pat. No. 4,987,071). All that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule. Thus the ribozyme constructs need not be limited to specific motifs mentioned herein.

Ribozymes may be designed as described in Int. Pat. Appl. Publ. No. WO 93/23569 and Int. Pat. Appl. Publ. No. WO 94/02595, each specifically incorporated herein by reference) and synthesized to be tested in vitro and in vivo, as described. Such ribozymes can also be optimized for delivery. While specific examples are provided, those in the art will recognize that equivalent RNA targets in other species can be utilized when necessary.

Ribozyme activity can be optimized by altering the length of the ribozyme binding arms, or chemically synthesizing ribozymes with modifications that prevent their degradation by serum ribonucleases (see e.g., Int. Pat. Appl. Publ. No. WO 92/07065; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

Sullivan et al. (Int. Pat. Appl. Publ. No. WO 94/02595) describes the general methods for delivery of enzymatic RNA molecules. Ribozymes may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, ribozymes may be directly delivered ex vivo to cells or tissues with or without the aforementioned vehicles. Alternatively, the RNA/vehicle combination may be locally delivered by direct inhalation, by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of ribozyme delivery and administration are provided in Int. Pat. Appl. Publ. No. WO 94/02595 and Int. Pat. Appl. Publ. No. WO 93/23569, each specifically incorporated herein by reference.

Another means of accumulating high concentrations of a ribozyme(s) within cells is to incorporate the ribozyme-encoding sequences into a DNA expression vector. Transcription of the ribozyme sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters may also be used, providing that the prokaryotic RNA polymerase enzyme is expressed in the appropriate cells Ribozymes expressed from such promoters have been shown to function in mammalian cells. Such transcription units can be incorporated into a variety of vectors for introduction into mammalian cells, including but not restricted to, plasmid DNA vectors, viral DNA vectors (such as adenovirus or adeno-associated vectors), or viral RNA vectors (such as retroviral, semliki forest virus, sindbis virus vectors).

In another embodiment of the invention, peptide nucleic acids (PNAs) compositions are provided. PNA is a DNA mimic in which the nucleobases are attached to a pseudopeptide backbone (Good and Nielsen, Antisense Nucleic Acid Drug Dev. 1997 7(4) 431-37). PNA is able to be utilized in a number methods that traditionally have used RNA or DNA. Often PNA sequences perform better in techniques than the corresponding RNA or DNA sequences and have utilities that are not inherent to RNA or DNA. A review of PNA including methods of making, characteristics of, and methods of using, is provided by Corey (Trends Biotechnol June 1997;15(6):224-9). As such, in certain embodiments, one may prepare PNA sequences that are complementary to one or more portions of the ACE mRNA sequence, and such PNA compositions may be used to regulate, alter, decrease, or reduce the translation of ACE-specific mRNA, and thereby alter the level of ACE activity in a host cell to which such PNA compositions have been administered.

PNAs have 2-aminoethyl-glycine linkages replacing the normal phosphodiester backbone of DNA (Nielsen et al., Science Dec. 6, 1991;254(5037):1497-500; Hanvey et al., Science. Nov. 27, 1992;258(5087):1481-5; Hyrup and Nielsen, Bioorg Med Chem. January 1996;4(1):5-23). This chemistry has three important consequences: firstly, in contrast to DNA or phosphorothioate oligonucleotides, PNAs are neutral molecules; secondly, PNAs are achiral, which avoids the need to develop a stereoselective synthesis; and thirdly, PNA synthesis uses standard Boc or Fmoc protocols for solid-phase peptide synthesis, although other methods, including a modified Merrifield method, have been used.

PNA monomers or ready-made oligomers are commercially available from PerSeptive Biosystems (Framingham, Mass.). PNA syntheses by either Boc or Fmoc protocols are straightforward using manual or automated protocols (Norton et al., Bioorg Med Chem. April 1995;3(4):437-45). The manual protocol lends itself to the production of chemically modified PNAs or the simultaneous synthesis of families of closely related PNAs.

As with peptide synthesis, the success of a particular PNA synthesis will depend on the properties of the chosen sequence. For example, while in theory PNAs can incorporate any combination of nucleotide bases, the presence of adjacent purines can lead to deletions of one or more residues in the product. In expectation of this difficulty, it is suggested that, in producing PNAs with adjacent purines, one should repeat the coupling of residues likely to be added inefficiently. This should be followed by the purification of PNAs by reverse-phase high-pressure liquid chromatography, providing yields and purity of product similar to those observed during the synthesis of peptides.

Modifications of PNAs for a given application may be accomplished by coupling amino acids during solid-phase synthesis or by attaching compounds that contain a carboxylic acid group to the exposed N-terminal amine. Alternatively, PNAs can be modified after synthesis by coupling to an introduced lysine or cysteine. The ease with which PNAs can be modified facilitates optimization for better solubility or for specific functional requirements. Once synthesized, the identity of PNAs and their derivatives can be confirmed by mass spectrometry. Several studies have made and utilized modifications of PNAs (for example, Norton et al., Bioorg Med Chem. April 1995;3(4):437-45; Petersen et al., J Pept Sci. May-June 1995;1(3):175-83; Orum et al., Biotechniques. Sep. 1995;19(3):472-80; Footer et al., Biochemistry. Aug. 20, 1996;35(33):10673-9; Griffith et al., Nucleic Acids Res. Aug. 11, 1995;23(15):3003-8; Pardridge et al., Proc Natl Acad Sci USA. Jun. 6, 1995;92(12):5592-6; Boffa et al., Proc Natl Acad Sci USA. Mar. 14, 1995; 92(6):1901-5; Gambacorti-Passerini et al., Blood. Aug. 15, 1996;88(4):1411-7; Armitage et al., Proc Natl Acad Sci USA. Nov. 11, 1997;94(23):12320-5; Seeger et al., Biotechniques. September 1997;23(3):512-7). U.S. Pat. No. 5,700,922 discusses PNA-DNA-PNA chimeric molecules and their uses in diagnostics, modulating protein in organisms, and treatment of conditions susceptible to therapeutics.

Methods of characterizing the antisense binding properties of PNAs are discussed in Rose (Anal Chem. Dec. 15, 1993;65(24):3545-9) and Jensen et al (Biochemistry. Apr. 22, 1997;36(16):5072-7). Rose uses capillary gel electrophoresis to determine binding of PNAs to their complementary oligonucleotide, measuring the relative binding kinetics and stoichiometry. Similar types of measurements were made by Jensen et al. using BIAcore™ technology.

Other applications of PNAs that have been described and will be apparent to the skilled artisan include use in DNA strand invasion, antisense inhibition, mutational analysis, enhancers of transcription, nucleic acid purification, isolation of transcriptionally active genes, blocking of transcription factor binding, genome cleavage, biosensors, in situ hybridization, and the like.

Polynucleotide Identification, Characterization and Expression

Polynucleotides compositions of the present invention may be identified, prepared and/or manipulated using any of a variety of well established techniques (see generally, Sambrook et al., Molecular Cloning: A Laboratory Manual, Cold Spring Harbor Laboratories, Cold Spring Harbor, N.Y., 1989, and other like references). For example, a polynucleotide may be identified, as described in more detail below, by screening a microarray of cDNAs for tumor-associated expression (i.e., expression that is at least two fold greater in a tumor than in normal tissue, as determined using a representative assay provided herein). Such screens may be performed, for example, using the microarray technology of Affymetrix, Inc. (Santa Clara, Calif.) according to the manufacturer's instructions (and essentially as described by Schena et al., Proc. Natl. Acad. Sci. USA 93:10614-10619, 1996 and Heller et al., Proc. Natl. Acad. Sci. USA 94:2150-2155, 1997). Alternatively, polynucleotides may be amplified from cDNA prepared from cells expressing the proteins described herein, such as tumor cells.

Many template dependent processes are available to amplify a target sequences of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCR™, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

Any of a number of other template dependent processes, many of which are variations of the PCR™ amplification technique, are readily known and available in the art. Illustratively, some such methods include the ligase chain reaction (referred to as LCR), described, for example, in Eur. Pat. Appl. Publ. No. 320,308 and U.S. Pat. No. 4,883,750; Qbeta Replicase, described in PCT Intl. Pat. Appl. Publ. No. PCT/US87/00880; Strand Displacement Amplification (SDA) and Repair Chain Reaction (RCR). Still other amplification methods are described in Great Britain Pat. Appl. No. 2 202 328, and in PCT Intl. Pat. Appl. Publ. No. PCT/US89/01025. Other nucleic acid amplification procedures include transcription-based amplification systems (TAS) (PCT Intl. Pat. Appl. Publ. No. WO 88/10315), including nucleic acid sequence based amplification (NASBA) and 3SR. Eur. Pat. Appl. Publ. No. 329,822 describes a nucleic acid amplification process involving cyclically synthesizing single-stranded RNA (“ssRNA”), ssDNA, and double-stranded DNA (dsDNA). PCT Intl. Pat. Appl. Publ. No. WO 89/06700 describes a nucleic acid sequence amplification scheme based on the hybridization of a promoter/primer sequence to a target single-stranded DNA (“ssDNA”) followed by transcription of many RNA copies of the sequence. Other amplification methods such as “RACE” (Frohman, 1990), and “one-sided PCR” (Ohara, 1989) are also well-known to those of skill in the art.

An amplified portion of a polynucleotide of the present invention may be used to isolate a full length gene from a suitable library (e.g., a tumor cDNA library) using well known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

For hybridization techniques, a partial sequence may be labeled (e.g., by nick-translation or end-labeling with ³²p) using well known techniques. A bacterial or bacteriophage library is then generally screened by hybridizing filters containing denatured bacterial colonies (or lawns containing phage plaques) with the labeled probe (see Sambrook et al., Molecular Cloning: A Laboratory Manual, Cold Spring Harbor Laboratories, Cold Spring Harbor, N.Y., 1989). Hybridizing colonies or plaques are selected and expanded, and the DNA is isolated for further analysis. cDNA clones may be analyzed to determine the amount of additional sequence by, for example, PCR using a primer from the partial sequence and a primer from the vector. Restriction maps and partial sequences may be generated to identify one or more overlapping clones. The complete sequence may then be determined using standard techniques, which may involve generating a series of deletion clones. The resulting overlapping sequences can then assembled into a single contiguous sequence. A full length cDNA molecule can be generated by ligating suitable fragments, using well known techniques.

Alternatively, amplification techniques, such as those described above, can be useful for obtaining a full length coding sequence from a partial cDNA sequence. One such amplification technique is inverse PCR (see Triglia et al., Nucl. Acids Res. 16:8186, 1988), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111-19, 1991) and walking PCR (Parker et al., Nucl. Acids. Res. 19:3055-60, 1991). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence. Full length DNA sequences may also be obtained by analysis of genomic fragments.

In other embodiments of the invention, polynucleotide sequences or fragments thereof which encode polypeptides of the invention, or fusion proteins or functional equivalents thereof, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present invention can be engineered using methods generally known in the art in order to alter polypeptide encoding sequences for a variety of reasons, including but not limited to, alterations which modify the cloning, processing, and/or expression of the gene product. For example, DNA shuffling by random fragmentation and PCR reassembly of gene fragments and synthetic oligonucleotides may be used to engineer the nucleotide sequences. In addition, site-directed mutagenesis may be used to insert new restriction sites, alter glycosylation patterns, change codon preference, produce splice variants, or introduce mutations, and so forth.

In another embodiment of the invention, natural, modified, or recombinant nucleic acid sequences may be ligated to a heterologous sequence to encode a fusion protein. For example, to screen peptide libraries for inhibitors of polypeptide activity, it may be useful to encode a chimeric protein that can be recognized by a commercially available antibody. A fusion protein may also be engineered to contain a cleavage site located between the polypeptide-encoding sequence and the heterologous protein sequence, so that the polypeptide may be cleaved and purified away from the heterologous moiety.

Sequences encoding a desired polypeptide may be synthesized, in whole or in part, using chemical methods well known in the art (see Caruthers, M. H. et al. (1980) Nucl. Acids Res. Symp. Ser. 215-223, Horn, T. et al. (1980) Nucl. Acids Res. Symp. Ser. 225-232). Alternatively, the protein itself may be produced using chemical methods to synthesize the amino acid sequence of a polypeptide, or a portion thereof. For example, peptide synthesis can be performed using various solid-phase techniques (Roberge, J. Y. et al. (1995) Science 269:202-204) and automated synthesis may be achieved, for example, using the ABI 431A Peptide Synthesizer (Perkin Elmer, Palo Alto, Calif.).

A newly synthesized peptide may be substantially purified by preparative high performance liquid chromatography (e.g., Creighton, T. (1983) Proteins, Structures and Molecular Principles, WH Freeman and Co., New York, N.Y.) or other comparable techniques available in the art. The composition of the synthetic peptides may be confirmed by amino acid analysis or sequencing (e.g., the Edman degradation procedure). Additionally, the amino acid sequence of a polypeptide, or any part thereof, may be altered during direct synthesis and/or combined using chemical methods with sequences from other proteins, or any part thereof, to produce a variant polypeptide.

In order to express a desired polypeptide, the nucleotide sequences encoding the polypeptide, or functional equivalents, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y.

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid 1acZ promoter of the pBLUESCRIPT phagemid (Stratagene, La Jolla, Calif.) or pSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, any of a number of expression vectors may be selected depending upon the use intended for the expressed polypeptide. For example, when large quantities are needed, for example for the induction of antibodies, vectors which direct high level expression of fusion proteins that are readily purified may be used. Such vectors include, but are not limited to, the multifunctional E. coli cloning and expression vectors such as pBLUESCRIPT (Stratagene), in which the sequence encoding the polypeptide of interest may be ligated into the vector in frame with sequences for the amino-terminal Met and the subsequent 7 residues of .beta.-galactosidase so that a hybrid protein is produced; pIN vectors (Van Heeke, G. and S. M. Schuster (1989) J. Biol. Chem; 264:5503-5509); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al. (1987) Methods Enzymol. 153:516-544.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, N. (1987) EMBO J. 6:307-311. Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; and Winter, J. et al. (1991) Results Probl. Cell Differ. ]7:85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard, E. K. et al. (1994) Proc. Natl. Acad. Sci. 91 :3224-3227).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

In addition, a host cell strain may be chosen for its ability to modulate the expression of the inserted sequences or to process the expressed protein in the desired fashion. Such modifications of the polypeptide include, but are not limited to, acetylation, carboxylation. glycosylation, phosphorylation, lipidation, and acylation. Post-translational processing which cleaves a “prepro” form of the protein may also be used to facilitate correct insertion, folding and/or function. Different host cells such as CHO, COS, HeLa, MDCK, HEK293, and W138, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler, M. et al. (1977) Cell 11:223-32) and adenine phosphoribosyltransferase (Lowy, I. et al. (1990) Cell 22:817-23) genes which can be employed in tk.sup.- or aprt.sup.-cells, respectively. Also, antimetabolite, antibiotic or herbicide resistance can be used as the basis for selection; for example, dhfr which confers resistance to methotrexate (Wigler, M. et al. (1980) Proc. Natl. Acad. Sci. 77:3567-70); npt, which confers resistance to the aminoglycosides, neomycin and G-418 (Colbere-Garapin, F. et al (1981) J. Mol. Biol. 150:1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman, S. C. and R. C. Mulligan (1988) Proc. Natl. Acad. Sci. 85:8047-51). The use of visible markers has gained popularity with such markers as anthocyanins, beta-glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter. Expression of the marker gene in response to induction or selection usually indicates expression of the tandem gene as well.

Alternatively, host cells that contain and express a desired polynucleotide sequence may be identified by a variety of procedures known to those of skill in the art. These procedures include, but are not limited to, DNA-DNA or DNA-RNA hybridizations and protein bioassay or immunoassay techniques which include, for example, membrane, solution, or chip based technologies for the detection and/or quantification of nucleic acid or protein.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton, R. et al. (1990; Serological Methods, a Laboratory Manual, APS Press, St Paul. Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen. San Diego, Calif.) between the purification domain and the encoded polypeptide may be used to facilitate purification. One such expression vector provides for expression of a fusion protein containing a polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath, J. et al. (1992, Prot. Exp. Purif. 3:263-281) while the enterokinase cleavage site provides a means for purifying the desired polypeptide from the fusion protein. A discussion of vectors which contain fusion proteins is provided in Kroll, D. J. et al. (1993; DNA Cell Biol. 12:441-453).

In addition to recombinant production methods, polypeptides of the invention, and fragments thereof, may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

Antibody Compositions, Fragments Thereof and Other Binding Agents

According to another aspect, the present invention further provides binding agents, such as antibodies and antigen-binding fragments thereof, that exhibit immunological binding to a tumor polypeptide disclosed herein, or to a portion, variant or derivative thereof. An antibody, or antigen-binding fragment thereof, is said to “specifically bind,” “immunogically bind,” and/or is “immunologically reactive” to a polypeptide of the invention if it reacts at a detectable level (within, for example, an ELISA assay) with the polypeptide, and does not react detectably with unrelated polypeptides under similar conditions.

Immunological binding, as used in this context, generally refers to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant K_{d}. See, generally, Davies et al. (1990) Annual Rev. Biochem. 59:439-473.

An “antigen-binding site,” or “binding portion” of an antibody refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Binding agents may be further capable of differentiating between patients with and without a cancer, such as breast cancer, using the representative assays provided herein. For example, antibodies or other binding agents that bind to a tumor protein will preferably generate a signal indicating the presence of a cancer in at least about 20% of patients with the disease, more preferably at least about 30% of patients. Alternatively, or in addition, the antibody will generate a negative signal indicating the absence of the disease in at least about 90% of individuals without the cancer. To determine whether a binding agent satisfies this requirement, biological samples (e.g., blood, sera, sputum, urine and/or tumor biopsies) from patients with and without a cancer (as determined using standard clinical tests) may be assayed as described herein for the presence of polypeptides that bind to the binding agent. Preferably, a statistically significant number of samples with and without the disease will be assayed. Each binding agent should satisfy the above criteria; however, those of ordinary skill in the art will recognize that binding agents may be used in combination to improve sensitivity.

Any agent that satisfies the above requirements may be a binding agent. For example, a binding agent may be a ribosome, with or without a peptide component, an RNA molecule or a polypeptide. In a preferred embodiment, a binding agent is an antibody or an antigen-binding fragment thereof. Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J. Immunol. 6:511-519, 1976, and improvements thereto. Briefly, these methods involve the preparation of,immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of therapeutically useful molecules are known in the art which comprise antigen-binding sites that are capable of exhibiting immunological binding properties of an antibody molecule. The proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment which comprises both antigen-binding sites. An “Fv” fragment can be produced by preferential proteolytic cleavage of an IgM, and on rare occasions IgG or IgA immunoglobulin molecule. Fv fragments are, however, more commonly derived using recombinant techniques known in the art. The Fv fragment includes a non-covalent V_{H}::V_{L} heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single chain Fv (“sFv”) polypeptide is a covalently linked V_{H}::V_{L} heterodimer which is expressed from a gene fusion including V_{H}- and V_{L}-encoding genes linked by a peptide-encoding linker. Huston et al. (1988) Proc. Nat. Acad. Sci. USA 85(16):5879-5883. A number of methods have been described to discern chemical structures for converting the naturally aggregated—but chemically separated—light and heavy polypeptide chains from an antibody V region into an sFv molecule which will fold into a three dimensional structure substantially similar to the structure of an antigen-binding site. See, e.g., U.S. Pat. No. Nos. 5,091,513 and 5,132,405, to Huston et al.; and U.S. Pat. No. 4,946,778, to Ladner et al.

Each of the above-described molecules includes a heavy chain and a light chain CDR set, respectively interposed between a heavy chain and a light chain FR set which provide support to the CDRS and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRS. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

A number of “humanized” antibody molecules comprising an antigen-binding site derived from a non-human immunoglobulin have been described, including chimeric antibodies having rodent V regions and their associated CDRs fused to human constant domains (Winter et al. (1991) Nature 349:293-299; Lobuglio et al. (1989) Proc. Nat. Acad. Sci. USA 86:4220-4224; Shaw et al. (1987) J Immunol. 138:4534-4538; and Brown et al. (1987) Cancer Res. 47:3577-3583), rodent CDRs grafted into a human supporting FR prior to fusion with an appropriate human antibody constant domain (Riechmann et al. (1988) Nature 332:323-327; Verhoeyen et al. (1988) Science 239:1534-1536; and Jones et al. (1986) Nature 321:522-525), and rodent CDRs supported by recombinantly veneered rodent FRs (European Patent Publication No. 519,596, published Dec. 23, 1992). These “humanized” molecules are designed to minimize unwanted immunological response toward rodent antihuman antibody molecules which limits the duration and effectiveness of therapeutic applications of those moieties in human recipients.

As used herein, the terms “veneered FRs” and “recombinantly veneered FRs” refer to the selective replacement of FR residues from, e.g., a rodent heavy or light chain V region, with human FR residues in order to provide a xenogeneic molecule comprising an antigen-binding site which retains substantially all of the native FR polypeptide folding structure. Veneering techniques are based on the understanding that the ligand binding characteristics of an antigen-binding site are determined primarily by the structure and relative disposition of the heavy and light chain CDR sets within the antigen-binding surface. Davies et al. (1990) Ann. Rev. Biochem. 59:439-473. Thus, antigen binding specificity can be preserved in a humanized antibody only wherein the CDR structures, their interaction with each other, and their interaction with the rest of the V region domains are carefully maintained. By using veneering techniques, exterior (e.g., solvent-accessible) FR residues which are readily encountered by the immune system are selectively replaced with human residues to provide a hybrid molecule that comprises either a weakly immunogenic, or substantially non-immunogenic veneered surface.

The process of veneering makes use of the available sequence data for human antibody variable domains compiled by Kabat et al., in Sequences of Proteins of Immunological Interest, 4th ed., (U.S. Dept. of Health and Human Services, U.S. Government Printing Office, 1987), updates to the Kabat database, and other accessible U.S. and foreign databases (both nucleic acid and protein). Solvent accessibilities of V region amino acids can be deduced from the known three-dimensional structure for human and murine antibody fragments. There are two general steps in veneering a murine antigen-binding site. Initially, the FRs of the variable domains of an antibody molecule of interest are compared with corresponding FR sequences of human variable domains obtained from the above-identified sources. The most homologous human V regions are then compared residue by residue to corresponding murine amino acids. The residues in the murine FR which differ from the human counterpart are replaced by the residues present in the human moiety using recombinant techniques well known in the art. Residue switching is only carried out with moieties which are at least partially exposed (solvent accessible), and care is exercised in the replacement of amino acid residues which may have a significant effect on the tertiary structure of V region domains, such as proline, glycine and charged amino acids.

In this manner, the resultant “veneered” murine antigen-binding sites are thus designed to retain the murine CDR residues, the residues substantially adjacent to the CDRs, the residues identified as buried or mostly buried (solvent inaccessible), the residues believed to participate in non-covalent (e.g., electrostatic and hydrophobic) contacts between heavy and light chain domains, and the residues from conserved structural regions of the FRs which are believed to influence the “canonical” tertiary structures of the CDR loops. These design criteria are then used to prepare recombinant nucleotide sequences which combine the CDRs of both the heavy and light chain of a murine antigen-binding site into human-appearing FRs that can be used to transfect mammalian cells for the expression of recombinant human antibodies which exhibit the antigen specificity of the murine antibody molecule.

In another embodiment of the invention, monoclonal antibodies of the present invention may be coupled to one or more therapeutic agents. Suitable agents in this regard include radionuclides, differentiation inducers, drugs, toxins, and derivatives thereof. Preferred radionuclides include ⁹⁰Y, ¹²³I, ¹²⁵I, ¹³¹I, ¹⁸⁶Re, ¹⁸⁸Re, ²¹¹At, and ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may,be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers that provide multiple sites for attachment can be used. Alternatively, a carrier can be used.

A carrier may bear the agents in a variety of ways, including covalent bonding either directly or via a linker group. Suitable carriers include proteins such as albumins (e.g., U.S. Pat. No. 4,507,234, to Kato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis.

T Cell Compositions

The present invention, in another aspect, provides T cells specific for a tumor polypeptide disclosed herein, or for a variant or derivative thereof. Such cells may generally be prepared in vitro or ex vivo, using standard procedures. For example, T cells may be isolated from bone marrow, peripheral blood, or a fraction of bone marrow or peripheral blood of a patient, using a commercially available cell separation system, such as the Isolex™ System, available from Nexell Therapeutics, Inc. (Irvine, Calif.; see also U.S. Pat. No. 5,240,856; U.S. Pat. No. 5,215,926; WO 89/06280; WO 91/16116 and WO 92/07243). Alternatively, T cells may be derived from related or unrelated humans, non-human mammals, cell lines or cultures.

T cells may be stimulated with a polypeptide, polynucleotide encoding a polypeptide and/or an antigen presenting cell (APC) that expresses such a polypeptide. Such stimulation is performed under conditions and for a time sufficient to permit the generation of T cells that are specific for the polypeptide of interest. Preferably, a tumor polypeptide or polynucleotide of the invention is present within a delivery vehicle, such as a microsphere, to facilitate the generation of specific T cells.

T cells are considered to be specific for a polypeptide of the present invention if the T cells specifically proliferate, secrete cytokines or kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070, 1994. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a tumor polypeptide (100 ng/ml-100 μg/ml, preferably 200 ng/ml-25 μg/ml) for 3-7 days will typically result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1, Wiley Interscience (Greene 1998)). T cells that have been activated in response to a tumor polypeptide, polynucleotide or polypeptide-expressing APC may be CD4⁺ and/or CD8⁺. Tumor polypeptide-specific T cells may be expanded using standard techniques. Within preferred embodiments, the T cells are derived from a patient, a related donor or an unrelated donor, and are administered to the patient following stimulation and expansion.

For therapeutic purposes, CD4⁺ or CD8⁺ T cells that proliferate in response to a tumor polypeptide, polynucleotide or APC can be expanded in number either in vitro or in vivo. Proliferation of such T cells in vitro may be accomplished in a variety of ways. For example, the T cells can be re-exposed to a tumor polypeptide, or a short peptide corresponding to an immunogenic portion of such a polypeptide, with or without the addition of T cell growth factors, such as interleukin-2, and/or stimulator cells that synthesize a tumor polypeptide. Alternatively, one or more T cells that proliferate in the presence of the tumor polypeptide can be expanded in number by cloning. Methods for cloning cells are well known in the art, and include limiting dilution.

T Cell Receptor Compositions

The T cell receptor (TCR) consists of 2 different, highly variable polypeptide chains, termed the T-cell receptor α and β chains, that are linked by a disulfide bond (Janeway, Travers, Walport. Immunobiology. Fourth Ed., 148-159. Elsevier Science Ltd/Garland Publishing. 1999). The α/β heterodimer complexes with the invariant CD3 chains at the cell membrane. This complex recognizes specific antigenic peptides bound to MHC molecules. The enormous diversity of TCR specificities is generated much like inmunoglobulin diversity, through somatic gene rearrangement. The β chain genes contain over 50 variable (V), 2 diversity (D), over 10 joining (J) segments, and 2 constant region segments (C). The α chain genes contain over 70 V segments, and over 60 J segments but no D segments, as well as one C segment. During T cell development in the thymus, the D to J gene rearrangement of the β chain occurs, followed by the V gene segment rearrangement to the DJ. This functional VDJβ exon is transcribed and spliced to join to a Cβ. For the α chain, a Vα gene segment rearranges to a Jα gene segment to create the functional exon that is then transcribed and spliced to the Cα. Diversity is further increased during the recombination process by the random addition of P and N-nucleotides between the V, D, and J segments of the β chain and between the V and J segments in the □ chain (Janeway, Travers, Walport. Immunobiology. Fourth Ed., 98 and 150. Elsevier Science Ltd/Garland Publishing. 1999).

The present invention, in another aspect, provides TCRs specific for a polypeptide disclosed herein, or for a variant or derivative thereof. In accordance with the present invention, polynucleotide and amino acid sequences are provided for the V-J or V-D-J junctional regions or parts thereof for the alpha and beta chains of the T-cell receptor which recognize tumor polypeptides described herein. In general, this aspect of the invention relates to T-cell receptors which recognize or bind tumor polypeptides presented in the context of MHC. In a preferred embodiment the tumor antigens recognized by the T-cell receptors comprise a polypeptide of the present invention. For example, cDNA encoding a TCR specific for a breast tumor peptide can be isolated from T cells specific for a tumor polypeptide using standard molecular biological and recombinant DNA techniques.

This invention further includes the T-cell receptors or analogs thereof having substantially the same function or activity as the T-cell receptors of this invention which recognize or bind tumor polypeptides. Such receptors include, but are not limited to, a fragment of the receptor, or a substitution, addition or deletion mutant of a T-cell receptor provided herein. This invention also encompasses polypeptides or peptides that are substantially homologous to the T-cell receptors provided herein or that retain substantially the same activity. The term “analog” includes any protein or polypeptide having an amino acid residue sequence substantially identical to the T-cell receptors provided herein in which one or more residues, preferably no more than 5 residues, more preferably no more than 25 residues have been conservatively substituted with a functionally similar residue and which displays the functional aspects of the T-cell receptor as described herein.

The present invention further provides for suitable mammalian host cells, for example, non-specific T cells, that are transfected with a polynucleotide encoding TCRs specific for a polypeptide described herein, thereby rendering the host cell specific for the polypeptide. The α and β chains of the TCR may be contained on separate expression vectors or alternatively, on a single expression vector that also contains an internal ribosome entry site (IRES) for cap-independent translation of the gene downstream of the IRES. Said host cells expressing TCRs specific for the polypeptide may be used, for example, for adoptive immunotherapy of breast cancer as discussed further below.

In further aspects of the present invention, cloned TCRs specific for a polypeptide recited herein may be used in a kit for the diagnosis of breast cancer. For example, the nucleic acid sequence or portions thereof, of tumor-specific TCRs can be used as probes or primers for the detection of expression of the rearranged genes encoding the specific TCR in a biological sample. Therefore, the present invention further provides for an assay for detecting messenger RNA or DNA encoding the TCR specific for a polypeptide.

Pharmaceutical Compositions

In additional embodiments, the present invention concerns formulation of one or more of the polynucleotide, polypeptide, T-cell, TCR, and/or antibody compositions disclosed herein in pharmaceutically-acceptable carriers for administration to a cell or an animal, either alone, or in combination with one or more other modalities of therapy.

It will be understood that, if desired, a composition as disclosed herein may be administered in combination with other agents as well, such as, e.g., other proteins or polypeptides or various pharmaceutically-active agents. In fact, there is virtually no limit to other components that may also be included, given that the additional agents do not cause a significant adverse effect upon contact with the target cells or host tissues. The compositions may thus be delivered along with various other agents as required in the particular instance. Such compositions may be purified from host cells or other biological sources, or alternatively may be chemically synthesized as described herein. Likewise, such compositions may further comprise substituted or derivatized RNA or DNA compositions.

Therefore, in another aspect of the present invention, pharmaceutical compositions are provided comprising one or more of the polynucleotide, polypeptide, antibody, TCR, and/or T-cell compositions described herein in combination with a physiologically acceptable carrier. In certain preferred embodiments, the pharmaceutical compositions of the invention comprise immunogenic polynucleotide and/or polypeptide compositions of the invention for use in prophylactic and theraputic vaccine applications. Vaccine preparation is generally described in, for example, M. F. Powell and M. J. Newman, eds., “Vaccine Design (the subunit and adjuvant approach),” Plenum Press (NY, 1995). Generally, such compositions will comprise one or more polynucleotide and/or polypeptide compositions of the present invention in combination with one or more immunostimulants.

It will be apparent that any of the pharmaceutical compositions described herein can contain pharmaceutically acceptable salts of the polynucleotides and polypeptides of the invention. Such salts can be prepared, for example, from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

In another embodiment, illustrative immunogenic compositions, e.g., vaccine compositions, of the present invention comprise DNA encoding one or more of the polypeptides as described above, such that the polypeptide is generated in situ. As noted above, the polynucleotide may be administered within any of a variety of delivery systems known to those of ordinary skill in the art. Indeed, numerous gene delivery techniques are well known in the art, such as those described by Rolland, Crit. Rev. Therap. Drug Carrier Systems 15:143-198, 1998, and references cited therein. Appropriate polynucleotide expression systems will, of course, contain the necessary regulatory DNA regulatory sequences for expression in a patient (such as a suitable promoter and terminating signal). Alternatively, bacterial delivery systems may involve the administration of a bacterium (such as Bacillis-Calmette-Guerrin) that expresses an immunogenic portion of the polypeptide on its cell surface or secretes such an epitope.

Therefore, in certain embodiments, polynucleotides encoding immunogenic polypeptides described herein are introduced into suitable mammalian host cells for expression using any of a number of known viral-based systems. In one illustrative embodiment, retroviruses provide a convenient and effective platform for gene delivery systems. A selected nucleotide sequence encoding a polypeptide of the present invention can be inserted into a vector and packaged in retroviral particles using techniques known in the art. The recombinant virus can then be isolated and delivered to a subject. A number of illustrative retroviral systems have been described (e.g., U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

In addition, a number of illustrative adenovirus-based systems have also been described. Unlike retroviruses which integrate into the host genome, adenoviruses persist extrachromosomally thus minimizing the risks associated with insertional mutagenesis (Haj-Ahmad and Graham (1986) J. Virol. 57:267-274; Bett et al. (1993) J. Virol. 67:5911-5921; Mittereder et al. (1994) Human Gene Therapy 5:717-729; Seth et al. (1994) J. Virol. 68:933-940; Barr et al. (1994) Gene Therapy 1:51-58; Berkner, K. L. (1988) BioTechniques 6:616-629; and Rich et al. (1993) Human Gene Therapy 4:461-476).

Various adeno-associated virus (AAV) vector systems have also been developed for polynucleotide delivery. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Additional viral vectors useful for delivering the polynucleotides encoding polypeptides of the present invention by gene transfer include those derived from the pox family of viruses, such as vaccinia virus and avian poxvirus. By way of example, vaccinia virus recombinants expressing the novel molecules can be constructed as follows. The DNA encoding a polypeptide is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the polypeptide of interest into the viral genome. The resulting TK.sup.(−) recombinant can be selected by culturing the cells in the presence of 5-bromodeoxyuridine and picking viral plaques resistant thereto.

A vaccinia-based infection/transfection system can be conveniently used to provide for inducible, transient expression or coexpression of one or more polypeptides described herein in host cells of an organism. In this particular system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide or polynucleotides of interest, driven by a T7 promoter. The polymerase expressed in the cytoplasm from the vaccinia virus recombinant transcribes the transfected DNA into RNA which is then translated into polypeptide by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al. Proc. Natl. Acad. Sci. USA (1986) 83:8122-8126.

Alternatively, avipoxviruses, such as the fowlpox and canarypox viruses, can also be used to deliver the coding sequences of interest. Recombinant avipox viruses, expressing immunogens from mammalian pathogens, are known to confer protective immunity when administered to non-avian species. The use of an Avipox vector is particularly desirable in human and other mammalian species since members of the Avipox genus can only productively replicate in susceptible avian species and therefore are not infective in mammalian cells. Methods for producing recombinant Avipoxviruses are known in the art and employ genetic recombination, as described above with respect to the production of vaccinia viruses. See, e.g., WO 91/12882; WO 89/03429; and WO 92/03545.

Any of a number of alphavirus vectors can also be used for delivery of polynucleotide compositions of the present invention, such as those vectors described in U.S. Pat. Nos. 5,843,723; 6,015,686; 6,008,035 and 6,015,694. Certain vectors based on Venezuelan Equine Encephalitis (VEE) can also be used, illustrative examples of which can be found in U.S. Pat. Nos. 5,505,947 and 5,643,576.

Moreover, molecular conjugate vectors, such as the adenovirus chimeric vectors described in Michael et al. J. Biol. Chem. (1993) 268:6866-6869 and Wagner et al. Proc. Natl. Acad. Sci. USA (1992) 89:6099-6103, can also be used for gene delivery under the invention.

Additional illustrative information on these and other known viral-based delivery systems can be found, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993.

In certain embodiments, a polynucleotide may be integrated into the genome of a target cell. This integration may be in the specific location and orientation via homologous recombination (gene replacement) or it may be integrated in a random, non-specific location (gene augmentation). In yet further embodiments, the polynucleotide may be stably maintained in the cell as a separate, episomal segment of DNA. Such polynucleotide segments or “episomes” encode sequences sufficient to permit maintenance and replication independent of or in synchronization with the host cell cycle. The manner in which the expression construct is delivered to a cell and where in the cell the polynucleotide remains is dependent on the type of expression construct employed.

In another embodiment of the invention, a polynucleotide is administered/delivered as “naked” DNA, for example as described in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

In still another embodiment, a composition of the present invention can be delivered via a particle bombardment approach, many of which have been described. In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide or polypeptide particles, are accelerated to high speed within a helium gas jet generated by a hand held device, propelling the particles into a target tissue of interest.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

According to another embodiment, the pharmaceutical compositions described herein will comprise one or more immunostimulants in addition to the immunogenic polynucleotide, polypeptide, antibody, T-cell, TCR, and/or APC compositions of this invention. An immunostimulant refers to essentially any substance that enhances or potentiates an immune response (antibody and/or cell-mediated) to an exogenous antigen. One preferred type of immunostimulant comprises an adjuvant. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Within certain embodiments of the invention, the adjuvant composition is preferably one that induces an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Certain preferred adjuvants for eliciting a predominantly Th1-type response include, for example, a combination of monophosphoryl lipid A, preferably 3-de-O-acylated monophosphoryl lipid A, together with an aluminum salt. MPL® adjuvants are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other preferred formulations include more than one saponin in the adjuvant combinations of the present invention, for example combinations of at least two of the following group comprising QS21, QS7, Quil A, β-escin, or digitonin.

Alternatively the saponin formulations may be combined with vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM. The saponins may also be formulated with excipients such as Carbopol® to increase viscosity, or may be formulated in a dry powder form with a powder excipient such as lactose.

In one preferred embodiment, the adjuvant system includes the combination of a monophosphoryl lipid A and a saponin derivative, such as the combination of QS21 and 3D-MPL® adjuvant, as described in WO 94/00153, or a less reactogenic composition where the QS21 is quenched with cholesterol, as described in WO 96/33739. Other preferred formulations comprise an oil-in-water emulsion and tocopherol. Another particularly preferred adjuvant formulation employing QS21, 3D-MPL® adjuvant and tocopherol in an oil-in-water emulsion is described in WO 95/17210.

Another enhanced adjuvant system involves the combination of a CpG-containing oligonucleotide and a saponin derivative particularly the combination of CpG and QS21 is disclosed in WO 00/09159. Preferably the formulation additionally comprises an oil in water emulsion and tocopherol.

Additional illustrative adjuvants for use in the pharmaceutical compositions of the invention include Montanide ISA 720 (Seppic, France), SAF (Chiron, California, United States), ISCOMS (CSL), MF-59 (Chiron), the SBAS series of adjuvants (e.g., SBAS-2 or SBAS-4, available from SmithKline Beecham, Rixensart, Belgium), Detox (Enhanzyn®) (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

Other preferred adjuvants include adjuvant molecules of the general formula
HO(CH₂CH₂O)ₙ-A-R, (I)

wherein, n is 1-50, A is a bond or —C(O)—, R is C₁₋₅₀ alkyl or Phenyl C₁₋₅₀ alkyl.

One embodiment of the present invention consists of a vaccine formulation comprising a polyoxyethylene ether of general formula (I), wherein n is between 1 and 50, preferably 4-24, most preferably 9; the R component is C₁₋₅₀, preferably C₄-C₂₀ alkyl and most preferably C₁₂ alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12^{th} edition: entry 7717). These adjuvant molecules are described in WO 99/52549.

The polyoxyethylene ether according to the general formula (I) above may, if desired, be combined with another adjuvant. For example, a preferred adjuvant combination is preferably with CpG as described in the pending UK patent application GB 9820956.2.

According to another embodiment of this invention, an immunogenic composition described herein is delivered to a host via antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic antitumor immunity (see Timmerman and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate naïve T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide of the invention (or portion or other variant thereof) such that the encoded polypeptide, or an immunogenic portion thereof, is expressed on the cell surface. Such transfection may take place ex vivo, and a pharmaceutical composition comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with the tumor polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

While any suitable carrier known to those of ordinary skill in the art may be employed in the pharmaceutical compositions of this invention, the type of carrier will typically vary depending on the mode of administration. Compositions of the present invention may be formulated for any appropriate manner of administration, including for example, topical, oral, nasal, mucosal, intravenous, intracranial, intraperitoneal, subcutaneous and intramuscular administration.

Carriers for use within such pharmaceutical compositions are biocompatible, and may also be biodegradable. In certain embodiments, the formulation preferably provides a relatively constant level of active component release. In other embodiments, however, a more rapid rate of release immediately upon administration may be desired. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

In another illustrative embodiment, biodegradable microspheres (e.g., polylactate polyglycolate) are employed as carriers for the compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344, 5,407,609 and 5,942,252. Modified hepatitis B core protein carrier systems, such as described in WO/99 40934, and references cited therein, will also be useful for many applications. Another illustrative carrier/delivery system employs a carrier comprising particulate-protein complexes, such as those described in U.S. Pat. No. 5,928,647, which are capable of inducing a class I-restricted cytotoxic T lymphocyte responses in a host.

In another illustrative embodiment, calcium phosphate core particles are employed as carriers, vaccine adjuvants, or as controlled release matrices for the compositions of this invention. Exemplary calcium phosphate particles are disclosed, for example, in published patent application No. WO/0046147.

The pharmaceutical compositions of the invention will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate.

The pharmaceutical compositions described herein may be presented in unit-dose or multi-dose containers, such as sealed ampoules or vials. Such containers are typically sealed in such a way to preserve the sterility and stability of the formulation until use. In general, formulations may be stored as suspensions, solutions or emulsions in oily or aqueous vehicles. Alternatively, a pharmaceutical composition may be stored in a freeze-dried condition requiring only the addition of a sterile liquid carrier immediately prior to use.

The development of suitable dosing and treatment regimens for using the particular compositions described herein in a variety of treatment regimens, including e.g., oral, parenteral, intravenous, intranasal, and intramuscular administration and formulation, is well known in the art, some of which are briefly discussed below for general purposes of illustration.

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like (see, for example, Mathiowitz et al., Nature Mar. 27, 1997; 386(6623):410-4; Hwang et al., Crit Rev Ther Drug Carrier Syst 1998;15(3):243-84; U.S. Pat. No. 5,641,515; U.S. Pat. No. 5,580,579 and U.S. Pat. No. 5,792,451). Tablets, troches, pills, capsules and the like may also contain any of a variety of additional components, for example, a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Of course, any material used in preparing any dosage unit form should be pharmaceutically pure and substantially non-toxic in the amounts employed. In addition, the active compounds may be incorporated into sustained-release preparation and formulations.

Typically, these formulations will contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the present invention may alternatively be incorporated with one or more excipients in the form of a mouthwash, dentifrice, buccal tablet, oral spray, or sublingual orally-administered formulation. Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

In certain circumstances it will be desirable to deliver the pharmaceutical compositions disclosed herein parenterally, intravenously, intramuscularly, or even intraperitoneally. Such approaches are well known to the skilled artisan, some of which are further described, for example, in U.S. Pat. No. 5,543,158; U.S. Pat. No. 5,641,515 and U.S. Pat. No. 5,399,363. In certain embodiments, solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations generally will contain a preservative to prevent the growth of microorganisms.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. Moreover, for human administration, preparations will of course preferably meet sterility, pyrogenicity, and the general safety and purity standards as required by FDA Office of Biologics standards.

In another embodiment of the invention, the compositions disclosed herein may be formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described, e.g., in U.S. Pat. No. 5,756,353 and U.S. Pat. No. 5,804,212. Likewise, the delivery of drugs using intranasal microparticle resins (Takenaga et al., J Controlled Release Mar. 2, 1998;52(1-2):81-7) and lysophosphatidyl-glycerol compounds (U.S. Pat. No. 5,725,871) are also well-known in the pharmaceutical arts. Likewise, illustrative transmucosal drug delivery in the form of a polytetrafluoroetheylene support matrix is described in U.S. Pat. No. 5,780,045.

In certain embodiments, liposomes, nanocapsules, microparticles, lipid particles, vesicles, and the like, are used for the introduction of the compositions of the present invention into suitable host cells/organisms. In particular, the compositions of the present invention may be formulated for delivery either encapsulated in a lipid particle, a liposome, a vesicle, a nanosphere, or a nanoparticle or the like. Alternatively, compositions of the present invention can be bound, either covalently or non-covalently, to the surface of such carrier vehicles.

The formation and use of liposome and liposome-like preparations as potential drug carriers is generally known to those of skill in the art (see for example, Lasic, Trends Biotechnol July 1998;16(7):307-21; Takakura, Nippon Rinsho March 1998;56(3):691-5; Chandran et al., Indian J Exp Biol. August 1997;35(8):801-9; Margalit, Crit Rev Ther Drug Carrier Syst. 1995;12(2-3):233-61; U.S. Pat. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used successfully with a number of cell types that are normally difficult to transfect by other procedures, including T cell suspensions, primary hepatocyte cultures and PC 12 cells (Renneisen et al., J Biol Chem. Sep. 25, 1990;265(27):16337-42; Muller et al., DNA Cell Biol. April 1990;9(3):221-9). In addition, liposomes are free of the DNA length constraints that are typical of viral-based delivery systems. Liposomes have been used effectively to introduce genes, various drugs, radiotherapeutic agents, enzymes, viruses, transcription factors, allosteric effectors and the like, into a variety of cultured cell lines and animals. Furthermore, he use of liposomes does not appear to be associated with autoimmnune responses or unacceptable toxicity after systemic delivery.

In certain embodiments, liposomes are formed from phospholipids that are dispersed in an aqueous medium and spontaneously form multilamellar concentric bilayer vesicles (also termed multilamellar vesicles (MLVs).

Alternatively, in other embodiments, the invention provides for pharmaceutically-acceptable nanocapsule formulations of the compositions of the present invention. Nanocapsules can generally entrap compounds in a stable and reproducible way (see, for example, Quintanar-Guerrero et al., Drug Dev Ind Pharm. December 1998;24(12):1113-28). To avoid side effects due to intracellular polymeric overloading, such ultrafine particles (sized around 0.1 μm) may be designed using polymers able to be degraded in vivo. Such particles can be made as described, for example, by Couvreur et al., Crit Rev Ther Drug Carrier Syst. 1988;5(1):1-20; zur Muhlen et al., Eur J Pharm Biopharm. March 1998; 45(2):149-55; Zambaux et al. J Controlled Release. Jan. 2, 1998;50(1-3):31-40; and U.S. Pat. No. 5,145,684.

Cancer Therapeutic Methods

Immunologic approaches to cancer therapy are based on the recognition that cancer cells can often evade the body's defenses against aberrant or foreign cells and molecules, and that these defenses might be therapeutically stimulated to regain the lost ground, e.g. pgs. 623-648 in Klein, Immunology (Wiley-Interscience, New York, 1982). Numerous recent observations that various immune effectors can directly or indirectly inhibit growth of tumors has led to renewed interest in this approach to cancer therapy, e.g. Jager, et al., Oncology 2001;60(1):1-7; Renner, et al., Ann Hematol December 2000;79(12):651-9.

Four-basic cell types whose function has been associated with antitumor cell immunity and the elimination of tumor cells from the body are: i) B-lymphocytes which secrete immunoglobulins into the blood plasma for identifying and labeling the nonself invader cells; ii) monocytes which secrete the complement proteins that are responsible for lysing and processing the immunoglobulin-coated target invader cells; iii) natural killer lymphocytes having two mechanisms for the destruction of tumor cells, antibody-dependent cellular cytotoxicity and natural killing; and iv) T-lymphocytes possessing antigen-specific receptors and having the capacity to recognize a tumor cell carrying complementary marker molecules (Schreiber, H., 1989, in Fundamental Immunology (ed). W. E. Paul, pp. 923-955).

Cancer immunotherapy generally focuses on inducing humoral immune responses, cellular immune responses, or both. Moreover, it is well established that induction of CD4⁺ T helper cells is necessary in order to secondarily induce either antibodies or cytotoxic CD8⁺ T cells. Polypeptide antigens that are selective or ideally specific for cancer cells, particularly breast cancer cells, offer a powerful approach for inducing immune responses against breast cancer, and are an important aspect of the present invention.

Therefore, in further aspects of the present invention, the pharmaceutical compositions described herein may be used to stimulate an immune response against cancer, particularly for the immunotherapy of breast cancer. Within such methods, the pharmaceutical compositions described herein are administered to a patient, typically a warm-blooded animal, preferably a human. A patient may or may not be afflicted with cancer. Pharmaceutical compositions and vaccines may be administered either prior to or following surgical removal of primary tumors and/or treatment such as administration of radiotherapy or conventional chemotherapeutic drugs. As discussed above, administration of the pharmaceutical compositions may be by any suitable method, including administration by intravenous, intraperitoneal, intramuscular, subcutaneous, intranasal, intradermal, anal, vaginal, topical and oral routes.

Within certain embodiments, immunotherapy may be active immunotherapy, in which treatment relies on the in vivo stimulation of the endogenous host immune system to react against tumors with the administration of immune response-modifying agents (such as polypeptides and polynucleotides as provided herein).

Within other embodiments, immunotherapy may be passive immunotherapy, in which treatment involves the delivery of agents with established tumor-immune reactivity (such as effector cells or antibodies) that can directly or indirectly mediate antitumor effects and does not necessarily depend on an intact host immune system. Examples of effector cells include T cells as discussed above, T lymphocytes (such as CD8⁺ cytotoxic T lymphocytes and CD4⁺ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein. T cell receptors and antibody receptors specific for the polypeptides recited herein may be cloned, expressed and transferred into other vectors or effector cells for adoptive immunotherapy. The polypeptides provided herein may also be used to generate antibodies or anti-idiotypic antibodies (as described above and in U.S. Pat. No. 4,918,164) for passive immunotherapy.

Monoclonal antibodies may be labeled with any of a variety of labels for desired selective usages in detection, diagnostic assays or therapeutic applications (as described in U.S. Pat. Nos. 6,090,365; 6,015,542; 5,843,398; 5,595,721; and 4,708,930, hereby incorporated by reference in their entirety as if each was incorporated individually). In each case, the binding of the labelled monoclonal antibody to the determinant site of the antigen will signal detection or delivery of a particular therapeutic agent to the antigenic determinant on the non-normal cell. A further object of this invention is to provide the specific monoclonal antibody suitably labelled for achieving such desired selective usages thereof.

Effector cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to rapidly expand antigen-specific T cell cultures in order to generate a sufficient number of cells for immunotherapy. In particular, antigen-presenting cells, such as dendritic, macrophage, monocyte, fibroblast and/or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., Immunological Reviews 157:177, 1997).

Alternatively, a vector expressing a polypeptide recited herein may be introduced into antigen presenting cells taken from a patient and clonally propagated ex vivo for transplant back into the same patient. Transfected cells may be reintroduced into the patient using any means known in the art, preferably in sterile form by intravenous, intracavitary, intraperitoneal or intratumor administration.

Routes and frequency of administration of the therapeutic compositions described herein, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response, and is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent remissions, complete or partial or longer disease-free survival) in vaccinated patients as compared to non-vaccinated patients. In general, for pharmaceutical compositions and vaccines comprising one or more polypeptides, the amount of each polypeptide present in a dose ranges from about 25 μg to 5 mg per kg of host. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to a tumor protein generally correlate with an improved clinical outcome. Such immune responses may generally be evaluated using standard proliferation, cytotoxicity or cytokine assays, which may be performed using samples obtained from a patient before and after treatment.

Cancer Detection and Diagnostic Compositions, Methods and Kits

In general, a cancer may be detected in a patient based on the presence of one or more breast tumor proteins and/or polynucleotides encoding such proteins in a biological sample (for example, blood, sera, sputum urine and/or tumor biopsies) obtained from the patient. In other words, such proteins may be used as markers to indicate the presence or absence of a cancer such as breast cancer. In addition, such proteins may be useful for the detection of other cancers. The binding agents provided herein generally permit detection of the level of antigen that binds to the agent in the biological sample.

Polynucleotide primers and probes may be used to detect the level of mRNA encoding a tumor protein, which is also indicative of the presence or absence of a cancer. In general, a tumor sequence should be present at a level that is at least two-fold, preferably three-fold, and more preferably five-fold or higher in tumor tissue than in normal tissue of the same type from which the tumor arose. Expression levels of a particular tumor sequence in tissue types different from that in which the tumor arose are irrelevant in certain diagnostic embodiments since the presence of tumor cells can be confirmed by observation of predetermined differential expression levels, e.g., 2-fold, 5-fold, etc, in tumor tissue to expression levels in normal tissue of the same type.

Other differential expression patterns can be utilized advantageously for diagnostic purposes. For example, in one aspect of the invention, overexpression of a tumor sequence in tumor tissue and normal tissue of the same type, but not in other normal tissue types, e.g. PBMCs, can be exploited diagnostically. In this case, the presence of metastatic tumor cells, for example in a sample taken from the circulation or some other tissue site different from that in which the tumor arose, can be identified and/or confirmed by detecting expression of the tumor sequence in the sample, for example using RT-PCR analysis. In many instances, it will be desired to enrich for tumor cells in the sample of interest, e.g., PBMCs, using cell capture or other like techniques.

There are a variety of assay formats known to those of ordinary skill in the art for using a binding agent to detect polypeptide markers in a sample. See, e.g., Harlow and Lane, Antibodies: A Laboratory Manual, Cold Spring Harbor Laboratory, 1988. In general, the presence or absence of a cancer in a patient may be determined by (a) contacting a biological sample obtained from a patient with a binding agent; (b) detecting in the sample a level of polypeptide that binds to the binding agent; and (c) comparing the level of polypeptide with a predetermined cut-off value.

In a preferred embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length breast tumor proteins and polypeptide portions thereof to which the binding agent binds, as described above.

The solid support may be any material known to those of ordinary skill in the art to which the tumor protein may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo.). The immobilized antibody is then incubated with the sample, and polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of polypeptide within a sample obtained from an individual with breast cancer at least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. The second antibody, which contains a reporter group, may then be added to the solid support. Preferred reporter groups include those groups recited above.

The detection reagent is then incubated with the immobilized antibody-polypeptide complex for an amount of time sufficient to detect the bound polypeptide. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a cancer, such as breast cancer, the signal detected from the reporter group that remains bound to the solid support is generally compared to a signal that corresponds to a predetermined cut-off value. In one preferred embodiment, the cut-off value for the detection of a cancer is the average mean signal obtained when the immobilized antibody is incubated with samples from patients without the cancer. In general, a sample generating a signal that is three standard deviations above the predetermined cut-off value is considered positive for the cancer. In an alternate preferred embodiment, the cut-off value is determined using a Receiver Operator Curve, according to the method of Sackett et al., Clinical Epidemiology. A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a cancer. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

Of course, numerous other assay protocols exist that are suitable for use with the tumor proteins or binding agents of the present invention. The above descriptions are intended to be exemplary only. For example, it will be apparent to those of ordinary skill in the art that the above protocols may be readily modified to use tumor polypeptides to detect antibodies that bind to such polypeptides in a biological sample. The detection of such tumor protein specific antibodies may correlate with the presence of a cancer.

A cancer may also, or alternatively, be detected based on the presence of T cells that specifically react with a tumor protein in a biological sample. Within certain methods, a biological sample comprising CD4⁺ and/or CD8⁺ T cells isolated from a patient is incubated with a tumor polypeptide, a polynucleotide encoding such a polypeptide and/or an APC that expresses at least an immunogenic portion of such a polypeptide, and the presence or absence of specific activation of the T cells is detected. Suitable biological samples include, but are not limited to, isolated T cells. For example, T cells may be isolated from a patient by routine techniques (such as by Ficoll/Hypaque density gradient centrifugation of peripheral blood lymphocytes). T cells may be incubated in vitro for 2-9 days (typically 4 days) at 37° C. with polypeptide (e.g., 5-25 μg/ml). It may be desirable to incubate another aliquot of a T cell sample in the absence of tumor polypeptide to serve as a control. For CD4⁺ T cells, activation is preferably detected by evaluating proliferation of the T cells. For CD8⁺ T cells, activation is preferably detected by evaluating cytolytic activity. A level of proliferation that is at least two fold greater and/or a level of cytolytic activity that is at least 20% greater than in disease-free patients indicates the presence of a cancer in the patient.

As noted above, a cancer may also, or alternatively, be detected based on the level of mRNA encoding a tumor protein in a biological sample. For example, at least two oligonucleotide primers may be employed in a polymerase chain reaction (PCR) based assay to amplify a portion of a tumor cDNA derived from a biological sample, wherein at least one of the oligonucleotide primers is specific for (i.e., hybridizes to) a polynucleotide encoding the tumor protein. The amplified cDNA is then separated and detected using techniques well known in the art, such as gel electrophoresis.

Similarly, oligonucleotide probes that specifically hybridize to a polynucleotide encoding a tumor protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding a tumor protein of the invention that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence as disclosed herein. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989).

One preferred assay employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A two-fold or greater increase in expression in several dilutions of the test patient sample as compared to the same dilutions of the non-cancerous sample is typically considered positive.

In another aspect of the present invention, cell capture technologies may be used in conjunction, with, for example, real-time PCR to provide a more sensitive tool for detection of metastatic cells expressing breast tumor antigens. Detection of breast cancer cells in biological samples, e.g., bone marrow samples, peripheral blood, and small needle aspiration samples is desirable for diagnosis and prognosis in breast cancer patients.

Immunomagnetic beads coated with specific monoclonal antibodies to surface cell markers, or tetrameric antibody complexes, may be used to first enrich or positively select cancer cells in a sample. Various commercially available kits may be used, including Dynabeads® Epithelial Enrich (Dynal Biotech, Oslo, Norway), StemSep™ (StemCell Technologies, Inc., Vancouver, BC), and RosetteSep (StemCell Technologies). A skilled artisan will recognize that other methodologies and kits may also be used to enrich or positively select desired cell populations. Dynabeads® Epithelial Enrich contains magnetic beads coated with mAbs specific for two glycoprotein membrane antigens expressed on normal and neoplastic epithelial tissues. The coated beads may be added to a sample and the sample then applied to a magnet, thereby capturing the cells bound to the beads. The unwanted cells are washed away and the magnetically isolated cells eluted from the beads and used in further analyses.

RosetteSep can be used to enrich cells directly from a blood sample and consists of a cocktail of tetrameric antibodies that targets a variety of unwanted cells and crosslinks them to glycophorin A on red blood cells (RBC) present in the sample, forming rosettes. When centrifuged over Ficoll, targeted cells pellet along with the free RBC. The combination of antibodies in the depletion cocktail determines which cells will be removed and consequently which cells will be recovered. Antibodies that are available include, but are not limited to: CD2, CD3, CD4, CD5, CD8, CD10, CD11b, CD14, CD15, CD16, CD19, CD20, CD24, CD25, CD29, CD33, CD34, CD36, CD38, CD41, CD45, CD45RA, CD45RO, CD56, CD66B, CD66e, HLA-DR, IgE, and TCRαβ.

Additionally, it is contemplated in the present invention that mAbs specific for breast tumor antigens can be generated and used in a similar manner. For example, mAbs that bind to tumor-specific cell surface antigens may be conjugated to magnetic beads, or formulated in a tetrameric antibody complex, and used to enrich or positively select metastatic breast tumor cells from a sample. Once a sample is enriched or positively selected, cells may be lysed and RNA isolated. RNA may then be subjected to RT-PCR analysis using breast tumor-specific primers in a real-time PCR assay as described herein. One skilled in the art will recognize that enriched or selected populations of cells may be analyzed by other methods (e.g. in situ hybridization or flow cytometry).

In another embodiment, the compositions described herein may be used as markers for the progression of cancer. In this embodiment, assays as described above for the diagnosis of a cancer may be performed over time, and the change in the level of reactive polypeptide(s) or polynucleotide(s) evaluated. For example, the assays may be performed every 24-72 hours for a period of 6 months to 1 year, and thereafter performed as needed. In general, a cancer is progressing in those patients in whom the level of polypeptide or polynucleotide detected increases over time. In contrast, the cancer is not progressing when the level of reactive polypeptide or polynucleotide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications. Alternatively, polynucleotide probes may be used within such applications.

As noted above, to improve sensitivity, multiple tumor protein markers may be assayed within a given sample. It will be apparent that binding agents specific for different proteins provided herein may be combined within a single assay. Further, multiple primers or probes may be used concurrently. The selection of tumor protein markers may be based on routine experiments to determine combinations that results in optimal sensitivity. In addition, or alternatively, assays for tumor proteins provided herein may be combined with assays for other known tumor antigens.

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to a tumor protein. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may be designed to detect the level of mRNA encoding a tumor protein in a biological sample. Such kits generally comprise at least one oligonucleotide probe or primer, as described above, that hybridizes to a polynucleotide encoding a tumor protein. Such an oligonucleotide may be used, for example, within a PCR or hybridization assay. Additional components that may be present within such kits include a second oligonucleotide and/or a diagnostic reagent or container to facilitate the detection of a polynucleotide encoding a tumor protein.

The following Examples are offered by way of illustration and not by way of limitation.

### EXAMPLES

### Example 1

### Preparation of Breast Tumor-Specific cDNAs Using Differential Display RT-PCR

This Example illustrates the preparation of cDNA molecules encoding breast tumor-specific polypeptides using a differential display screen.

A. Preparation of B18Ag1 cDNA and Characterization of mRNA Expression

Tissue samples were prepared from breast tumor and normal tissue of a patient with breast cancer that was confirmed by pathology after removal from the patient. Normal RNA and tumor RNA was extracted from the samples and mRNA was isolated and converted into cDNA using a (dT)₁₂AG (SEQ ID NO:130) anchored 3′ primer. Differential display PCR was then executed using a randomly chosen primer (CTTCAACCTC) (SEQ ID NO:103). Amplification conditions were standard buffer containing 1.5 mM MgCl₂, 20 pmol of primer, 500 pmol dNTP, and 1 unit of Taq DNA polymerase (Perkin-Elmer, Branchburg, N.J.). Forty cycles of amplification were performed using 94° C. denaturation for 30 seconds, 42° C. annealing for 1 minute, and 72° C. extension for 30 seconds. An RNA fingerprint containing 76 amplified products was obtained. Although the RNA fingerprint of breast tumor tissue was over 98% identical to that of the normal breast tissue, a band was repeatedly observed to be specific to the RNA fingerprint pattern of the tumor. This band was cut out of a silver stained gel, subcloned into the T-vector (Novagen, Madison, Wis.) and sequenced.

The sequence of the cDNA, referred to as B18Ag1, is provided in SEQ ID NO:1. A database search of GENBANK and EMBL revealed that the B18Ag1 fragment initially cloned is 77% identical to the endogenous human retroviral element S71, which is a truncated retroviral element homologous to the Simian Sarcoma Virus (SSV). S71 contains an incomplete gag gene, a portion of the pol gene and an LTR-like structure at the 3′ terminus (see Werner et al., Virology 174:225-238 (1990)). B18Ag1 is also 64% identical to SSV in the region corresponding to the P30 (gag) locus. B18Ag1 contains three separate and incomplete reading frames covering a region which shares considerable homology to a wide variety of gag proteins of retroviruses which infect mammals. In addition, the homology to S71 is not just within the gag gene, but spans several kb of sequence including an LTR.

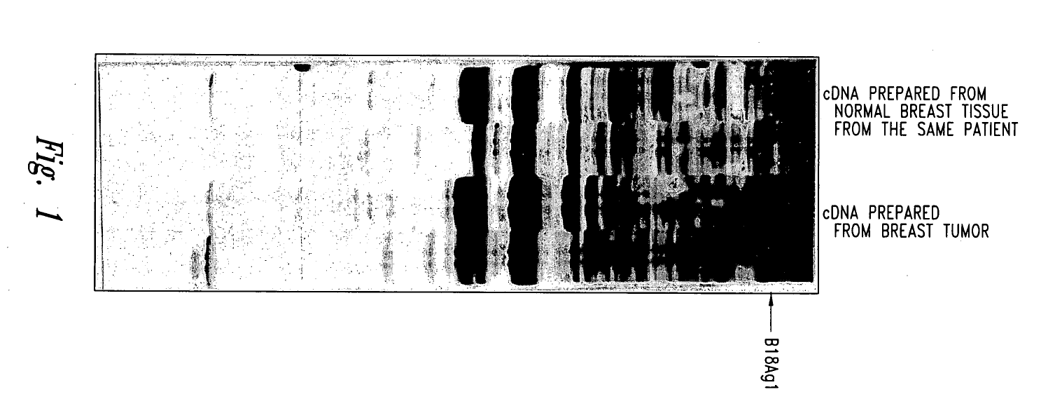

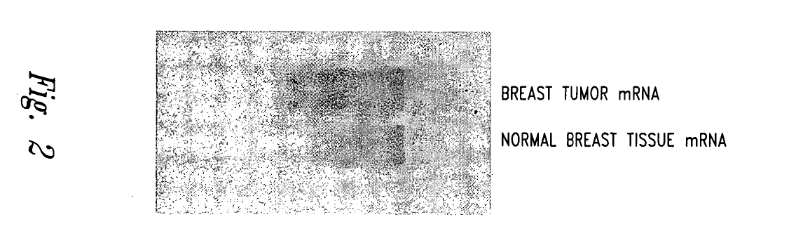

B18Ag1-specific PCR primers were synthesized using computer analysis guidelines. RT-PCR amplification (94° C., 30 seconds; 60° C.→42° C., 30 seconds; 72° C., 30 seconds for 40 cycles) confirmed that B18Ag1 represents an actual mRNA sequence present at relatively high levels in the patient's breast tumor tissue. The primers used in amplification were B18Ag1-1 (CTG CCT GAG CCA CAA ATG) (SEQ ID NO:128) and B18Ag1-4 (CCG GAG GAG GAA GCT AGA GGA ATA) (SEQ ID NO:129) at a 3.5 mM magnesium concentration and a pH of 8.5, and B18Ag1-2 (ATG GCT ATT TTC GGG GCC TGA CA) (SEQ ID NO:126) and B18Ag1-3 (CCG GTA TCT CCT CGT GGG TAT T) (SEQ ID NO:127) at 2 mM magnesium at pH 9.5. The same experiments showed exceedingly low to nonexistent levels of expression in this patient's normal breast tissue (see FIG. 1). RT-PCR experiments were then used to show that B18Ag1 mRNA is present in nine other breast tumor samples (from Brazilian and American patients) but absent in, or at exceedingly low levels in, the normal breast tissue corresponding to each cancer patient. RT-PCR analysis has also shown that the B18Ag1 transcript is not present in various normal tissues (including lymph node, myocardium and liver) and present at relatively low levels in PBMC and lung tissue. The presence of B18Ag1 mRNA in breast tumor samples, and its absence from normal breast tissue, has been confirmed by Northern blot analysis, as shown in FIG. 2.

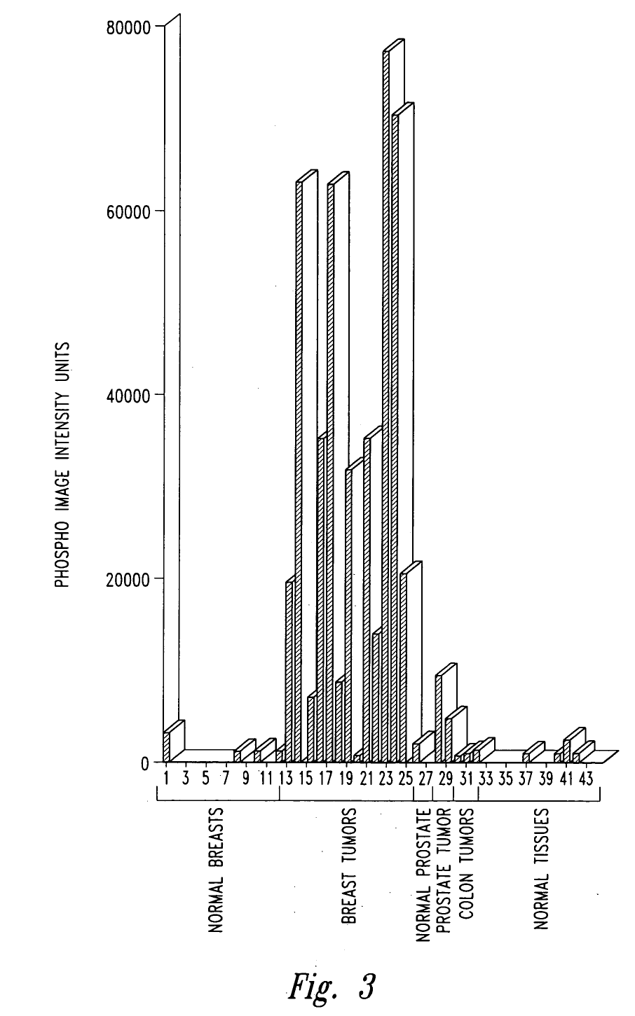

The differential expression of B18Ag1 in breast tumor tissue was also confirmed by RNase protection assays. FIG. 3 shows the level of B18Ag1 mRNA in various tissue types as determined in four different RNase protection assays. Lanes 1-12 represent various normal breast tissue samples, lanes 13-25 represent various breast tumor samples; lanes 26-27 represent normal prostate samples; lanes 28-29 represent prostate tumor samples; lanes 30-32 represent colon tumor samples; lane 33 represents normal aorta; lane 34 represents normal small intestine; lane 35 represents normal skin, lane 36 represents normal lymph node; lane 37 represents normal ovary; lane 38 represents normal liver; lane 39 represents normal skeletal muscle; lane 40 represents a first normal stomach sample, lane 41 represents a second normal stomach sample; lane 42 represents a normal lung; lane 43 represents normal kidney; and lane 44 represents normal pancreas. Interexperimental comparison was facilitated by including a positive control RNA of known β-actin message abundance in each assay and normalizing the results of the different assays with respect to this positive control.

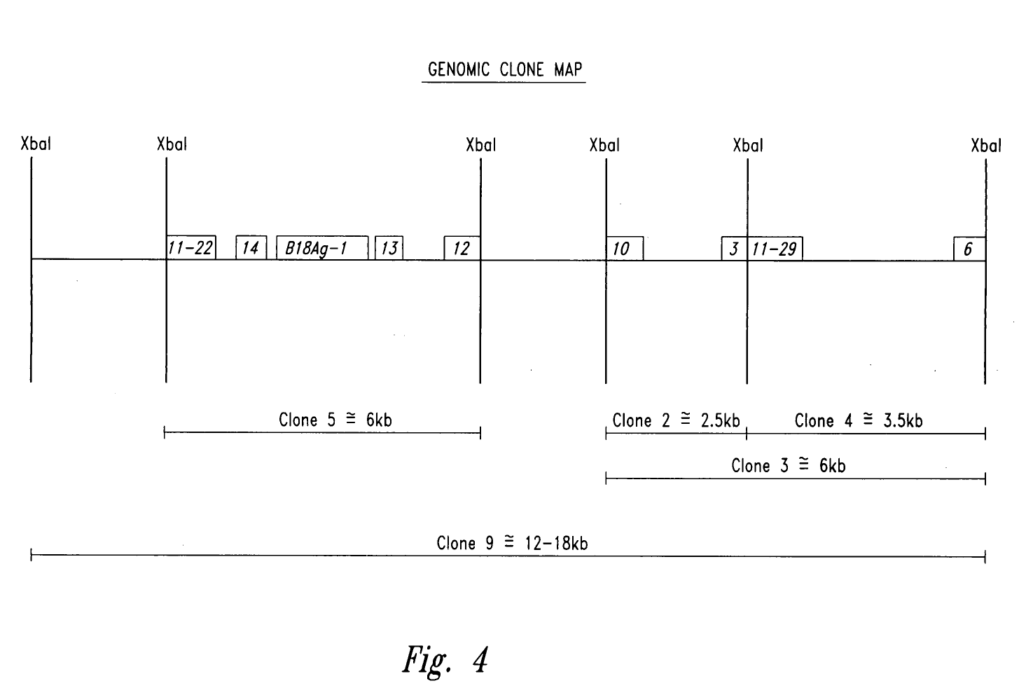

RT-PCR and Southern Blot analysis has shown the B18Ag1 locus to be present in human genomic DNA as a single copy endogenous retroviral element. A genomic clone of approximately 12-18 kb was isolated using the initial B18Ag1 sequence as a probe. Four additional subclones were also isolated by XbaI digestion. Additional retroviral sequences obtained from the ends of the XbaI digests of these clones (located as shown in FIG. 4) are shown as SEQ ID NO:3-SEQ ID NO:10, where SEQ ID NO:3 shows the location of the sequence labeled 10 in FIG. 4, SEQ ID NO:4 shows the location of the sequence labeled 11-29, SEQ ID NO:5 shows the location of the sequence labeled 3, SEQ ID NO:6 shows the location of the sequence labeled 6, SEQ ID NO:7 shows the location of the sequence labeled 12, SEQ ID NO:8 shows the location of the sequence labeled 13, SEQ ID NO:9 shows the location of the sequence labeled 14 and SEQ ID NO:10 shows the location of the sequence labeled 11 -22.

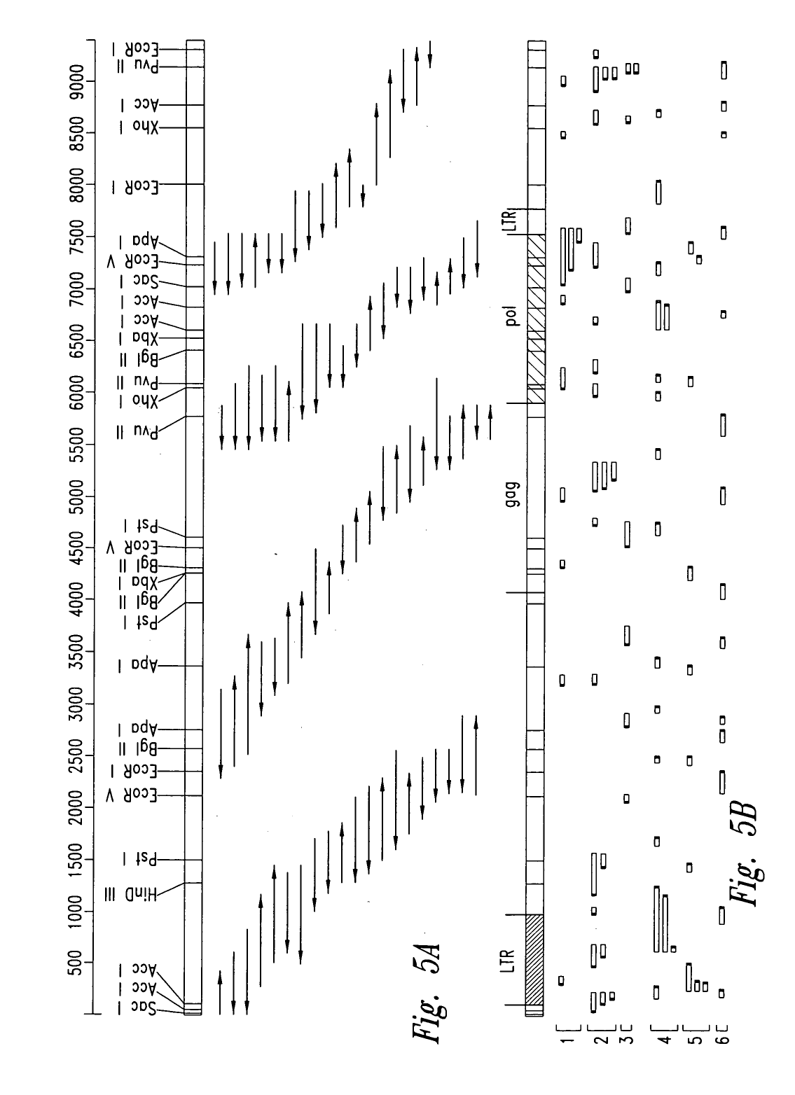

Subsequent studies demonstrated that the 12-18 kb genomic clone contains a retroviral element of about 7.75 kb, as shown in FIGS. 5A and 5B. The sequence of this retroviral element is shown in SEQ ID NO:141. The numbered line at the top of FIG. 5A represents the sense strand sequence of the retroviral genomic clone. The box below this line shows the position of selected restriction sites. The arrows depict the different overlapping clones used to sequence the retroviral element. The direction of the arrow shows whether the single-pass subclone sequence corresponded to the sense or anti-sense strand. FIG. 5B is a schematic diagram of the retroviral element containing B18Ag1 depicting the organization of viral genes within the element. The open boxes correspond to predicted reading frames, starting with a methionine, found throughout the element. Each of the six likely reading frames is shown, as indicated to the left of the boxes, with frames 1-3 corresponding to those found on the sense strand.

Using the cDNA of SEQ ID NO:1 as a probe, a longer cDNA was obtained (SEQ ID NO:227) which contains minor nucleotide differences (less than 1%) compared to the genomic sequence shown in SEQ ID NO:141.

B. Preparation of cDNA Molecules Encoding Other Breast Tumor-Specific Polypeptides

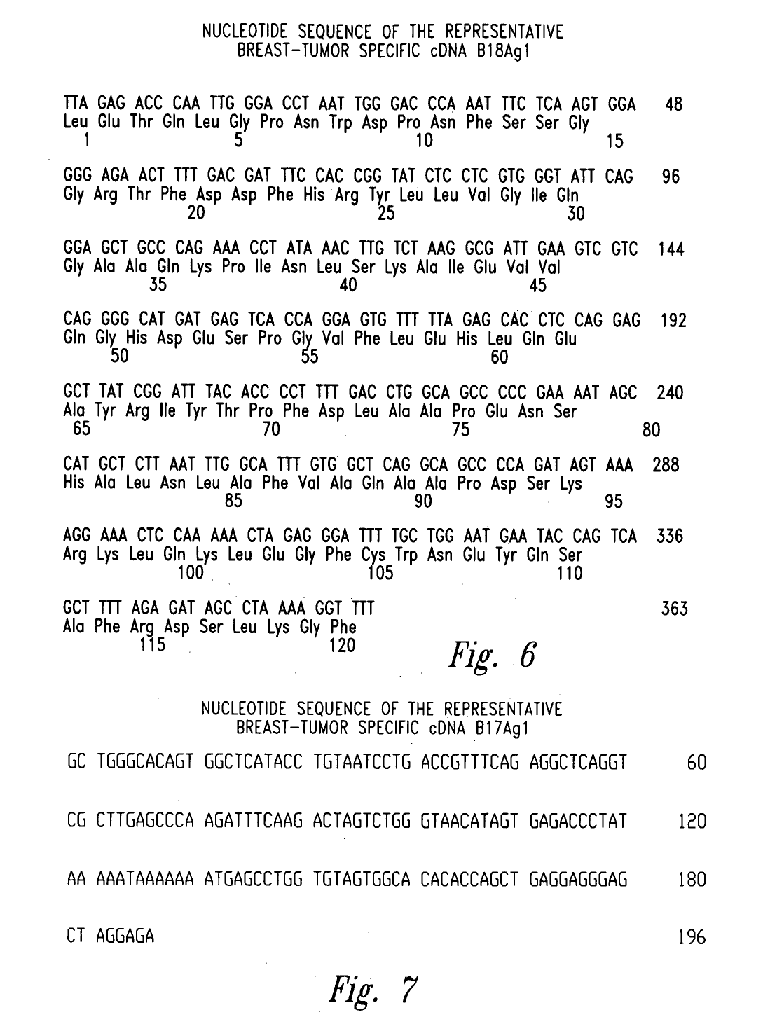

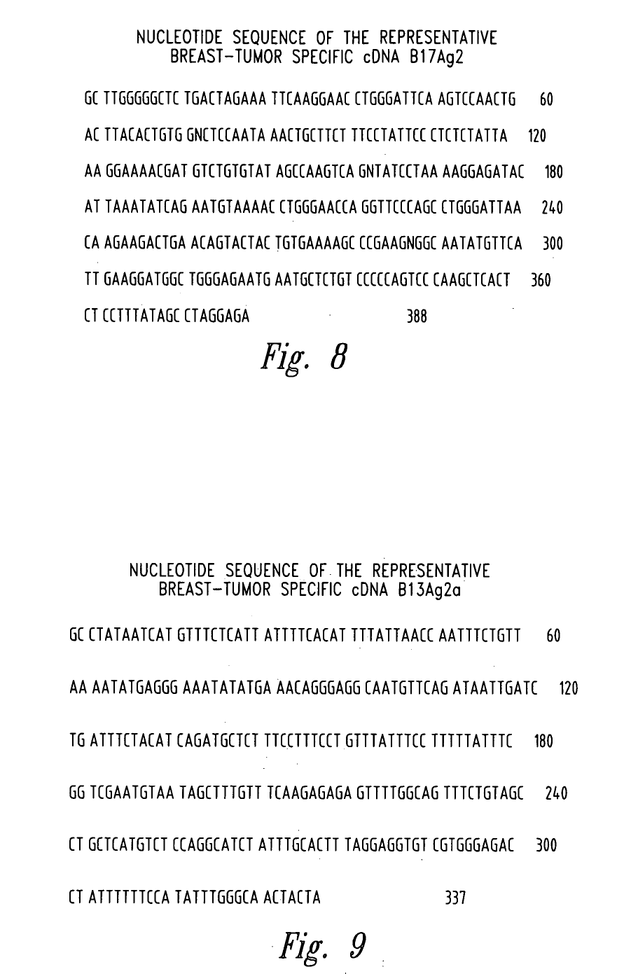

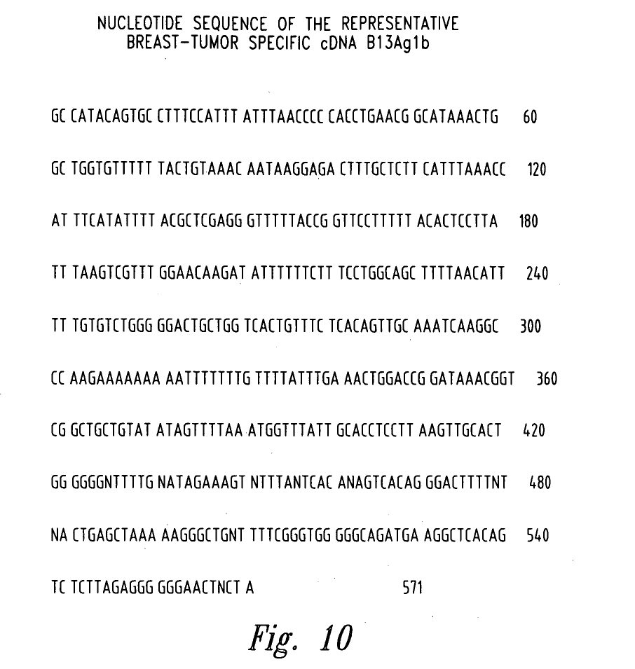

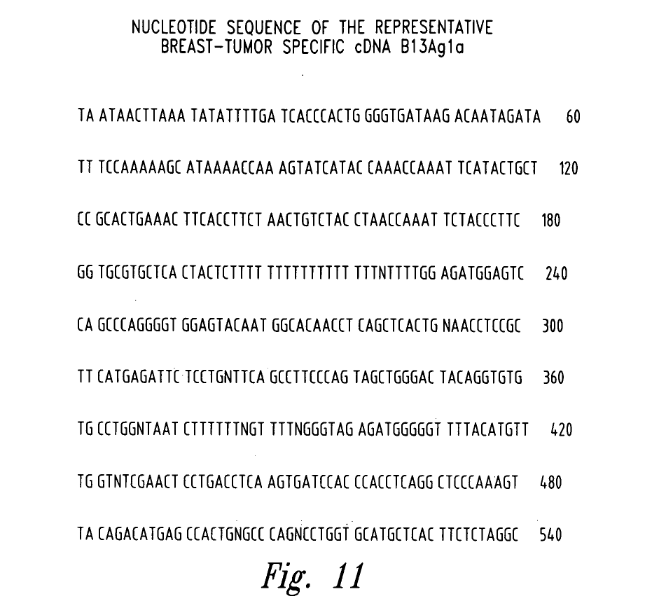

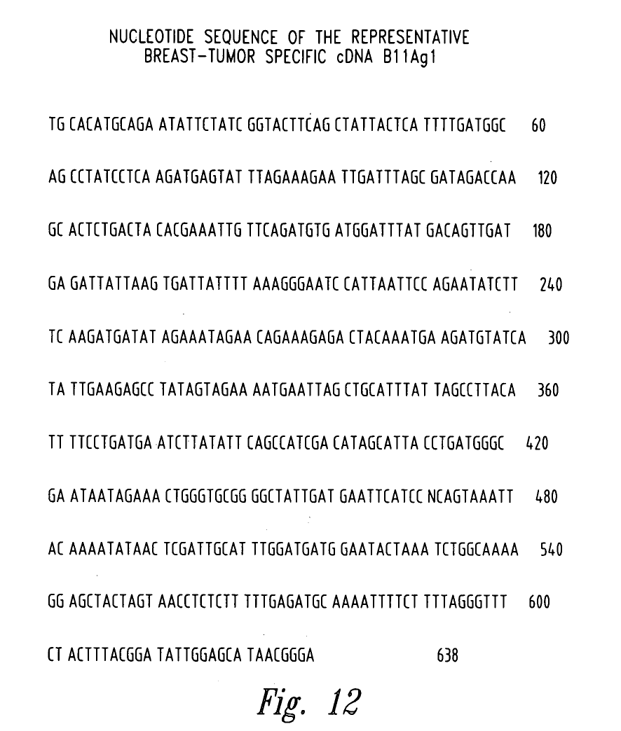

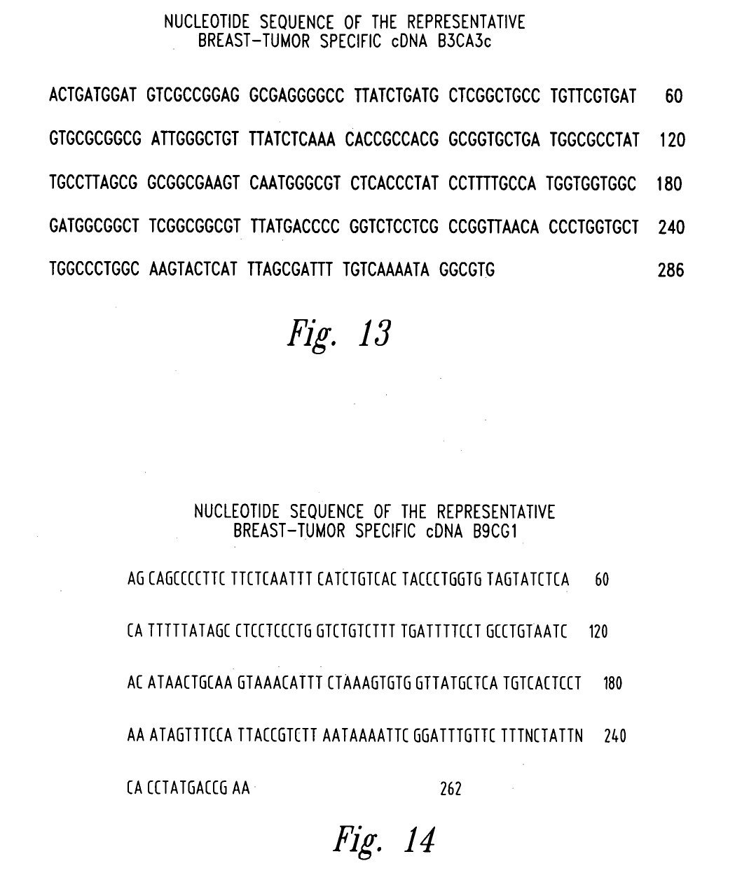

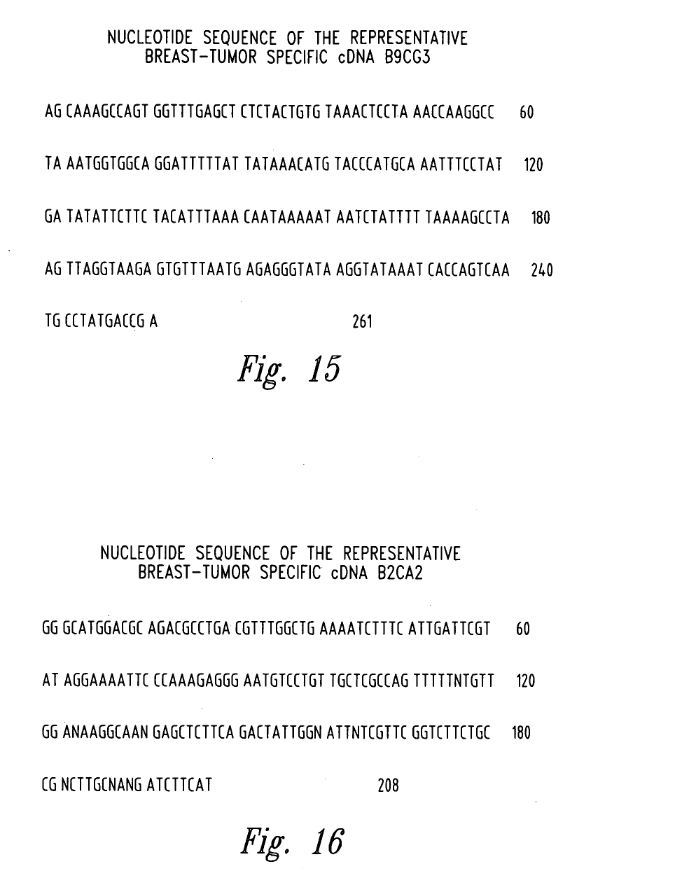

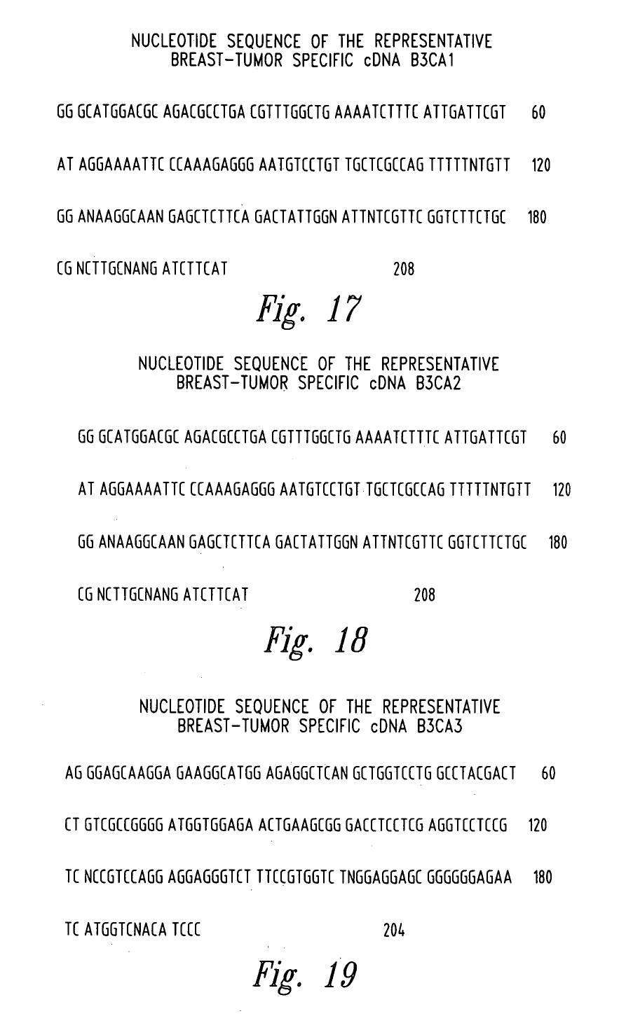

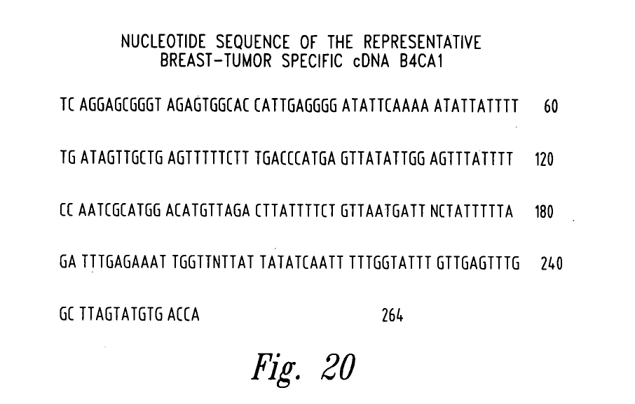

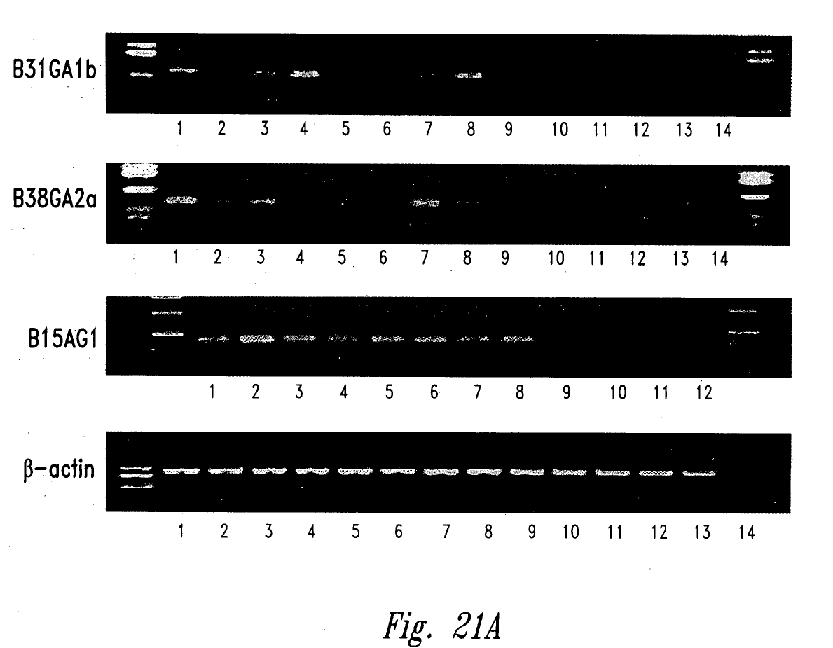

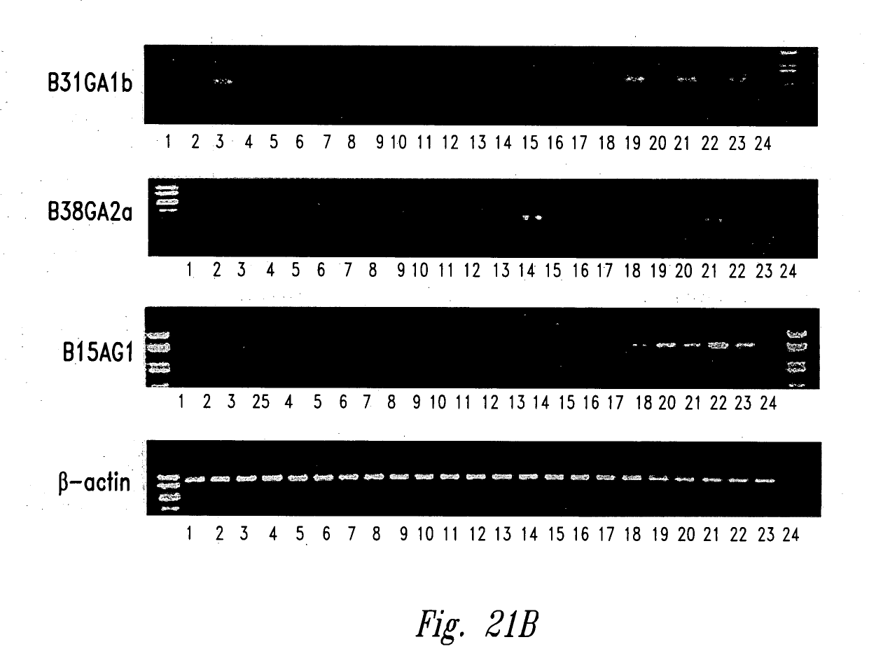

Normal RNA and tumor RNA was prepared and mRNA was isolated and converted into cDNA using a (dT)₁₂AG anchored 3′ primer, as described above. Differential display PCR was then executed using the randomly chosen primers of SEQ ID NO:87-125. Amplification conditions were as noted above, and bands observed to be specific to the RNA fingerprint pattern of the tumor were cut out of a silver stained gel, subcloned into either the T-vector (Novagen, Madison, Wis.) or the pCRII vector (Invitrogen, San Diego, Calif.) and sequenced. The sequences are provided in SEQ ID NO:11-SEQ ID NO:86. Of the 79 sequences isolated, 67 were found to be novel (SEQ ID NO:11-26 and 28-77) (see also FIGS. 6-20).

An extended DNA sequence (SEQ ID NO:290) for the antigen B15Ag1 (originally identified partial sequence provided in SEQ ID NO:27) was obtained in further studies. Comparison of the sequence of SEQ ID NO:290 with those in the gene bank as described above, revealed homology to the known human β-A activin gene. Further studies led to the isolation of the full-length cDNA sequence for the antigen B21 GT2 (also referred to as B311D; originally identified partial cDNA sequence provided in SEQ ID NO:56). The full-length sequence is provided in SEQ ID NO:307, with the corresponding amino acid sequence being provided in SEQ ID NO:308. Further studies led to the isolation of a splice variant of B311D. The B311D clone of SEQ ID NO:316 was sequenced and a XhoI/NotI fragment from this clone was gel purified and 32P-cDTP labeled by random priming for use as a probe for further screening to obtain additional B311D gene sequence. Two fractions of a human breast tumor cDNA bacterial library were screened using standard techniques. One of the clones isolated in this manner yielded additional sequence which includes a poly A+ tail. The determined cDNA sequence of this clone (referred to as B311D_BT1_{—}1A) is provided in SEQ ID NO:317. The sequences of SEQ ID NO:316 and 317 were found to share identity over a 464 bp region, with the sequences diverging near the poly A+ sequence of SEQ ID NO:317.

Subsequent studies identified an additional 146 sequences (SEQ ID NO:142-289), of which 115 appeared to be novel (SEQ ID NO:142, 143, 146-152, 154-166, 168-176, 178-192, 194-198, 200-204, 206, 207, 209-214, 216, 218, 219, 221-240, 243-245, 247, 250, 251, 253, 255, 257-266, 268, 269, 271-273, 275, 276, 278, 280, 281, 284, 288 and 291). To the best of the inventors' knowledge none of the previously identified sequences have heretofore been shown to be expressed at a greater level in human breast tumor tissue than in normal breast tissue.

In further studies, several different splice forms of the antigen B11Ag1 (also referred to as B305D) were isolated, with each of the various splice forms containing slightly different versions of the B11Ag1 coding frame. Splice junction sequences define individual exons which, in various patterns and arrangements, make up the various splice forms. Primers were designed to examine the expression pattern of each of the exons using RT-PCR as described below. Each exon was found to show the same expression pattern as the original B11Ag1 clone, with expression being breast tumor-, normal prostate- and normal testis-specific. The determined cDNA sequences for the isolated protein coding exons are provided in SEQ ID NO:292-298, respectively. The predicted amino acid sequences corresponding to the sequences of SEQ ID NO:292 and 298 are provided in SEQ ID NO:299 and 300. Additional studies using rapid amplification of cDNA ends (RACE), a 5′ specific primer to one of the splice forms of B11Ag1 provided above and a breast adenocarcinoma, led to the isolation of three additional, related, splice forms referred to as isoforms B11C-15, B11C-8 and B11C-9,16. The determined cDNA sequences for these isoforms are provided in SEQ ID NO: 301-303, with the corresponding predicted amino acid sequences being provided in SEQ ID-NO:304-306.

The protein coding region of B11C-15 (SEQ ID NO: 301; also referred to as B305D isoform C) was used as a query sequence in a BLASTN search of the Genbank DNA database. A match was found to a genomic clone from chromosome 21 (Accessson no. AP001465). The pairwise alignments provided in the BLASTN output were used to identify the putative exon, or coding, sequence of the chromosome 21 sequence that corresponds to the B305D sequence. Based on the BlastN pairwise alignments, the following pieces of GenBank record AP001465 were put together: base pairs 67978-68499, 72870-72987, 73144-73335, 76085-76206, 77905-78085, 80520-80624, 87602-87633. This sequence was then aligned with the B305D isoform C sequence using the DNA Star Seqman program and excess sequence was deleted in such a way as to maintain the sequence most similar to B305D. The final edited form of the chromosome 21 sequence was 96.5% identical to B305D. This resulting edited sequence from chromosome 21 was then translated and found to contain no stop codons other than the final stop codon in the same position as that for B305D. As with B305D, the chromosome 21 sequence (provided in SEQ ID NO: 325) encoded a protein (SEQ ID NO: 326) with 384 amino acids. An alignment of this protein with the B305D isoform C protein (SEQ ID NO: 304) showed 90% amino acid identity.

The cDNA sequence of B305D isoform C (SEQ ID NO: 301) was used to identify homologs by searching the High Throughput Genome Sequencing (HTGS) database (NCBI, National Institutes for Health, Bethesda, Md.). Homologs were identified on Chromosome 2 (Clone ID 9838181), Chromosome 10 (Clone ID 10933022), Chromosome 15 (Clone ID 11560284). These homologs shared greater than 90% identity with B305D isoform C at the nucleic acid level. All three of these homologs encode 384 amino acid ORFs that share greater than 90% identity with the amino acid sequence of SEQ ID NO: 304. Further searching of the GenBank database with the sequence of SEQ ID NO: 301 yielded a partial sequence homolog on Chromosome 22 (Clone ID 5931507). cDNA sequences for the Chromosome 2, 10, 15 and 22 homologs were constructed based on the homology with B305D isoform C and the conserved sequences at intron-exon junctions. The cDNA sequences for the Chromosome 22, 2, 15 and 10 homologs are provided in SEQ ID NO: 327-330, respectively, with the corresponding amino acid sequences being provided in SEQ ID NO: 331, 334, 333 and 332, respectively.

In subsequent studies on B305D isoform A (cDNA sequence provided in SEQ ID NO:292), the cDNA sequence (provided in SEQ ID NO:313) was found to contain an additional guanine residue at position 884, leading to a frameshift in the open reading frame. The determined DNA sequence of this ORF is provided in SEQ ID NO:314. This frameshift generates a protein sequence (provided in SEQ ID NO:315) of 293 amino acids that contains the C-terminal domain common to the other isoforms of B305D but that differs in the N-terminal region.

### Example 2

### Preparation of B18Ag1 DNA from Human Genomic DNA

This Example illustrates the preparation of B18Ag1 DNA by amplification from human genomic DNA.

B18Ag1 DNA may be prepared from 250 ng human genomic DNA using 20 pmol of B18Ag1 specific primers, 500 pmol dNTPS and 1 unit of Taq DNA polymerase (Perkin Elmer, Branchburg, N.J.) using the following amplification parameters: 94° C. for 30 seconds denaturing, 30 seconds 60° C. to 42° C. touchdown annealing in 2° C. increments every two cycles and 72° C. extension for 30 seconds. The last increment (a 42° C. annealing temperature) should cycle 25 times. Primers were selected using computer analysis. Primers synthesized were B18Ag1-1, B18Ag1-2, B18Ag1-3, and B18Ag1-4. Primer pairs that may be used are 1+3, 1+4, 2+3, and 2+4.

Following gel electrophoresis, the band corresponding to B18Ag1 DNA may be excised and cloned into a suitable vector.

### Example 3

### Preparation of B18Ag1 DNA from Breast Tumor CDNA

This Example illustrates the preparation of B18Ag1 DNA by amplification from human breast tumor cDNA.

First strand cDNA is synthesized from RNA prepared from human breast tumor tissue in a reaction mixture containing 500 ng poly A+ RNA, 200 pmol of the primer (T)₁₂AG (i.e., TTT TTT TTT TTT AG) (SEQ ID NO:130), 1× first strand reverse transcriptase buffer, 6.7 mM DTT, 500 mmol dNTPs, and 1 unit AMV or MMLV reverse transcriptase (from any supplier, such as Gibco-BRL (Grand Island, N.Y.)) in a final volume of 30 μl. After first strand synthesis, the cDNA is diluted approximately 25 fold and 1 μl is used for amplification as described in Example 2. While some primer pairs can result in a heterogeneous population of transcripts, the primers B18Ag1-2 (5′ ATG GCT ATT TTC GGG GGC TGA CA) (SEQ ID NO:126) and B18Ag1-3 (5′ CCG GTA TCT CCT CGT GGG TAT T) (SEQ ID NO:127) yield a single 151 bp amplification product.

### Example 4

### Identification of B-cell and T-cell Epitopes of B18Ag1

This Example illustrates the identification of B18Ag1 epitopes.

The B18Ag1 sequence can be screened using a variety of computer algorithms. To determine B-cell epitopes, the sequence can be screened for hydrophobicity and hydrophilicity values using the method of Hopp, Prog. Clin. Biol. Res. 172B:367-77 (1985) or, alternatively, Cease et al., J. Exp. Med. 164:1779-84 (1986) or Spouge et al., J. Immunol. 138:204-12 (1987). Additional Class II MHC (antibody or B-cell) epitopes can be predicted using programs such as AMPHI (e.g., Margalit et al., J. Immunol. 138:2213 (1987)) or the methods of Rothbard and Taylor (e.g., EMBO J. 7:93 (1988)).

Once peptides (15-20 amino acids long) are identified using these techniques, individual peptides can be synthesized using automated peptide synthesis equipment (available from manufacturers such as Perkin Elmer/Applied Biosystems Division, Foster City, Calif.) and techniques such as Merrifield synthesis. Following synthesis, the peptides can used to screen sera harvested from either normal or breast cancer patients to determine whether patients with breast cancer possess antibodies reactive with the peptides. Presence of such antibodies in breast cancer patient would confirm the immunogenicity of the specific B-cell epitope in question. The peptides can also be tested for their ability to generate a serologic or humoral immune in animals (mice, rats, rabbits, chimps etc.) following immunization in vivo. Generation of a peptide-specific antiserum following such immunization further confirms the immunogenicity of the specific B-cell epitope in question.

To identify T-cell epitopes, the B18Ag1 sequence can be screened using different computer algorithms which are useful in identifying 8-10 amino acid motifs within the B18Ag1 sequence which are capable of binding to HLA Class I MHC molecules. (see, e.g., Rammensee et al., Immunogenetics 41:178-228 (1995)). Following synthesis such peptides can be tested for their ability to bind to class I MHC using standard binding assays (e.g., Sette et al., J. Immunol. 153:5586-92 (1994)) and more importantly can be tested for their ability to generate antigen reactive cytotoxic T-cells following in vitro stimulation of patient or normal peripheral mononuclear cells using, for example, the methods of Bakker et al., Cancer Res. 55:5330-34 (1995); Visseren et al., J. Immunol. 154:3991-98 (1995); Kawakami et al., J. Immunol. 154:3961-68 (1995); and Kast et al., J. Immunol. 152:3904-12 (1994). Successful in vitro generation of T-cells capable of killing autologous (bearing the same Class I MHC molecules) tumor cells following in vitro peptide stimulation further confirms the immunogenicity of the B18Ag1 antigen. Furthermore, such peptides may be used to generate murine peptide and B18Ag1 reactive cytotoxic T-cells following in vivo immunization in mice rendered transgenic for expression of a particular human MHC Class I haplotype (Vitiello et al., J. Exp. Med. 173:1007-15 (1991).

A representative list of predicted B18Ag1 B-cell and T-cell epitopes, broken down according to predicted HLA Class I MHC binding antigen, is shown below:

(SEQ ID NOS.: 131-133)

Predicted Th Motifs (B-cell epitopes)
SSGGRTFDDFHRYLLVGI
QGAAQKPINLSKXIEVVQGHDE
SPGVFLEHLQEAYRIYTPFDLSA

(SEQ ID NOS.: 134-140)

Predicted HLA A2.1 Motifs (T-cell epitopes)
YLLVGIQGA
GAAQKPINL
NLSKXIEVV
EVVQGHDES
HLQEAYRIY
NLAFVAQAA
FVAQAAPDS

### Example 5

### Identification of T-cell Epitopes of B11Ag1

This Example illustrates the identification of B11Ag1 (also referred to as B305D) epitopes. Four peptides, referred to as B11-8, B11-1, B11-5 and B11-12 (SEQ ID NO:309-312, respectfully) were derived from the B11Ag1 gene.

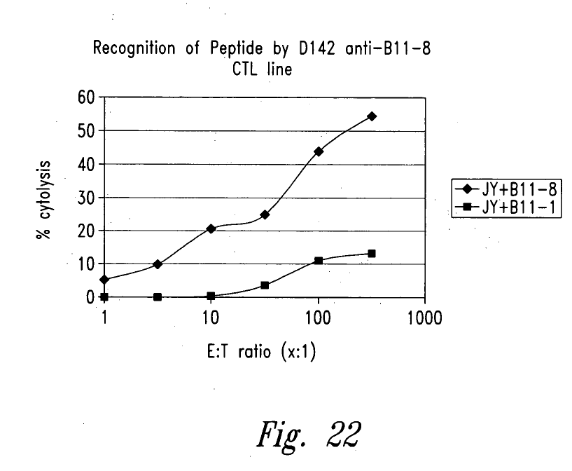

Human CD8 T cells were primed in vitro to the peptide B11-8 using dendritic cells according to the protocol of Van Tsai et al. (Critical Reviews in Immunology 18:65-75, 1998). The resulting CD8 T cell cultures were tested for their ability to recognize the B11-8 peptide or a negative control peptide, presented by the B-LCL line, JY. Briefly, T cells were incubated with autologous monocytes in the presence of 10 μg/ml peptide, 10 ng/ml IL-7 and 10 μg/ml IL-2, and assayed for their ability to specifically lyse target cells in a standard 51-Cr release assay. As shown in FIG. 22, the bulk culture line demonstrated strong recognition of the B11-8 peptide with weaker recognition of the peptide B11-1.

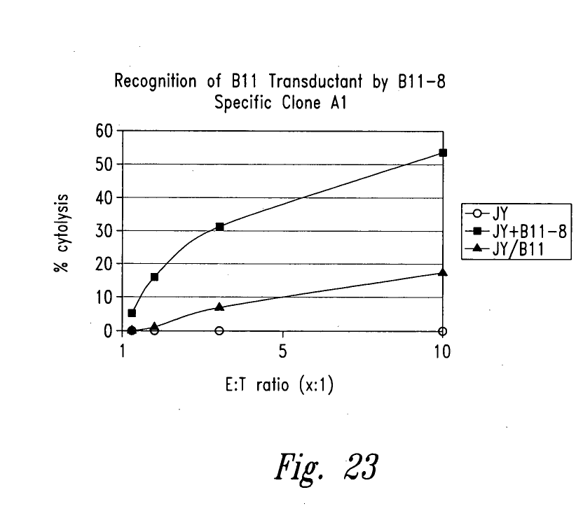

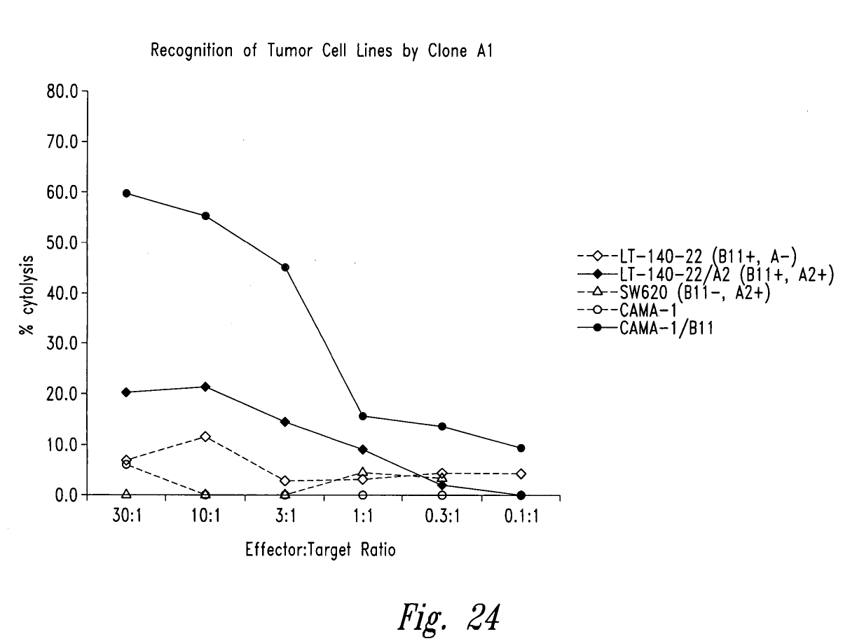

A clone from this CTL line was isolated following rapid expansion using the monoclonal antibody OKT3 and human IL-2. As shown in FIG. 23, this clone (referred to as A1), in addition to being able to recognize specific peptide, recognized JY LCL transduced with the B11Ag1 gene. This data demonstrates that B11-8 is a naturally processed epitope of the B11Ag1 gene. In addition these T cells were further found to recognize and lyse, in an HLA-A2 restricted manner, an established tumor cell line naturally expressing B11Ag1 (FIG. 24). The T cells strongly recognize a lung adenocarcinoma (LT-140-22) naturally expressing B11Ag1 transduced with HLA-A2, as well as an A2+ breast carcinoma (CAMA-1) transduced with B11Ag1, but not untransduced lines or another negative tumor line (SW620).

These data clearly demonstrate that these human T cells recognize not only B11-specific peptides but also transduced cells, as well as naturally expressing tumor lines.

CTL lines raised against the antigens B11-5 and B11-12, using the procedures described above, were found to recognize corresponding peptide-coated targets.

### Example 6

### Characterization of Breast Tumor Genes Disocvered by Differential Display PCR

The specificity and sensitivity of the breast tumor genes discovered by differential display PCR were determined using RT-PCR. This procedure enabled the rapid evaluation of breast tumor gene mRNA expression semiquantitatively without using large amounts of RNA. Using gene specific primers, mRNA expression levels in a variety of tissues were examined, including 8 breast tumors, 5 normal breasts, 2 prostate tumors, 2 colon tumors, 1 lung tumor, and 14 other normal adult human tissues, including normal prostate, colon, kidney, liver, lung, ovary, pancreas, skeletal muscle, skin, stomach and testes.

To ensure the semiquantitative nature of the RT-PCR, β-actin was used as internal control for each of the tissues examined. Serial dilutions of the first strand cDNAs were prepared and RT-PCR assays performed using β-actin specific primers. A dilution was then selected that enabled the linear range amplification of β-actin template, and which was sensitive enough to reflect the difference in the initial copy number. Using this condition, the β-actin levels were determined for each reverse transcription reaction from each tissue. DNA contamination was minimized by DNase treatment and by assuring a negative result when using first strand cDNA that was prepared without adding reverse transcriptase.

Using gene specific primers, the mRNA expression levels were determined in a variety of tissues. To date, 38 genes have been successfully examined by RT-PCR, five of which exhibit good specificity and sensitivity for breast tumors (B15AG-1, B31GA1b, B38GA2a, B11A1a and B18AG1a). FIGS. 21A and 21B depict the results for three of these genes: B15AG-1 (SEQ ID NO:27), B31GA1b (SEQ ID NO:148) and B38GA2a (SEQ ID NO:157). Table I summarizes the expression level of all the genes tested in normal breast tissue and breast tumors, and also in other tissues.

TABLE I
Percentage of Breast Cancer Antigens that are Expressed in Various
Tissues

| | | |
|---|---|---|
| Breast Tissues | Over-expressed in Breast Tumors | 84% |
| | Equally Expressed in Normals and Tumor | 16% |
| Other Tissues | Over-expressed in Breast Tumors but | 9% |
| | not in any Normal Tissues | |
| | Over-expressed in Breast Tumors but | 30% |
| | Expressed in Some Normal Tissues | |
| | Over-expressed in Breast Tumors but | 61% |
| | Equally Expressed in All Other Tissues | |

### Example 7

### Preparation and Characterization of Antibodies Against Breast Tumor Polypeptides

Polyclonal antibodies against the breast tumor antigen B305D were prepared as follows.

The breast tumor antigen expressed in an E. coli recombinant expression system was grown overnight in LB broth with the appropriate antibiotics at 37 ° C. in a shaking incubator. The next morning, 10 ml of the overnight culture was added to 500 ml to 2x YT plus appropriate antibiotics in a 2L-baffled Erlenmeyer flask. When the Optical Density (at 560 nm) of the culture reached 0.4-0.6, the cells were induced with IPTG (1 mM). Four hours after induction with IPTG, the cells were harvested by centrifugation. The cells were then washed with phosphate buffered saline and centrifuged again. The supernatant was discarded and the cells were either frozen for future use or immediately processed. Twenty ml of lysis buffer was added to the cell pellets and vortexed. To break open the E. coli cells, this mixture was then run through the French Press at a pressure of 16,000 psi. The cells were then centrifuged again and the supernatant and pellet were checked by SDS-PAGE for the partitioning of the recombinant protein. For proteins that localized to the cell pellet, the pellet was resuspended in 10 mM Tris pH 8.0, 1% CHAPS and the inclusion body pellet was washed and centrifuged again. This procedure was repeated twice more. The washed inclusion body pellet was solubilized with either 8 M urea or 6 M guanidine HCl containing 10 mM Tris pH 8.0 plus 10 mM imidazole. The solubilized protein was added to 5 ml of nickel-chelate resin (Qiagen) and incubated for 45 min to 1 hour at room temperature with continuous agitation. After incubation, the resin and protein mixture were poured through a disposable column and the flow through was collected. The column was then washed with 10-20 column volumes of the solubilization buffer. The antigen was then eluted from the column using 8M urea, 10 mM Tris pH 8.0 and 300 mM imidazole and collected in 3 ml fractions. A SDS-PAGE gel was run to determine which fractions to pool for further purification.

As a final purification step, a strong anion exchange resin such as HiPrepQ (Biorad) was equilibrated with the appropriate buffer and the pooled fractions from above were loaded onto the column. Antigen was eluted off the column with a increasing salt gradient. Fractions were collected as the column was run and another SDS-PAGE gel was run to determine which fractions from the column to pool. The pooled fractions were dialyzed against 10 mM Tris pH 8.0. The protein was then vialed after filtration through a 0.22 micron filter and the antigens were frozen until needed for immunization.

Four hundred micrograms of B305D antigen was combined with 100 micrograms of muramyldipeptide (MDP). Every four weeks rabbits were boosted with 100 micrograms mixed with an equal volume of Incomplete Freund's Adjuvant (IFA). Seven days following each boost, the animal was bled. Sera was generated by incubating the blood at 4° C. for 12-24 hours followed by centrifugation.

Ninety-six well plates were coated with B305D antigen by incubating with 50 microliters (typically 1 microgram) of recombinant protein at 4° C. for 20 hours. 250 microliters of BSA blocking buffer was added to the wells and incubated at room temperature for 2 hours. Plates were washed 6 times with PBS/0.01% Tween. Rabbit sera was diluted in PBS. Fifty microliters of diluted sera was added to each well and incubated at room temperature for 30 min. Plates were washed as described above before 50 microliters of goat anti-rabbit horse radish peroxidase (HRP) at a 1:10000 dilution was added and incubated at room temperature for 30 min. Plates were again washed as described above and 100 microliters of TMB microwell peroxidase substrate was added to each well. Following a 15 min incubation in the dark at room temperature, the colorimetric reaction was stopped with 100 microliters of 1N H₂SO₄ and read-immediately at 450 nm. The polyclonal antibodies showed immunoreactivity to B305D.

Immunohistochemical (IHC) analysis of B305D expression in breast cancer and normal breast specimens was performed as follows. Paraffin-embedded formal fixed tissue was sliced into 8 micron sections. Steam heat induced epitope retrieval (SHIER) in 0.1 M sodium citrate buffer (pH 6.0) was used for optimal staining conditions. Sections were incubated with 10% serum/PBS for 5 minutes. Primary antibody was added to each section for 25 min at indicated concentrations followed by a 25 min incubation with either an anti-rabbit or anti-mouse biotinylated antibody. Endogenous peroxidase activity was blocked by three 1.5 min incubations with hydrogen peroxide. The avidin biotin complex/horseradish peroxidase (ABC/HRP) systems was used along with DAB chromagen to visualize antigen expression. Slides were counterstained with hematoxylin. B305D expression was detected in both breast tumor and normal breast tissue. However, the intensity of staining was much less in normal samples than in tumor samples and surface expression of B305D was observed only in breast tumor tissues.

A summary of real-time PCR and immunohistochemical analysis of B305D expression in an extensive panel of normal tissues is presented in Table II below. These results demonstrate minimal expression of B305D in testis, inconclusive results in gall bladder, and no detection in all other tissues tested.

TABLE II

| mRNA | IHC staining | Tissue type | Summary |
|---|---|---|---|
| Moderately positive | Positive | Testis | Nuclear staining of small |
| | | | minority of spermatids; |
| | | | spermatozoa negative; |
| | | | siminoma negative |
| Negative | Negative | Thymus | No expression |
| N/A | Negative | Artery | No expression |
| Negative | Negative | Skeletal muscle | No expression |
| Negative | Positive (weak staining) | Small bowel | No expression |
| Negative | Positive (weak staining) | Ovary | No expression |
| Negative | | Pituitary | No expression |
| Negative | Positive (weak staining) | Stomach | No expression |
| Negative | Negative | Spinal cord | No expression |
| Negative | Negative | Spleen | No expression |
| Negative | Negative | Ureter | No expression |
| N/A | Negative | Gall bladder | Inconclusive |
| N/A | Negative | Placenta | No expression |
| Negative | Negative | Thyroid | No expression |
| Negative | Negative | Heart | No expression |
| Negative | Negative | Kidney | No expression |
| Negative | Negative | Liver | No expression |
| Negative | Negative | Brain-cerebellum | No expression |
| Negative | Negative | Colon | No expression |
| Negative | Negative | Skin | No expression |
| Negative | Negative | Bone marrow | No expression |
| N/A | Negative | Parathyroid | No expression |
| Negative | Negative | Lung | No expression |
| Negative | Negative | Esophagus | No expression |
| Negative | Positive (weak staining) | Uterus | No expression |
| Negative | Negative | Adrenal | No expression |
| Negative | Negative | Pancreas | No expression |
| N/A | Negative | Lymph node | No expression |
| Negative | Negative | Brain-cortex | No expression |
| N/A | Negative | Fallopian tube | No expression |
| Negative | Positive (weak staining) | Bladder | No expression |
| Negative | N/A | Bone | No expression |
| Negative | N/A | Salivary gland | No expression |
| Negative | N/A | Activated PBMC | No expression |
| Negative | N/A | Resting PBMC | No expression |
| Negative | N/A | Trachea | No expression |
| Negative | N/A | Vena cava | No expression |
| Negative | N/A | Retina | No expression |
| Negative | N/A | Cartilage | No expression |

### Example 8

### Protein Expression of Breast Tumor Antigens

This example describes the expression and purification of the breast tumor antigen B305D in E. coli and in mammalian cells.

Expression of B305D isoform C-15 (SEQ ID NO:301; translated to 384 amino acids) in E. coli was achieved by cloning the open reading frame of B305D isoform C-15 downstream of the first 30 amino acids of the M tuberculosis antigen Ra12 (SEQ ID NO:318) in pET17b. First, the internal EcoRI site in the B305D ORF was mutated without changing the protein sequence so that-the gene could be cloned at the EcoRI site with Ra12. The PCR primers used for site-directed mutagenesis are shown in SEQ ID NO:319 (referred to as AW012) and SEQ ID NO:320 (referred to as AW013). The ORF of EcoRI site-modified B305D was then amplified by PCR using the primers AW014 (SEQ ID NO:321) and AW015 (SEQ ID NO:322). The PCR product was digested with EcoRI and ligated to the Ra12/pET17b vector at the EcoRI site. The sequence of the resulting fusion construct (referred to as Ra12mB11C) was confirmed by DNA sequencing. The determined cDNA sequence for the fusion construct is provided in SEQ ID NO:323, with the amino acid sequence being provided in SEQ ID NO:324.

The fusion construct was transformed into BL21(DE3)CodonPlus-RIL E. coli (Stratagene) and grown overnight in LB broth with kanamycin. The resulting culture was induced with IPTG. Protein was transferred to PVDF membrane and blocked with 5% non-fat milk (in PBS-Tween buffer), washed three times and incubated with mouse anti-His tag antibody (Clontech) for 1 hour. The membrane was washed 3 times and probed with HRP-Protein A (Zymed) for 30 min. Finally, the membrane was washed 3 times and developed with ECL (Amersham). Expression was detected by Western blot.

For recombinant expression in mammalian cells, B305D isoform C-15 (SEQ ID NO:301; translated to 384 amino acids) was subcloned into the mammalian expression vectors pCEP4 and pcDNA3.1 (Invitrogen). These constructs were transfected into HEK293 cells (ATCC) using Fugene 6 reagent (Roche). Briefly, the HEK cells were plated at a density of 100,000 cells/ml in DMEM (Gibco) containing 10% FBS (Hyclone) and grown overnight. The following day, 2 ul of Fugene 6 was added to 100 ul of DMEM containing no FBS and incubated for 15 minutes at room temperature. The Fugene 6/DMEM mixture was added to 1 ug of B305D/pCEP4 or B305D/pcDNA plasmid DNA and incubated for 15 minutes at room temperature. The Fugene/DNA mix was then added to the HEK293 cells and incubated for 48-72 hours at 37° C. with 7% CO₂. Cells were rinsed with PBS, the collected and pelleted by centrifugation.

For Western blot analysis, whole cell lysates were generated by incubating the cells in Triton-X100 containing lysis buffer for 30 minutes on ice. Lysates were then cleared by centrifugation at 10,000 rpm for 5 minutes at 4° C. Samples were diluted with SDS-PAGE loading buffer containing beta-mercaptoethanol, and boiled for 10 minutes prior to loading the SDS-PAGE gel. Proteins were transferred to nitrocellulose and probed using Protein A purified anti-B305D rabbit polyclonal sera (prepared as described above) at a concentration of 1 ug/ml. The blot was revealed with a goat anti-rabbit Ig coupled to HRP followed by incubation in ECL substrate. Expression of B305D was detected in the the HEK293 lysates transfected with B305D, but not in control HEK293 cells transfected with vector alone.

For FACS analysis, cells were washed further with ice cold staining buffer and then incubated with a 1:100 dilution of a goat anti-rabbit Ig (H+L)-FITC reagent (Southern Biotechnology) for 30 minutes on ice. Following 3 washes, the cells were resuspended in staining buffer containing Propidium Iodide (PI), a vital stain that allows for identification of permeable cells, and then analyzed by FACS. The FACS analysis showed surface expression of B305D protein.

### Example 9

### Expression of Full-Length B305D In Insect Cells Using A Baculovirus Expression System

The cDNA for the full-length breast tumor antigen, B305D isoform C (SEQ ID NO:301), with a C-terminal His Tag was made by PCR using B11C15/pBib as a template and the following primers:

B305DF1

(SEQ ID NO:337)

5′CGGCGGATCCACCATGGTGGTTGAGGTTGATTCC
B305DRV1:

(SEQ ID NO:338)

5′CGGCTCTAGATTAATGGTGATGGTGATGATGATGGTGATGATGTTTAT
TTCTGGTTCTTGAGACATTTTCTGGA.

The PCR product with the expected size was recovered from an agarose gel, digested with the Bam HI and Xba I restriction enzymes, and ligated into the transfer plasmid pFastBac1 which was digested with the same restriction enzymes. The sequence of the insert was confirmed by DNA sequencing and is set forth in SEQ ID NO:335. The predicted amino acid sequence of B305D with the C-terminal His tag is set forth in SEQ ID NO:336. The recombinant transfer plasmid pFBB305D was used to make recombinant bacmid DNA and virus by the Bac-To-Bac baculovirus expression system (Invitrogen Life Technologies, Carlsbad, Calif.). The recombinant BVB305D virus was amplified in Sf9 insect cells and used to infect High Five insect cells. Infected cells were harvested at 24-30 hours post-infection. The identity of the recombinant protein was confirmed by Western blot with a rabbit polyclonal antibody against B305D. Recombinant protein was further analyzed by SDS-PAGE followed by Coomassie blue staining.

### Example 10

### Identification of an Additional B305D Homolog Discovered by Bioinformatic Search

The High Throughput Genome Sequencing (HTGS) database was searched with the B305D C form sequence (SEQ ID NO:301) and revealed another highly related copy of the B305D gene, tentatively localized to Chromosome 14. The sequences identified were spliced together based on the B305D C form sequence and exon-intron splice sites. This predicted cDNA sequence (SEQ ID NO:339) was translated to generate the predicted amino acid sequence (SEQ ID NO:340). The B305D gene family members have been shown to be overexpressed in breast cancer, prostate cancer, and ovarian cancer.

### Example 11

### Immunohistochemical (IHC) Analysis of B305D Expression

Analysis suggests that B305D is a type II plasma membrane protein of about 43 kDa with 1 predicted trasmembrane spanning domain. There are no glycosylation sites and its function remains unknown. Disclosed herein is further examination of B305D expression by immunohistochemistry (IHC) analysis in a variety of tumor and normal tissues.

Methods and Materials

In order to determine which tissues express the breast cancer antigen B305D, IHC analysis was performed on a diverse range of tissue sections. Tissue samples were fixed in formalin solution for 12-24 hours and embedded in paraffin before being sliced into 8 micron sections. Steam heat induced epitope retrieval (SHIER) in 0.1 M sodium citrate buffer (pH 6.0) was used for optimal staining conditions. Sections were incubated with 10% serum/PBS for 5 minutes. Primary antibody was added to each section for 25 minutes at indicated concentrations followed by 25 minute incubation with anti-rabbit biotinylated antibody. Endogenous peroxidase activity was blocked by three 1.5 minute incubations with hydrogen peroxidase. The avidin biotin complex/horse radish peroxidase (ABC/HRP) system was used along with DAB chromogen to visualize antigen expression. Slides were counterstained with hematoxylin to visualize cell nuclei.

Rabbit polyclonal antibodies against B305D were shown in Example 7 to react in formalin fixed, paraffin-embedded tissues. The antibody was shown to label the plasma membrane of a subset of breast carcinomas. B305D was shown to label tissues that were positive for cerb-2, also called Her-2/neu. HER-2/neu (p 85) is the protein product of the HER-2/neu oncogene. The HER-2/neu gene is amplified and the HER-2/neu protein is overexpressed in a variety of cancers including breast, ovarian, colon, lung, prostate and hematological cancers. HER-2/neu is related to malignant transformation and is found in 50%-60% of ductal in situ carcinoma and 20%-40% of all breast cancers, as well as a substantial fraction of adenocarcinomas arising in the ovaries, prostate, colon and lung. HER-2/neu is intimately associated not only with the malignant phenotype, but also with the aggressiveness of the malignancy, being found in one-fourth of all invasive breast cancers. HER-2/neu overexpression is correlated with a poor prognosis in both breast and ovarian cancer. In this study breast carcinomas were tested from two age groups; women under 50 at the time of tumor removal and women over 50 at the time of tumor removal. B305D staining was evaluated for each. In addition to breast carcinomas ovarian carcinomas, normal pancreas, normal kidney and normal stomach were tested for B305D reactivity.

Formalin-fixed, paraffin-embedded breast carcinomas from 23 different patients were tested for B305D reactivity. The age of the patient at the time of tumor removal was available in all cases to determine whether patient age is associated with B305D staining. In many cases, estrogen receptor/progesterone receptor (ER/PR) data and cerb2 data was available from the pathology reports. Breast patients were chosen simply based on age. These patients in the ‘younger’ group are close to the age of 40. We also obtained tumors from patients that were closer to the age of 70. This group is referred to as the ‘older’ group.

In addition to breast carcinomas, 17 different ovarian carcinomas were immunohistochemically analyzed for B305D staining. Five samples each of normal stomach, kidney and pancreas were also tested. For most of the tissues, the B305D antibody was tested with two different detection systems, one with ABC as the Horseradish Peroxidase (HRP) enzyme-linked reagent and another with strept-avidin as the HRP reagent. In all cases, rabbit IgG was run as a negative control in parallel with the B305D antibody. B305D was tested at 2.5 μg/ml using SHIER II heat pretreatment. Breast carcinoma multi-tissue block, QMTB21, was used as a positive control for the antibody. Tumor #5 in the block was previously shown to label with a membrane pattern with the B305D antibody.

Results: Breast Carcinomas (Results Shown in Table III)

The avidin-biotin complex (ABC) stained slides were lighter than expected, although membrane staining was detected in the positive control. To make sure that no positive staining was overlooked, the slides were tested with the strept-avidin (SA) detection. Upon the analysis of the ABC slides, only one tumor labeled with a membrane pattern. This tumor was from a 42 yr old patient who also demonstrated membrane staining for cerb2. When retested with SA, an older patient that was cerb2 membrane positive was included. This tumor was from an 80 yr old patient. Breast cancer staining results are outlined in Table III below. The staining data presented in tables III-V is from the SA-HRP staining. The B305D antibody labels breast carcinomas in the cytoplasm and on the plasma membrane. Membrane staining is limited to tumor cells, whereas cytoplasmic staining is also often present in the normal ductal epithelium. Among the SA labeled tissues, only the positive control and the 42 yr old and the 80 yr old that were cerb2 positive labeled membrane positive for B305D. Two other cases labeled with light membrane staining in a minority of tumor cells. One case was from a 28 yr old patient, the other from a 73 yr old patient; cerb2 status was not available for either of these cases. The limited staining in these two cases with lighter staining may be due to tissue fixation as positive cells were found on the periphery of the tissue.

Thus, 4 cases of 23 (less than 20%) labeled with a membrane pattern for B305D. Less than 10% of the tumors (2 of 23) labeled with definitive membrane staining. In a previous random study, 3 of 15 cases demonstrated membrane staining for B305D. Cerb2 data was not available for all of the tissues tested but for the two cases that were definitively positive for B305D, both were strongly positive for cerb2. B305D membrane positive cases were split evenly across the ‘younger’ and ‘older’ groups. The younger group included 11 patients under 50 and the older group included 12 patients 50 or older. Of this older group, 9 of the patients are 66 or older, and 7 were in their 70's and 80's (one tumor from a 50 year old had only a small amount of tumor in the block and may be discounted—thus 4 of 22 positive). ER/PR data was available for most cases but no association with B305D could be determined. Thus, based on this and previous IHC data, B305D expression is closely associated with cerb2 expression. Further B305D testing of cerb2 positive breast tumors may strengthen this correlation. From the results of this study, patient age at the time of tumor removal does not appear to correlate with B305D staining.

TABLE III
Age Related B305D Reactivity in Breast Carcinomas

| Accession No. | Age | B305D IHC Reactivity | Diagnosis | ER/PR Status |
|---|---|---|---|---|
| S86-2763 (slide 1) | 29 | Cytoplasmic staining | Infiltrating Ductal | ER/PR negative |
| S00-9327 (slide 2) | 28 | Marginal membrane | Infiltrating | N/A |
| | | staining | Lobular | |
| S00-4786 (slide 3) | 43 | Light cytoplasmic | Infiltrating Mixed | ER positive 2-3+ |
| | | staining | Ductal/Lobular | PR positive 2-3+ |
| | | | | Cerb2 Negative 1+ |
| S86-1877 (slide 4) | 40 | Cytoplasmic staining | Infiltrating Ductal | ER positive |
| | | | | PR strongly positive |
| S84-2015 (slide 5) | 40 | Light cytoplasmic | Infiltrating Ductal | N/A |
| | | staining | | |
| S88-1981 (slide 6) | 40 | Cytoplasmic staining | Infiltrating Ductal | N/A |
| S84-2915 (slide 7) | 38 | Light cytoplasmic | Infiltrating Ductal | ER strongly positive |
| | | staining | | PR positive |
| S86-1510 (slide 8) | 41 | | Infiltrating Ductal | ER positive |
| | | | | PR strongly positive |
| S01-31 (slide 9) | 42 | Membrane staining; | Infiltrating Ductal | Cerb2 positive 3+ |
| | | cytoplasmic staining | | |
| S84-855 (slide 10) | 48 | Light cytoplasmic | Infiltrating ducal | ER Positive |
| | | staining | | PR strongly positive |
| 00-1826 (slide 50) | 46 | Light cytoplasmic | Infiltrating ducal | ER-positive 3+ |
| | | staining | | PR-positive 3+ |
| S00-2297 (slide 11) | 50 | Light cytoplasmic | Infiltrating ducal | ER-negative |
| | | staining | | PR-positive 1+ |
| | | | | Cerb2 negative 1+ |
| S00-3232A (slide | 50 | Light cytoplasmic | Infiltrating ducal | ER-positive 3+ |
| 12) | | staining (very little | | PR-positive 3+ |
| | | tumor) | | Cerb2-negative 1+ |
| S00-8096 (slide 13) | 54 | | Infiltrating ducal | ER-Negative |
| | | | | PR-Negative |
| | | | | Cerb2-negative 1+ |
| S00-2097 (slide 14) | 66 | Very little tumor | Infiltrating ducal | ER-positive 3+ |
| | | | | PR-positive 2-3+ |
| | | | | Cerb2-negative 2+ |
| S88-2476 (slide 15) | 79 | | Infiltrating ducal | ER-strongly positive |
| | | | | PR-strongly positive |
| S88-2551 (slide 16) | 81 | Very light cytoplasmic | Infiltrating ducal | ER-strongly positive |
| | | staining | | PR-positive |
| S88-2665 (slide 17) | 73 | Marginal membrane | Infiltrating ducal | ER-positive |
| | | staining; cytoplasmic | | PR-negative |
| | | staining | | |
| S88-2476 (slide 18) | 79 | Light membrane | Infiltrating ducal | ER-strongly positive |
| | | staining | | PR-strongly positive |
| S00-2491 (slide 19) | 77 | Light cytoplasmic | Lobular | ER-positive 1-3+ |
| | | staining | Infiltrating | PR-positive 1-3+ |
| | | Little tumor present | | Cerb2-negative 3+ |
| S85-2667 (slide 20) | 68 | Cytoplasmic staining | Infiltrating ducal | ER-strongly positive |
| | | | | PR-strongly positive |
| 00-6606A (slide 49) | 80 | Membrane staining; | Infiltrating ducal | ER-negative |
| | | cytoplasmic staining | | PR-negative |
| | | | | Cerb2-positive 3+ |
| S88-1146 (slide 50, | 88 | Light cytoplasmic | Infiltrating ducal | ER-strongly positive |
| in box 1) | | staining | | PR-negative |

Ovarian Carcinomas (Results Outlined in Table IV)

None of the 17 ovarian carcinomas tested with the B305D antibody labeled with a membrane pattern. About half of the tissues labeled with a cytoplasmic staining pattern.

TABLE IV
B305D Staining of Ovarian Carcinomas

| | Tissue (slide #) | Age | Diagnosis | IHC Reactivity/Comments |
|---|---|---|---|---|
| 1. | 73-1808 (slide 37) | 73 | Papillary mucinous | |
| | | | adenocarcinoma | |
| 2. | 76-1076 (slide 38) | 50 | Serous adenocarcinoma | |
| 3. | 81-1910 (slide 39) | 51 | Serous adenocarcinoma | Cytoplasmic staining; not |
| | | | | uniform |
| 4. | 88-220 (slide 40) | 40 | Mucinous cystadenocarcinoma | Light cytoplasmic staining |
| 5. | 88-2207 (slide 41) | 75 | Papillary Serious | |
| | | | cystadenocarcinoma | |
| 6. | 88-2527 (slide 42) | 29 | Malignant teratoma | Light cytoplasmic staining; |
| | | | | not uniform |
| 7. | 00-5294 (slide 43) | 55 | Papillary adenocarcinoma | Light cytoplasmic staining |
| 8. | 84-779 (slide 44) | 48 | Endometriod carcinoma | Light cytoplasmic staining |
| 9. | 84-1843 (slide 45) | 32 | Papillary serious | Cytoplasmic staining |
| | | | adenocarcinoma | |
| 10. | 85-2373 (slide 46) | 47 | Granulosa cell tumor | Light cytoplasmic staining |
| 11. | 86-813 (slide 47) | 74 | Clear cell carcinoma | |
| 12. | QMTB#26 (slide | | Five different ovarian | All negative |
| | 48) | | carcinomas | |

Normal Tissues (Results Outlined in Table V)

Of the five stomach cases tested, all had staining above background in the glands below the gastric epithelium. Staining was cytoplasmic and grainy and was present with both detection systems. There was some staining in the negative control but this staining was diffuse and not grainy. Background staining was common in these cells. The B305D staining appeared to be due to the antibody binding and not the detection system.

Five different kidney cases were tested. The medulla region was represented in each case. There was staining in the tubules throughout the kidney, but this appears to be due to endogenous biotin as similar but lighter staining was present in the negative controls. There was much less staining in the ABC stained slides compared with the strept-avidin slides, which is also consistent with endogenous biotin. The SHIER II pretreatment required to obtain staining with the antibody tended to give more background staining, particularly due to endogenous biotin.

Of the five different pancreas tissues tested, no specific staining was detected. A subset of acinar cells gave staining in both the B305D and the rabbit IgG control. Once again this staining was non-specific. Pancreas often gave non-specific staining, possibly due to the enzymatic activity of the tissue.

A variety of other normal tissues (not shown in Table V) were tested including skin, testis, colon, heart, thymus, artery, skeletal muscle, small bowel, pituitary, spinal cord, spleen, ureter, gall bladder, placenta, thyroid, liver, brain-cerebellum, bone marrow, parathyroid, lung esophagus, uterus, adrenal, lymph node, brain-cortex, fallopian tude, bladder, and prostate. Weak IHC staining was observed in small bowel, uterus, and bladder. However, no mRNA expression was seen in these tissues. Thus, this weak staining likely does not represent protein expression in these tissues. The gall bladder stained positive and will be analyzed further. Half of the prostate samples stained positive as well as the single testis sample examined.

B305D expression was also analyzed in prostate tumor samples. One of 5 grade 3+3 samples stained positive while none of the grade 3+4 samples stained positive. One additional sample of 3 unknown grade samples stained positive. However, an additional array of 55 primary and primary metastatic prostate tumor samples was tested and no staining was observed.

TABLE V
B305D Staining of Other Tissues (Normal Kidney, Stomach and Pancreas)

| | | B305D IHC | |
|---|---|---|---|
| | Tissue (Slide #) | Reactivity | Comments |
| | Stomach | | |
| 1. | Blk 85-568 (slide 22) | cytomplasmic | Grainy cytomplasmic staining of glands |
| | | | below epithelium (not in neg control) |
| 2. | Blk 85-587 (slide 23) | cytomplasmic | Graining staining of glands below |
| | | | epithelium, some background in negative |
| | | | control |
| 3. | Blk 85-1206 (slide 24) | cytomplasmic | Graining staining of glands below |
| | | | epithelium, lighter background in negative |
| | | | control |
| 4. | Blk 85-1225 (slide 25) | cytomplasmic | Marginal staining |
| 5. | Blk 85-1426 (slide 26) | cytomplasmic | Grainy staining of glands below |
| | | | epithelium, some background in negative |
| | | | control |
| | Kidney | | |
| 1. | Blk 00-7008 (slide 27) | Inconclusive (most | Staining of tubules; also present in neg |
| | | likely negative) | control (lighter) —mostly likely due to |
| | | | endogenous biotin |
| 2. | Blk 00-5638 (slide 28) | Same as above | Same as above |
| 3. | Blk 00-1711 (slide 29) | Same as above | Same as above |
| 4. | Blk 00-3859 (slide 30) | Same as above | Same as above |
| 5. | Blk 00-7651 (slide 31) | Same as above | Same as above |
| | Pancreas | | |
| 1. | Blk Q965 (slide 32) | Negative | Non-specific staining in negative control |
| 2. | Blk 00-2287 (slide 33) | Negative | Non-specific staining in negative control |
| 3. | Blk 00-2790 (slide 34) | Negative | Non-specific staining in negative control |
| 4. | Blk 00-6899 (slide 35) | Negative | Non-specific staining in negative control |
| 5. | Blk 00-7053 (slide 36) | Negative | Non-specific staining in negative control |

In summary, B305D was only observed in less than 20% of breast carcinomas. Staining was observed in half of the normal prostate samples however, membrane staining was not detected in normal breast, in ovarian carcinomas or in normal pancreas, kidney, stomach or a panel of other normal tissues.

### Example 12

### Analysis of Breast-Tumor Specific B305D Sequences

Numerous forms of the breast tumor antigen, B305D have been isolated. To date, isoforms A (DNA SEQ ID NO:291, 292, 296, 313, 314) A variant (DNA SEQ ID NO:299), B (DNA SEQ ID NO:294, 297), and C (DNA SEQ ID NO:295, 301, 302, 303) have been identified. Using B305D gene specific 5′ and 3′ primers representing all known forms of B305D, specific forms of this gene expressed in breast tumors were amplified. Disclosed herein in SEQ ID NO:341-348 are 4 D305D nucleotide sequences and their corresponding amino acid sequences identified specifically in breast tumors as described below.

Two PCR reactions were carried out using primers specific to B305D. The products were then analyzed and full-length sequences were compiled. For the first reaction, primers were designed to regions common to all B305D forms near the 5′ and 3′ ends of the gene. The second set of PCR reactions used primers specific to each of the start sites specific to each of the forms. Three 5′ primers were designed to amplify from the B305D A form, A form frameshift and C form start sites. 3′ reverse primers were designed to a common region of all B305D forms, slightly upstream of the 3′ primer used in the first PCR reaction. PCR was carried out using these primers and cDNA derived from breast tumor RNA numbers 443, 23B, and S76. All products were sequenced, analyzed and compiled.

Two variants of the B305D A isoform were identified in the breast tumor samples. The nucleotide sequence of these 2 variants is set forth in SEQ ID NO:341 and 342 and the corresponding amino acid sequence is set forth in SEQ ID NO:345 and 346. One of these variants (SEQ ID NO:341) is identical to a previously identified variant of B305D A isoform described in Example 1 and set forth in SEQ ID NO:314. The other variant (SEQ ID NO:342) differs from SEQ ID NO:314 by 2 base-pairs and encodes an amino acid sequence (SEQ ID NO:346) that differs by one amino acid from the previously identified A isoform set forth in SEQ ID NO:315.

Two new variants of the B305D C isoform were also identified from the breast tumor samples. The nucleotide sequence of these two variants is provided in SEQ ID NO:343 and 344 and the corresponding amino acid sequence is set forth in SEQ ID NO:347 and 348. The 5′ end of the 2 C isoform variants appears to be a truncated C isoform that is missing one of the two 4 base pair repeats normally seen in the C isoform. The 3′ end of these variants aligns well to the A isoforms. More specifically, there is a splice junction at around base 297. It is at this junction where SEQ IDs 343 and 344 diverge from the standard C form and the remaining 3′ end being the A form. Upstream (5′ of) of this junction the sequence of B305D isoforms set forth in SEQ ID NO:343 and 344 are missing 111 base pairs of standard B305D C form respeat sequence. The variant set forth in SEQ ID 343 is the shortest, having an additional 6 base pair deletion in the large missing repeat. Thus, in summary, SEQ ID NO:343 and 344 begin with the ATG of the standard B305D C isoform. The sequence continues as the C isoform for about 185 base pairs for SEQ ID NO:344 and 179 base pairs for SEQ ID NO:343. Both sequences then have about a 112 base pair deletion of repeat sequence just prior to the splice junction. Following the splice junction, both variants follow the A form.

### Example 13

### Identification of CD4 T cell Epitopes for B305D

This example demonstrates the identification of CD4+ T cell epitopes of the C form of B305D (full-length cDNA and amino acid sequence of B305D are set forth in SEQ ID NO:301 and 304, respectively).

CD4+ T cell responses were generated using PBMC of normal donors using dendritic cells (DC) pulsed with overlapping 20-mer peptides spanning the entire B305D C isoform protein. Briefly, CD4+ T cells were stimulated 3-4 times with DC pulsed with a mixture of overlapping peptides in IMDM media containing IL-6 and IL-12 in the primary stimulation, and IL-2+IL-7 in all other stimulations. These lines were subsequently assayed using a standard proliferation assay (measuring tritiated thymidine uptake) for reactivity with the priming peptides or recombinant E. coli derived B305D.

A number of different peptides elicited B305D specific T cells. These CD4+ T cell epitopes are contained in the following sequences:

VNKKDKQKRTALHLASANGNSEVVKLLLDR (SEQ ID NO:349): (peptides 34-46 corresponding to amino acids 166-195 of SEQ ID NO:304).

ALHLASANGNSEVVKLLLDRRCQLNVLDNK (SEQ ID NO:350) (peptides 36-38 corresponding to amino acids 176-205 of SEQ ID NO:304).

GSASIVSLLLEQNIDVSSQDLSGQT (SEQ ID NO:351) (peptides 64-65 corresponding to amino acids 316-340 of SEQ ID NO:304).

CD4+ T cells recognizing these peptides also recognize recombinant B305D protein, suggesting that these are naturally processed epitopes. Two of these lines (lines 31.9 and 31.10 recognizing peptides set forth in SEQ ID NO:349 and 350) also recognized mammalian sources of B305D including baculovirus protein, lysates from HEK cells transiently transfected with B305D and lysates from cells infected with adenovirus expressing B305D.

Thus, these studies demonstrate that CD4+ T cell immunity to B305D can be elicited and identify the peptides set forth in SEQ ID NO:349-351 as immunogenic, naturally processed CD4+ T cell epitopes.

### Example 14

### Autoantibodies to B305D In Breast Cancer Sera and Epitope Mapping of the Antigenic Sites

Autoantibodies to specific B305D peptide epitopes were identified in the sera of breast cancer patients. Overlapping peptides spanning the entire B305D sequence (cDNA and amino acid sequence of the C form of B305D set forth in SEQ ID NO:301 and 304, respectively) were synthesized and tested by ELISA with sera from patients with breast cancer to determine the presence of B305D-specific antibodies. Several immunoreactive regions were identified, including immunodominant regions encompasssing the ankyrin repeat portion of the molecule.

Seventy-four 20-mer peptides overlapping by 15 amino acids, spanning the entire open reading frame of B305D were synthesized (amino acid sequences set forth in SEQ ID NO:352-425). These 74 peptides were tested in ELISA to evaluate which epitopes reacted with breast cancer sera as well as control sera. Initially peptides were pooled and tested to locate regions of activity. Highest activity was obtained in peptides 1-24 (SEQ ID NO:352-375) and these were retested individually to determine the specific epitopes. Peptides 3,5,6,11,13,19 and 20 (SEQ ID NO:354, 356, 357, 362, 364, 370, 371, respectively) were then further tested with a complete panel of 74 breast, 50 ovarian and 55 prostate cancer sera as well as controls. 18 of 74 breast cancer sera were reactive with one or more peptides. Both breast and ovarian cancer sera showed reactivity and active epitopes appeared located in the ankyrin repeat regions of B305D. The amino acid sequence of the 3 ankyrin repeat sequences found in B305D are set forth in SEQ ID NO:426-428 and are present within the overlapping peptides set forth in SEQ ID NO:356-359, 363-366, and 368-376, respectively.

Detection of autoantibodies to B305D in breast cancer sera indicates that such patients can elicit an immune response to specific epitopes and indicates that B305D can be used either alone or in combination with other breast tumor antigens as a target for vaccine development. Knowing that antibodies to B305D are present in the serum of breast cancer patients strengthens the potential use of this antigen as a vaccine target. In addition, detection of antibodies to B305D can be used as a diagnostic for breast cancer alone or in combination with detecting antibodies to other antigens eg Her-2/neu or other tumor antigens. The presence of antibodies to B305D also indicates that B305D antigen is present in serum and could be used as a target for development of a specific antigen detection assay.

### Example 15

### Analysis of cDNA Expression Using Microarray Technology

In additional studies, sequences disclosed herein are evaluated for overexpression in specific tumor tissues by microarray analysis. Using this approach, cDNA sequences are PCR amplified and their mRNA expression profiles in tumor and normal tissues are examined using cDNA microarray technology essentially as described (Shena, M. et al., 1995 Science 270:467-70). In brief, the clones are arrayed onto glass slides as multiple replicas, with each location corresponding to a unique cDNA clone (as many as 5500 clones can be arrayed on a single slide, or chip). Each chip is hybridized with a pair of cDNA probes that are fluorescence-labeled with Cy3 and Cy5, respectively. Typically, 1 μg of polyA⁺ RNA is used to generate each cDNA probe. After hybridization, the chips are scanned and the fluorescence intensity recorded for both Cy3 and Cy5 channels. There are multiple built-in quality control steps. First, the probe quality is monitored using a panel of ubiquitously expressed genes. Secondly, the control plate also can include yeast DNA fragments of which complementary RNA may be spiked into the probe synthesis for measuring the quality of the probe and the sensitivity of the analysis. Currently, the technology offers a sensitivity of 1 in 100,000 copies of mRNA. Finally, the reproducibility of this technology can be ensured by including duplicated control cDNA elements at different locations.

### Example 16

### Analysis of cDNA Expression Using Real-Time PCR

Real-time PCR (see Gibson et al., Genome Research 6:995-1001, 1996; Heid et al., Genome Research 6:986-994, 1996) is a technique that evaluates the level of PCR product accumulation during amplification. This technique permits quantitative evaluation of mRNA levels in multiple samples. Briefly, mRNA is extracted from tumor and normal tissue and cDNA is prepared using standard techniques. Real-time PCR is performed, for example, using a Perkin Elmer/Applied Biosystems (Foster City, Calif.) 7700 Prism instrument. Matching primers and fluorescent probes are designed for genes of interest using, for example, the primer express program provided by Perkin Elmer/Applied Biosystems (Foster City, Calif.). Optimal concentrations of primers and probes are initially determined by those of ordinary skill in the art, and control (e.g., β-actin) primers and probes are obtained commercially from, for example, Perkin Elmer/Applied Biosystems (Foster City, Calif.). To quantitate the amount of specific RNA in a sample, a standard curve is generated using a plasmid containing the gene of interest. Standard curves are generated using the Ct values determined in the real-time PCR, which are related to the initial cDNA concentration used in the assay. Standard dilutions ranging from 10-10⁶ copies of the gene of interest are generally sufficient. In addition, a standard curve is generated for the control sequence. This permits standardization of initial RNA content of a tissue sample to the amount of control for comparison purposes.

An alternative real-time PCR procedure can be carried out as follows: The first-strand cDNA to be used in the quantitative real-time PCR is synthesized from 20 μg of total RNA that is first treated with DNase I (e.g., Amplification Grade, Gibco BRL Life Technology, Gaitherburg, Md.), using Superscript Reverse Transcriptase (RT) (e.g., Gibco BRL Life Technology, Gaitherburg, Md.). Real-time PCR is performed, for example, with a GeneAmp™ 5700 sequence detection system (PE Biosystems, Foster City, Calif.). The 5700 system uses SYBR™ green, a fluorescent dye that only intercalates into double stranded DNA, and a set of gene-specific forward and reverse primers. The increase in fluorescence is monitored during the whole amplification process. The optimal concentration of primers is determined using a checkerboard approach and a pool of cDNAs from breast tumors is used in this process. The PCR reaction is performed in 25 μl volumes that include 2.5 μl of SYBR green buffer, 2 μl of cDNA template and 2.5 μl each of the forward and reverse primers for the gene of interest. The cDNAs used for RT reactions are diluted approximately 1:10 for each gene of interest and 1:100 for the β-actin control. In order to quantitate the amount of specific cDNA (and hence initial mRNA) in the sample, a standard curve is generated for each run using the plasmid DNA containing the gene of interest. Standard curves are generated using the Ct values determined in the real-time PCR which are related to the initial cDNA concentration used in the assay. Standard dilution ranging from 20-2×10⁶ copies of the gene of interest are used for this purpose. In addition, a standard curve is generated for β-actin ranging from 200 fg-2000 fg. This enables standardization of the initial RNA content of a tissue sample to the amount of β-actin for comparison purposes. The mean copy number for each group of tissues tested is normalized to a constant amount of β-actin, allowing the evaluation of the over-expression levels seen with each of the genes.

### Example 17

### Peptide Priming of T-helper Lines

Generation of CD4⁺ T helper lines and identification of peptide epitopes derived from tumor-specific antigens that are capable of being recognized by CD4⁺ T cells in the context of HLA class II molecules, is carried out as follows:

Fifteen-mer peptides overlapping by 10 amino acids, derived from a tumor-specific antigen, are generated using standard procedures. Dendritic cells (DC) are derived from PBMC of a normal donor using GM-CSF and IL-4 by standard protocols. CD4⁺ T cells are generated from the same donor as the DC using MACS beads (Miltenyi Biotec, Auburn, Calif.) and negative selection. DC are pulsed overnight with pools of the 15-mer peptides, with each peptide at a final concentration of 0.25 μg/ml. Pulsed DC are washed and plated at 1×10⁴ cells/well of 96-well V-bottom plates and purified CD4⁺ T cells are added at 1×10⁵/well. Cultures are supplemented with 60 ng/ml IL-6 and 10 ng/ml IL-12 and incubated at 37° C. Cultures are restimulated as above on a weekly basis using DC generated and pulsed as above as antigen presenting cells, supplemented with 5 ng/ml IL-7 and 10 U/ml IL-2. Following 4 in vitro stimulation cycles, resulting CD4⁺ T cell lines (each line corresponding to one well) are tested for specific proliferation and cytokine production in response to the stimulating pools of peptide with an irrelevant pool of peptides used as a control.

### Example 18

### Generation of Tumor-Specific CTL Lines Using In Vitro Whole-Gene Priming

Using in vitro whole-gene priming with tumor antigen-vaccinia infected DC (see, for example, Yee et al, The Journal of Immunology, 157(9):4079-86, 1996), human CTL lines are derived that specifically recognize autologous fibroblasts transduced with a specific tumor antigen, as determined by interferon-γ ELISPOT analysis. Specifically, dendritic cells (DC) are differentiated from monocyte cultures derived from PBMC of normal human donors by growing for five days in RPMI medium containing 10% human serum, 50 ng/ml human GM-CSF and 30 ng/ml human IL-4. Following culture, DC are infected overnight with tumor antigen-recombinant vaccinia virus at a multiplicity of infection (M.O.I) of five, and matured overnight by the addition of 3 μg/ml CD40 ligand. Virus is then inactivated by UV irradiation. CD8+ T cells are isolated using a magnetic bead system, and priming cultures are initiated using standard culture techniques. Cultures are restimulated every 7-10 days using autologous primary fibroblasts retrovirally transduced with previously identified tumor antigens. Following four stimulation cycles, CD8+ T cell lines are identified that specifically produce interferon-γ when stimulated with tumor antigen-transduced autologous fibroblasts. Using a panel of HLA-mismatched B-LCL lines transduced with a vector expressing a tumor antigen, and measuring interferon-γ production by the CTL lines in an ELISPOT assay, the HLA restriction of the CTL lines is determined.

### Example 19

### Generation and Characterization of Anti-Tumor Antigen Monoclonal Antibodies

Mouse monoclonal antibodies are raised against E. coli derived tumor antigen proteins as follows: Mice are immunized with Complete Freund's Adjuvant (CFA) containing 50 μg recombinant tumor protein, followed by a subsequent intraperitoneal boost with Incomplete Freund's Adjuvant (IFA) containing 10μg recombinant protein. Three days prior to removal of the spleens, the mice are immunized intravenously with approximately 50 μg of soluble recombinant protein. The spleen of a mouse with a positive titer to the tumor antigen is removed, and a single-cell suspension made and used for fusion to SP2/O myeloma cells to generate B cell hybridomas. The supernatants from the hybrid clones are tested by ELISA for specificity to recombinant tumor protein, and epitope mapped using peptides that spanned the entire tumor protein sequence. The mAbs are also tested by flow cytometry for their ability to detect tumor protein on the surface of cells stably transfected with the cDNA encoding the tumor protein.

### Example 20

### Synthesis of Polypeptides

Polypeptides are synthesized on a Perkin Elmer/Applied Biosystems Division 430A peptide synthesizer using FMOC chemistry with HPTU (O-Benzotriazole-N,N,N′,N′-tetramethyluronium hexafluorophosphate) activation. A Gly-Cys-Gly sequence is attached to the amino terminus of the peptide to provide a method of conjugation, binding to an immobilized surface, or labeling of the peptide. Cleavage of the peptides from the solid support is carried out using the following cleavage mixture: trifluoroacetic acid:ethanedithiol:thioanisole:water:phenol (40:1:2:2:3). After cleaving for 2 hours, the peptides are precipitated in cold methyl-t-butyl-ether. The peptide pellets are then dissolved in water containing 0.1% trifluoroacetic acid (TFA) and lyophilized prior to purification by C18 reverse phase HPLC. A gradient of 0%-60% acetonitrile (containing 0.1% TFA) in water (containing 0.1% TFA) is used to elute the peptides. Following lyophilization of the pure fractions, the peptides are characterized using electrospray or other types of mass spectrometry and by amino acid analysis.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed:

1. An isolated polynucleotide comprising a sequence selected from the group consisting of:
(a) sequences provided in SEQ ID NO:341-344;
(b) complements of the sequences provided in SEQ ID NO:341-344;
(c) sequences consisting of at least 20 contiguous residues of a sequence provided-in SEQ ID NO:341-344;
(d) sequences that hybridize to a sequence provided in SEQ ID NO:341-344, under highly stringent conditions;
(e) sequences having at least 75% identity to a sequence of SEQ ID NO:341-344;
(f) sequences having at least 90% identity to a sequence of SEQ ID NO:341-344; and
(g) degenerate variants of a sequence provided in SEQ ID NO:341-344.

2. An isolated polypeptide comprising an amino acid sequence selected from the group consisting of:
(a) sequences encoded by a polynucleotide of claim 1; and
(b) sequences having at least 70% identity to a sequence encoded by a polynucleotide of claim 1; and
(c) sequences having at least 90% identity to a sequence encoded by a polynucleotide of claim 1;
(d) sequences set forth in SEQ ID NO:345-428;
(e) sequences having at least 70% identity to a sequence set forth in SEQ ID NO:345-428; and
(f) sequences having at least 90% identity to a sequence set forth in SEQ ID NO:345-428.

3. An expression vector comprising a polynucleotide of claim 1 operably linked to an expression control sequence.

4. A host cell transformed or transfected with an expression vector according to claim 3.

5. An isolated antibody, or antigen-binding fragment thereof, that specifically binds to a polypeptide of claim 2.

6. A method for detecting the presence of a cancer in a patient, comprising the steps of:
(a) obtaining a biological sample from the patient;
(b) contacting the biological sample with a binding agent that binds to a polypeptide of claim 2;
(c) detecting in the sample an amount of polypeptide that binds to the binding agent; and
(d) comparing the amount of polypeptide to a predetermined cut-off value and therefrom determining the presence of a cancer in the patient.

7. A fusion protein comprising at least one polypeptide according to claim 2.

8. An oligonucleotide that hybridizes to a sequence recited in SEQ ID NO:341-344 under highly stringent conditions.

9. A method for stimulating and/or expanding T cells specific for a tumor protein, comprising contacting T cells with at least one component selected from the group consisting of:
(a) polypeptides according to claim 2;
(b) polynucleotides according to claim 1; and
(c) antigen-presenting cells that express a polynucleotide according to claim 1,
under conditions and for a time sufficient to permit the stimulation and/or expansion of T cells.

10. An isolated T cell population, comprising T cells prepared according to the method of claim 9.

11. A composition comprising a first component selected from the group consisting of physiologically acceptable carriers and immunostimulants, and a second component selected from the group consisting of:
(a) polypeptides according to claim 2;
(b) polynucleotides according to claim 1;
(c) antibodies according to claim 5;
(d) fusion proteins according to claim 7;
(e) T cell populations according to claim 10; and
(f) antigen presenting cells that express a polypeptide according to claim 2.

12. A method for stimulating an immune response in a patient, comprising administering to the patient a composition of claim 11.

13. A method for the treatment of a breast cancer in a patient, comprising administering to the patient a composition of claim 11.

14. A method for determining the presence of a cancer in a patient, comprising the steps of:
(a) obtaining a biological sample from the patient;
(b) contacting the biological sample with an oligonucleotide according to claim 8;
(c) detecting in the sample an amount of a polynucleotide that hybridizes to the oligonucleotide; and
(d) comparing the amount of polynucleotide that hybridizes to the oligonucleotide to a predetermined cut-off value, and therefrom determining the presence of the cancer in the patient.

15. A diagnostic kit comprising at least one oligonucleotide according to claim 8.

16. A diagnostic kit comprising at least one antibody according to claim 5 and a detection reagent, wherein the detection reagent comprises a reporter group.

17. A method for the treatment of breast cancer in a patient, comprising the steps of:
(a) incubating CD4+ and/or CD8+ T cells isolated from a patient with at least one component selected from the group consisting of: (i) polypeptides according to claim 2; (ii) polynucleotides according to claim 1; and (iii) antigen presenting cells that express a polypeptide of claim 2, such that T cell proliferate;
(b) administering to the patient an effective amount of the proliferated T cells,
and thereby inhibiting the development of a cancer in the patient.
